(19) United States
(12) Patent Application Publication
(10) Pub. No.: US 2025/0100998 A1
(43) Pub. Date: Mar. 27, 2025

(54) PARP7 INHIBITORS

(71) Applicant: Gilead Sciences, Inc., Foster City, CA (US)

(72) Inventors: Jayaraman Chandrasekhar, Redmond, WA (US); Jonah J. Chang, Woodinville, WA (US); Kevin S. Currie, North Bend, WA (US); Stephen D. Holmbo, Kirkland, WA (US); Jesse M. Jacobsen, Seattle, WA (US); David L. Kukla, Germantown Hills, IL (US); Seung H. Lee, Sammamish, WA (US); Yasamin Moazami, Seattle, WA (US); Leena B. Patel, Seattle, WA (US); Thomas J. Paul, Snohomish, WA (US); Stephane Perreault, Brier, WA (US); Patrick J. Salvo, Burien, WA (US); Jennifer A. Treiberg, Redmond, WA (US); Heath A. Weaver, Seattle, WA (US)

(21) Appl. No.: 18/786,228

(22) Filed: Jul. 26, 2024

Related U.S. Application Data
(60) Provisional application No. 63/515,785, filed on Jul. 26, 2023.

(51) Int. Cl. C07D 401/14; A61K 31/506; A61K 31/517; A61K 31/5383; A61K 31/553; C07D 403/14; C07D 471/04; C07D 491/056; C07D 498/04

(52) U.S. Cl. CPC C07D 401/14; A61K 31/506; A61K 31/517; A61K 31/5383; A61K 31/553; C07D 403/14; C07D 471/04; C07D 491/056; C07D 498/04

(57) ABSTRACT

Provided herein is a compound of Formula I:

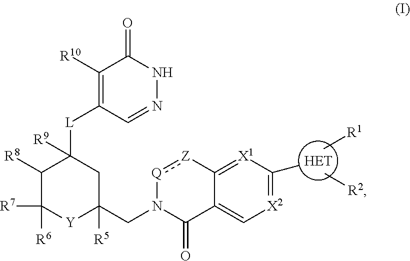

or a pharmaceutically acceptable salt, stereoisomer, mixture of stereoisomers, or deuterated analog thereof, a pharmaceutical composition comprising a compound of the present invention, together with a pharmaceutically acceptable excipient thereof, and a method of treating cancer with the same.

### CROSS REFERENCE TO RELATED APPLICATIONS

This application claims priority to U.S. Provisional Application No. 63/515,785, filed Jul. 26, 2023 which are incorporated herein in its entirety for all purposes.

### BACKGROUND OF THE INVENTION

Adenosine diphosphate (ADP)-ribosylation is a well conserved post-translational modification found in viruses, bacteria and eukaryotes. It is catalyzed by members of the ART superfamily of proteins, which transfer ADPr from nicotinamide adenine dinucleotide (NAD+) onto substrates via N-, O-, or S-glycosidic linkages on target molecules. One subset of ART's is the poly(adenosine diphosphate-ribose) polymerases (PARPs), that are members of a family of seventeen known enzymes that regulate fundamental cellular processes including gene Fexpression, protein degradation, and multiple cellular stress responses (M. S. Cohen, P. Chang, Insights into the biogenesis, function, and regulation of ADP-ribosylation. (Nat. Chem Biol 14, 236-243 (2018)). The ability of cancer cells to survive under stress is a fundamental cancer mechanism and an emerging approach for novel therapeutics.

Of particular interest is 2,3,7,8 tetrachlorodibenzo-p-dioxin (TCDD)-inducible poly(ADP ribose) polymerase (TIPARP), a CCCH-type zinc finger domain-containing protein. (Proc. Nat. Acad. Sci. 114 (10) 2681-2686 (2017)). TIPARP is also called PARP7 and ARTD14. PARP7 acts as negative regulator of certain aryl hydrocarbon receptor (AHR) transcriptional targets. AHR, in turn, is activated by many substrates, including cigarette smoke. PARP7 inhibitors have been shown to restore type I interferon (IFN) signaling responses to nucleic acids and causes tumor regression in a CT26 tumor-bearing, immunocompetent BALB/c mouse model. (Gozgit, et al., Cancer Cell 39, 1214-1226 (2021)).

There are currently no approved PARP7 inhibiting pharmaceuticals. Therefore, it would be useful to provide a PARP7 inhibiting compound with properties suitable for administration as a pharmaceutical agent to a mammal, particularly a human.

Thus, there is a need for improved PARP7 inhibitors for the treatment of cancer.

### BRIEF SUMMARY OF THE INVENTION

Provided herein are compounds and pharmaceutical compositions useful as inhibitors of PARP7. Some compounds of the disclosure may find use in pharmaceutical compositions, together with at least one pharmaceutically acceptable excipient, for treating a subject in need thereof.

In one embodiment of the present invention, there is provided a compound of Formula I:

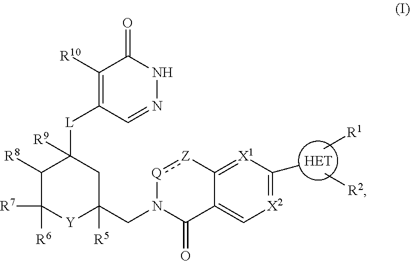

or a pharmaceutically acceptable salt or stereoisomer thereof, wherein:
HET is a 5-12 membered heteroaryl; C₆₋₁₀ aryl; C₃₋₁₂cycloalkyl; or 4-12 membered heterocyclyl; wherein any 5-12 membered heteroaryl, C₆₋₁₀ aryl, C₃₋₁₂ cycloalkyl, or 4-12 membered heterocyclyl, is monocyclic, bicyclic and 3-12 membered cycloalkyl or 4-12 membered heterocyclyl is monocyclic, bicyclic, fused bicyclic, spirocyclic or bridged.
Q is a bond to Z, CH₂, C═O, CR¹¹, or C(R¹¹)₂;
L is NH or O;
Y is NH, CH₂, CHR¹², CR¹²R¹² or O;
Z is N, CR¹¹ or C(R¹¹)₂,
X¹ is N or CR³;
X² is N or CR³;
X³ is N or CR⁴
R¹ and R² are each independently: H, OH, NH₂, CH₃, CHF₂, CF₃, —O—C₁₋₆ alkyl, —O—C₁₋₆ alkyl-F, —O—C₁₋₆ alkyl F₂, C₁₋₃ alkyl-O—C₁₋₃ alkyl, S(O)₂R¹¹, optionally substituted 5-12 membered heterocyclyl, or optionally substituted 6-10 membered heteroaryl;
R³ and R⁴ are each independently H, halo, —O(C₁₋₆ alkyl), OCH₃ or OCD₃;
R⁵ is H, halo, or OH;
R⁶ is H, halo or OH;
R⁷ is H. halo or OH;
R⁸ is H, halo or OH;
R⁹ is H or CH₃;
R¹⁰ is CH₃, CHF₂, or CF₃;
R¹¹ is H, halo, CHF₂ or CF₃, C₁₋₆ alkyl or C₃₋₁₀ cycloalkyl;
R¹² is H, halo, C₁₋₁₀ alkyl; C₃₋₁₀ cycloalkyl; 5-10 membered heteroaryl; C₆₋₁₀ aryl; 4-7 membered monocyclic heterocyclyl, 6-12 membered bicyclic or heterocyclyl;
or a pharmaceutically acceptable salt, tautomer or stereoisomer thereof.

Further, there is provided a method of treating cancer, in a subject in need thereof, comprising administering to said patient a compound of the present invention, or a pharmaceutical composition comprising a compound of the present invention.

### DETAILED DESCRIPTION OF THE INVENTION

### I. Definitions

The following description sets forth exemplary methods, parameters and the like. It should be recognized, however, that such description is not intended as a limitation on the scope of the present disclosure but is instead provided as a description of exemplary embodiments. The following description sets forth exemplary methods, parameters and the like. It should be recognized, however, that such description is not intended as a limitation on the scope of the present disclosure but is instead provided as a description of exemplary embodiments.

A dash (“-”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named.
A wavy line

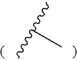

indicates a point of attachment.

The prefix “Cᵤ₋ᵥ” indicates that the following group has from u to v carbon atoms. For example, “C₁₋₆ alkyl” indicates that the alkyl group has from 1 to 6 carbon atoms.

Reference to “about” a value or parameter herein includes (and describes) embodiments that are directed to that value or parameter per se. In certain embodiments, the term “about” includes the indicated amount±10%. In other embodiments, the term “about” includes the indicated amount±5%. In certain other embodiments, the term “about” includes the indicated amount±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Alkyl” refers to an unbranched or branched saturated hydrocarbon chain. As used herein, alkyl has 1 to 20 carbon atoms (i.e., C₁₋₂₀ alkyl), 1 to 8 carbon atoms (i.e., C₁₋₈ alkyl), 1 to 6 carbon atoms (i.e., C₁₋₆ alkyl), or 1 to 4 carbon atoms (i.e., C₁₋₄ alkyl). Examples of alkyl groups include methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl, and 3-methylpentyl. When an alkyl residue having a specific number of carbons is named by chemical name or identified by molecular formula, all positional isomers having that number of carbons may be encompassed; thus, for example, “butyl” includes n-butyl (i.e. —(CH₂)₃CH₃), sec-butyl (i.e. —CH(CH₃)CH₂CH₃), isobutyl (i.e. —CH₂CH(CH₃)₂) and tert-butyl (i.e. —C(CH₃)₃); and “propyl” includes n-propyl (i.e. —(CH₂)₂CH₃) and isopropyl (i.e. —CH(CH₃)₂).

“Alkenyl” refers to an alkyl group containing at least one carbon-carbon double bond and having from 2 to 20 carbon atoms (i.e., C₂₋₂₀ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Examples of alkenyl groups include ethenyl, propenyl, butadienyl (including 1,2-butadienyl and 1,3-butadienyl). “Alkynyl” refers to an alkyl group containing at least one carbon-carbon triple bond and having from 2 to 20 carbon atoms (i.e., C₂₋₂₀ alkynyl), 2 to 8 carbon atoms (i.e., C₂₋₈ alkynyl), 2 to 6 carbon atoms (i.e., C₂₋₆ alkynyl), or 2 to 4 carbon atoms (i.e., C₂₋₄ alkynyl). The term “alkynyl” also includes those groups having one triple bond and one double bond.

“Alkoxy” refers to the group “alkyl-O—”. Examples of alkoxy groups include methoxy, ethoxy, n-propoxy, iso-propoxy, n-butoxy, tert-butoxy, sec-butoxy, n-pentoxy, n-hexoxy, and 1,2-dimethylbutoxy. “Haloalkoxy” refers to an alkoxy group as defined above, wherein one or more hydrogen atoms are replaced by a halogen.

“Alkylthio” refers to the group “alkyl-S—”. “Amino” refers to the group —NR^{y}R^{y} wherein each R^{y} is independently selected from the group consisting of hydrogen, alkyl, alkenyl, alkynyl, aryl, heterocyclyl, cycloalkyl or heteroaryl, each of which is optionally substituted, as defined herein.

“Aryl” refers to an aromatic carbocyclic group having a single ring (e.g. monocyclic) or multiple rings (e.g. bicyclic or tricyclic) including fused systems. As used herein, aryl has 6 to 20 ring carbon atoms (i.e., C₆₋₂₀ aryl), 6 to 12 carbon ring atoms (i.e., C₆₋₁₀ aryl), or 6 to 10 carbon ring atoms (i.e., C₆₋₁₀ aryl). Examples of aryl groups include phenyl, naphthyl, fluorenyl, and anthryl. Aryl, however, does not encompass or overlap in any way with heteroaryl defined below. If one or more aryl groups are fused with a heteroaryl, the resulting ring system is heteroaryl. If one or more aryl groups are fused with a heterocyclyl, the resulting ring system is heterocyclyl. “Cyano” refers to the group —CN.

“Keto” refers to a group C═O. “Carbamoyl” refers to both an “O-carbamoyl” group which refers to the group —O—C(O)NR^{y}R^{z} and an “N-carbamoyl” group which refers to the group —NR^{y}C(O)OR^{z}, wherein R^{y} and R^{z} are independently selected from the group consisting of hydrogen, alkyl, aryl, haloalkyl, or heteroaryl; each of which may be optionally substituted.

“Carboxyl” refers to —C(O)OH. “Ester” refers to both —OC(O)R and —C(O)OR, wherein R is a substituent; each of which may be optionally substituted, as defined herein.

“Cycloalkyl” refers to a saturated or partially unsaturated cyclic alkyl group having a single ring or multiple rings including fused, bridged, and spiro ring systems. The term “cycloalkyl” includes cycloalkenyl groups (i.e. the cyclic group having at least one double bond). As used herein, cycloalkyl has from 3 to 20 ring carbon atoms (i.e., C₃₋₂₀ cycloalkyl), 3 to 12 ring carbon atoms (i.e., C₃-12 cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Examples of cycloalkyl groups include cyclopropyl, cyclobutyl, cyclopentyl, and cyclohexyl.

“Halogen” or “halo” includes fluoro, chloro, bromo, and iodo. “Haloalkyl” refers to an unbranched or branched alkyl group as defined above, wherein one or more hydrogen atoms are replaced by a halogen. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include difluoromethyl (—CHF₂) and trifluoromethyl (—CF₃).

“Heteroalkyl” refers to an alkyl group in which one or more of the carbon atoms (and any associated hydrogen atoms) are each independently replaced with the same or different heteroatomic group. The term “heteroalkyl” includes unbranched or branched saturated chain having carbon and heteroatoms. By way of example, 1, 2 or 3 carbon atoms may be independently replaced with the same or different heteroatomic group. Heteroatomic groups include, but are not limited to, —NR—, —O—, —S—, —S(O)—, —S(O)₂—, and the like, where R is H, alkyl, aryl, cycloalkyl, heteroalkyl, heteroaryl or heterocyclyl, each of which may be optionally substituted. Examples of heteroalkyl groups include —OCH₃, —CH₂OCH₃, —SCH₃, —CH₂SCH₃, —NRCH₃, and —CH₂NRCH₃, where R is hydrogen, alkyl, aryl, arylalkyl, heteroalkyl, or heteroaryl, each of which may be optionally substituted. As used herein, heteroalkyl include 1 to 10 carbon atoms, 1 to 8 carbon atoms, or 1 to 4 carbon atoms; and 1 to 3 heteroatoms, 1 to 2 heteroatoms, or 1 heteroatom.

“Heteroaryl” refers to an aromatic group having a single ring, multiple rings, or multiple fused rings, with one or more ring heteroatoms independently selected from nitrogen, oxygen, and sulfur. As used herein, heteroaryl includes 1 to 20 ring carbon atoms (i.e., C₁₋₂₀ heteroaryl), 3 to 12 ring carbon atoms (i.e., C₃₋₁₂ heteroaryl), or 3 to 8 carbon ring atoms (i.e., C₃₋₈ heteroaryl); and 1 to 5 heteroatoms, 1 to 4 heteroatoms, 1 to 3 ring heteroatoms, 1 to 2 ring heteroatoms, or 1 ring heteroatom independently selected from nitrogen, oxygen, and sulfur. Examples of heteroaryl groups include pyrimidinyl, purinyl, pyridyl, pyridazinyl, benzothiazolyl, and pyrazolyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl, and imidazo[1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heterocyclyl” or “heterocycle” refers to a saturated or unsaturated cyclic alkyl group, with one or more ring heteroatoms independently selected from nitrogen, oxygen and sulfur. The term “heterocyclyl” includes heterocycloalkenyl groups (i.e. the heterocyclyl group having at least one double bond), bicyclic heterocyclyl groups, bridged-heterocyclyl groups, fused-heterocyclyl groups, and spiro-heterocyclyl groups. A heterocyclyl may be a single ring or multiple rings wherein the multiple rings may be fused, bridged, or spiro. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to an aryl or heteroaryl ring, regardless of the attachment to the remainder of the molecule. As used herein, heterocyclyl has 2 to 20 ring atoms (i.e., 4-20 membered heterocyclyl), 2 to ring atoms (i.e., 4-12 membered heterocyclyl), 4 to 10 ring atoms (i.e., 4-10 membered heterocyclyl), 4 to 8 ring atoms (i.e., 4-8 membered heterocyclyl), or 4 to 6 ring carbon atoms (i.e., 4-6 membered heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur or oxygen. A heterocyclyl may contain one or more C═O and/or thiC═O groups. Examples of heterocyclyl groups include pyrrolidinyl, piperidinyl, piperazinyl, oxetanyl, diC═Olanyl, azetidinyl, azetidinyl, morpholinyl, thiomorpholinyl, 4-7 membered sultam, 4-7 membered cyclic carbamate, 4-7 membered cyclic carbonate, 4-7 membered cyclic sulfide and morpholinyl. As used herein, the term “bridged-heterocyclyl” refers to a four- to ten-membered cyclic moiety connected at two non-adjacent atoms of the heterocyclyl with one or more (e.g. 1 or 2) four- to ten-membered cyclic moiety having at least one heteroatom where each heteroatom is independently selected from nitrogen, oxygen, and sulfur. As used herein, bridged-heterocyclyl includes bicyclic and tricyclic ring systems. Also used herein, the term “spiro-heterocyclyl” refers to a ring system in which a three- to ten-membered heterocyclyl has one or more additional ring, wherein the one or more additional ring is three- to ten-membered cycloalkyl or three- to ten-membered heterocyclyl, where a single atom of the one or more additional ring is also an atom of the three- to ten-membered heterocyclyl. Examples of the spiro-heterocyclyl rings include bicyclic and tricyclic ring systems, such as 2-oxa-7-azaspiro[3.5]nonanyl, 2-oxa-6-azaspiro[3.4]octanyl, and 6-oxa-1-azaspiro[3.3]heptanyl. Examples of the fused-heterocyclyl rings include, but are not limited to, 1,2,3,4-tetrahydroisoquinolinyl, 1-C═O-1,2,3,4-tetrahydroisoquinolinyl, 1-C═O-1,2-dihydroisoquinolinyl, 4,5,6,7-tetrahydrothieno[2,3-c]pyridinyl, indolinyl, and isoindolinyl, where the heterocyclyl can be bound via either ring of the fused system. As used herein, a bicyclic heterocyclyl group is a heterocyclyl group attached at two points to another cyclic group, wherein the other cyclic group may itself be a heterocyclic group, or a carbocyclic group.

As used herein, the term “nitrogen or sulfur containing heterocyclyl” means a heterocyclyl moiety that contains at least one nitrogen atom or at least one sulfur atom, or both a nitrogen atom and a sulfur atom within the ring structure. It is to be understood that other heteroatoms, including oxygen, may be present in addition to the nitrogen, sulfur, or combinations thereof. Examples of nitrogen or sulfur containing heterocyclyls include morpholinyl, thiomorpholinyl, thiazolyl, isothiazolyl, oxazolidinone 1,2 dithiolyl, piperidinyl, piperazinyl, and the like.

“Hydroxy” or “hydroxyl” refers to the group —OH. “Hydroxyalkyl” refers to an unbranched or branched alkyl group as defined above, wherein one or more hydrogen atoms are replaced by a hydroxyl. “Nitro” refers to the group —NO₂.

“Sulfonyl” refers to the group —S(O)₂R, where R is a substituent, or a defined group. “Alkylsulfonyl” refers to the group —S(O)₂R, where R is a substituent, or a defined group. “Alkylsulfinyl” refers to the group —S(O)R, where R is a substituent, or a defined group. “Thiocyanate” —SCN. “Thiol” refers to the group —SR, where R is a substituent, or a defined group. “Thione” refer to the group (C═S).

Certain commonly used alternative chemical names may be used. For example, a divalent group such as a divalent “alkyl” group, a divalent “aryl” group, etc., may also be referred to as an “alkylene” group or an “alkylenyl” group, an “arylene” group or an “arylenyl” group, respectively. Also, unless indicated explicitly otherwise, where combinations of groups are referred to herein as one moiety, e.g. arylalkyl, the last mentioned group contains the atom by which the moiety is attached to the rest of the molecule.

The terms “optional” or “optionally” means that the subsequently described event or circumstance may or may not occur, and that the description includes instances where said event or circumstance occurs and instances in which it does not. Also, the term “optionally substituted” refers to any one or more hydrogen atoms on the designated atom or group may or may not be replaced by a moiety other than hydrogen. “Optionally substituted” may be zero to the maximum number of possible substitutions, and each occurrence is independent. When the term “substituted” is used, then that substitution is required to be made at a substitutable hydrogen atom of the indicated substituent. An optional substitution may be the same or different from a (required) substitution.

When a moiety is “optionally substituted,” and reference is made to a general term, such as any “alkyl,” “alkenyl,” “alkynyl,” “haloalkyl,” “cycloalkyl,” “aryl” or “heteroaryl,” then the general term can refer to any antecedent specifically recited term, such as (C₁-3 alkyl), (C₄₆ alkyl), —O(C₁₋₄ alkyl), (C₃₋₁₀ cycloalkyl), O—(C₃₋₁₀ cycloalkyl) and the like. For example, “any aryl” includes both “aryl” and “—O(aryl) as well as examples of aryl, such as phenyl or naphthyl and the like. Also, the term “any heterocyclyl” includes both the terms “heterocyclyl” and O-(heterocyclyl),” as well as examples of heterocyclyls, such as oxetanyl, tetrahydropyranyl, morpholino, piperidinyl and the like. In the same manner, the term “any heteroaryl” includes the terms “heteroaryl” and “O-(heteroryl),” as well as specific heteroaryls, such as pyridine and the like.

Some of the compounds exist as tautomers. Tautomers are in equilibrium with one another. For example, amide containing compounds may exist in equilibrium with imidic acid tautomers. Regardless of which tautomer is shown, and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

Any formula or structure given herein, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to ²H (deuterium, D), ³H (tritium), ¹¹C, ¹³C, ¹⁴C, ¹⁵N, ¹⁸F, ³¹P, ³²P, ³⁵, ³⁶Cl and ¹²⁵I. Various isotopically labeled compounds of the present disclosure, for example those into which radioactive isotopes such as ³H, ¹³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The disclosure also includes “deuterated analogues” of compounds of Formula I in which from 1 to n hydrogens attached to a carbon atom is/are replaced by deuterium, in which n is the number of hydrogens in the molecule. Such compounds may exhibit increased resistance to metabolism and are thus useful for increasing the half-life of any compound of Formula I when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in the compound of Formula I.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

In many cases, the compounds of this disclosure are capable of forming acid and/or base salts by virtue of the presence of amino and/or carboxyl groups or groups similar thereto.

Provided are also pharmaceutically acceptable salts, hydrates, solvates, tautomeric forms, polymorphs, and prodrugs of the compounds described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound, and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines (i.e., NH₂(alkyl)), dialkyl amines (i.e., HN(alkyl)₂), trialkyl amines (i.e., N(alkyl)₃), substituted alkyl amines (i.e., NH₂(substituted alkyl)), di(substituted alkyl) amines (i.e., HN(substituted alkyl)₂), tri(substituted alkyl) amines (i.e., N(substituted alkyl)₃), alkenyl amines (i.e., NH₂(alkenyl)), dialkenyl amines (i.e., HN(alkenyl)₂), trialkenyl amines (i.e., N(alkenyl)₃), substituted alkenyl amines (i.e., NH₂(substituted alkenyl)), di(substituted alkenyl) amines (i.e., HN(substituted alkenyl)₂), tri(substituted alkenyl) amines (i.e., N(substituted alkenyl)₃, mono-, di- or tri-cycloalkyl amines (i.e., NH₂(cycloalkyl), HN(cycloalkyl)₂, N(cycloalkyl)₃), mono-, di- or tri-arylamines (i.e., NH₂(aryl), HN(aryl)₂, N(aryl)₃), or mixed amines, etc. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

The term “substituted” means that any one or more hydrogen atoms on the designated atom or group is replaced with one or more substituents other than hydrogen, provided that the designated atom's normal valence is not exceeded. The one or more substituents include, but are not limited to, alkyl, alkenyl, alkynyl, alkoxy, acyl, amino, amido, amidino, aryl, azido, carbamoyl, carboxyl, carboxyl ester, cyano, guanidino, halo, haloalkyl, haloalkoxy, heteroalkyl, heteroaryl, heterocyclyl, hydroxy, hydrazino, imino, C═O, nitro, alkylsulfinyl, sulfonic acid, alkylsulfonyl, thiocyanate, thiol, thione, or combinations thereof. Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl)substituted aryl) substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan. When used to modify a chemical group, the term “substituted” may describe other chemical groups defined herein. Unless specified otherwise, where a group is described as optionally substituted, any substituents of the group are themselves unsubstituted. For example, in some embodiments, the term “substituted alkyl” refers to an alkyl group having one or more substituents including hydroxyl, halo, alkoxy, cycloalkyl, heterocyclyl, aryl, and heteroaryl. In other embodiments, the one or more substituents may be further substituted with halo, alkyl, haloalkyl, hydroxyl, alkoxy, cycloalkyl, heterocyclyl, aryl, or heteroaryl, each of which is substituted. In other embodiments, the substituents may be further substituted with halo, alkyl, haloalkyl, alkoxy, hydroxyl, cycloalkyl, heterocyclyl, aryl, or heteroaryl, each of which is unsubstituted One skilled in the art will recognize that substituents and other moieties of the compounds of the generic formula herein should be selected in order to provide a compound which is sufficiently stable to provide a pharmaceutically useful compound which can be formulated into an acceptably stable pharmaceutical composition. Compounds which have such stability are contemplated as falling within the scope of the present invention. It should be understood by one skilled in the art that any combination of the definitions and substituents described above should not result in an inoperable species or compound.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

A “solvate” is formed by the interaction of a solvent and a compound. Solvates of salts of the compounds described herein are also provided. Hydrates of the compounds described herein are also provided.

Certain commonly used alternative chemical names may be used. For example, a divalent group such as a divalent “alkyl” group, a divalent “aryl” group, etc., may also be referred to as an “alkylene” group or an “alkylenyl” group, an “arylene” group or an “arylenyl” group, respectively. Also, unless indicated explicitly otherwise, where combinations of groups are referred to herein as one moiety, e.g. arylalkyl, the last mentioned group contains the atom by which the moiety is attached to the rest of the molecule.

The terms “optional” or “optionally” means that the subsequently described event or circumstance may or may not occur, and that the description includes instances where said event or circumstance occurs and instances in which it does not. Also, the term “optionally substituted” refers to any one or more hydrogen atoms on the designated atom or group may or may not be replaced by a moiety other than hydrogen. “Optionally substituted” may be zero to the maximum number of possible substitutions, and each occurrence is independent. When the term “substituted” is used, then that substitution is required to be made at a substitutable hydrogen atom of the indicated substituent. An optional substitution may be the same or different from a (required) substitution.

When a moiety is “optionally substituted,” and reference is made to a general term, such as any “alkyl,” “alkenyl,” “alkynyl,” “haloalkyl,” “cycloalkyl,” “aryl” or “heteroaryl,” then the general term can refer to any antecedent specifically recited term, such as (C₁₋₃ alkyl), (C₄₆ alkyl), —O(C₁₋₄ alkyl), (C₃₋₁₀ cycloalkyl), O—(C₃₋₁₀ cycloalkyl) and the like. For example, “any aryl” includes both “aryl” and “—O(aryl) as well as examples of aryl, such as phenyl or naphthyl and the like. Also, the term “any heterocyclyl” includes both the terms “heterocyclyl” and O-(heterocyclyl),” as well as examples of heterocyclyls, such as oxetanyl, tetrahydropyranyl, morpholino, piperidinyl and the like. In the same manner, the term “any heteroaryl” includes the terms “heteroaryl” and “O-(heteroryl),” as well as specific heteroaryls, such as pyridine and the like.

Some of the compounds exist as tautomers. Tautomers are in equilibrium with one another. For example, amide containing compounds may exist in equilibrium with imidic acid tautomers. Regardless of which tautomer is shown, and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

Any formula or structure given herein, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl and ¹²⁵I. Various isotopically labeled compounds of the present disclosure, for example those into which radioactive isotopes such as ³H, ¹³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The disclosure also includes “deuterated analogues” of compounds of Formula I in which from 1 to n hydrogens attached to a carbon atom is/are replaced by deuterium, in which n is the number of hydrogens in the molecule. Such compounds exhibit increased resistance to metabolism and are thus useful for increasing the half-life of any compound of Formula I when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in the compound of Formula I.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

In many cases, the compounds of this disclosure are capable of forming acid and/or base salts by virtue of the presence of amino and/or carboxyl groups or groups similar thereto.

Provided are also pharmaceutically acceptable salts, hydrates, solvates, tautomeric forms, polymorphs, and prodrugs of the compounds described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound, and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines (i.e., NH₂(alkyl)), dialkyl amines (i.e., HN(alkyl)₂), trialkyl amines (i.e., N(alkyl)₃), substituted alkyl amines (i.e., NH₂(substituted alkyl)), di(substituted alkyl) amines (i.e., HN(substituted alkyl)₂), tri(substituted alkyl) amines (i.e., N(substituted alkyl)₃), alkenyl amines (i.e., NH₂(alkenyl)), dialkenyl amines (i.e., HN(alkenyl)₂), trialkenyl amines (i.e., N(alkenyl)₃), substituted alkenyl amines (i.e., NH₂(substituted alkenyl)), di(substituted alkenyl) amines (i.e., HN(substituted alkenyl)₂), tri(substituted alkenyl) amines (i.e., N(substituted alkenyl)₃, mono-, di- or tri-cycloalkyl amines (i.e., NH₂(cycloalkyl), HN(cycloalkyl)₂, N(cycloalkyl)₃), mono-, di- or tri-arylamines (i.e., NH₂(aryl), HN(aryl)₂, N(aryl)₃), or mixed amines, etc. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

The term “substituted” means that any one or more hydrogen atoms on the designated atom or group is replaced with one or more substituents other than hydrogen, provided that the designated atom's normal valence is not exceeded. The one or more substituents include, but are not limited to, alkyl, alkenyl, alkynyl, alkoxy, acyl, amino, amido, amidino, aryl, azido, carbamoyl, carboxyl, carboxyl ester, cyano, guanidino, halo, haloalkyl, haloalkoxy, heteroalkyl, heteroaryl, heterocyclyl, hydroxy, hydrazino, imino, C═O, nitro, alkylsulfinyl, sulfonic acid, alkylsulfonyl, thiocyanate, thiol, thione, or combinations thereof. Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl)substituted aryl) substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan. When used to modify a chemical group, the term “substituted” may describe other chemical groups defined herein. Unless specified otherwise, where a group is described as optionally substituted, any substituents of the group are themselves unsubstituted. For example, in some embodiments, the term “substituted alkyl” refers to an alkyl group having one or more substituents including hydroxyl, halo, alkoxy, cycloalkyl, heterocyclyl, aryl, and heteroaryl. In other embodiments, the one or more substituents may be further substituted with halo, alkyl, haloalkyl, hydroxyl, alkoxy, cycloalkyl, heterocyclyl, aryl, or heteroaryl, each of which is substituted. In other embodiments, the substituents may be further substituted with halo, alkyl, haloalkyl, alkoxy, hydroxyl, cycloalkyl, heterocyclyl, aryl, or heteroaryl, each of which is unsubstituted One skilled in the art will recognize that substituents and other moieties of the compounds of the generic formula herein should be selected in order to provide a compound which is sufficiently stable to provide a pharmaceutically useful compound which can be formulated into an acceptably stable pharmaceutical composition. Compounds which have such stability are contemplated as falling within the scope of the present invention. It should be understood by one skilled in the art that any combination of the definitions and substituents described above should not result in an inoperable species or compound.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

A “solvate” is formed by the interaction of a solvent and a compound. Solvates of salts of the compounds described herein are also provided. Hydrates of the compounds described herein are also provided.

### II. Combinations

Patients being treated by administration of the PARP7 inhibitors of the disclosure often exhibit diseases or conditions that benefit from treatment with other therapeutic agents. These diseases or conditions can be of an oncology nature or can be related to Inflammation, metabolic disorders, gastrointestinal disorders and the like. Thus, one aspect of the disclosure is a method of treating cancer, comprising administering a compound of the in combination with one or more compounds useful for the treatment of such diseases to a subject, particularly a human subject, in need thereof.

In some embodiments, a compound of the present disclosure is co-formulated with the additional one or more active ingredients. In some embodiments, the other active ingredient is administered at approximately the same time, in a separate dosage form. In some embodiments, the other active ingredient is administered sequentially, and may be administered at different times in relation to a compound of the present disclosure.

In some embodiments, a compound, or pharmaceutical composition provided herein, is administered with one or more (e.g., one, two, three, or four) additional therapeutic agents. In some embodiments the additional therapeutic agent includes, e.g., an inhibitory immune checkpoint blocker or inhibitor, a stimulatory immune checkpoint stimulator, agonist or activator, a chemotherapeutic agent, an anti-cancer agent, a radiotherapeutic agent, an anti-neoplastic agent, an anti-proliferation agent, an anti-angiogenic agent, an anti-inflammatory agent, an immunotherapeutic agent, a therapeutic antigen-binding molecule (e.g., a mono- and multi-specific antibody, or fragment thereof, in any format, such as DART®, Duobody®, BiTE®, BiKE, TriKE, XmAb®, TandAb®, scFv, Fab, Fab derivative), a bi-specific antibody, a non-immunoglobulin antibody mimetic (e.g., including adnectin, affibody, affilin, affimer, affitin, alphabody, anticalin, peptide aptamer, armadillo repeat protein (ARM), atrimer, avimer, designed ankyrin repeat protein (DARPin®), fynomer, knottin, Kunitz domain peptide, monobody, and nanoCLAMPs), an antibody-drug conjugate (ADC), antibody-peptide conjugate), an oncolytic virus, a gene modifier or editor, a cell comprising a chimeric antigen receptor (CAR), e.g., including a T-cell immunotherapeutic agent, an NK-cell immunotherapeutic agent, or a macrophage immunotherapeutic agent, a cell comprising an engineered T-cell receptor (TCR-T), or any combination thereof.

### Illustrative Targets

In some embodiments, the one or more additional therapeutic agents include, e.g., an inhibitor, agonist, antagonist, ligand, modulator, stimulator, blocker, activator or suppressor of a target (e.g., polypeptide or polynucleotide), such as: 2′-5′-oligoadenylate synthetase (OAS1; NCBI Gene ID: 4938); 5′-3′ exoribonuclease 1 (XRN1; NCBI Gene ID: 54464); 5′-nucleotidase ecto (NT5E, CD73; NCBI Gene ID: 4907); ABL proto-oncogene 1, non-receptor tyrosine kinase (ABL1, BCR-ABL, c-ABL, v-ABL; NCBI Gene ID: 25); absent in melanoma 2 (AIM2; NCBI Gene ID: 9447); acetyl-CoA acyltransferase 2 (ACAA2; NCBI Gene ID: 10499); acid phosphatase 3 (ACP3; NCBI Gene ID: 55); adenosine deaminase (ADA, ADA1; NCBI Gene ID: 100); adenosine receptors (e.g., ADORA1 (A1), ADORA2A (A2a, A2AR), ADORA2B (A2b, A2BR), ADORA3 (A3); NCBI Gene IDs: 134, 135, 136, 137); AKT serine/threonine kinase 1 (AKT1, AKT, PKB; NCBI Gene ID: 207); alanyl aminopeptidase, membrane (ANPEP, CD13; NCBI Gene ID: 290); ALK receptor tyrosine kinase (ALK, CD242; NCBI Gene ID: 238); alpha fetoprotein (AFP; NCBI Gene ID: 174); amine oxidase copper containing (e.g., AOC1 (DAO1), AOC2, AOC3 (VAP1); NCBI Gene IDs: 26, 314, 8639); androgen receptor (AR; NCBI Gene ID: 367); angiopoietins (ANGPT1, ANGPT2; NCBI Gene IDs: 284, 285); angiotensin II receptor type 1 (AGTR1; NCBI Gene ID: 185); angiotensinogen (AGT; NCBI Gene ID: 183); apolipoprotein A1 (APOA1; NCBI Gene ID: 335); apoptosis inducing factor mitochondria associated 1 (AIFM1, AIF; NCBI Gene ID: 9131); arachidonate 5-lipoxygenase (ALOX5; NCBI Gene ID: 240); asparaginase (ASPG; NCBI Gene ID: 374569); asteroid homolog 1 (ASTE1; NCBI Gene ID: 28990); ATM serine/threonine kinase (ATM; NCBI Gene ID: 472); ATP binding cassette subfamily B member 1 (ABCB1, CD243, GP170; NCBI Gene ID: 5243); ATP-dependent Clp-protease (CLPP; NCBI Gene ID: 8192); ATR serine/threonine kinase (ATR; NCBI Gene ID: 545); AXL receptor tyrosine kinase (AXL; NCBI Gene ID: 558); B and T lymphocyte associated (BTLA, CD272; NCBI Gene ID: 151888); baculoviral IAP repeat containing proteins (BIRC2 (cIAP1), BIRC3 (cIAP2), XIAP (BIRC4, IAP3), BIRC5 (survivin); NCBI Gene IDs: 329, 330, 331, 332); basigin (Ok blood group) (BSG, CD147; NCBI Gene ID: 682); B-cell lymphoma 2 (BCL2; NCBI Gene ID: 596); BCL2 binding component 3 (BBC3, PUMA; NCBI Gene ID: 27113); BCL2 like (e.g., BCL2L1 (Bcl-x), BCL2L2 (BIM); Bcl-x; NCBI Gene IDs: 598, 10018); beta 3-adrenergic receptor (ADRB3; NCBI Gene ID: 155); bone gamma-carboxyglutamate protein (BGLAP; NCBI Gene ID: 632); bone morphogenetic protein-10 ligand (BMP10; NCBI Gene ID: 27302); bradykinin receptors (e.g., BDKRB1, BDKRB2; NCBI Gene IDs: 623, 624); B-RAF (BRAF; NCBI Gene ID: 273); breakpoint cluster region (BCR; NCBI Gene ID: 613); bromodomain and external domain (BET) bromodomain containing proteins (e.g., BRD2, BRD3, BRD4, BRDT; NCBI Gene IDs: 6046, 8019, 23476, 676); Bruton's tyrosine kinase (BTK; NCBI Gene ID: 695); cadherins (e.g., CDH3 (p-cadherin), CDH6 (k-cadherin); NCBI Gene IDs: 1001, 1004); cancer/testis antigens (e.g., CTAG1A, CTAG1B, CTAG2; NCBI Gene IDs: 1485, 30848, 246100); cannabinoid receptors (e.g., CNR1 (CB1), CNR2 (CB2); NCBI Gene IDs: 1268, 1269); carbohydrate sulfotransferase 15 (CHST15; NCBI Gene ID: 51363); carbonic anhydrases (e.g., CA1, CA2, CA3, CA4, CASA, CASB, CA6, CA7, CA8, CA9, CA10, CA11, CA12, CA13, CA14; NCBI Gene IDs: 759, 760, 761, 762, 763, 765, 766, 767, 768, 770, 771, 11238, 23632, 56934, 377677); carcinoembryonic antigen related cell adhesion molecules (e.g., CEACAM3 (CD66d), CEACAM5 (CD66e), CEACAM6 (CD66c); NCBI Gene IDs: 1048, 1084, 4680); casein kinases (e.g., CSNK1A1 (CK1), CSNK2A1 (CK2); NCBI Gene IDs: 1452, 1457); caspases (e.g., CASP3, CASP7, CASP8; NCBI Gene IDs: 836, 840, 841, 864); catenin beta 1 (CTNNB1; NCBI Gene ID: 1499); cathepsin G (CTSG; NCBI Gene ID: 1511); Cbl proto-oncogene B (CBLB, Cbl-b; NCBI Gene ID: 868); C-C motif chemokine ligand 21 (CCL21; NCBI Gene ID: 6366); C-C motif chemokine receptor 2 (CCR2; NCBI Gene ID: 729230); C-C motif chemokine receptors (e.g., CCR3 (CD193), CCR4 (CD194), CCR5 (CD195), CCR8 (CDw198); NCBI Gene IDs: 1232, 1233, 1234, 1237); CCAAT enhancer binding protein alpha (CEBPA, CEBP; NCBI Gene ID: 1050); cell adhesion molecule 1 (CADM1; NCBI Gene ID: 23705); cell division cycle 7 (CDC7; NCBI Gene ID: 8317); cellular communication network factor 2 (CCN2; NCBI Gene ID: 1490); cereblon (CRBN; NCBI Gene ID: 51185); checkpoint kinases (e.g., CHEK1 (CHK1), CHEK2 (CHK2); NCBI Gene IDs: 1111, 11200); cholecystokinin B receptor (CCKBR; NCBI Gene ID: 887); chorionic somatomammotropin hormone 1 (CSH1; NCBI Gene ID: 1442); claudins (e.g., CLDN6, CLDN18; NCBI Gene IDs: 9074, 51208); cluster of differentiation markers (e.g., CD1A, CD1C, CD1D, CD1E, CD2, CD3 alpha (TRA), CD beta (TRB), CD gamma (TRG), CD delta (TRD), CD4, CD8A, CD8B, CD19, CD20 (MS4A1), CD22, CD24, CD25 (IL2RA, TCGFR), CD28, CD33 (SIGLEC3), CD37, CD38, CD39 (ENTPD1), CD40 (TNFRSF5), CD44 (MIC4, PGP1), CD47 (IAP), CD48 (BLAST1), CD52, CD55 (DAF), CD58 (LFA3), CD74, CD79a, CD79b, CD80 (B7-1), CD84, CD86 (B7-2), CD96 (TACTILE), CD99 (MIC2), CD115 (CSF1R), CD116 (GMCSFR, CSF2RA), CD122 (IL2RB), CD123 (IL3RA), CD128 (IL8R1), CD132 (IL2RG), CD135 (FLT3), CD137 (TNFRSF9, 4-1BB), CD142 (TF, TFA), CD152 (CTLA4), CD160, CD182 (IL8R2), CD193 (CCR3), CD194 (CCR4), CD195 (CCR5), CD207, CD221 (IGF1R), CD222 (IGF2R), CD223 (LAG3), CD226 (DNAM1), CD244, CD247, CD248, CD276 (B7-H3), CD331 (FGFR1), CD332 (FGFR2), CD333 (FGFR3), CD334 (FGFR4); NCBI Gene IDs: 909, 911, 912, 913, 914, 919, 920, 923, 925, 926, 930, 931, 933, 940, 941, 942, 945, 951, 952, 953, 958,960, 961, 962, 965, 972, 973, 974, 1043, 1232, 1233, 1234, 1237, 1436, 1438, 1493, 1604, 2152, 2260, 2261, 2263, 2322, 3480, 3482, 3559, 3560, 3561, 3563, 3577, 3579, 3604, 3902, 4267, 6955, 6957, 6964, 6965, 8832, 10666, 11126, 50489, 51744, 80381, 100133941); clusterin (CLU; NCBI Gene ID: 1191); coagulation factors (e.g., F7, FXA, NCBI Gene IDs: 2155, 2159); collagen type IV alpha chains (e.g., COL4A1, COL4A2, COL4A3, COL4A4, COL4A5; NCBI Gene IDs: 1282, 1284, 1285, 1286, 1287); collectin subfamily member 10 (COLEC10; NCBI Gene ID: 10584); colony stimulating factors (e.g., CSF1 (MCSF), CSF2 (GMCSF), CSF3 (GCSF); NCBI Gene IDs: 1435, 1437, 1440); complement factors (e.g., C3, C5; NCBI Gene IDs: 718, 727); COP9 signalosome subunit 5 (COPS5; NCBI Gene ID: 10987); C-type lectin domain family member (e.g., CLEC4C (CD303), CLEC9A (CD370), CLEC12A (CD371); CD371; NCBI Gene ID: 160364, 170482, 283420); C-X-C motif chemokine ligand 12 (CXCL12; NCBI Gene ID: 6387); C-X-C motif chemokine receptors (CXCR1 (IL8R1, CD128), CXCR2 (IL8R2, CD182), CXCR3 (CD182, CD183, IP-10R), CXCR4 (CD184); NCBI Gene ID: 2833, 3577, 3579, 7852); cyclin D1 (CCND1, BCL1; NCBI Gene ID: 595); cyclin dependent kinases (e.g., CDK1, CDK2, CDK3, CDK4, CDK5, CDK6, CDK7, CDK8, CDK9, CDK10, CDK12; NCBI Gene ID: 983, 1017, 1018, 1019, 1020, 1021, 1022, 1024, 1025, 8558, 51755); cyclin G1 (CCNG1; NCBI Gene ID: 900); cytochrome P450 family members (e.g., CYP2D6, CYP3A4, CYP11A1, CYP11B2, CYP17A1, CYP19A1, CYP51A1; NCBI Gene IDs: 1565, 1576, 1583, 1585, 1586, 1588, 1595); cytochrome P450 oxidoreductase (POR; NCBI Gene ID: 5447); cytokine inducible SH2 containing protein (CISH; NCBI Gene ID: 1154); cytotoxic T-lymphocyte associated protein 4 (CTLA4, CD152; NCBI Gene ID: 1493); DEAD-box helicases (e.g., DDX5, DDX6, DDX58; NCBI Gene IDs: 1655, 1656, 23586); delta like canonical Notch ligands (e.g., DLL3, DLL4; NCBI Gene IDs: 10683, 54567); diablo IAP-binding mitochondrial protein (DIABLO, SMAC; NCBI Gene ID: 56616); diacylglycerol kinases (e.g., DGKA, DGKZ; NCBI Gene IDs: 1606, 8525); dickkopf WNT signaling pathway inhibitors (e.g., DKK1, DKK3; NCBI Gene ID: 22943, 27122); dihydrofolate reductase (DHFR; NCBI Gene ID: 1719); dihydropyrimidine dehydrogenase (DPYD; NCBI Gene ID: 1806); dipeptidyl peptidase 4 (DPP4; NCBI Gene ID: 1803); discoidin domain receptor tyrosine kinases (e.g., DDR1 (CD167), DDR2; CD167; NCBI Gene ID: 780, 4921); DNA dependent protein kinase (PRKDC; NCBI Gene ID: 5591); DNA topoisomerases (e.g., TOP1, TOP2A, TOP2B, TOP3A, TOP3B; NCBI Gene ID: 7150, 7153, 7155, 7156, 8940); dopachrome tautomerase (DCT; NCBI Gene ID: 1638); dopamine receptor D2 (DRD2; NCBI Gene ID: 1318); DOT1 like histone lysine methyltransferase (DOT1L; NCBI Gene ID: 84444); ectonucleotide pyrophosphatase/phosphodiesterase 3 (ENPP3, CD203c; NCBI Gene ID: 5169); EMAP like 4 (EML4; NCBI Gene ID: 27436); endoglin (ENG; NCBI Gene ID: 2022); endoplasmic reticulum aminopeptidases (e.g., ERAP1, ERAP2; NCBI Gene ID: 51752, 64167); enhancer of zeste 2 polycomb repressive complex 2 subunit (EZH2; NCBI Gene ID: 2146); ephrin receptors (e.g., EPHA1, EPHA2EPHA3, EPHA4, EPHA5, EPHA7, EPHB4; NCBI Gene ID: 1969, 2041, 2042, 2043, 2044, 2045, 2050); ephrins (e.g., EFNA1, EFNA4, EFNB2; NCBI Gene ID: 1942, 1945, 1948); epidermal growth factor receptors (e.g., ERBB1 (HER1, EGFR), ERBB1 variant III (EGFRvIII), ERBB2 (HER2, NEU, CD340), ERBB3 (HER3), ERBB4 (HER4); NCBI Gene ID: 1956, 2064, 2065, 2066); epithelial cell adhesion molecule (EPCAM; NCBI Gene ID: 4072); epithelial mitogen (EPGN; NCBI Gene ID: 255324); eukaryotic translation elongation factors (e.g., EEF1A2, EEF2; NCBI Gene ID: 1917, 1938); eukaryotic translation initiation factors (e.g., EIF4A1, EIFSA; NCBI Gene ID: 1973, 1984); exportin-1 (XPO1; NCBI Gene ID: 7514); farnesoid X receptor (NR1H4, FXR; NCBI Gene ID: 9971); Fas ligand (FASLG, FASL, CD95L, CD178, TNFSF6; NCBI Gene ID: 356); fatty acid amide hydrolase (FAAH; NCBI Gene ID: 2166); fatty acid synthase (FASN; FAS; NCBI Gene ID: 2194); Fc fragment of Ig receptors (e.g., FCER1A, FCGRT, FCGR3A (CD16); NCBI Gene IDs: 2205, 2214, 2217); Fc receptor like 5 (FCRL5, CD307; NCBI Gene ID: 83416); fibroblast activation protein alpha (FAP; NCBI Gene ID: 2191); fibroblast growth factor receptors (e.g., FGFR1 (CD331), FGFR2 (CD332), FGFR3 (CD333), FGFR4 (CD334); NCBI Gene IDs: 2260, 2261, 2263, 2264); fibroblast growth factors (e.g., FGF1 (FGF alpha), FGF2 (FGF beta), FGF4, FGF5; NCBI Gene IDs: 2246, 2247, 2249, 2250); fibronectin 1 (FN1, MSF; NCBI Gene ID: 2335); fins related receptor tyrosine kinases (e.g., FLT1 (VEGFR1), FLT3 (STK1, CD135), FLT4 (VEGFR2); NCBI Gene IDs: 2321, 2322, 2324); fms related receptor tyrosine kinase 3 ligand (FLT3LG; NCBI Gene ID: 2323); focal adhesion kinase 2 (PTK2, FAK1; NCBI Gene ID: 5747); folate hydrolase 1 (FOLH1, PSMA; NCBI Gene ID: 2346); folate receptor 1 (FOLR1; NCBI Gene ID: 2348); forkhead box protein M1 (FOXM1; NCBI Gene ID: 2305); FURIN (FURIN, PACE; NCBI Gene ID: 5045); FYN tyrosine kinase (FYN, SYN; NCBI Gene ID: 2534); galectins (e.g., LGALS3, LGALS8 (PCTA1), LGALS9; NCBI Gene ID: 3958, 3964, 3965); glucocorticoid receptor (NR3C1, GR; NCBI Gene ID: 2908); glucuronidase beta (GUSB; NCBI Gene ID: 2990); glutamate metabotropic receptor 1 (GRM1; NCBI Gene ID: 2911); glutaminase (GLS; NCBI Gene ID: 2744); glutathione S-transferase Pi (GSTP1; NCBI Gene ID: 2950); glycogen synthase kinase 3 beta (GSK3B; NCBI Gene ID: 2932); glypican 3 (GPC3; NCBI Gene ID: 2719); gonadotropin releasing hormone 1 (GNRH1; NCBI Gene ID: 2796); gonadotropin releasing hormone receptor (GNRHR; NCBI Gene ID: 2798); GPNMB glycoprotein nmb (GPNMB, osteoactivin; NCBI Gene ID: 10457); growth differentiation factor 2 (GDF2, BMP9; NCBI Gene ID: 2658); growth factor receptor-bound protein 2 (GRB2, ASH; NCBI Gene ID: 2885); guanylate cyclase 2C (GUCY2C, STAR, MECIL, MUCIL, NCBI Gene ID: 2984); H19 imprinted maternally expressed transcript (H19; NCBI Gene ID: 283120); HCK proto-oncogene, Src family tyrosine kinase (HCK; NCBI Gene ID: 3055); heat shock proteins (e.g., HSPA5 (HSP70, BIP, GRP78), HSPB1 (HSP27), HSP90B1 (GP96); NCBI Gene IDs: 3309, 3315, 7184); heme oxygenases (e.g., HMOX1 (HO1), HMOX2 (HO1); NCBI Gene ID: 3162, 3163); heparanase (HPSE; NCBI Gene ID: 10855); hepatitis A virus cellular receptor 2 (HAVCR2, TIM3, CD366; NCBI Gene ID: 84868); hepatocyte growth factor (HGF; NCBI Gene ID: 3082); HERV-H LTR-associating 2 (HHLA2, B7-H7; NCBI Gene ID: 11148); histamine receptor H2 (HRH2; NCBI Gene ID: 3274); histone deacetylases (e.g., HDAC1, HDAC7, HDAC9; NCBI Gene ID: 3065, 9734, 51564); HRas proto-oncogene, GTPase (HRAS; NCBI Gene ID: 3265); hypoxia-inducible factors (e.g., HIF1A, HIF2A (EPAS1); NCBI Gene IDs: 2034, 3091); I-Kappa-B kinase (IKK beta; NCBI Gene IDs: 3551, 3553); IKAROS family zinc fingers (IKZF1 (LYF1), IKZF3; NCBI Gene ID: 10320, 22806); immunoglobulin superfamily member 11 (IGSF11; NCBI Gene ID: 152404); indoleamine 2,3-dioxygenases (e.g., IDO1, IDO2; NCBI Gene IDs: 3620, 169355); inducible T cell costimulator (ICOS, CD278; NCBI Gene ID: 29851); inducible T cell costimulator ligand (ICOSLG, B7-H2; NCBI Gene ID: 23308); insulin like growth factor receptors (e.g., IGF1R, IGF2R; NCBI Gene ID: 3480, 3482); insulin like growth factors (e.g., IGF1, IGF2; NCBI Gene IDs: 3479, 3481); insulin receptor (INSR, CD220; NCBI Gene ID: 3643); integrin subunits (e.g., ITGA5 (CD49e), ITGAV (CD51), ITGB1 (CD29), ITGB2 (CD18, LFA1, MAC1), ITGB7; NCBI Gene IDs: 3678, 3685, 3688, 3695, 3698); intercellular adhesion molecule 1 (ICAM1, CD54; NCBI Gene ID: 3383); interleukin 1 receptor associated kinase 4 (IRAK4; NCBI Gene ID: 51135); interleukin receptors (e.g., IL2RA (TCGFR, CD25), IL2RB (CD122), IL2RG (CD132), IL3RA, IL6R, IL13RA2 (CD213A2), IL22RA1; NCBI Gene IDs: 3598, 3559, 3560, 3561, 3563, 3570, 58985); interleukins (e.g., IL1A, IL1B, IL2, IL3, IL6 (HGF), IL7, IL8 (CXCL8), IL10 (TGIF), IL12A, IL12B, IL15, IL17A (CTLA8), IL18, IL23A, IL24, IL-29 (IFNL1); NCBI Gene IDs: 3552, 3553, 3558, 3562, 3565, 3569, 3574, 3586, 3592, 3593, 3600, 3605, 3606, 11009, 51561, 282618); isocitrate dehydrogenases (NADP(+)1) (e.g., IDH1, IDH2; NCBI Gene IDs: 3417, 3418); Janus kinases (e.g., JAK1, JAK2, JAK3; NCBI Gene IDs: 3716, 3717, 3718); kallikrein related peptidase 3 (KLK3; NCBI Gene ID: 354); killer cell immunoglobulin like receptor, Ig domains and long cytoplasmic tails (e.g., KIR2DL1 (CD158A), KIR2DL2 (CD158B1), KIR2DL3 (CD158B), KIR2DL4 (CD158D), KIR2DL5A (CD158F), KIR2DL5B, KIR3DL1 (CD158E1), KIR3DL2 (CD158K), KIR3DP1 (CD158c), KIR2DS2 (CD158J); NCBI Gene IDs: 3802, 3803, 3804, 3805, 3811, 3812, 57292, 553128, 548594, 100132285); killer cell lectin like receptors (e.g., KLRC1 (CD159A), KLRC2 (CD159c), KLRC3, KLRRC4, KLRD1 (CD94), KLRG1, KLRK1 (NKG2D, CD314); NCBI Gene IDs: 3821, 3822, 3823, 3824, 8302, 10219, 22914); kinase insert domain receptor (KDR, CD309, VEGFR2; NCBI Gene ID: 3791); kinesin family member 11 (KIF11; NCBI Gene ID: 3832); KiSS-1 metastasis suppressor (KISS1; NCBI Gene ID: 3814); KIT proto-oncogene, receptor tyrosine kinase (KIT, C-KIT, CD117; NCBI Gene ID: 3815); KRAS proto-oncogene, GTPase (KRAS; NCBI Gene ID: 3845); lactotransferrin (LTF; NCBI Gene ID: 4057); LCK proto-oncogene, Src family tyrosine kinase (LCK; NCBI Gene ID: 3932); LDL receptor related protein 1 (LRP1, CD91, IGFBP3R; NCBI Gene ID: 4035); leucine rich repeat containing 15 (LRRC15; NCBI Gene ID: 131578); leukocyte immunoglobulin like receptors (e.g., LILRB1 (ILT2, CD85J), LILRB2 (ILT4, CD85D); NCBI Gene ID: 10288, 10859); leukotriene A4 hydrolase (LTA4H; NCBI Gene ID: 4048); linker for activation of T-cells (LAT; NCBI Gene ID: 27040); luteinizing hormone/choriogonadotropin receptor (LHCGR; NCBI Gene ID: 3973); LY6/PLAUR domain containing 3 (LYPD3; NCBI Gene ID: 27076); lymphocyte activating 3 (LAG3; CD223; NCBI Gene ID: 3902); lymphocyte antigens (e.g., LY9 (CD229), LY75 (CD205); NCBI Gene IDs: 4063, 17076); LYN proto-oncogene, Src family tyrosine kinase (LYN; NCBI Gene ID: 4067); lymphocyte cytosolic protein 2 (LCP2; NCBI Gene ID: 3937); lysine demethylase 1A (KDM1A; NCBI Gene ID: 23028); lysophosphatidic acid receptor 1 (LPAR1, EDG2, LPA1, GPR26; NCBI Gene ID: 1902); lysyl oxidase (LOX; NCBI Gene ID: 4015); lysyl oxidase like 2 (LOXL2; NCBI Gene ID: 4017); macrophage migration inhibitory factor (MIF, GIF; NCBI Gene ID: 4282); macrophage stimulating 1 receptor (MST1R, CD136; NCBI Gene ID: 4486); MAGE family members (e.g., MAGEA1, MAGEA2, MAGEA2B, MAGEA3, MAGEA4, MAGEA5, MAGEA6, MAGEA10, MAGEA11, MAGEC1, MAGEC2, MAGED1, MAGED2; NCBI Gene IDs: 4100, 4101, 4102, 4103, 4104, 4105, 4109, 4110, 9500, 9947, 10916, 51438, 266740); major histocompatibility complexes (e.g., HLA-A, HLA-E, HLA-F, HLA-G; NCBI Gene IDs: 3105, 3133, 3134, 3135); major vault protein (MVP, VAULTI; NCBI Gene ID: 9961); MALT1 paracaspase (MALT1; NCBI Gene ID: 10892); MAPK activated protein kinase 2 (MAPKAPK2; NCBI Gene ID: 9261); MAPK interacting serine/threonine kinases (e.g., MKNK1, MKNK2; NCBI Gene IDs: 2872, 8569); matrix metallopeptidases (e.g., MMP1, MMP2, MMP3, MMP7, MMP8, MMP9, MMP10, MMP11, MMP12, MMP13, MMP14, MMP15, MMP16, MMP17, MMP19, MMP20, MMP21, MMP24, MMP25, MMP26, MMP27, MMP28; NCBI Gene IDs: 4312, 4313, 4314, 4316, 4317, 4318, 4319, 4320, 4321, 4322, 4323, 4324, 4325, 4326, 4327, 9313, 10893, 56547, 64066, 64386, 79148, 118856); MCL1 apoptosis regulator, BCL2 family member (MCL1; NCBI Gene ID: 4170); MDM2 proto-oncogene (MDM2; NCBI Gene ID: 4193); MDM4 regulator of p53 (MDM4; BMFS6; NCBI Gene ID: 4194); mechanistic target of rapamycin kinase (MTOR, FRAPI; NCBI Gene ID: 2475); melan-A (MLANA; NCBI Gene ID: 2315); melanocortin receptors (MCIR, MC2R; NCBI Gene IDs: 4157, 4148); MER proto-oncogene, tyrosine kinase (MERTK; NCBI Gene ID: 10461); mesothelin (MSLN; NCBI Gene ID: 10232); MET proto-oncogene, receptor tyrosine kinase (MET, c-Met, HGFR; NCBI Gene ID: 4233); methionyl aminopeptidase 2 (METAP2, MAP2; NCBI Gene ID: 10988); MHC class I polypeptide-related sequences (e.g., MICA, MICB; NCBI Gene IDs: 4277, 100507436); mitogen activated protein kinases (e.g., MAPK1 (ERK2), MAPK3 (ERK1), MAPK8 (JNK1), MAPK9 (JNK2), MAPK10 (JNK3), MAPK11 (p38 beta), MAPK12; NCBI Gene IDs: 5594, 5595, 5599, 5600, 5601, 5602, 819251); mitogen-activated protein kinase kinase kinases (e.g., MAP3K5 (ASK1), MAP3K8 (TPL2, AURA2); NCBI Gene IDs: 4217, 1326); mitogen-activated protein kinase kinase kinase kinase 1 (MAP4K1, HPK1; NCBI Gene ID: 11184); mitogen-activated protein kinase kinases (e.g., MAP2K1 (MEK1), MAP2K2 (MEK2), MAP2K7 (MEK7); NCBI Gene IDs: 5604, 5605, 5609); MPL proto-oncogene, thrombopoietin receptor (MPL; NCBI Gene ID: 4352); mucins (e.g., MUC1 (including splice variants thereof (e.g., including MUC1/A, C, D, X, Y, Z and REP)), MUC5AC, MUC16 (CA125); NCBI Gene IDs: 4582, 4586, 94025); MYC proto-oncogene, bHLH transcription factor (MYC; NCBI Gene ID: 4609); myostatin (MSTN, GDF8; NCBI Gene ID: 2660); myristoylated alanine rich protein kinase C substrate (MARCKS; NCBI Gene ID: 4082); natriuretic peptide receptor 3 (NPR3; NCBI Gene ID: 4883); natural killer cell cytotoxicity receptor 3 ligand 1 (NCR3LG1, B7-H6; NCBI Gene ID: 374383); necdin, MAGE family member (NDN; NCBI Gene ID: 4692); nectin cell adhesion molecules (e.g., NECTIN2 (CD112, PVRL2), NECTIN4 (PVRL4); NCBI Gene IDs: 5819, 81607); neural cell adhesion molecule 1 (NCAM1, CD56; NCBI Gene ID: 4684); neuropilins (e.g., NRP1 (CD304, VEGF165R), NRP2 (VEGF165R2); NCBI Gene IDs: 8828, 8829); neurotrophic receptor tyrosine kinases (e.g., NTRK1 (TRKA), NTRK2 (TRKB), NTRK3 (TRKC); NCBI Gene IDs: 4914, 4915, 4916); NFKB activating protein (NKAP; NCBI Gene ID: 79576); NIMA related kinase 9 (NEK9; NCBI Gene ID: 91754); NLR family pyrin domain containing 3 (NLRP3, NALP3; NCBI Gene ID: 114548); notch receptors (e.g., NOTCH1, NOTCH2, NOTCH3, NOTCH4; NCBI Gene IDs: 4851, 4853, 4854, 4855); NRAS proto-oncogene, GTPase (NRAS; NCBI Gene ID: 4893); nuclear factor kappa B (NFKB1, NFKB2; NCBI Gene IDs: 4790, 4791); nuclear factor, erythroid 2 like 2 (NFE2L2; NRF2; NCBI Gene ID: 4780); nuclear receptor subfamily 4 group A member 1 (NR4A1; NCBI Gene ID: 3164); nucleolin (NCL; NCBI Gene ID: 4691); nucleophosmin 1 (NPM1; NCBI Gene ID: 4869); nucleotide binding oligomerization domain containing 2 (NOD2; NCBI Gene ID: 64127); nudix hydrolase 1 (NUDT1; NCBI Gene ID: 4521); O-6-methylguanine-DNA methyltransferase (MGMT; NCBI Gene ID: 4255); opioid receptor delta 1 (OPRD1; NCBI Gene ID: 4985); ornithine decarboxylase 1 (ODC1; NCBI Gene ID: 4953); C═Oglutarate dehydrogenase (OGDH; NCBI Gene ID: 4967); parathyroid hormone (PTH; NCBI Gene ID: 5741); PD-L1 (CD274; NCBI Gene ID: 29126); periostin (POSTN; NCBI Gene ID: 10631); peroxisome proliferator activated receptors (e.g., PPARA (PPAR alpha), PPARD (PPAR delta), PPARG (PPAR gamma); NCBI Gene IDs: 5465, 5467, 5468); phosphatase and tensin homolog (PTEN; NCBI Gene ID: 5728); phosphatidylinositol-4,5-bisphosphate 3-kinases (PIK3CA (PI3K alpha), PIK3CB (PI3K beta), PIK3CD (PI3K delta), PIK3CG (PI3K gamma); NCBI Gene IDs: 5290, 5291, 5293, 5294); phospholipases (e.g., PLA2G1B, PLA2G2A, PLA2G2D, PLA2G3, PLA2G4A, PLA2G5, PLA2G7, PLA2G10, PLA2G12A, PLA2G12B, PLA2G15; NCBI Gene IDs: 5319, 5320, 5321, 5322, 7941, 8399, 50487, 23659, 26279, 81579, 84647); Pim proto-oncogene, serine/threonine kinases (e.g., PIM1, PIM2, PIM3; NCBI Gene IDs: 5292, 11040, 415116); placenta growth factor (PGF; NCBI Gene ID: 5228); plasminogen activator, urokinase (PLAU, u-PA, ATF; NCBI Gene ID: 5328); platelet derived growth factor receptors (e.g., PDGFRA (CD140A, PDGFR2), FDGFRB (CD140B, PDGFR1); NCBI Gene IDs: 5156, 5159); plexin B1 (PLXNB1; NCBI Gene ID: 5364); poliovirus receptor (PVR) cell adhesion molecule (PVR, CD155; NCBI Gene ID: 5817); polo like kinase 1 (PLK1; NCBI Gene ID: 5347); poly(ADP-ribose) polymerases (e.g., PARP1, PARP2, PARP3; NCBI Gene IDs: 142, 10038, 10039); polycomb protein EED (EED; NCBI Gene ID: 8726); porcupine O-acyltransferase (PORCN; NCBI Gene ID: 64840); PRAME nuclear receptor transcriptional regulator (PRAME; NCBI Gene ID: 23532); premelanosome protein (PMEL; NCBI Gene ID: 6490); progesterone receptor (PGR; NCBI Gene ID: 5241); programmed cell death 1 (PDCD1, PD-1, CD279; NCBI Gene ID: 5133); programmed cell death 1 ligand 2 (PDCD1LG2, CD273, PD-L2; NCBI Gene ID: 80380); prominin 1 (PROM1, CD133; NCBI Gene ID: 8842); promyelocytic leukemia (PML; NCBI Gene ID: 5371); prosaposin (PSAP; NCBI Gene ID: 5660); prostaglandin E receptor 4 (PTGER4; NCBI Gene ID: 5734); prostaglandin E synthase (PTGES; NCBI Gene ID: 9536); prostaglandin-endoperoxide synthases (PTGS1 (COX1), PTGS2 (COX2); NCBI Gene ID: 5742, 5743); proteasome 20S subunit beta 9 (PSMB9; NCBI Gene ID: 5698); protein arginine methyltransferases (e.g., PRMT1, PRMTS; NCBI Gene ID: 3276, 10419); protein kinase N3 (PKN3; NCBI Gene ID: 29941); protein phosphatase 2A (PPP2CA; NCBI Gene ID: 5515); protein tyrosine kinase 7 (inactive) (PTK7; NCBI Gene ID: 5754); protein tyrosine phosphatase receptors (PTPRB (PTPB), PTPRC (CD45R); NCBI Gene ID: 5787, 5788); prothymosin alpha (PTMA; NCBI Gene ID: 5757); purine nucleoside phosphorylase (PNP; NCBI Gene ID: 4860); purinergic receptor P2X 7 (P2RX7; NCBI Gene ID: 5027); PVR related immunoglobulin domain containing (PVRIG, CD112R; NCBI Gene ID: 79037); Raf-1 proto-oncogene, serine/threonine kinase (RAFI, c-Raf; NCBI Gene ID: 5894); RAR-related orphan receptor gamma (RORC; NCBI Gene ID: 6097); ras homolog family member C (RHOC); NCBI Gene ID: 389); Ras homolog, mTORC1 binding (RHEB; NCBI Gene ID: 6009); RB transcriptional corepressor 1 (RB1; NCBI Gene ID: 5925); receptor-interacting serine/threonine protein kinase 1 (RIPK1; NCBI Gene ID: 8737); ret proto-oncogene (RET; NCBI Gene ID: 5979); retinoic acid early transcripts (e.g., RAET1E, RAET1G, RAET1L; NCBI Gene IDs: 135250, 154064, 353091); retinoic acid receptors alpha (e.g., RARA, RARG; NCBI Gene IDs: 5914, 5916); retinoid X receptors (e.g., RXRA, RXRB, RXRG; NCBI Gene IDs: 6256, 6257, 6258); Rho associated coiled-coil containing protein kinases (e.g., ROCK1, ROCK2; NCBI Gene IDs: 6093, 9475); ribosomal protein S6 kinase B1 (RPS6KB1, S6K-beta 1; NCBI Gene ID: 6198); ring finger protein 128 (RNF128, GRAIL; NCBI Gene ID: 79589); ROS proto-oncogene 1, receptor tyrosine kinase (ROS1; NCBI Gene ID: 6098); roundabout guidance receptor 4 (ROBO4; NCBI Gene ID: 54538); RUNX family transcription factor 3 (RUNX3; NCBI Gene ID: 864); S100 calcium binding protein A9 (S100A9; NCBI Gene ID: 6280); secreted frizzled related protein 2 (SFRP2; NCBI Gene ID: 6423); secreted phosphoprotein 1 (SPP1; NCBI Gene ID: 6696); secretoglobin family 1A member 1 (SCGB1A1; NCBI Gene ID: 7356); selectins (e.g., SELE, SELL (CD62L), SELP (CD62); NCBI Gene IDs: 6401, 6402, 6403); semaphorin 4D (SEMA4D; CD100; NCBI Gene ID: 10507); sialic acid binding Ig like lectins (SIGLEC7 (CD328), SIGLEC9 (CD329), SIGLEC10; NCBI Gene ID: 27036, 27180, 89790); signal regulatory protein alpha (SIRPA, CD172A; NCBI Gene ID: 140885); signal transducer and activator of transcription (e.g., STAT1, STAT3, STAT5A, STAT5B; NCBI Gene IDs: 6772, 6774, 6776, 6777); sirtuin-3 (SIRT3; NCBI Gene ID: 23410); signaling lymphocytic activation molecule (SLAM) family members (e.g., SLAMF1 (CD150), SLAMF6 (CD352), SLAMF7 (CD319), SLAMF8 (CD353), SLAMF9; NCBI Gene IDs: 56833, 57823, 89886, 114836); SLIT and NTRK like family member 6 (SLITRK6; NCBI Gene ID: 84189); smoothened, frizzled class receptor (SMO; NCBI Gene ID: 6608); soluble epoxide hydrolase 2 (EPHX2; NCBI Gene ID: 2053); solute carrier family members (e.g., SLC3A2 (CD98), SLC5A5, SLC6A2, SLC10A3, SLC34A2, SLC39A6, SLC43A2 (LAT4), SLC44A4; NCBI Gene IDs: 6520, 6528, 6530, 8273, 10568, 25800, 80736, 124935); somatostatin receptors (e.g., SSTR1, SSTR2, SSTR3, SSTR4, SSTR5; NCBI Gene IDs: 6751, 6752, 6753, 6754, 6755); sonic hedgehog signaling molecule (SHH; NCBI Gene ID: 6469); Sp1 transcription factor (SP1; NCBI Gene ID: 6667); sphingosine kinases (e.g., SPHK1, SPHK2; NCBI Gene IDs: 8877, 56848); sphingosine-1-phosphate receptor 1 (S1PR1, CD363; NCBI Gene ID: 1901); spleen associated tyrosine kinase (SYK; NCBI Gene ID: 6850); splicing factor 3B factor 1 (SF3B1; NCBI Gene ID: 23451); SRC proto-oncogene, non-receptor tyrosine kinase (SRC; NCBI Gene ID: 6714); stabilin 1 (STAB1, CLEVER-1; NCBI Gene ID: 23166); STEAP family member 1 (STEAP1; NCBI Gene ID: 26872); steroid sulfatase (STS; NCBI Gene ID: 412); stimulator of interferon response cGAMP interactor 1 (STING1; NCBI Gene ID: 340061); superoxide dismutase 1 (SOD1, ALS1; NCBI Gene ID: 6647); suppressors of cytokine signaling (SOCS1 (CISH1), SOCS3 (CISH3); NCBI Gene ID: 8651, 9021); synapsin 3 (SYN3; NCBI Gene ID: 8224); syndecan 1 (SDC1, CD138, syndecan; NCBI Gene ID: 6382); synuclein alpha (SNCA, PARK1; NCBI Gene ID: 6622); T cell immunoglobulin and mucin domain containing 4 (TIMD4, SMUCKLER; NCBI Gene ID: 91937); T cell immunoreceptor with Ig and ITIM domains (TIGIT; NCBI Gene ID: 201633); tachykinin receptors (e.g., TACR1, TACR3; NCBI Gene ID: 6869, 6870); TANK binding kinase 1 (TBK1; NCBI Gene ID: 29110); tankyrase (TNKS; NCBI Gene ID: 8658); TATA-box binding protein associated factor, RNA polymerase I subunit B (TAF1B; NCBI Gene ID: 9014); T-box transcription factor T (TBXT; NCBI Gene ID: 6862); TCDD inducible poly(ADP-ribose) polymerase (TIPARP, PAPR7; NCBI Gene ID: 25976); tec protein tyrosine kinase (TEC; NCBI Gene ID: 7006); TEK receptor tyrosine kinase (TEK, CD202B, TIE2; NCBI Gene ID: 7010); telomerase reverse transcriptase (TERT; NCBI Gene ID: 7015); tenascin C (TNC; NCBI Gene ID: 3371); three prime repair exonucleases (e.g., TREX1, TREX2; NCBI Gene ID: 11277, 11219); thrombomodulin (THBD, CD141; NCBI Gene ID: 7056); thymidine kinases (e.g., TK1, TK2; NCBI Gene IDs: 7083, 7084); thymidine phosphorylase (TYMP; NCBI Gene ID: 1890); thymidylate synthase (TYMS; NCBI Gene ID: 7298); thyroid hormone receptor (THRA, THRB; NCBI Gene IDs: 7606, 7608); thyroid stimulating hormone receptor (TSHR; NCBI Gene ID: 7253); TNF superfamily members (e.g., TNFSF4 (OX40L, CD252), TNFSF5 (CD40L), TNFSF7 (CD70), TNFSF8 (CD153, CD30L), TNFSF9 (4-1BB-L, CD137L), TNFSF10 (TRAIL, CD253, APO2L), TNFSF11 (CD254, RANKL2, TRANCE), TNFSF13 (APRIL, CD256, TRAIL2), TNFSF13b (BAFF, BLYS, CD257), TNFSF14 (CD258, LIGHT), TNFSF18 (GITRL); NCBI Gene IDs: 944, 959, 970, 7292, 8600, 8740, 8741, 8743, 8744, 8995); toll like receptors (e.g., TLR1 (CD281), TLR2 (CD282), TLR3 (CD283), TLR4 (CD284), TLR5, TLR6 (CD286), TLR7, TLR8 (CD288), TLR9 (CD289), TLR10 (CD290); NCBI Gene IDs: 7096, 7097, 7098, 7099, 10333, 51284, 51311, 54106, 81793); transferrin (TF; NCBI Gene ID: 7018); transferrin receptor (TFRC, CD71; NCBI Gene ID: 7037); transforming growth factors (e.g., TGFA, TGFB1; NCBI Gene ID: 7039, 7040); transforming growth factor receptors (e.g., TGFBR1, TGFBR2, TGFBR3; NCBI Gene ID: 7046, 7048, 7049); transforming protein E7 (E7; NCBI Gene ID: 1489079); transglutaminase 5 (TGM5; NCBI Gene ID: 9333); transient receptor potential cation channel subfamily V member 1 (TRPV1, VR1; NCBI Gene ID: 7442); transmembrane and immunoglobulin domain containing 2 (TMIGD2, CD28H, IGPR1; NCBI Gene ID: 126259); triggering receptors expressed on myeloid cells (e.g., TREM1 (CD354), TREM2; NCBI Gene ID: 54209, 54210); trophinin (TRO, MAGED3; NCBI Gene ID: 7216); trophoblast glycoprotein (TPBG; NCBI Gene ID: 7162); tryptophan 2,3-dioxygenase (TDO2; NCBI Gene ID: 6999); tryptophan hydroxylases (e.g., TPH1, TPH2; NCBI Gene ID: 7166, 121278); tumor associated calcium signal transducer 2 (TACSTD2, TROP2, EGP1; NCBI Gene ID: 4070); tumor necrosis factor (TNF; NCBI Gene ID: 7124); tumor necrosis factor (TNF) receptor superfamily members (e.g., TNFRSF1A (CD120a), TNFRSF1B (CD120b), TNFRSF4 (OX40), TNFRSF5 (CD40), TNFRSF6 (CD95, FAS receptor), TNFRSF7 (CD27), TNFRSF8 (CD30), TNFRSF9 (CD137, 4-1BB), TNFRSF10A (CD261), TNFRSF10B (TRAIL, DR5, CD262), TNFRSF10C, TNFRSF10D, TNFRSF11A, TNFRSF11B (OPG), TNFRSF12A, TNFRSF13B, TNFR13C (CD268, BAFFR), TNFRSF14 (CD270, LIGHTR), TNFRSF16, TNFRSF17 (CD269, BCMA), TNFRSF18 (GITR, CD357), TNFRSF19, TNFRSF21, TNFRSF25, NCBI Gene IDs: 355, 608, 939, 943, 958, 3604, 4804, 4982, 7132, 7133, 7293, 8718, 8764, 8784, 8792, 8793, 8794, 8795, 8797, 23495, 27242, 51330, 55504); tumor protein p53 (TP53; NCBI Gene ID: 7157); tumor suppressor 2, mitochondrial calcium regulator (TUSC2; NCBI Gene ID: 11334); TYRO3 protein tyrosine kinase (TYRO3; BYK; NCBI Gene ID: 7301); tyrosinase (TYR; NCBI Gene ID: 7299); tyrosine hydroxylase (TH; NCBI Gene ID: 7054); tyrosine kinase with immunoglobulin like and EGF like domains 1 (e.g., TIE1, TIE1; NCBI Gene ID: 7075); tyrosine-protein phosphatase non-receptor type 11 (PTPN11, SHP2; NCBI Gene ID: 5781); ubiquitin conjugating enzyme E2 I (UBE2I, UBC9; NCBI Gene ID: 7329); ubiquitin C-terminal hydrolase L5 (UCHL5; NCBI Gene ID: 51377); ubiquitin specific peptidase 7 (USP7; NCBI Gene ID: 7874); ubiquitin-like modifier activating enzyme 1 (UBA1; NCBI Gene ID: 7317); UL16 binding proteins (e.g., ULBP1, ULBP2, ULBP3; NCBI Gene ID: 79465, 80328, 80328); valosin-containing protein (VCP, CDC48; NCBI Gene ID: 7415); vascular cell adhesion molecule 1 (VCAM1, CD106; NCBI Gene ID: 7412); vascular endothelial growth factors (e.g., VEGFA, VEGFB; NCBI Gene ID: 7422, 7423); vimentin (VIM; NCBI Gene ID: 7431); vitamin D receptor (VDR; NCBI Gene ID: 7421); V-set domain containing T cell activation inhibitor 1 (VTCN1, B7-H4; NCBI Gene ID: 79679); V-set immunoregulatory receptor (VSIR, VISTA, B7-H5; NCBI Gene ID: 64115); WEE1 G2 checkpoint kinase (WEE1; NCBI Gene ID: 7465); WRN RecQ like helicase (WRN; RECQ3; NCBI Gene ID: 7486); WT1 transcription factor (WT1; NCBI Gene ID: 7490); WW domain containing transcription regulator 1 (WWTR1; TAZ; NCBI Gene ID: 25937); X-C motif chemokine ligand 1 (XCL1, ATAC; NCBI Gene ID: 6375); X-C motif chemokine receptor 1 (XCR1, GPR5, CCXCR1; NCBI Gene ID: 2829); Yes1 associated transcriptional regulator (YAP1; NCBI Gene ID: 10413); zeta chain associated protein kinase 70 (ZAP70; NCBI Gene ID: 7535).

In some embodiments, the one or more additional therapeutic agents include, e.g., an agent targeting 5′-nucleotidase ecto (NT5E or CD73; NCBI Gene ID: 4907); adenosine A_{2A} receptor (ADORA2A; NCBI Gene ID: 135); adenosine A_{2B} receptor (ADORA2B; NCBI Gene ID: 136); C-C motif chemokine receptor 8 (CCR8, CDw198; NCBI Gene ID: 1237); cytokine inducible SH2 containing protein (CISH; NCBI Gene ID: 1154); diacylglycerol kinase alpha (DGKA, DAGK, DAGK1 or DGK-alpha; NCBI Gene ID: 1606); fms like tyrosine kinase 3 (FLT3, CD135; NCBI Gene ID: 2322); integrin associated protein (IAP, CD47; NCBI Gene ID: 961); interleukine-2 (IL2; NCBI Gene ID: 3558); interleukine 2 receptor (IL2RA, IL2RB, IL2RG; NCBI Gene IDs: 3559, 3560, 3561); Kirsten rat sarcoma virus (KRAS; NCBI Gene ID: 3845; including mutations, such as KRAS G12C or G12D); mitogen-activated protein kinase kinase kinase kinase 1 (MAP4K1) (also called Hematopoietic Progenitor Kinase 1 (HPK1), NCBI Gene ID: 11184); myeloid cell leukemia sequence 1 apoptosis regulator (MCL1; NCBI Gene ID: 4170); phosphatidylinositol-4,5-bisphosphate 3-kinase, catalytic subunit delta (PIK3CD; NCBI Gene ID: 5293); programmed death-ligand 1 (PD-L1, CD274; NCBI Gene ID 29126); programmed cell death protein 1 (PD-1, CD279; NCBI Gene ID: 5133); proto-oncogen c-KIT (KIT, CD117; NCBI Gene ID: 3815); signal-regulatory protein alpha (SIRPA, CD172A; NCBI Gene ID: 140885); TCDD inducible poly(ADP-ribose) polymerase (TIPARP, PARP7; NCBI Gene ID: 25976); T cell immunoreceptor with Ig and ITIM domains (TIGIT; NCBI Gene ID: 201633); triggering receptor expressed on myeloid cells 1 (TREM1; NCBI Gene ID: 54210); triggering receptor expressed on myeloid cells 2 (TREM2; NCBI Gene ID: 54209); tumor-associated calcium signal transducer 2 (TACSTD2, TROP2, EGP1; NCBI Gene ID: 4070); tumor necrosis factor receptor superfamily, member 4 (TNFRSF4, CD134, OX40; NCBI Gene ID: 7293); tumor necrosis factor receptor superfamily, member 9 (TNFRSF9, 4-1BB, CD137; NCBI Gene ID: 3604); tumor necrosis factor receptor superfamily, member 18 (TNFRSF18, CD357, GITR; NCBI Gene ID: 8784); WRN RecQ like helicase (WRN; NCBI Gene ID: 7486); zinc finger protein Helios (IKZF2; NCBI Gene ID: 22807).

### Illustrative Mechanisms of Action

### Immune Checkpoint Modulators

In some embodiments an compound provided herein is administered with one or more blockers or inhibitors of inhibitory immune checkpoint proteins or receptors and/or with one or more stimulators, activators or agonists of one or more stimulatory immune checkpoint proteins or receptors. Blockade or inhibition of inhibitory immune checkpoints can positively regulate T-cell or NK cell activation and prevent immune escape of cancer cells within the tumor microenvironment. Activation or stimulation of stimulatory immune check points can augment the effect of immune checkpoint inhibitors in cancer therapeutics. In some embodiments, the immune checkpoint proteins or receptors regulate T cell responses (e.g., reviewed in Xu, et al., J Exp Clin Cancer Res. (2018) 37:110). In some embodiments, the immune checkpoint proteins or receptors regulate NK cell responses (e.g., reviewed in Davis, et al., Semin Immunol. (2017) 31:64-75 and Chiossone, et al., Nat Rev Immunol. (2018) 18(11):671-688). Inhibition of regulatory T-cells (Treg) or Treg depletion can alleviate their suppression of antitumor immune responses and have anticancer effects (e.g., reviewed in Plitas and Rudensky, Annu. Rev. Cancer Biol. (2020) 4:459-77; Tanaka and Sakaguchi, Eur. J. Immunol. (2019) 49:1140-1146).

Examples of immune checkpoint proteins or receptors include CD27 (NCBI Gene ID: 939), CD70 (NCBI Gene ID: 970); CD40 (NCBI Gene ID: 958), CD40LG (NCBI Gene ID: 959); CD47 (NCBI Gene ID: 961), SIRPA (NCBI Gene ID: 140885); CD48 (SLAMF2; NCBI Gene ID: 962), transmembrane and immunoglobulin domain containing 2 (TMIGD2, CD28H; NCBI Gene ID: 126259), CD84 (LY9B, SLAMF5; NCBI Gene ID: 8832), CD96 (NCBI Gene ID: 10225), CD160 (NCBI Gene ID: 11126), MS4A1 (CD20; NCBI Gene ID: 931), CD244 (SLAMF4; NCBI Gene ID: 51744); CD276 (B7H3; NCBI Gene ID: 80381); V-set domain containing T cell activation inhibitor 1 (VTCN1, B7H4); V-set immunoregulatory receptor (VSIR, B7H5, VISTA; NCBI Gene ID: 64115); immunoglobulin superfamily member 11 (IGSF11, VSIG3; NCBI Gene ID: 152404); natural killer cell cytotoxicity receptor 3 ligand 1 (NCR3LG1, B7H6; NCBI Gene ID: 374383); HERV-H LTR-associating 2 (HHLA2, B7H7; NCBI Gene ID: 11148); inducible T cell co-stimulator (ICOS, CD278; NCBI Gene ID: 29851); inducible T cell co-stimulator ligand (ICOSLG, B7H2; NCBI Gene ID: 23308); TNF receptor superfamily member 4 (TNFRSF4, OX40; NCBI Gene ID: 7293); TNF superfamily member 4 (TNFSF4, OX40L; NCBI Gene ID: 7292); TNFRSF8 (CD30; NCBI Gene ID: 943), TNFSF8 (CD30L; NCBI Gene ID: 944); TNFRSF10A (CD261, DR4, TRAILR1; NCBI Gene ID: 8797), TNFRSF9 (CD137; NCBI Gene ID: 3604), TNFSF9 (CD137L; NCBI Gene ID: 8744); TNFRSF10B (CD262, DR5, TRAILR2; NCBI Gene ID: 8795), TNFRSF10 (TRAIL; NCBI Gene ID: 8743); TNFRSF14 (HVEM, CD270; NCBI Gene ID: 8764), TNFSF14 (HVEML; NCBI Gene ID: 8740); CD272 (B and T lymphocyte associated (BTLA); NCBI Gene ID: 151888); TNFRSF17 (BCMA, CD269; NCBI Gene ID: 608), TNFSF13B (BAFF; NCBI Gene ID: 10673); TNFRSF18 (GITR; NCBI Gene ID: 8784), TNFSF18 (GITRL; NCBI Gene ID: 8995); MHC class I polypeptide-related sequence A (MICA; NCBI Gene ID: 100507436); MHC class I polypeptide-related sequence B (MICB; NCBI Gene ID: 4277); CD274 (CD274, PDL1, PD-L1; NCBI Gene ID: 29126); programmed cell death 1 (PDCD1, PD1, PD-1; NCBI Gene ID: 5133); cytotoxic T-lymphocyte associated protein 4 (CTLA4, CD152; NCBI Gene ID: 1493); CD80 (B7-1; NCBI Gene ID: 941), CD28 (NCBI Gene ID: 940); nectin cell adhesion molecule 2 (NECTIN2, CD112; NCBI Gene ID: 5819); CD226 (DNAM-1; NCBI Gene ID: 10666); Poliovirus receptor (PVR) cell adhesion molecule (PVR, CD155; NCBI Gene ID: 5817); PVR related immunoglobulin domain containing (PVRIG, CD112R; NCBI Gene ID: 79037); T cell immunoreceptor with Ig and ITIM domains (TIGIT; NCBI Gene ID: 201633); T cell immunoglobulin and mucin domain containing 4 (TIMD4; TIM4; NCBI Gene ID: 91937); hepatitis A virus cellular receptor 2 (HAVCR2, TIMD3, TIM3; NCBI Gene ID: 84868); galectin 9 (LGALS9; NCBI Gene ID: 3965); lymphocyte activating 3 (LAG3, CD223; NCBI Gene ID: 3902); signaling lymphocytic activation molecule family member 1 (SLAMF1, SLAM, CD150; NCBI Gene ID: 6504); lymphocyte antigen 9 (LY9, CD229, SLAMF3; NCBI Gene ID: 4063); SLAM family member 6 (SLAMF6, CD352; NCBI Gene ID: 114836); SLAM family member 7 (SLAMF7, CD319; NCBI Gene ID: 57823); UL16 binding protein 1 (ULBP1; NCBI Gene ID: 80329); UL16 binding protein 2 (ULBP2; NCBI Gene ID: 80328); UL16 binding protein 3 (ULBP3; NCBI Gene ID: 79465); retinoic acid early transcript 1E (RAET1E; ULBP4; NCBI Gene ID: 135250); retinoic acid early transcript 1G (RAET1G; ULBP5; NCBI Gene ID: 353091); retinoic acid early transcript 1L (RAET1L; ULBP6; NCBI Gene ID: 154064); killer cell immunoglobulin like receptor, three Ig domains and long cytoplasmic tail 1 (KIR, CD158E1; NCBI Gene ID: 3811, e.g., lirilumab (IPH-2102, IPH-4102)); killer cell lectin like receptor C1 (KLRC1, NKG2A, CD159A; NCBI Gene ID: 3821); killer cell lectin like receptor K1 (KLRK1, NKG2D, CD314; NCBI Gene ID: 22914); killer cell lectin like receptor C2 (KLRC2, CD159c, NKG2C; NCBI Gene ID: 3822); killer cell lectin like receptor C3 (KLRC3, NKG2E; NCBI Gene ID: 3823); killer cell lectin like receptor C4 (KLRC4, NKG2F; NCBI Gene ID: 8302); killer cell immunoglobulin like receptor, two Ig domains and long cytoplasmic tail 1 (KIR2DL1; NCBI Gene ID: 3802); killer cell immunoglobulin like receptor, two Ig domains and long cytoplasmic tail 2 (KIR2DL2; NCBI Gene ID: 3803); killer cell immunoglobulin like receptor, two Ig domains and long cytoplasmic tail 3 (KIR2DL3; NCBI Gene ID: 3804); killer cell immunoglobulin like receptor, three Ig domains and long cytoplasmic tail 1 (KIR3DL1); killer cell lectin like receptor D1 (KLRD1; NCBI Gene ID: 3824); killer cell lectin like receptor G1 (KLRG1; CLEC15A, MAFA, 2F1; NCBI Gene ID: 10219); sialic acid binding Ig like lectin 7 (SIGLEC7; NCBI Gene ID: 27036); and sialic acid binding Ig like lectin 9 (SIGLEC9; NCBI Gene ID: 27180).

In some embodiments an compound provided herein is administered with one or more blockers or inhibitors of one or more T-cell inhibitory immune checkpoint proteins or receptors. Illustrative T-cell inhibitory immune checkpoint proteins or receptors include CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSFI4 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In some embodiments, the compound provided herein is administered with one or more agonist or activators of one or more T-cell stimulatory immune checkpoint proteins or receptors. Illustrative T-cell stimulatory immune checkpoint proteins or receptors include without limitation CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); CD244 (2B4, SLAMF4), Poliovirus receptor (PVR) cell adhesion molecule (PVR, CD155). See, e.g., Xu, et al., J Exp Clin Cancer Res. (2018) 37:110.

In some embodiments the compound provided herein is administered with one or more blockers or inhibitors of one or more NK-cell inhibitory immune checkpoint proteins or receptors. Illustrative NK-cell inhibitory immune checkpoint proteins or receptors include killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor C1 (KLRC1, NKG2A, CD159A); killer cell lectin like receptor D1 (KLRD1, CD94), killer cell lectin like receptor G1 (KLRG1; CLEC15A, MAFA, 2F1); sialic acid binding Ig like lectin 7 (SIGLEC7); and sialic acid binding Ig like lectin 9 (SIGLEC9). In some embodiments the compound provided herein is administered with one or more agonist or activators of one or more NK-cell stimulatory immune checkpoint proteins or receptors. Illustrative NK-cell stimulatory immune checkpoint proteins or receptors include CD16, CD226 (DNAM-1); CD244 (2B4, SLAMF4); killer cell lectin like receptor K1 (KLRK1, NKG2D, CD314); SLAM family member 7 (SLAMF7). See, e.g., Davis, et al., Semin Immunol. (2017) 31:64-75; Fang, et al., Semin Immunol. (2017) 31:37-54; and Chiossone, et al., Nat Rev Immunol. (2018) 18(11):671-688.

In some embodiments the one or more immune checkpoint inhibitors comprises a proteinaceous (e.g., antibody or fragment thereof, or antibody mimetic) inhibitor of PD-L1 (CD274), PD-1 (PDCD1), CTLA4, or TIGIT. In some embodiments the one or more immune checkpoint inhibitors comprises a small organic molecule inhibitor of PD-L1 (CD274), PD-1 (PDCD1), CTLA4, or TIGIT. In some embodiments the one or more immune checkpoint inhibitors comprises a proteinaceous (e.g., antibody or fragment thereof, or antibody mimetic) inhibitor of LAG3.

Examples of inhibitors of CTLA4 that can be co-administered include ipilimumab, tremelimumab, BMS-986218, AGEN1181, zalifrelimab (AGEN1884), BMS-986249, MK-1308, REGN-4659, ADU-1604, CS-1002 (ipilimumab biosimilar), BCD-145, APL-509, JS-007, BA-3071, ONC-392, AGEN-2041, HBM-4003, JHL-1155, KN-044, CG-0161, ATOR-1144, PBI-5D3H5, BPI-002, as well as multi-specific inhibitors FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), and AK-104 (CTLA4/PD-1).

Examples of inhibitors of PD-L1 (CD274) or PD-1 (PDCD1) that can be co-administered include pembrolizumab, nivolumab, cemiplimab, pidilizumab, AMP-224, MEDI0680 (AMP-514), spartalizumab, atezolizumab, avelumab, durvalumab, BMS-936559, cosibelimab (CK-301), sasanlimab (PF-06801591), tislelizumab (BGB-A317), GLS-010 (WBP-3055), AK-103 (HX-008), AK-105, CS-1003, HLX-10, retifanlimab (MGA-012), BI-754091, balstilimab (AGEN-2034), AMG-404, toripalimab (JS-001), cetrelimab (JNJ-63723283), genolimzumab (CBT-501), LZM-009, prolgolimab (BCD-100), lodapolimab (LY-3300054), SHR-1201, camrelizumab (SHR-1210), Sym-021, budigalimab (ABBV-181), PD1-PIK, BAT-1306, avelumab (MSB0010718C), CX-072, CBT-502, dostarlimab (TSR-042), MSB-2311, JTX-4014, BGB-A333, SHR-1316, CS-1001 (WBP-3155, envafolimab (KN-035), sintilimab (IBI-308), HLX-20, KL-A167, STI-A1014, STI-A1015 (IMC-001), BCD-135, FAZ-053, TQB-2450, MDX1105-01, GS-4224, GS-4416, INCB086550, MAX10181, zimberelimab (AB122), spartalizumab (PDR-001), and compounds disclosed in WO2018195321, WO2020014643, WO2019160882, or WO2018195321, as well as multi-specific inhibitors FPT-155 (CTLA4/PD-L1/CD28), PF-06936308 (PD-1/CTLA4), MGD-013 (PD-1/LAG-3), FS-118 (LAG-3/PD-L1), RO-7247669 (PD-1/LAG-3), MGD-019 (PD-1/CTLA4), KN-046 (PD-1/CTLA4), MEDI-5752 (CTLA4/PD-1), RO-7121661 (PD-1/TIM-3), RG7769 (PD-1/TIM-3), TAK-252 (PD-1/OX40L), XmAb-20717 (PD-1/CTLA4), AK-104 (CTLA4/PD-1), FS-118 (LAG-3/PD-L1), FPT-155 (CTLA4/PD-L1/CD28), GEN-1046 (PD-L1/4-1BB), bintrafusp alpha (M7824; PD-L1/TGFβ-EC domain), CA-170 (PD-L1/VISTA), CDX-527 (CD27/PD-L1), LY-3415244 (TIM3/PDL1), and INBRX-105 (4-1BB/PDL1). In some embodiments the PD-L1 inhibitor is a small molecule inhibitor, such as CA-170, GS-4224, GS-4416 and lazertinib (GNS-1480; PD-L1/EGFR).

Examples of inhibitors of TIGIT that can be co-administered include tiragolumab (RG-6058), vibostolimab, domvanalimab, domvanalimab (AB154), AB308, BMS-986207, AGEN-1307, COM-902, or etigilimab.

Examples of inhibitors of LAG3 that can be co-administered include leramilimab (LAG525).

Inhibition of regulatory T-cell (Treg) activity or Treg depletion can alleviate their suppression of antitumor immune responses and have anticancer effects. See, e.g., Plitas and Rudensky, Annu. Rev. Cancer Biol. (2020) 4:459-77; Tanaka and Sakaguchi, Eur. J. Immunol. (2019) 49:1140-1146. In some embodiments, an compound provided herein is administered with one or more inhibitors of Treg activity or a Treg depleting agent. Treg inhibition or depletion can augment the effect of immune checkpoint inhibitors in cancer therapeutics.

In some embodiments an compound provided herein is administered with one or more Treg inhibitors. In some embodiments the Treg inhibitor can suppress the migration of Tregs into the tumor microenvironment. In some embodiments Treg inhibitor can reduce the immunosuppressive function of Tregs. In some embodiments, the Treg inhibitor can modulate the cellular phenotype and induce production of proinflammatory cytokines. Exemplary Treg inhibitors include without limitation, CCR4 (NCBI Gene ID: 1233) antagonists and degraders of Ikaros zinc-finger proteins (e.g., Ikaros (IKZF1; NCBI Gene ID: 10320), Helios (IKZF2; NCBI Gene ID: 22807), Aiolos (IKZF3; NCBI Gene ID: 22806), and Eos (IKZF4; NCBI Gene ID: 64375).

Examples of Helios degraders that can be co-administered include without limitation I-57 (Novartis) and compounds disclosed in WO2019038717, WO2020012334, WO20200117759, and WO2021101919.

In some embodiments an compound provided herein is administered with one or more Treg depleting agents. In some embodiments the Treg depleting agent is an antibody. In some embodiments the Treg depleting antibody has antibody-dependent cytotoxic (ADCC) activity. In some embodiments, the Treg depleting antibody is Fc-engineered to possess an enhanced ADCC activity. In some embodiments the Treg depleting antibody is an antibody-drug conjugate (ADC). Illustrative targets for Treg depleting agents include without limitation CD25 (IL2RA; NCBI Gene ID: 3559), CTLA4 (CD152; NCBI Gene ID: 1493); GITR (TNFRSF18; NCBI Gene ID: 8784); 4-1BB (CD137; NCBI Gene ID: 3604), OX-40 (CD134; NCBI Gene ID: 7293), LAG3 (CD223; NCBI Gene ID: 3902), TIGIT (NCBI Gene ID: 201633), CCR4 (NCBI Gene ID: 1233), and CCR8 (NCBI Gene ID: 1237).

In some embodiments the Treg inhibitor or Treg depleting agent that can be co-administered comprises an antibody or antigen-binding fragment thereof that selectively binds to a cell surface receptor selected from the group consisting of C-C motif chemokine receptor 4 (CCR4), C-C motif chemokine receptor 7 (CCR7), C-C motif chemokine receptor 8 (CCR8), C-X-C motif chemokine receptor 4 (CXCR4; CD184), TNFRSF4 (OX40), TNFRSF18 (GITR, CD357), TNFRSF9 (4-1BB, CD137), cytotoxic T-lymphocyte associated protein 4 (CTLA4, CD152), programmed cell death 1 (PDCD1, PD-1), Sialyl Lewis x (CD15s), CD27, ectonucleoside triphosphate diphosphohydrolase 1 (ENTPD1; CD39), protein tyrosine phosphatase receptor type C (PTPRC; CD45), neural cell adhesion molecule 1 (NCAM1; CD56), selectin L (SELL; CD62L), integrin subunit alpha E (ITGAE; CD103), interleukin 7 receptor (IL7R; CD127), CD40 ligand (CD40LG; CD154), folate receptor alpha (FOLR1), folate receptor beta (FOLR2), leucine rich repeat containing 32 (LRRC32; GARP), IKAROS family zinc finger 2 (IKZF2; HELIOS), inducible T cell costimulatory (ICOS; CD278), lymphocyte activating 3 (LAG3; CD223), transforming growth factor beta 1 (TGFB1), hepatitis A virus cellular receptor 2 (HAVCR2; CD366; TIM3), T cell immunoreceptor with Ig and ITIM domains (TIGIT), TNF receptor superfamily member 1B (CD120b; TNFR2), IL2RA (CD25) or a combination thereof.

Examples of Treg depleting anti-CCR8 antibodies that can be administered include without limitation JTX-1811 (GS-1811) (Jounce Therapeutics, Gilead Sciences), BMS-986340 (Bristol Meyers Squibb), S-531011 (Shionogi), FPA157 (Five Prime Therapeutics), SRF-114 (Surface Oncology), HBM1022 (Harbor BioMed), IO-1 (Oncurious), and antibodies disclosed in WO2021163064, WO2020138489, and WO2021152186.

Examples of Treg depleting anti-CCR4 antibodies that can be administered include mogamulizumab.

Inhibiting, depleting, or reprogramming of non-stimulatory myeloid cells in the tumor microenvironment can enhance anti-cancer immune responses (see, e.g., Binnewies et al., Nat. Med. (2018) 24(5): 541-550; WO2016049641). Illustrative targets for depleting or reprogramming non-stimulatory myeloid cells include triggering receptors expressed on myeloid cells, TREM-1 (CD354, NCBI Gene ID: 54210) and TREM-2 (NCBI Gene ID: 54209). In some embodiments an compound provided herein is administered with one or more myeloid cell depleting or reprogramming agents, such as an anti-TREM-1 antibody (e.g. PY159; antibodies disclosed in WO2019032624) or an anti-TREM-2 antibody (e.g., PY314; antibodies disclosed in WO2019118513).

### Cluster of Differentiation Agonists or Activators

In some embodiments, the compound provided herein is administered with agents targeting a cluster of differentiation (CD) marker. Exemplary CD marker targeting agents that can be co-administered include without limitation A6, AD-IL24, neratinib, tucatinib (ONT 380), mobocertinib (TAK-788), tesevatinib, trastuzumab (HERCEPTIN®), trastuzumab biosimimar (HLX-02), margetuximab, BAT-8001, pertuzumab (Perjeta), pegfilgrastim, RG6264, zanidatamab (ZW25), cavatak, AIC-100, tagraxofusp (SL-401), HLA-A2402/HLA-A0201 restricted epitope peptide vaccine, dasatinib, imatinib, nilotinib, sorafenib, lenvatinib mesylate, ofranergene obadenovec, cabozantinib malate, AL-8326, ZLJ-33, KBP-7018, sunitinib malate, pazopanib derivatives, AGX-73, rebastinib, NMS-088, lucitanib hydrochloride, midostaurin, cediranib, dovitinib, sitravatinib, tivozanib, masitinib, regorafenib, olverembatinib dimesylate (HQP-1351), cabozantinib, ponatinib, and famitinib L-malate, CX-2029 (ABBV-2029), SCB-313, CA-170, COM-701, CDX-301, GS-3583, asunercept (APG-101), APO-010, and compounds disclosed in WO2016196388, WO2016033570, WO2015157386, WO199203459, WO199221766, WO2004080462, WO2005020921, WO2006009755, WO2007078034, WO2007092403, WO2007127317, WO2008005877, WO2012154480, WO2014100620, WO2014039714, WO2015134536, WO2017167182, WO2018112136, WO2018112140, WO2019155067, WO2020076105, PCT/US2019/063091, WO19173692, WO2016179517, WO2017096179, WO2017096182, WO2017096281, WO2018089628, WO2017096179, WO2018089628, WO2018195321, WO2020014643, WO2019160882, WO2018195321, WO200140307, WO2002092784, WO2007133811, WO2009046541, WO2010083253, WO2011076781, WO2013056352, WO2015138600, WO2016179399, WO2016205042, WO2017178653, WO2018026600, WO2018057669, WO2018107058, WO2018190719, WO2018210793, WO2019023347, WO2019042470, WO2019175218, WO2019183266, WO2020013170, WO2020068752, Cancer Discov. 2019 Jan. 9(1):8; and Gariepy J., et al. 106th Annu Meet Am Assoc Immunologists (AAI) (May 9-13, San Diego, 2019, Abst 71.5).

In some embodiments the CD marker targeting agent that can be co-administered include small molecule inhibitors, such as PBF-1662, BLZ-945, pemigatinib (INCB-054828), rogaratinib (BAY-1163877), AZD4547, roblitinib (FGF-401), quizartinib dihydrochloride, SX-682, AZD-5069, PLX-9486, avapritinib (BLU-285), ripretinib (DCC-2618), imatinib mesylate, JSP-191, BLU-263, CD117-ADC, AZD3229, telatinib, vorolanib, GO-203-2C, AB-680, PSB-12379, PSB-12441, PSB-12425, CB-708, HM-30181A, motixafortide (BL-8040), LY2510924, burixafor (TG-0054), X4P-002, mavorixafor (X4P-001-IO), plerixafor, CTX-5861, or REGN-5678 (PSMA/CD28).

In some embodiments the CD marker targeting agent that can be co-administered include small molecule agonists, such as interleukin 2 receptor subunit gamma, eltrombopag, rintatolimod, poly-ICLC (NSC-301463), Riboxxon, Apoxxim, RIBOXXIM®, MCT-465, MCT-475, G100, PEPA-10, eftozanermin alfa (ABBV-621), E-6887, motolimod, resiquimod, selgantolimod (GS-9688), VTX-1463, NKTR-262, AST-008, CMP-001, cobitolimod, tilsotolimod, litenimod, MGN-1601, BB-006, IMO-8400, IMO-9200, agatolimod, DIMS-9054, DV-1079, lefitolimod (MGN-1703), CYT-003, and PUL-042.

In some embodiments the CD marker targeting agent that can be co-administered include antibodies, such as tafasitamab (MOR208; MorphoSys AG), Inebilizumab (MEDI-551), obinutuzumab, IGN-002, rituximab biosimilar (PF-05280586), varlilumab (CDX-1127), AFM-13 (CD16/CD30), AMG330, otlertuzumab (TRU-016), isatuximab, felzartamab (MOR-202), TAK-079, TAK573, daratumumab (DARZALEX®), TTX-030, selicrelumab (RG7876), APX-005M, ABBV-428, ABBV-927, mitazalimab (JNJ-64457107), lenziluma, alemtuzuma, emactuzumab, AMG-820, FPA-008 (cabiralizumab), PRS-343 (CD-137/Her2), AFM-13 (CD16/CD30), belantamab mafodotin (GSK-2857916), AFM26 (BCMA/CD16A), simlukafusp alfa (RG7461), urelumab, utomilumab (PF-05082566), AGEN2373, ADG-106, BT-7480, PRS-343 (CD-137/HER2), FAP-4-IBBL (4-1BB/FAP), ramucirumab, CDX-0158, CDX-0159 and FSI-174, relatlimab (ONO-4482), LAG-525, MK-4280, fianlimab (REGN-3767), INCAGN2385, encelimab (TSR-033), atipotuzumab, BrevaRex (Mab-AR-20.5), MEDI-9447 (oleclumab), CPX-006, IPH-53, BMS-986179, NZV-930, CPI-006, PAT-SCI, lirilumab (IPH-2102), lacutamab (IPH-4102), monalizumab, BAY-1834942, NEO-201 (CEACAM 5/6), Iodine (131I) apamistamab (131I-BC8 (lomab-B)), MEDI0562 (tavolixizumab), GSK-3174998, INCAGN1949, BMS-986178, GBR-8383, ABBV-368, denosumab, BION-1301, MK-4166, INCAGN-1876, TRX-518, BMS-986156, MK-1248, GWN-323, CTB-006, INBRX-109, GEN-1029, pepinemab (VX-15), vopratelimab (JTX-2011), GSK3359609, cobolimab (TSR-022), MBG-453, INCAGN-2390, and compounds disclosed in WO 2017096179, WO2017096276, WO2017096189, and WO2018089628.

In some embodiments the CD marker targeting agent that can be co-administered include cell therapies, such as CD19-ARTEMIS, TBI-1501, CTL-119 huCART-19 T cells, 1 iso-cel, lisocabtagene maraleucel (JCAR-017), axicabtagene ciloleucel (KTE-C19, Yescarta®), axicabtagene ciloleucel (KTE-X19), U.S. Pat. Nos. 7,741,465, 6,319,494, UCART-19, tabelecleucel (EBV-CTL), T tisagenlecleucel-T (CTL019), CD19CAR-CD28-CD3zeta-EGFRt-expressing T cells, CD19/4-1BBL armored CAR T cell therapy, C-CAR-011, CIK-CAR.CD19, CD19CAR-28-zeta T cells, PCAR-019, MatchCART, DSCAR-01, IM19 CAR-T, TC-110, anti-CD19 CAR T-cell therapy (B-cell acute lymphoblastic leukemia, Universiti Kebangsaan Malaysia), anti-CD19 CAR T-cell therapy (acute lymphoblastic leukemia/Non-Hodgkin's lymphoma, University Hospital Heidelberg), anti-CD19 CAR T-cell therapy (silenced IL-6 expression, cancer, Shanghai Unicar-Therapy Bio-medicine Technology), MB-CART2019.1 (CD19/CD20), GC-197 (CD19/CD7), CLIC-1901, ET-019003, anti-CD19-STAR-T cells, AVA-001, BCMA-CD19 cCAR (CD19/APRIL), ICG-134, ICG-132 (CD19/CD20), CTA-101, WZTL-002, dual anti-CD19/anti-CD20 CAR T-cells (chronic lymphocytic leukemia/B-cell lymphomas), HY-001, ET-019002, YTB-323, GC-012 (CD19/APRIL), GC-022 (CD19/CD22), CD19CAR-CD28-CD3zeta-EGFRt-expressing Tn/mem, UCAR-011, ICTCAR-014, GC-007F, PTG-01, CC-97540, GC-007G, TC-310, GC-197, tisagenlecleucel-T, CART-19, tisagenlecleucel (CTL-019)), anti-CD20 CAR T-cell therapy (non-Hodgkin's lymphoma), MB-CART2019.1 (CD19/CD20), WZTL-002 dual anti-CD19/anti-CD20 CAR-T cells, ICG-132 (CD19/CD20), ACTR707 ATTCK-20, PBCAR-20A, LB-1905, CIK-CAR.CD33, CD33CART, dual anti-BCMA/anti-CD38 CAR T-cell therapy, CART-ddBCMA, MB-102, IM-23, JEZ-567, UCART-123, PD-1 knockout T cell therapy (esophageal cancer/NSCLC), ICTCAR-052, Tn MUC-1 CAR-T, ICTCAR-053, PD-1 knockout T cell therapy (esophageal cancer/NSCLC), AUTO-2, anti-BCMA CAR T-cell therapy, Descartes-011, anti-BCMA/anti-CD38 CAR T-cell therapy, CART-ddBCMA, BCMA-CSi cCAR, CYAD-01 (NKG2D LIGAND MODULATOR), KD-045, PD-L1 t-haNK, BCMA-CSi cCAR, MEDI5083, anti-CD276 CART, and therapies disclosed in WO2012079000 or WO2017049166.

### Cluster of Differentiation 47 (CD47) Inhibitors

In some embodiments the compound provided herein is administered with an inhibitor of CD47 (IAP, MER6, OA3; NCBI Gene ID: 961). Examples of CD47 inhibitors include anti-CD47 mAbs (Vx-1004), anti-human CD47 mAbs (CNTO-7108), CC-90002, CC-90002-ST-001, humanized anti-CD47 antibody or a CD47-blocking agent, NI-1701, NI-1801, RCT-1938, ALX148, SG-404, SRF-231, and TTI-621. Additional exemplary anti-CD47 antibodies include CC-90002, magrolimab (Hu5F9-G4), AO-176 (Vx-1004), letaplimab (IBI-188) (letaplimab), lemzoparlimab (TJC-4), SHR-1603, HLX-24, LQ-001, IMC-002, ZL-1201, IMM-01, B6H12, GenSci-059, TAY-018, PT-240, 1F8-GMCSF, SY-102, KD-015, ALX-148, AK-117, TTI-621, TTI-622, or compounds disclosed in WO199727873, WO199940940, WO2002092784, WO2005044857, WO2009046541, WO2010070047, WO2011143624, WO2012170250, WO2013109752, WO2013119714, WO2014087248, WO2015191861, WO2016022971, WO2016023040, WO2016024021, WO2016081423, WO2016109415, WO2016141328, WO2016188449, WO2017027422, WO2017049251, WO2017053423, WO2017121771, WO2017194634, WO2017196793, WO2017215585, WO2018075857, WO2018075960, WO2018089508, WO2018095428, WO2018137705, WO2018233575, WO2019027903, WO2019034895, WO2019042119, WO2019042285, WO2019042470, WO2019086573, WO2019108733, WO2019138367, WO2019144895, WO2019157843, WO2019179366, WO2019184912, WO2019185717, WO2019201236, WO2019238012, WO2019241732, WO2020019135, WO2020036977, WO2020043188, and WO2020009725. In some embodiments, the CD47 inhibitor is RRx-001, DSP-107, VT-1021, IMM-02, SGN-CD47M, or SIRPa-Fc-CD40L (SL-172154). In some embodiments the CD47 inhibitor is magrolimab.

In some embodiments, the CD47 inhibitor is a bispecific antibodies targeting CD47, such as IBI-322 (CD47/PD-L1), IMM-0306 (CD47/CD20), TJ-L1C4 (CD47/PD-L1), HX-009 (CD47/PD-1), PMC-122 (CD47/PD-L1), PT-217, (CD47/DLL3), IMM-26011 (CD47/FLT3), IMM-0207 (CD47/VEGF), IMM-2902 (CD47/HER2), BH29xx (CD47/PD-L1), IMM-03 (CD47/CD20), IMM-2502 (CD47/PD-L1), HMBD-004B (CD47/BCMA), HMBD-004A (CD47/CD33), TG-1801 (NI-1701), or NI-1801.

### SIRPα Targeting Agents

In some embodiments the compound provided herein is administered with a SIRPα targeting agent (NCBI Gene ID: 140885; UniProt P78324). Examples of SIRPα targeting agents include SIRPα inhibitors, such as AL-008, RRx-001, and CTX-5861, and anti-SIRPα antibodies, such as FSI-189 (GS-0189), ES-004, BI-765063, ADU1805, CC-95251, Q-1801 (SIRPα/PD-L1). Additional SIRPα-targeting agents of use are described, for example, in WO200140307, WO2002092784, WO2007133811, WO2009046541, WO2010083253, WO2011076781, WO2013056352, WO2015138600, WO2016179399, WO2016205042, WO2017178653, WO2018026600, WO2018057669, WO2018107058, WO2018190719, WO2018210793, WO2019023347, WO2019042470, WO2019175218, WO2019183266, WO2020013170 and WO2020068752.

### FLT3R Agonists

In some embodiments the compound provided herein is administered with a FLT3R agonist. In some embodiments, the compound provided herein is administered with a FLT3 ligand. In some embodiments, the compound provided herein is administered with a FLT3L-Fc fusion protein, e.g., as described in WO2020263830. In some embodiments the compound provided herein is administered with GS-3583 or CDX-301. In some embodiments the compound provided herein is administered with GS-3583.

### TNF Receptor Superfamily (TNFRSF) Member Agonists or Activators

In some embodiments, the compound provided herein is administered with an agonist of one or more TNF receptor superfamily (TNFRSF) members, e.g., an agonist of one or more of TNFRSF1A (NCBI Gene ID: 7132), TNFRSF1B (NCBI Gene ID: 7133), TNFRSF4 (OX40, CD134; NCBI Gene ID: 7293), TNFRSF5 (CD40; NCBI Gene ID: 958), TNFRSF6 (FAS, NCBI Gene ID: 355), TNFRSF7 (CD27, NCBI Gene ID: 939), TNFRSF8 (CD30, NCBI Gene ID: 943), TNFRSF9 (4-1BB, CD137, NCBI Gene ID: 3604), TNFRSF10A (CD261, DR4, TRAILR1, NCBI Gene ID: 8797), TNFRSF10B (CD262, DR5, TRAILR2, NCBI Gene ID: 8795), TNFRSF10C (CD263, TRAILR3, NCBI Gene ID: 8794), TNFRSF10D (CD264, TRAILR4, NCBI Gene ID: 8793), TNFRSF11A (CD265, RANK, NCBI Gene ID: 8792), TNFRSF11B (NCBI Gene ID: 4982), TNFRSF12A (CD266, NCBI Gene ID: 51330), TNFRSF13B (CD267, NCBI Gene ID: 23495), TNFRSF13C (CD268, NCBI Gene ID: 115650), TNFRSF16 (NGFR, CD271, NCBI Gene ID: 4804), TNFRSF17 (BCMA, CD269, NCBI Gene ID: 608), TNFRSF18 (GITR, CD357, NCBI Gene ID: 8784), TNFRSF19 (NCBI Gene ID: 55504), TNFRSF21 (CD358, DR6, NCBI Gene ID: 27242), and TNFRSF25 (DR3, NCBI Gene ID: 8718).

Example anti-TNFRSF4 (OX40) antibodies that can be co-administered include MEDI6469, MEDI6383, tavolixizumab (MEDI0562), MOXR0916, PF-04518600, RG-7888, GSK-3174998, INCAGN1949, BMS-986178, GBR-8383, ABBV-368, and those described in WO2016179517, WO2017096179, WO2017096182, WO2017096281, and WO2018089628.

Example anti-TNFRSF5 (CD40) antibodies that can be co-administered include RG7876, SEA-CD40, APX-005M, and ABBV-428. In some embodiments, the anti-TNFRSF7 (CD27) antibody varlilumab (CDX-1127) is co-administered.

Example anti-TNFRSF9 (4-1BB, CD137) antibodies that can be co-administered include urelumab, utomilumab (PF-05082566), AGEN-2373, and ADG-106.

In some embodiments the anti-TNFRSF17 (BCMA) antibody GSK-2857916 is co-administered.

Example anti-TNFRSF18 (GITR) antibodies that can be co-administered include MEDI1873, FPA-154, INCAGN-1876, TRX-518, BMS-986156, MK-1248, GWN-323, and those described in WO2017096179, WO2017096276, WO2017096189, and WO2018089628. In some embodiments, an antibody, or fragment thereof, co-targeting TNFRSF4 (OX40) and TNFRSF18 (GITR) is co-administered. Such antibodies are described, e.g., in WO2017096179 and WO2018089628.

Bi-specific antibodies targeting TNFRSF family members that can be co-administered include PRS-343 (CD-137/HER2), AFM26 (BCMA/CD16A), AFM-13 (CD16/CD30), odronextamab (REGN-1979; CD20/CD3), AMG-420 (BCMA/CD3), INHIBRX-105 (4-1BB/PDL1), FAP-4-IBBL (4-1BB/FAP), plamotamab (XmAb-13676; CD3/CD20), RG-7828 (CD20/CD3), CC-93269 (CD3/BCMA), REGN-5458 (CD3/BCMA), and IMM-0306 (CD47/CD20).

### Bi-Specific T-Cell Engagers

In some embodiments compound provided herein is administered with a bi-specific T-cell engager (e.g., not having an Fc) or an anti-CD3 bi-specific antibody (e.g., having an Fc). Illustrative anti-CD3 bi-specific antibodies or BiTEs that can be co-administered include duvortuxizumab (JNJ-64052781; CD19/CD3), AMG-211 (CEA/CD3), AMG-160 (PSMA/CD3), RG7802 (CEA/CD3), ERY-974 (CD3/GPC3), PF-06671008 (Cadherins/CD3), APV0436 (CD123/CD3), flotetuzumab (CD123/CD3), odronextamab (REGN-1979; CD20/CD3), MCLA-117 (CD3/CLEC12A), JNJ-0819 (heme/CD3), JNJ-7564 (CD3/heme), AMG-757 (DLL3-CD3), AMG-330 (CD33/CD3), AMG-420 (BCMA/CD3), AMG-427 (FLT3/CD3), AMG-562 (CD19/CD3), AMG-596 (EGFRvIII/CD3), AMG-673 (CD33/CD3), AMG-701 (BCMA/CD3), AMG-757 (DLL3/CD3), AMG-211 (CEA/CD3), blinatumomab (CD19/CD3), huGD2-BsAb (CD3/GD2), ERY974 (GPC3/CD3), GEMoab (CD3/PSCA), RG6026 (CD20/CD3), RG6194 (HER2/CD3), PF-06863135 (BCMA/CD3), SAR440234 (CD3/CDw123), JNJ-9383 (MGD-015), AMG-424 (CD38/CD3), tidutamab (XmAb-18087 (SSTR2/CD3)), JNJ-63709178 (CD123/CD3), MGD-007 (CD3/gpA33), MGD-009 (CD3/B7H3), IMCgp100 (CD3/gp100), XmAb-14045 (CD123/CD3), XmAb-13676 (CD3/CD20), tidutamab (XmAb-18087; SSTR2/CD3), catumaxomab (CD3/EpCAM), REGN-4018 (MUC16/CD3), mosunetuzumab (RG-7828; CD20/CD3), CC-93269 (CD3/BCMA), REGN-5458 (CD3/BCMA), GRB-1302 (CD3/Erbb2), GRB-1342 (CD38/CD3), GEM-333 (CD3/CD33). As appropriate, the anti-CD3 binding bi-specific molecules may or may not have an Fc. Illustrative bi-specific T-cell engagers that can be co-administered target CD3 and a tumor-associated antigen as described herein, including, e.g., CD19 (e.g., blinatumomab); CD33 (e.g., AMG330); CEA (e.g., MEDI-565); receptor tyrosine kinase-like orphan receptor 1 (ROR1) (Gohil, et al., Oncoimmunology. (2017) May 17; 6(7):e1326437); PD-L1 (Horn, et al., Oncotarget. 2017 Aug. 3; 8(35):57964-57980); and EGFRvIII (Yang, et al., Cancer Lett. 2017 Sep. 10; 403:224-230).

### Bi-and Tri-Specific Natural Killer (NK)-Cell Engagers

In some embodiments the compound provided herein is administered with a bi-specific NK-cell engager (BiKE) or a tri-specific NK-cell engager (TriKE) (e.g., not having an Fc) or bi-specific antibody (e.g., having an Fc) against an NK cell activating receptor, e.g., CD16A, C-type lectin receptors (CD94/NKG2C, NKG2D, NKG2E/H and NKG2F), natural cytotoxicity receptors (NKp30, NKp44 and NKp46), killer cell C-type lectin-like receptor (NKp65, NKp80), Fc receptor FcγR (which mediates antibody-dependent cell cytotoxicity), SLAM family receptors (e.g., 2B4, SLAM6 and SLAM7), killer cell immunoglobulin-like receptors (KIR) (KIR-2DS and KIR-3DS), DNAM-1 and CD137 (41BB). Illustrative anti-CD16 bi-specific antibodies, BiKEs or TriKEs that can be co-administered include AFM26 (BCMA/CD16A) and AFM-13 (CD16/CD30). As appropriate, the anti-CD16 binding bi-specific molecules may or may not have an Fc. Illustrative bi-specific NK-cell engagers that can be co-administered target CD16 and one or more tumor-associated antigens as described herein, including, e.g., CD19, CD20, CD22, CD30, CD33, CD123, EGFR, EpCAM, ganglioside GD2, HER2/neu, HLA Class II and FOLR1. BiKEs and TriKEs are described, e.g., in Felices, et al., Methods Mol Biol. (2016) 1441:333-346; Fang, et al., Semin Immunol. (2017) 31:37-54.

### MCL1 Apoptosis Regulator, BCL2 Family Member (MCL1) Inhibitors

In some embodiments the compound provided herein is administered with an inhibitor of MCL1 apoptosis regulator, BCL2 family member (MCL1, TM; EAT; MCL1L; MCL1S; Mcl-1; BCL2L3; MCL1-ES; bcl2-L-3; mcl1/EAT; NCBI Gene ID: 4170). Examples of MCL1 inhibitors include tapotoclax (AMG-176), AMG-397, 5-64315, AZD-5991, 483-LM, A-1210477, UMI-77, JKY-5-037, PRT-1419, GS-9716, and those described in WO2018183418, WO2016033486, and WO2017147410.

### SHP2 Inhibitors

In some embodiments compound provided herein is administered with an inhibitor of protein tyrosine phosphatase non-receptor type 11 (PTPN11; BPTP3, CFC, JMML, METCDS, NS1, PTP-1D, PTP2C, SH-PTP2, SH-PTP3, SHP2; NCBI Gene ID: 5781). Examples of SHP2 inhibitors include TNO155 (SHP-099), RMC-4550, JAB-3068, RMC-4630, and those described in WO2018172984 and WO2017211303.

### Hematopoietic Progenitor Kinase 1 (HPK1) Inhibitors and Degraders

In some embodiments, the compound provided herein is administered with an inhibitor of mitogen-activated protein kinase kinase kinase kinase 1 (MAP4K1, HPK1; NCBI Gene ID: 11184). Examples of Hematopoietic Progenitor Kinase 1 (HPK1) inhibitors include without limitation, those described in WO2020092621, WO2018183956, WO2018183964, WO2018167147, WO2018049152, WO2020092528, WO2016205942, WO2016090300, WO2018049214, WO2018049200, WO2018049191, WO2018102366, WO2018049152, and WO2016090300.

### Apoptosis Signal-Regulating Kinase (ASK) Inhibitors

In some embodiments the compound provided herein is administered with an ASK inhibitor, e.g., mitogen-activated protein kinase kinase kinase 5 (MAP3K5; ASK1, MAPKKK5, MEKK5; NCBI Gene ID: 4217). Examples of ASK1 inhibitors include those described in WO2011008709 (Gilead Sciences) and WO 2013112741 (Gilead Sciences).

### Bruton Tyrosine Kinase (BTK) Inhibitors

In some embodiments the compound provided herein is administered with an inhibitor of Bruton tyrosine kinase (BTK, AGMX1, AT, ATK, BPK, IGHD3, IMD1, PSCTK1, XLA; NCBI Gene ID: 695). Examples of BTK inhibitors include (S)-6-amino-9-(1-(but-2-ynoyl)pyrrolidin-3-yl)-7-(4-phenoxyphenyl)-7H-purin-8(9H)-one, acalabrutinib (ACP-196), zanubrutinib (BGB-3111), CB988, HM71224, ibrutinib, M-2951 (evobrutinib), M7583, tirabrutinib (ONO-4059), PRN-1008, spebrutinib (CC-292), TAK-020, vecabrutinib, ARQ-531, SHR-1459, DTRMWXHS-12, PCI-32765, and TAS-5315.

### Cyclin-Dependent Kinase (CDK) Inhibitors

In some embodiments the compound provided herein is administered with an inhibitor of cyclin dependent kinase 1 (CDK1, CDC2; CDC28A; P34CDC2; NCBI Gene ID: 983); cyclin dependent kinase 2 (CDK2, CDKN2; p33(CDK2); NCBI Gene ID: 1017); cyclin dependent kinase 3 (CDK3, NCBI Gene ID: 1018); cyclin dependent kinase 4 (CDK4, CMM3; PSK-J3; NCBI Gene ID: 1019); cyclin dependent kinase 6 (CDK6, MCPH12; PLSTIRE; NCBI Gene ID: 1021); cyclin dependent kinase 7 (CDK7, CAK; CAK1; HCAK; MO15; STK1; CDKN7; p39MO15; NCBI Gene ID: 1022), or cyclin dependent kinase 9 (CDK9, TAK; C-2k; CTK1; CDC2L4; PITALRE; NCBI Gene ID: 1025). Inhibitors of CDK 1, 2, 3, 4, 6, 7 and/or 9, include abemaciclib, alvocidib (HMR-1275, flavopiridol), AT-7519, dinaciclib, ibrance, FLX-925, LEE001, palbociclib, samuraciclib, ribociclib, rigosertib, selinexor, UCN-01, SY1365, CT-7001, SY-1365, G1T38, milciclib, trilaciclib, simurosertib hydrate (TAK931), and TG-02.

### Discoidin Domain Receptor (DDR) Inhibitors

In some embodiments the compound provided herein is combined with an inhibitor of discoidin domain receptor tyrosine kinase 1 (DDR1, CAK, CD167, DDR, EDDR1, HGK2, MCK10, NEP, NTRK4, PTK3, PTK3A, RTK6, TRKE; NCBI Gene ID: 780); and/or discoidin domain receptor tyrosine kinase 2 (DDR2, MIG20a, NTRKR3, TKT, TYRO10, WRCN; NCBI Gene ID: 4921). Examples of DDR inhibitors include dasatinib and those disclosed in WO2014/047624 (Gilead Sciences), US 2009-0142345 (Takeda Pharmaceutical), US 2011-0287011 (Oncomed Pharmaceuticals), WO 2013/027802 (Chugai Pharmaceutical), and WO2013/034933 (Imperial Innovations).

### Targeted E3 Ligase Ligand Conjugates

In some embodiments the compound provided herein is administered with a targeted E3 ligase ligand conjugate. Such conjugates have a target protein binding moiety and an E3 ligase binding moiety (e.g., an inhibitor of apoptosis protein (IAP) (e.g., XIAP, c-IAP1, c-IAP2, NIL-IAP, Bruce, and surviving) E3 ubiquitin ligase binding moiety, Von Hippel-Lindau E3 ubiquitin ligase (VHL) binding moiety, a cereblon E3 ubiquitin ligase binding moiety, mouse double minute 2 homolog (MDM2) E3 ubiquitin ligase binding moiety), and can be used to promote or increase the degradation of targeted proteins, e.g., via the ubiquitin pathway. In some embodiments the targeted E3 ligase ligand conjugates comprise a targeting or binding moiety that targets or binds a protein described herein, and an E3 ligase ligand or binding moiety. In some embodiments the targeted E3 ligase ligand conjugates comprise a targeting or binding moiety that targets or binds a protein selected from Cbl proto-oncogene B (CBLB; Cbl-b, Nbla00127, RNF56; NCBI Gene ID: 868) and hypoxia inducible factor 1 subunit alpha (HIF1A; NCBI Gene ID: 3091). In some embodiments the targeted E3 ligase ligand conjugates comprise a kinase inhibitor (e.g., a small molecule kinase inhibitor, e.g., of BTK and an E3 ligase ligand or binding moiety. See, e.g., WO2018098280. In some embodiments the targeted E3 ligase ligand conjugates comprise a binding moiety targeting or binding to Interleukin-1 (IL-1) Receptor-Associated Kinase-4 (IRAK-4); Rapidly Accelerated Fibrosarcoma (RAF, such as c-RAF, A-RAF and/or B-RAF), c-Met/p38, or a BRD protein; and an E3 ligase ligand or binding moiety. See, e.g., WO2019099926, WO2018226542, WO2018119448, WO2018223909, WO2019079701. Additional targeted E3 ligase ligand conjugates that can be co-administered are described, e.g., in WO2018237026, WO2019084026, WO2019084030, WO2019067733, WO2019043217, WO2019043208, and WO2018144649.

### Histone Deacetylase (HDAC) Inhibitors

In some embodiments the compound provided herein is administered with an inhibitor of a histone deacetylase, e.g., histone deacetylase 9 (HDAC9, HD7, HD7b, HD9, HDAC, HDAC7, HDAC7B, HDAC9B, HDAC9FL, HDRP, MITR; Gene ID: 9734). Examples of HDAC inhibitors include abexinostat, ACY-241, AR-42, BEBT-908, belinostat, CKD-581, CS-055 (HBI-8000), CUDC-907 (fimepinostat), entinostat, givinostat, mocetinostat, panobinostat, pracinostat, quisinostat (JNJ-26481585), resminostat, ricolinostat, SHP-141, valproic acid (VAL-001), vorinostat, tinostamustine, remetinostat, and entinostat. Indoleamine-pyrrole-2,3-dioxygenase (IDO1) inhibitors

In some embodiments the compound provided herein is administered with an inhibitor of indoleamine 2,3-dioxygenase 1 (IDO1; NCBI Gene ID: 3620). Examples of IDO1 inhibitors include BLV-0801, epacadostat, linrodostat (F-001287, BMS-986205), GBV-1012, GBV-1028, GDC-0919, indoximod, NKTR-218, NLG-919-based vaccine, PF-06840003, pyranonaphthoquinone derivatives (SN-35837), resminostat, SBLK-200802, and shIDO-ST, EOS-200271, KHK-2455, and LY-3381916.

### Janus Kinase (JAK) Inhibitors

In some embodiments, the compound provided herein is administered with an inhibitor of Janus kinase 1 (JAK1, JAK1A, JAK1B, JTK3; NCBI Gene ID: 3716); Janus kinase 2 (JAK2, JTK10, THCYT3; NCBI Gene ID: 3717); and/or Janus kinase 3 (JAK3, JAK-3, JAK3_HUMAN, JAKL, L-JAK, LJAK; NCBI Gene ID: 3718). Examples of JAK inhibitors include AT9283, AZD1480, baricitinib, BMS-911543, fedratinib, filgotinib (GLPG0634), gandotinib (LY2784544), INCB039110 (itacitinib), lestaurtinib, momelotinib (CYT0387), ilginatinib maleate (NS-018), pacritinib (SB1518), peficitinib (ASP015K), ruxolitinib, tofacitinib (formerly tasocitinib), INCB052793, and XL019.

### Lysyl Oxidase-Like Protein (LOXL) Inhibitors

In some embodiments the compound provided herein is administered with an inhibitor of a LOXL protein, e.g., LOXL1 (NCBI Gene ID: 4016), LOXL2 (NCBI Gene ID: 4017), LOXL3 (NCBI Gene ID: 84695), LOXL4 (NCBI Gene ID: 84171), and/or LOX (NCBI Gene ID: 4015). Examples of LOXL2 inhibitors include the antibodies described in WO 2009017833 (Arresto Biosciences), WO 2009035791 (Arresto Biosciences), and WO 2011097513 (Gilead Biologics).

### Matrix Metalloprotease (MMP) Inhibitors

In some embodiments the compound provided herein is administered with an inhibitor of a matrix metallopeptidase (MMP), e.g., an inhibitor of MMP1 (NCBI Gene ID: 4312), MMP2 (NCBI Gene ID: 4313), MMP3 (NCBI Gene ID: 4314), MMP7 (NCBI Gene ID: 4316), MMP8 (NCBI Gene ID: 4317), MMP9 (NCBI Gene ID: 4318); MMP10 (NCBI Gene ID: 4319); MMP11 (NCBI Gene ID: 4320); MMP12 (NCBI Gene ID: 4321), MMP13 (NCBI Gene ID: 4322), MMP14 (NCBI Gene ID: 4323), MMP15 (NCBI Gene ID: 4324), MMP16 (NCBI Gene ID: 4325), MMP17 (NCBI Gene ID: 4326), MMP19 (NCBI Gene ID: 4327), MMP20 (NCBI Gene ID: 9313), MMP21 (NCBI Gene ID: 118856), MMP24 (NCBI Gene ID: 10893), MMP25 (NCBI Gene ID: 64386), MMP26 (NCBI Gene ID: 56547), MMP27 (NCBI Gene ID: 64066) and/or MMP28 (NCBI Gene ID: 79148). Examples of MMP9 inhibitors include marimastat (BB-2516), cipemastat (Ro 32-3555), GS-5745 (andecaliximab), and those described in WO 2012027721 (Gilead Biologics).

### RAS and RAS Pathway Inhibitors

In some embodiments the compound provided herein is administered with an inhibitor of KRAS proto-oncogene, GTPase (KRAS; a.k.a., NS; NS3; CFC2; RALD; K-Ras; KRAS1; KRAS2; RASK2; KI-RAS; C-K-RAS; K-RAS2A; K-RAS2B; K-RAS4A; K-RAS4B; c-Ki-ras2; NCBI Gene ID: 3845); NRAS proto-oncogene, GTPase (NRAS; a.k.a., NS6; CMNS; NCMS; ALPS4; N-ras; NRAS1; NCBI Gene ID: 4893) or HRAS proto-oncogene, GTPase (HRAS; a.k.a., CTLO; KRAS; HAMSV; HRAS1; KRAS2; RASH1; RASK2; Ki-Ras; p21ras; C-H-RAS; c-K-ras; H-RASIDX; c-Ki-ras; C-BAS/HAS; C-HA-RAS1; NCBI Gene ID: 3265). The Ras inhibitors can inhibit Ras at either the polynucleotide (e.g., transcriptional inhibitor) or polypeptide (e.g., GTPase enzyme inhibitor) level. In some embodiments, the inhibitors target one or more proteins in the Ras pathway, e.g., inhibit one or more of EGFR, Ras, Raf (A-Raf, B-Raf, C-Raf), MEK (MEK1, MEK2), ERK, PI3K, AKT and mTOR. Illustrative K-Ras inhibitors that can be co-administered include sotorasib (AMG-510), COTI-219, ARS-3248, WDB-178, BI-3406, BI-1701963, SML-8-73-1 (G12C), adagrasib (MRTX-849), ARS-1620 (G12C), SML-8-73-1 (G12C), Compound 3144 (G12D), Kobe0065/2602 (Ras GTP), RT11, MRTX-849 (G12C) and K-Ras(G12D)-selective inhibitory peptides, including KRpep-2 and KRpep-2d. Illustrative KRAS mRNA inhibitors include anti-KRAS U1 adaptor, AZD-4785, siG12D-LODER™, and siG12D exosomes. Illustrative MEK inhibitors that can be co-administered include binimetinib, cobimetinib, PD-0325901, pimasertib, RG-7304, selumetinib, trametinib, and those described below and herein. Illustrative Raf dimer inhibitors that can be co-administered include BGB-283, HM-95573, LXH-254, LY-3009120, RG7304 and TAK-580. Illustrative ERK inhibitors that can be co-administered include LTT-462, LY-3214996, MK-8353, ravoxertinib and ulixertinib. Illustrative Ras GTPase inhibitors that can be co-administered include rigosertib. Illustrative PI3K inhibitors that can be co-administered include idelalisib (Zydelig®), alpelisib, buparlisib, pictilisib, inavolisib (RG6114), ASN-003. Illustrative AKT inhibitors that can be co-administered include capivasertib and GSK2141795. Illustrative PI3K/mTOR inhibitors that can be co-administered include dactolisib, omipalisib, voxtalisib. gedatolisib, GSK2141795, GSK-2126458, inavolisib (RG6114), sapanisertib, ME-344, sirolimus (oral nano-amorphous formulation, cancer), racemetyrosine (TYME-88 (mTOR/cytochrome P450 3A4)), temsirolimus (TORISEL®, CCI-779), CC-115, onatasertib (CC-223), SF-1126, and PQR-309 (bimiralisib). In some embodiments, Ras-driven cancers (e.g., NSCLC) having CDKN2A mutations can be inhibited by co-administration of the MEK inhibitor selumetinib and the CDK4/6 inhibitor palbociclib. See, e.g., Zhou, et al., Cancer Lett. 2017 Nov. 1; 408:130-137. Also, K-RAS and mutant N-RAS can be reduced by the irreversible ERBB1/2/4 inhibitor neratinib. See, e.g., Booth, et al., Cancer Biol Ther. 2018 Feb. 1; 19(2):132-137.

### Mitogen-Activated Protein Kinase (MEK) Inhibitors

In some embodiments the compound provided herein is administered with an inhibitor of mitogen-activated protein kinase kinase 7 (MAP2K7, JNKK2, MAPKK7, MEK, MEK 7, MKK7, PRKMK7, SAPKK-4, SAPKK4; NCBI Gene ID: 5609). Examples of MEK inhibitors include antroquinonol, binimetinib, cobimetinib (GDC-0973, XL-518), MT-144, selumetinib (AZD6244), sorafenib, trametinib (GSK1120212), uprosertib+trametinib, PD-0325901, pimasertib, LTT462, AS703988, CC-90003, and refametinib.

### Phosphatidylinositol 3-Kinase (PI3K) Inhibitors

In some embodiments compound provided herein is administered with an inhibitor of a phosphatidylinositol-4,5-bisphosphate 3-kinase catalytic subunit, e.g., phosphatidylinositol-4,5-bisphosphate 3-kinase catalytic subunit alpha (PIK3CA, CLAPO, CLOVE, CWS5, MCAP, MCM, MCMTC, PI3K, PI3K-alpha, p110-alpha; NCBI Gene ID: 5290); phosphatidylinositol-4,5-bisphosphate 3-kinase catalytic subunit beta (PIK3CB, P10BETA, PI3K, PI3KBETA, PIK3C1; NCBI Gene ID: 5291); phosphatidylinositol-4,5-bisphosphate 3-kinase catalytic subunit gamma (PIK3CG, PI3CG, PI3K, PI3Kgamma, PIK3, p110gamma, p120-PI3K; Gene ID: 5494); and/or phosphatidylinositol-4,5-bisphosphate 3-kinase catalytic subunit delta (PIK3CD, APDS, IMD14, P10DELTA, PI3K, p110D, NCBI Gene ID: 5293). In some embodiments the PI3K inhibitor is a pan-PI3K inhibitor. Examples of PI3K inhibitors include ACP-319, AEZA-129, AMG-319, AS252424, AZD8186, BAY 10824391, BEZ235, buparlisib (BKM120), BYL719 (alpelisib), CH5132799, copanlisib (BAY 80-6946), duvelisib, GDC-0032, GDC-0077, GDC-0941, GDC-0980, GSK2636771, GSK2269557, idelalisib (Zydelig®), INCB50465, IPI-145, IPI-443, IPI-549, KAR4141, LY294002, LY3023414, MLN1117, OXY111A, PA799, PX-866, RG7604, rigosertib, RP5090, RP6530, SRX3177, taselisib, TG100115, TGR-1202 (umbralisib), TGX221, WX-037, X-339, X-414, XL147 (SAR245408), XL499, XL756, wortmannin, ZSTK474, and the compounds described in WO2005113556 (ICOS), WO 2013/052699 (Gilead Calistoga), WO2013116562 (Gilead Calistoga), WO2014100765 (Gilead Calistoga), WO2014100767 (Gilead Calistoga), and WO2014201409 (Gilead Sciences).

### Spleen Tyrosine Kinase (SYK) Inhibitors

In some embodiments the compound provided herein is administered with an inhibitor of spleen associated tyrosine kinase (SYK, p72-Syk, NCBI Gene ID: 6850). Examples of SYK inhibitors include 6-(1H-indazol-6-yl)-N-(4-morpholinophenyl)imidazo[1,2-a]pyrazin-8-amine, BAY-61-3606, cerdulatinib (PRT-062607), entospletinib, fostamatinib (R788), HMPL-523, NVP-QAB 205 AA, R112, R343, tamatinib (R406), gusacitinib (ASN-002), and those described in U.S. Pat. No. 8,450,321 (Gilead Connecticut) and US20150175616.

### Toll-Like Receptor (TLR) Agonists

In some embodiments compound provided herein is administered with an agonist of a toll-like receptor (TLR), e.g., an agonist of TLR1 (NCBI Gene ID: 7096), TLR2 (NCBI Gene ID: 7097), TLR3 (NCBI Gene ID: 7098), TLR4 (NCBI Gene ID: 7099), TLR5 (NCBI Gene ID: 7100), TLR6 (NCBI Gene ID: 10333), TLR7 (NCBI Gene ID: 51284), TLR8 (NCBI Gene ID: 51311), TLR9 (NCBI Gene ID: 54106), and/or TLR10 (NCBI Gene ID: 81793). Example TLR7 agonists that can be co-administered include DS-0509, GS-9620 (vesatolimod), vesatolimod analogs, LHC-165, TMX-101 (imiquimod), GSK-2245035, resiquimod, DSR-6434, DSP-3025, IMO-4200, MCT-465, MEDI-9197, 3M-051, SB-9922, 3M-052, Limtop, TMX-30X, TMX-202, RG-7863, RG-7795, BDB-001, DSP-0509, and the compounds disclosed in US20100143301 (Gilead Sciences), US20110098248 (Gilead Sciences), and US20090047249 (Gilead Sciences), US20140045849 (Janssen), US20140073642 (Janssen), WO2014056953 (Janssen), WO2014076221 (Janssen), WO2014128189 (Janssen), US20140350031 (Janssen), WO2014023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). An TLR7/TLR8 agonist that can be co-administered is NKTR-262. Example TLR8 agonists that can be co-administered include E-6887, IMO-4200, IMO-8400, IMO-9200, MCT-465, MEDI-9197, motolimod, resiquimod, GS-9688, VTX-1463, VTX-763, 3M-051, 3M-052, and the compounds disclosed in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). Example TLR9 agonists that can be co-administered include AST-008, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, leftolimod (MGN-1703), CYT-003, CYT-003-QbG10 and PUL-042. Examples of TLR3 agonist include rintatolimod, poly-ICLC, RIBOXXON®, Apoxxim, RIBOXXIM®, IPH-33, MCT-465, MCT-475, and ND-1.1.

### Tyrosine-Kinase Inhibitors (TKIs)

In some embodiments the compound provided herein is administered with a tyrosine kinase inhibitor (TKI). TKIs may target epidermal growth factor receptors (EGFRs) and receptors for fibroblast growth factor (FGF), platelet-derived growth factor (PDGF), and vascular endothelial growth factor (VEGF). Examples of TKIs include without limitation afatinib, ARQ-087 (derazantinib), asp5878, AZD3759, AZD4547, bosutinib, brigatinib, cabozantinib, cediranib, crenolanib, dacomitinib, dasatinib, dovitinib, E-6201, erdafitinib, erlotinib, gefitinib, gilteritinib (ASP-2215), FP-1039, HM61713, icotinib, imatinib, KX2-391 (Src), lapatinib, lestaurtinib, lenvatinib, midostaurin, nintedanib, ODM-203, osimertinib (AZD-9291), ponatinib, poziotinib, quizartinib, radotinib, rociletinib, sulfatinib (HMPL-012), sunitinib, famitinib L-malate, (MAC-4), tivoanib, TH-4000, and MEDI-575 (anti-PDGFR antibody). Exemplary EGFR targeting agents include neratinib, tucatinib (ONT-380), tesevatinib, mobocertinib (TAK-788), DZD-9008, varlitinib, abivertinib (ACEA-0010), EGF816 (nazartinib), olmutinib (BI-1482694), osimertinib (AZD-9291), AMG-596 (EGFRvIII/CD3), lifirafenib (BGB-283), vectibix, lazertinib (LECLAZA®), and compounds disclosed in Booth, et al., Cancer Biol Ther. 2018 Feb. 1; 19(2):132-137. Antibodies targeting EGFR include without limitation modotuximab, cetuximab sarotalocan (RM-1929), seribantumab, necitumumab, depatuxizumab mafodotin (ABT-414), tomuzotuximab, depatuxizumab (ABT-806), and cetuximab.

### Chemotherapeutic Agents

In some embodiments the compound provided herein is administered with a chemotherapeutic agent or anti-neoplastic agent.

As used herein, the term “chemotherapeutic agent” or “chemotherapeutic” (or “chemotherapy” in the case of treatment with a chemotherapeutic agent) is meant to encompass any non-proteinaceous (e.g., non-peptidic) chemical compound useful in the treatment of cancer. Examples of chemotherapeutic agents include but not limited to: alkylating agents such as thiotepa and cyclophosphamide (CYTOXAN®); alkyl sulfonates such as busulfan, improsulfan, and piposulfan; aziridines such as benzodepa, carboquone, meturedepa, and uredepa; ethylenimines and methylamelamines including altretamine, triethylenemelamine, triethylenephosphoramide, triethylenethiophosphoramide, and trimemylolomelamine; acetogenins, e.g., bullatacin and bullatacinone; a camptothecin, including synthetic analog topotecan; bryostatin, callystatin; CC-1065, including its adozelesin, carzelesin, and bizelesin synthetic analogs; cryptophycins, particularly cryptophycin 1 and cryptophycin 8; dolastatin; duocarmycin, including the synthetic analogs KW-2189 and CBI-TMI; eleutherobin; 5-azacytidine; pancratistatin; a sarcodictyin; spongistatin; nitrogen mustards such as chlorambucil, chlornaphazine, cyclophosphamide, glufosfamide, evofosfamide, bendamustine, estramustine, ifosfamide, mechlorethamine, mechlorethamine oxide hydrochloride, melphalan, novembichin, phenesterine, prednimustine, trofosfamide, and uracil mustard; nitrosoureas such as carmustine, chlorozotocin, foremustine, lomustine, nimustine, and ranimustine; antibiotics such as the enediyne antibiotics (e.g., calicheamicin, especially calicheamicin gammaII and calicheamicin phiI1), dynemicin including dynemicin A, bisphosphonates such as clodronate, an esperamicin, neocarzinostatin chromophore and related chromoprotein enediyne antibiotic chromomophores, aclacinomycins, actinomycin, authramycin, azaserine, bleomycins, cactinomycin, carabicin, carrninomycin, carzinophilin, chromomycins, dactinomycin, daunorubicin, detorubicin, 6-diazo-5-C═O-L-norleucine, dC═Orubicin (including morpholino-dC═Orubicin, cyanomorpholino-dC═Orubicin, 2-pyrrolino-dC═Orubicin, and deoxydC═Orubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, and zorubicin; anti-metabolites such as methotrexate and 5-fluorouracil (5-FU); folic acid analogs such as demopterin, methotrexate, pteropterin, and trimetrexate; purine analogs such as cladribine, pentostatin, fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, and floxuridine; androgens such as calusterone, dromostanolone propionate, epitiostanol, mepitiostane, and testolactone; anti-adrenals such as aminoglutethimide, mitotane, and trilostane; folic acid replenishers such as frolinic acid; radiotherapeutic agents such as Radium-223; trichothecenes, especially T-2 toxin, verracurin A, roridin A, and anguidine; taxoids such as paclitaxel (TAXOL®), abraxane, docetaxel (TAXOTERE®), cabazitaxel, BIND-014, tesetaxel; sabizabulin (Veru-111); platinum analogs such as cisplatin and carboplatin, NC-6004 nanoplatin; aceglatone; aldophosphamide glycoside; aminolevulinic acid; eniluracil; amsacrine; hestrabucil; bisantrene; edatraxate; defofamine; demecolcine; diaziquone; elformthine; elliptinium acetate; an epothilone; etoglucid; gallium nitrate; hydroxyurea; lentinan; leucovorin; lonidamine; maytansinoids such as maytansine and ansamitocins; mitoguazone; mitoxantrone; mopidamol; nitracrine; pentostatin; phenamet; pirarubicin; losoxantrone; fluoropyrimidine; folinic acid; podophyllinic acid; 2-ethylhydrazide; procarbazine; polysaccharide-K (PSK); razoxane; rhizoxin; sizofiran; spirogermanium; tenuazonic acid; trabectedin, triaziquone; 2,2′,2″-trichlorotriemylamine; urethane; vindesine; dacarbazine; mannomustine; mitobronitol; mitolactol; pipobroman; gacytosine; arabinoside (“Ara-C”); cyclophosphamide; thiopeta; chlorambucil; gemcitabine (GEMZAR®); 6-thioguanine; mercaptopurine; methotrexate; vinblastine; platinum; etoposide (VP-16); ifosfamide; mitroxantrone; vancristine; vinorelbine (NAVELBINE®); novantrone; teniposide; edatrexate; daunomycin; aminopterin; xeoloda; ibandronate; CPT-11; topoisomerase inhibitor RFS 2000; difluoromethylornithine (DFMO); retinoids such as retinoic acid; capecitabine; NUC-1031; FOLFOX (folinic acid, 5-fluorouracil, oxaliplatin); FOLFIRI (folinic acid, 5-fluorouracil, irinotecan); FOLFOXIRI (folinic acid, 5-fluorouracil, oxaliplatin, irinotecan), FOLFIRINOX (folinic acid, 5-fluorouracil, irinotecan, oxaliplatin), and pharmaceutically acceptable salts, acids, or derivatives of any of the above. Such agents can be conjugated onto an antibody or any targeting agent described herein to create an antibody-drug conjugate (ADC) or targeted drug conjugate.

### Anti-Hormonal Agents

Also included in the definition of “chemotherapeutic agent” are anti-hormonal agents such as anti-estrogens and selective estrogen receptor modulators (SERMs), inhibitors of the enzyme aromatase, anti-androgens, and pharmaceutically acceptable salts, acids or derivatives of any of the above that act to regulate or inhibit hormone action on tumors.

Examples of anti-estrogens and SERMs include tamoxifen (including NOLVADEX™), raloxifene, droloxifene, 4-hydroxytamoxifen, trioxifene, keoxifene, LY117018, onapristone, and toremifene (FARESTON®).

Inhibitors of the enzyme aromatase regulate estrogen production in the adrenal glands. Examples include 4(5)-imidazoles, aminoglutethimide, megestrol acetate (MEGACE®), exemestane, formestane, fadrozole, vorozole (RIVISOR®), letrozole (FEMARA®), and anastrozole (ARIMIDEX®).

Examples of anti-androgens include apalutamide, abiraterone, enzalutamide, flutamide, galeterone, nilutamide, bicalutamide, leuprolide, goserelin, ODM-201, APC-100, ODM-204, enobosarm (GTX-024), darolutamide, and IONIS-AR-2.5Rx (antisense).

An example progesterone receptor antagonist includes onapristone. Additional progesterone targeting agents include TRI-CYCLEN LO (norethindrone+ethinyl estradiol), norgestimate+ethinylestradiol (Tri-Cyclen) and levonorgestrel.

### Anti-Angiogenic Agents

In some embodiments the compound provided herein is administered with an anti-angiogenic agent. Anti-angiogenic agents that can be co-administered include retinoid acid and derivatives thereof, 2-methoxyestradiol, ANGIOSTATIN®, ENDOSTATIN®, regorafenib, necuparanib, suramin, squalamine, tissue inhibitor of metalloproteinase-1, tissue inhibitor of metalloproteinase-2, plasminogen activator inhibitor-1, plasminogen activator inhibitor-2, cartilage-derived inhibitor, paclitaxel (nab-paclitaxel), platelet factor 4, protamine sulphate (clupeine), sulphated chitin derivatives (prepared from queen crab shells), sulphated polysaccharide peptidoglycan complex (sp-pg), staurosporine, modulators of matrix metabolism including proline analogs such as 1-azetidine-2-carboxylic acid (LACA), cishydroxyproline, d,I-3,4-dehydroproline, thiaproline, α,α′-dipyridyl, beta-aminopropionitrile fumarate, 4-propyl-5-(4-pyridinyl)-2(3h)-oxazolone, methotrexate, mitoxantrone, heparin, interferons, 2 macroglobulin-serum, chicken inhibitor of metalloproteinase-3 (ChIMP-3), chymostatin, beta-cyclodextrin tetradecasulfate, eponemycin, fumagillin, gold sodium thiomalate, d-penicillamine, beta-1-anticollagenase-serum, alpha-2-antiplasmin, bisantrene, lobenzarit disodium, n-2-carboxyphenyl-4-chloroanthronilic acid disodium or “CCA”, thalidomide, angiostatic steroid, carboxy aminoimidazole, metalloproteinase inhibitors such as BB-94, inhibitors of S100A9 such as tasquinimod. Other anti-angiogenesis agents include antibodies, preferably monoclonal antibodies against these angiogenic growth factors: beta-FGF, alpha-FGF, FGF-5, VEGF isoforms, VEGF-C, HGF/SF, and Ang-1/Ang-2. Examples for anti-VEGFA antibodies that can be co-administered include bevacizumab, vanucizumab, faricimab, dilpacimab (ABT-165; DLL4/VEGF), or navicixizumab (OMP-305B83; DLL4/VEGF).

### Anti-Fibrotic Agents

In some embodiments the compound provided herein is administered with an anti-fibrotic agent. Anti-fibrotic agents that can be co-administered include the compounds such as beta-aminoproprionitrile (BAPN), as well as the compounds disclosed in U.S. Pat. No. 4,965,288 relating to inhibitors of lysyl oxidase and their use in the treatment of diseases and conditions associated with the abnormal deposition of collagen and U.S. Pat. No. 4,997,854 relating to compounds which inhibit LOX for the treatment of various pathological fibrotic states, which are herein incorporated by reference. Further exemplary inhibitors are described in U.S. Pat. No. 4,943,593 relating to compounds such as 2-isobutyl-3-fluoro-, chloro-, or bromo-allylamine, U.S. Pat. Nos. 5,021,456, 5,059,714, 5,120,764, 5,182,297, 5,252,608 relating to 2-(1-naphthyloxymemyl)-3-fluoroallylamine, and US 20040248871, which are herein incorporated by reference.

Exemplary anti-fibrotic agents also include the primary amines reacting with the carbonyl group of the active site of the lysyl oxidases, and more particularly those which produce, after binding with the carbonyl, a product stabilized by resonance, such as the following primary amines: emylenemamine, hydrazine, phenylhydrazine, and their derivatives; semicarbazide and urea derivatives; aminonitriles such as BAPN or 2-nitroethylamine; unsaturated or saturated haloamines such as 2-bromo-ethylamine, 2-chloroethylamine, 2-trifluoroethylamine, 3-bromopropylamine, and p-halobenzylamines; and selenohomocysteine lactone.

Other anti-fibrotic agents are copper chelating agents penetrating or not penetrating the cells. Exemplary compounds include indirect inhibitors which block the aldehyde derivatives originating from the oxidative deamination of the lysyl and hydroxylysyl residues by the lysyl oxidases. Examples include the thiolamines, particularly D-penicillamine, and its analogs such as 2-amino-5-mercapto-5-methylhexanoic acid, D-2-amino-3-methyl-3-((2-acetamidoethyl)dithio)butanoic acid, p-2-amino-3-methyl-3-((2-aminoethyl)dithio)butanoic acid, sodium-4-((p-1-dimethyl-2-amino-2-carboxyethyl)dithio)butane sulphurate, 2-acetamidoethyl-2-acetamidoethanethiol sulphanate, and sodium-4-mercaptobutanesulphinate trihydrate.

### Anti-Inflammatory Agents

In some embodiments the compound provided herein is administered with an anti-inflammatory agent. Example anti-inflammatory agents include without limitation inhibitors of one or more of arginase (ARG1 (NCBI Gene ID: 383), ARG2 (NCBI Gene ID: 384)), carbonic anhydrase (CA1 (NCBI Gene ID: 759), CA2 (NCBI Gene ID: 760), CA3 (NCBI Gene ID: 761), CA4 (NCBI Gene ID: 762), CA5A (NCBI Gene ID: 763), CA5B (NCBI Gene ID: 11238), CA6 (NCBI Gene ID: 765), CA7 (NCBI Gene ID: 766), CA8 (NCBI Gene ID: 767), CA9 (NCBI Gene ID: 768), CA10 (NCBI Gene ID: 56934), CA11 (NCBI Gene ID: 770), CA12 (NCBI Gene ID: 771), CA13 (NCBI Gene ID: 377677), CA14 (NCBI Gene ID: 23632)), prostaglandin-endoperoxide synthase 1 (PTGS1, COX-1; NCBI Gene ID: 5742), prostaglandin-endoperoxide synthase 2 (PTGS2, COX-2; NCBI Gene ID: 5743), secreted phospholipase A2, prostaglandin E synthase (PTGES, PGES; Gene ID: 9536), arachidonate 5-lipoxygenase (ALOX5, 5-LOX; NCBI Gene ID: 240), soluble epoxide hydrolase 2 (EPHX2, SEH; NCBI Gene ID: 2053) and/or mitogen-activated protein kinase kinase kinase 8 (MAP3K8, TPL2; NCBI Gene ID: 1326). In some embodiments, the inhibitor is a dual inhibitor, e.g., a dual inhibitor of COX-2/COX-1, COX-2/SEH, COX-2/CA, COX-2/5-LOX.

Examples of inhibitors of prostaglandin-endoperoxide synthase 1 (PTGS1, COX-1; NCBI Gene ID: 5742) that can be co-administered include mofezolac, GLY-230, and TRK-700.

Examples of inhibitors of prostaglandin-endoperoxide synthase 2 (PTGS2, COX-2; NCBI Gene ID: 5743) that can be co-administered include diclofenac, meloxicam, parecoxib, etoricoxib, AP-101, celecoxib, AXS-06, diclofenac potassium, DRGT-46, AAT-076, meisuoshuli, lumiracoxib, meloxicam, valdecoxib, zaltoprofen, nimesulide, anitrazafen, apricoxib, cimicoxib, deracoxib, flumizole, firocoxib, mavacoxib, NS-398, pamicogrel, parecoxib, robenacoxib, rofecoxib, rutecarpine, tilmacoxib, and zaltoprofen. Examples of dual COX1/COX2 inhibitors that can be co-administered include HP-5000, lornoxicam, ketorolac tromethamine, bromfenac sodium, ATB-346, HP-5000. Examples of dual COX-2/carbonic anhydrase (CA) inhibitors that can be co-administered include polmacoxib and imrecoxib.

Examples of inhibitors of secreted phospholipase A2, prostaglandin E synthase (PTGES, PGES; Gene ID: 9536) that can be co-administered include LY3023703, GRC 27864, and compounds described in WO2015158204, WO2013024898, WO2006063466, WO2007059610, WO2007124589, WO2010100249, WO2010034796, WO2010034797, WO2012022793, WO2012076673, WO2012076672, WO2010034798, WO2010034799, WO2012022792, WO2009103778, WO2011048004, WO2012087771, WO2012161965, WO2013118071, WO2013072825, WO2014167444, WO2009138376, WO2011023812, WO2012110860, WO2013153535, WO2009130242, WO2009146696, WO2013186692, WO2015059618, WO2016069376, WO2016069374, WO2009117985, WO2009064250, WO2009064251, WO2009082347, WO2009117987, and WO2008071173. Metformin has further been found to repress the COX2/PGE2/STAT3 axis, and can be co-administered. See, e.g., Tong, et al., Cancer Lett. (2017) 389:23-32; and Liu, et al., Oncotarget. (2016) 7(19):28235-46.

Examples of inhibitors of carbonic anhydrase (e.g., one or more of CA1 (NCBI Gene ID: 759), CA2 (NCBI Gene ID: 760), CA3 (NCBI Gene ID: 761), CA4 (NCBI Gene ID: 762), CASA (NCBI Gene ID: 763), CA5B (NCBI Gene ID: 11238), CA6 (NCBI Gene ID: 765), CA7 (NCBI Gene ID: 766), CA8 (NCBI Gene ID: 767), CA9 (NCBI Gene ID: 768), CA10 (NCBI Gene ID: 56934), CA11 (NCBI Gene ID: 770), CA12 (NCBI Gene ID: 771), CA13 (NCBI Gene ID: 377677), CA14 (NCBI Gene ID: 23632)) that can be co-administered include acetazolamide, methazolamide, dorzolamide, zonisamide, brinzolamide and dichlorphenamide. A dual COX-2/CA1/CA2 inhibitor that can be co-administered includes CG100649.

Examples of inhibitors of arachidonate 5-lipoxygenase (ALOX5, 5-LOX; NCBI Gene ID: 240) that can be co-administered include meclofenamate sodium, zileuton.

Examples of inhibitors of soluble epoxide hydrolase 2 (EPHX2, SEH; NCBI Gene ID: 2053) that can be co-administered include compounds described in WO2015148954. Dual inhibitors of COX-2/SEH that can be co-administered include compounds described in WO2012082647. Dual inhibitors of SEH and fatty acid amide hydrolase (FAAH; NCBI Gene ID: 2166) that can be co-administered include compounds described in WO2017160861.

Examples of inhibitors of mitogen-activated protein kinase kinase kinase 8 (MAP3K8, tumor progression loci-2, TPL2; NCBI Gene ID: 1326) that can be co-administered include GS-4875, GS-5290, BHM-078 and those described in WO2006124944, WO2006124692, WO2014064215, WO2018005435, Teli, et al., J Enzyme Inhib Med Chem. (2012) 27(4):558-70; Gangwall, et al., Curr Top Med Chem. (2013) 13(9):1015-35; Wu, et al., Bioorg Med Chem Lett. (2009) 19(13):3485-8; Kaila, et al., Bioorg Med Chem. (2007) 15(19):6425-42; and Hu, et al., Bioorg Med Chem Lett. (2011) 21(16):4758-61.

### Tumor Oxygenation Agents

In some embodiments the compound provided herein is administered with an agent that promotes or increases tumor oxygenation or reoxygenation, or prevents or reduces tumor hypoxia. Illustrative agents that can be co-administered include, e.g., Hypoxia inducible factor-1 alpha (HIF-1α) inhibitors, such as PT-2977, PT-2385; VEGF inhibitors, such as bevasizumab, IMC-3C5, GNR-011, tanibirumab, LYN-00101, ABT-165; and/or an oxygen carrier protein (e.g., a heme nitric oxide and/or oxygen binding protein (HNOX)), such as OMX-302 and HNOX proteins described in WO2007137767, WO2007139791, WO2014107171, and WO2016149562.

### Immunotherapeutic Agents

In some embodiments the compound provided herein is administered with an immunotherapeutic agent. In some embodiments the immunotherapeutic agent is an antibody. Example immunotherapeutic agents that can be co-administered include abagovomab, AB308, ABP-980, adecatumumab, afutuzumab, alemtuzumab, altumomab, amatuximab, anatumomab, arcitumomab, atezolizumab, bavituximab, bectumomab, bevacizumab, bivatuzumab, blinatumomab, brentuximab, camidanlumab, cantuzumab, catumaxomab, CC49, cetuximab, citatuzumab, cixutumumab, clivatuzumab, conatumumab, dacetuzumab, dalotuzumab, daratumumab, detumomab, dinutuximab, domvanalimab, drozitumab, duligotumab, dusigitumab, ecromeximab, elotuzumab, emibetuzumab, ensituximab, ertumaxomab, etaracizumab, farletuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab (YERVOY®, MDX-010, BMS-734016, and MDX-101), iratumumab, labetuzumab, lexatumumab, lintuzumab, lorvotuzumab, lucatumumab, mapatumumab, matuzumab, milatuzumab, minretumomab, mitumomab, mogamulizumab, moxetumomab, naptumomab, narnatumab, necitumumab, nimotuzumab, nofetumomab, OBI-833, obinutuzumab, ocaratuzumab, ofatumumab, olaratumab, onartuzumab, oportuzumab, oregovomab, panitumumab, parsatuzumab, pasudotox, patritumab, pemtumomab, pertuzumab, pintumomab, pritumumab, racotumomab, radretumab, ramucirumab (Cyramza®), rilotumumab, rituximab, robatumumab, samalizumab, satumomab, sibrotuzumab, siltuximab, solitomab, simtuzumab, tacatuzumab, taplitumomab, tenatumomab, teprotumumab, tigatuzumab, tositumomab, trastuzumab, tucotuzumab, ubilituximab, veltuzumab, vorsetuzumab, votumumab, zalutumumab, zimberelimab, and 3F8. Rituximab can be used for treating indolent B-cell cancers, including marginal-zone lymphoma, WM, CLL, and small lymphocytic lymphoma. A combination of rituximab and chemotherapy agents is especially effective.

The exemplified therapeutic antibodies can be further labeled or combined with a radioisotope particle such as indium-111, yttrium-90 (90Y-clivatuzumab), or iodine-131.

In some embodiments, the immunotherapeutic agent is an antibody-drug conjugate (ADC). Illustrative ADCs that can be co-administered include without limitation drug-conjugated antibodies, fragments thereof, or antibody mimetics targeting the proteins or antigens listed above and herein. Example ADCs that can be co-administered include gemtuzumab, brentuximab, belantamab (e.g., belantamab mafodotin), camidanlumab (e.g., camidanlumab tesirine), trastuzumab (e.g., trastuzumab deruxtecan; trasuzumab emtansine), inotuzumab, glembatumumab, anetumab, mirvetuximab (e.g., mirvetuximab soravtansine), depatuxizumab, vadastuximab, labetuzumab, ladiratuzumab (e.g., ladiratuzumab vedotin), loncastuximab (e.g., loncastuximab tesirine), sacituzumab (e.g., sacituzumab govitecan), datopotamab (e.g., datopotamab deruxtecan; DS-1062; Dato-DXd), patritumab (e.g., patritumab deruxtecan), lifastuzumab, indusatumab, polatuzumab (e.g., polatuzumab vedotin), pinatuzumab, coltuximab, upifitamab (e.g., upifitamab rilsodotin), indatuximab, milatuzumab, rovalpituzumab (e.g., rovalpituzumab tesirine), enfortumab (e.g., enfortumab vedotin), tisotumab (e.g., tisotumab vedotin), tusamitamab (e.g., tusamitamab ravtansine), disitamab (e.g., disitamab vedotin), telisotuzumab vedotin (ABBV-399), AGS-16C3F, ASG-22ME, AGS67E, AMG172, AMG575, BAY1129980, BAY1187982, BAY94-9343, GSK2857916, Humax-TF-ADC, IMGN289, IMGN151, IMGN529, IMGN632, IMGN853, IMGC936, LOP628, PCA062, MDX-1203 (BMS936561), MEDI-547, PF-06263507, PF-06647020, PF-06647263, PF-06664178, RG7450, RG7458, RG7598, SAR566658, SGN-CD19A, SGN-CD33A, SGN-CD70A, SGN-LIV1A, SYD985, DS-7300, XMT-1660, IMMU-130, and IMMU-140. ADCs that can be co-administered are described, e.g., in Lambert, et al., Adv Ther (2017) 34:1015-1035 and in de Goeij, Current Opinion in Immunology (2016) 40:14-23.

Illustrative therapeutic agents (e.g., anticancer or antineoplastic agents) that can be conjugated to the drug-conjugated antibodies, fragments thereof, or antibody mimetics include without limitation monomethyl auristatin E (MMAE), monomethyl auristatin F (MMAF), a calicheamicin, ansamitocin, maytansine or an analog thereof (e.g., mertansine/emtansine (DM1), ravtansine/soravtansine (DM4)), an anthracyline (e.g., dC═Orubicin, daunorubicin, epirubicin, idarubicin), pyrrolobenzodiazepine (PBD) DNA cross-linking agent SC-DR002 (D6.5), duocarmycin, a microtubule inhibitors (MTI) (e.g., a taxane, a vinca alkaloid, an epothilone), a pyrrolobenzodiazepine (PBD) or dimer thereof, a duocarmycin (A, B1, B2, C1, C2, D, SA, CC-1065), and other anticancer or anti-neoplastic agents described herein. In some embodiments, the therapeutic agent conjugated to the drug-conjugated antibody is a topoisomerase I inhibitor (e.g., a camptothecin analog, such as irinotecan or its active metabolite SN38). In some embodiments, the therapeutic agents (e.g., anticancer or antineoplastic agents) that can be conjugated to the drug-conjugated antibodies, fragments thereof, or antibody mimetics include an immune checkpoint inhibitor. In some embodiments the conjugated immune checkpoint inhibitor is a conjugated small molecule inhibitor of CD274 (PDL1, PD-L1), programmed cell death 1 (PDCD1, PD1, PD-1) or CTLA4. In some embodiments the conjugated small molecule inhibitor of CD274 or PDCD1 is selected from the group consisting of GS-4224, GS-4416, INCB086550 and MAX10181. In some embodiments the conjugated small molecule inhibitor of CTLA4 comprises BPI-002.

In some embodiments the ADCs that can be co-administered include an antibody targeting tumor-associated calcium signal transducer 2 (TROP-2; TACSTD2; EGP-1; NCBI Gene ID: 4070). Illustrative anti-TROP-2 antibodies include without limitation TROP2-XPAT (Amunix), BAT-8003 (Bio-Thera Solutions), TROP-2-IR700 (Chiome Bioscience), datopotamab deruxtecan (Daiichi Sankyo, AstraZeneca), GQ-1003 (Genequantum Healthcare, Samsung BioLogics), DAC-002 (Hangzhou DAC Biotech, Shanghai Junshi Biosciences), sacituzumab govitecan (Gilead Sciences), E1-3s (Immunomedics/Gilead, IBC Pharmaceuticals), TROP2-TRACTr (Janux Therapeutics), LIV-2008 (LivTech/Chiome, Yakult Honsha, Shanghai Henlius BioTech), LIV-2008b (LivTech/Chiome), anti-TROP-2a (Oncoxx), anti-TROP-2b (Oncoxx), OXG-64 (Oncoxx), OXS-55 (Oncoxx), humanized anti-Trop2-SN38 antibody conjugate (Shanghai Escugen Biotechnology, TOT Biopharma), anti-Trop2 antibody-CLB-SN-38 conjugate (Shanghai Fudan-Zhangjiang Bio-Pharmaceutical), SKB-264 (Sichuan Kelun Pharmaceutical/Klus Pharma), TROP2-Ab8 (Abmart), Trop2-IgG (Nanjing Medical University (NMU)), 90Y-DTPA-AF650 (Peking University First Hospital), hRS7-CM (SynAffix), 89Zr-DFO-AF650 (University of Wisconsin-Madison), anti-Trop2 antibody (Mediterranea Theranostic, LegoChem Biosciences), KD-065 (Nanjing KAEDI Biotech), and those described in WO2020016662 (Abmart), WO2020249063 (Bio-Thera Solutions), US20190048095 (Bio-Thera Solutions), WO2013077458 (LivTech/Chiome), EP20110783675 (Chiome), WO2015098099 (Daiichi Sankyo), WO2017002776 (Daiichi Sankyo), WO2020130125 (Daiichi Sankyo), WO2020240467 (Daiichi Sankyo), US2021093730 (Daiichi Sankyo), U.S. Pat. No. 9,850,312 (Daiichi Sankyo), CN112321715 (Biosion), US2006193865 (Immunomedics/Gilead), WO2011068845 (Immunomedics/Gilead), US2016296633 (Immunomedics/Gilead), US2017021017 (Immunomedics/Gilead), US2017209594 (Immunomedics/Gilead), US2017274093 (Immunomedics/Gilead), US2018110772 (Immunomedics/Gilead), US2018185351 (Immunomedics/Gilead), US2018271992 (Immunomedics/Gilead), WO2018217227 (Immunomedics/Gilead), US2019248917 (Immunomedics/Gilead), CN111534585 (Immunomedics/Gilead), US2021093730 (Immunomedics/Gilead), US2021069343 (Immunomedics/Gilead), U.S. Pat. No. 8,435,539 (Immunomedics/Gilead), U.S. Pat. No. 8,435,529 (Immunomedics/Gilead), U.S. Pat. No. 9,492,566 (Immunomedics/Gilead), WO2003074566 (Gilead), WO2020257648 (Gilead), US2013039861 (Gilead), WO2014163684 (Gilead), U.S. Pat. No. 9,427,464 (LivTech/Chiome), U.S. Ser. No. 10/501,555 (Abruzzo Theranostic/Oncoxx), WO2018036428 (Sichuan Kelun Pharma), WO2013068946 (Pfizer), WO2007095749 (Roche), and WO2020094670 (SynAffix). In some embodiments, the anti-Trop-2 antibody is selected from hRS7, Trop-2-XPAT, and BAT-8003. In some embodiments, the anti-Trop-2 antibody is hRS7. In some embodiments, hRS7 is as disclosed in U.S. Pat. Nos. 7,238,785; 7,517,964 and 8,084,583, which are incorporated herein by reference. In some embodiments, the antibody-drug conjugate comprises an anti-Trop-2 antibody and an anticancer agent linked by a linker. In some embodiments, the linker includes the linkers disclosed in U.S. Pat. No. 7,999,083. In some embodiments, the linker is CL2A. In some embodiments, the drug moiety of antibody-drug conjugate is a chemotherapeutic agent. In some embodiments, the chemotherapeutic agent is selected from dC═Orubcin (DOX), epirubicin, morpholinodC═Orubicin (morpholino-DOX), cyanomorpholino-dC═Orubicin (cyanomorpholinoDOX), 2-pyrrolino-dC═Orubicin (2-PDOX), CPT, 10-hydroxy camptothecin, SN-38, topotecan, lurtotecan, 9-aminocamptothecin, 9-nitrocamptothecin, taxanes, geldanamycin, ansamycins, and epothilones. In some embodiments, the chemotherapeutic moiety is SN-38. In some embodiments the compound provided herein is administered with sacituzumab govitecan.

In some embodiments the ADCs that can be co-administered include an antibody targeting carcinoembryonic antigen-related cell adhesion molecule 1 (CEACAM1; CD66a; NCBI Gene ID: 634). In some embodiments the CEACAM1 antibody is hMN-14 (e.g., as described in WO1996011013). In some embodiments the CEACAM1-ADC is as described in WO2010093395 (anti-CEACAM-1-CL2A-SN38). In some embodiments the compound provided herein is administered with the CEACAM1-ADC IMMU-130.

In some embodiments the ADCs that can be co-administered include an antibody targeting MHC class II cell surface receptor encoded by the human leukocyte antigen complex (HLA-DR). In some embodiments the HLA-DR antibody is hL243 (e.g., as described in WO2006094192). In some embodiments the HLA-DR-ADC is as described in WO2010093395 (anti-HLA-DR-CL2A-SN38). In some embodiments the compound provided herein is administered with the HLA-DR-ADC IMMU-140.

### Cancer Gene Therapy and Cell Therapy

In some embodiments the compound provided herein is administered with a cancer gene therapy and cell therapy. Cancer gene therapies and cell therapies include the insertion of a normal gene into cancer cells to replace a mutated or altered gene; genetic modification to silence a mutated gene; genetic approaches to directly kill the cancer cells; including the infusion of immune cells designed to replace most of the patient's own immune system to enhance the immune response to cancer cells, or activate the patient's own immune system (T cells or Natural Killer cells) to kill cancer cells, or find and kill the cancer cells; genetic approaches to modify cellular activity to further alter endogenous immune responsiveness against cancer.

### Cellular Therapies

In some embodiments the compound provided herein is administered with one or more cellular therapies. Illustrative cellular therapies include without limitation co-administration of one or more of a population of natural killer (NK) cells, NK-T cells, T cells, cytokine-induced killer (CIK) cells, macrophage (MAC) cells, tumor infiltrating lymphocytes (TILs) and/or dendritic cells (DCs). In some embodiments, the cellular therapy entails a T cell therapy, e.g., co-administering a population of alpha/beta TCR T cells, gamma/delta TCR T cells, regulatory T (Treg) cells and/or TRuC™ T cells. In some embodiments, the cellular therapy entails a NK cell therapy, e.g., co-administering NK-92 cells. As appropriate, a cellular therapy can entail the co-administration of cells that are autologous, syngeneic or allogeneic to the subject.

In some embodiments the cellular therapy entails co-administering cells comprising chimeric antigen receptors (CARs). In such therapies, a population of immune effector cells engineered to express a CAR, wherein the CAR comprises a tumor antigen-binding domain. In T cell therapies, the T cell receptors (TCRs) are engineered to target tumor derived peptides presented on the surface of tumor cells.

With respect to the structure of a CAR, in some embodiments, the CAR comprises an antigen binding domain, a transmembrane domain, and an intracellular signaling domain. In some embodiments, the intracellular domain comprises a primary signaling domain, a costimulatory domain, or both of a primary signaling domain and a costimulatory domain. In some embodiments, the primary signaling domain comprises a functional signaling domain of one or more proteins selected from the group consisting of CD3 zeta, CD3 gamma, CD3 delta, CD3 epsilon, common FcR gamma (FCERIG), FcR beta (Fc Epsilon Rlb), CD79a, CD79b, Fcgamma RIIa, DAP10, and DAP12.

In some embodiments, the costimulatory domain comprises a functional domain of one or more proteins selected from the group consisting of CD27, CD28, 4-1BB(CD137), OX40, CD30, CD40, PD-1, ICOS, CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRFI), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, ITGAE, CD103, ITGAL, CD1A (NCBI Gene ID: 909), CD1B (NCBI Gene ID: 910), CD1C (NCBI Gene ID: 911), CD1D (NCBI Gene ID: 912), CD1E (NCBI Gene ID: 913), ITGAM, ITGAX, ITGB1, CD29, ITGB2 (CD18, LFA-1), ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D.

In some embodiments, the transmembrane domain comprises a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, ICOS (CD278), 4-1BB(CD137), GITR, CD40, BAFFR, HVEM (LIGHTR), SLAMF7, NKp80 (KLRF1), CD160, CD19, IL2R beta, IL2R gamma, IL7R, ITGA1, VLA1, CD49a, ITGA4, IA4, CD49D, ITGA6, VLA-6, CD49f, ITGAD, CD1A, CD1B, CD1C, CD1D, CD1E, ITGAE, CD103, ITGAL, ITGAM, ITGAX, ITGB1, CD29, ITGB2 (LFA-1, CD18), ITGB7, TNFR2, DNAM1 (CD226), SLAMF4 (CD244, 2B4), CD84, CD96 (TACTILE), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and NKG2C.

In some embodiments, the TCR or CAR antigen binding domain or the immunotherapeutic agent described herein (e.g., monospecific or multi-specific antibody or antigen-binding fragment thereof or antibody mimetic) binds a tumor-associated antigen (TAA). In some embodiments, the tumor-associated antigen is selected from the group consisting of: CD19; CD123; CD22; CD30; CD171; CS-1 (also referred to as CD2 subset 1, CRACC, SLAMF7, CD319, and 19A24); C-type lectin-like molecule-1 (CLL-1 or CLECLI); CD33; epidermal growth factor receptor variant III (EGFRvIII); ganglioside G2 (GD2); ganglioside GD3 (αNeuSAc(2-8)αNeuSAc(2-3)βDGaip(1-4)bDGIcp(1-1)Cer); ganglioside GM3 (αNeuSAc(2-3)βDGalp(1-4)βDGlcp(1-1)Cer); TNF receptor superfamily member 17 (TNFRSF17, BCMA); Tn antigen ((Tn Ag) or (GaINAcu-Ser/Thr)); prostate-specific membrane antigen (PSMA); receptor tyrosine kinase-like orphan receptor 1 (RORI); tumor-associated glycoprotein 72 (TAG72); CD38; CD44v6; Carcinoembryonic antigen (CEA); epithelial cell adhesion molecule (EPCAM); B7H3 (CD276); KIT (CD117); interleukin-13 receptor subunit alpha-2 (IL-13Ra2 or CD213A2); mesothelin; interleukin 11 receptor alpha (IL-IIRa); prostate stem cell antigen (PSCA); protease serine 21 (Testisin or PRSS21); vascular endothelial growth factor receptor 2 (VEGFR2); Lewis(Y)antigen; CD24; platelet-derived growth factor receptor beta (PDGFR-beta); stage-specificembryonic antigen-4 (SSEA-4); CD20; delta like 3 (DLL3); folate receptor alpha; receptor tyrosine-protein kinase, ERBB2 (Her2/neu); mucin 1, cell surface associated (MUC1); epidermal growth factor receptor (EGFR); neural cell adhesion molecule (NCAM); prostase; prostatic acid phosphatase (PAP); elongation factor 2 mutated (ELF2M); ephrin B2; fibroblast activation protein alpha (FAP); insulin-like growth factor 1 receptor (IGF-I receptor), carbonic anhydrase IX (CAIX); proteasome (Prosome, Macropain) subunit, beta type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); fucosyl GM1; sialyl Lewis adhesion molecule (sLe); transglutaminase 5 (TGS5); high molecular weight-melanomaassociatedantigen (HMWMAA); o-acetyl-GD2 ganglioside (OAcGD2); folate receptor beta; tumor endothelial marker 1 (TEM1/CD248); tumor endothelial marker 7-related (TEM7R); six transmembrane epithelial antigen of the prostate I (STEAP1); claudin 6 (CLDN6); thyroid stimulating hormone receptor (TSHR); G protein-coupled receptor class C group 5, member D (GPRCSD); chromosome X open reading frame 61 (CXORF61); CD97; CD179a; anaplastic lymphoma kinase (ALK); polysialic acid; placenta-specific 1 (PLAC1); hexasaccharide portion of globoH glycoceramide (GloboH); mammary gland differentiation antigen (NY-BR-1); uroplakin 2 (UPK2); hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); olfactory receptor 51E2 (ORS IE2); TCR Gamma Alternate Reading Frame Protein (TARP); Wilms tumor protein (WT1); cancer/testis antigen 1 (NY-ESO-1); cancer/testis antigen 2 (LAGE-1a); melanoma associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MADCT-1); melanoma cancer testis antigen-2 (MAD-CT-2); fos-related antigen 1; tumor protein p53, (p53); p53 mutant; prostein; survivin; telomerase; prostate carcinoma tumor antigen-1 (PCTA-1 or Galectin 8), melanoma antigen recognized by T cells 1 (MelanA or MARTI); rat sarcoma (Ras) mutant; human telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); androgen receptor; cyclin B1; v-myc avian myelocytomatosis viral oncogene neuroblastoma derived homolog (MYCN); ras homolog family member C (RhoC); tyrosinase-related protein 2 (TRP-2); cytochrome P450 1B1(CYP IBI); CCCTC-Binding Factor (Zinc Finger Protein)-Like (BORIS or Brother of the Regulator of Imprinted Sites), squamous cell carcinoma antigen recognized by T-cells 3 (SART3); paired box protein Pax-5 (PAX5); proacrosin binding protein sp32 (OY-TES I); lymphocyte-specific protein tyrosine kinase (LCK); A kinase anchor protein 4 (AKAP-4); synovial sarcoma, X breakpoint 2 (SSX2); receptor for advanced glycation endproducts (RAGE-I); renal ubiquitous 1 (RUI); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; leukocyte-associated immunoglobulin-like receptor 1 (LAIRI); Fc fragment of IgA receptor (FCAR or CD89); leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1). In some embodiments, the target is an epitope of the tumor associated antigen presented in an MHC.

In some embodiments, the tumor antigen is selected from CD150, 5T4, ActRIIA, B7, TNF receptor superfamily member 17 (TNFRSF17, BCMA), CA-125, CCNA1, CD123, CD126, CD138, CD14, CD148, CD15, CD19, CD20, CD200, CD21, CD22, CD23, CD24, CD25, CD26, CD261, CD262, CD30, CD33, CD362, CD37, CD38, CD4, CD40, CD40L, CD44, CD46, CD5, CD52, CD53, CD54, CD56, CD66a-d, CD74, CD8, CD80, CD92, CE7, CS-1, CSPG4, ED-B fibronectin, EGFR, EGFRvIII, EGP-2, EGP-4, EPHa2, ErbB2, ErbB3, ErbB4, FBP, HER1-HER2 in combination, HER2-HER3 in combination, HERV-K, HIV-1 envelope glycoprotein gp120, HIV-1 envelope glycoprotein gp41, HLA-DR, HM1.24, HMW-MAA, Her2, Her2/neu, IGF-1R, IL-11Ralpha, IL-13R-alpha2, IL-2, IL-22R-alpha, IL-6, IL-6R, Ia, Ii, L1-CAM, L1-cell adhesion molecule, Lewis Y, L1-CAM, MAGE A3, MAGE-A1, MART-1, MUC1, NKG2C ligands, NKG2D Ligands, NYESO-1, OEPHa2, PIGF, PSCA, PSMA, ROR1, T101, TAC, TAG72, TIM-3, TRAIL-R1, TRAIL-R1 (DR4), TRAIL-R2 (DR5), VEGF, VEGFR2, WT-I, a G-protein coupled receptor, alphafetoprotein (AFP), an angiogenesis factor, an exogenous cognate binding molecule (ExoCBM), oncogene product, anti-folate receptor, c-Met, carcinoembryonic antigen (CEA), cyclin (D 1), ephrinB2, epithelial tumor antigen, estrogen receptor, fetal acetylcholine e receptor, folate binding protein, gp100, hepatitis B surface antigen, kappa chain, kappa light chain, kdr, lambda chain, livin, melanoma-associated antigen, mesothelin, mouse double minute 2 homolog (MDM2), mucin 16 (MUC16), mutated p53, mutated ras, necrosis antigens, oncofetal antigen, ROR2, progesterone receptor, prostate specific antigen, tEGFR, tenascin, P2-Microgiobuiin, Fc Receptor-like 5 (FcRL5).

In some embodiments, the antigen binding domain binds to an epitope of a target or tumor associated antigen (TAA) presented in a major histocompatibility complex (MHC) molecule. In some embodiments, the TAA is a cancer testis antigen. In some embodiments, the cancer testis antigen is selected from the group consisting of acrosin binding protein (ACRBP; CT23, OY-TES-1, SP32; NCBI Gene ID: 84519), alpha fetoprotein (AFP; AFPD, FETA, HPAFP; NCBI Gene ID: 174); A-kinase anchoring protein 4 (AKAP4; AKAP 82, AKAP-4, AKAP82, CT99, FSC1, HI, PRKA4, hAKAP82, p82; NCBI Gene ID: 8852), ATPase family AAA domain containing 2 (ATAD2; ANCCA, CT137, PRO2000; NCBI Gene ID: 29028), kinetochore scaffold 1 (KNL1; AF15Q14, CASC5, CT29, D40, MCPH4, PPP1R55, Spc7, hKNL-1, hSpc105; NCBI Gene ID: 57082), centrosomal protein 55 (CEP55; C10orf3, CTiii, MARCH, URCC6; NCBI Gene ID: 55165), cancer/testis antigen 1A (CTAG1A; ESO1; CT6.1; LAGE-2; LAGE2A; NY-ESO-1; NCBI Gene ID: 246100), cancer/testis antigen 1B (CTAG1B; CT6.1, CTAG, CTAG1, ESO1, LAGE-2, LAGE2B, NY-ESO-1; NCBI Gene ID: 1485), cancer/testis antigen 2 (CTAG2; CAMEL, CT2, CT6.2, CT6.2a, CT6.2b, ESO2, LAGE-1, LAGE2B; NCBI Gene ID: 30848), CCCTC-binding factor like (CTCFL; BORIS, CT27, CTCF-T, HMGB1L1, dJ579F20.2; NCBI Gene ID: 140690), catenin alpha 2 (CTNNA2; CAP-R, CAPR, CDCBM9, CT114, CTNR; NCBI Gene ID: 1496), cancer/testis antigen 83 (CT83; CXorf61, KK-LC-1, KKLC1; NCBI Gene ID: 203413), cyclin A1 (CCNA1; CT146; NCBI Gene ID: 8900), DEAD-box helicase 43 (DDX43; CT13, HAGE; NCBI Gene ID: 55510), developmental pluripotency associated 2 (DPPA2; CT100, ECAT15-2, PESCRG1; NCBI Gene ID: 151871), fetal and adult testis expressed 1 (FATE1; CT43, FATE; NCBI Gene ID: 89885), FMR1 neighbor (FMR1NB; CT37, NY-SAR-35, NYSAR35; NCBI Gene ID: 158521), HORMA domain containing 1 (HORMAD1; CT46, NOHMA; NCBI Gene ID: 84072), insulin like growth factor 2 mRNA binding protein 3 (IGF2BP3; CT98, IMP-3, IMP3, KOC, KOC1, VICKZ3; NCBI Gene ID: 10643), leucine zipper protein 4 (LUZP4; CT-28, CT-8, CT28, HOM-TES-85; NCBI Gene ID: 51213), lymphocyte antigen 6 family member K (LY6K; CT97, HSJ001348, URLC10, ly-6K; NCBI Gene ID: 54742), maelstrom spermatogenic transposon silencer (MAEL; CT128, SPATA35; NCBI Gene ID: 84944), MAGE family member A1 (MAGEA1; CT1.1, MAGE1; NCBI Gene ID: 4100); MAGE family member A3 (MAGEA3; CT1.3, HIP8, HYPD, MAGE3, MAGEA6; NCBI Gene ID: 4102); MAGE family member A4 (MAGEA4; CT1.4, MAGE-41, MAGE-X2, MAGE4, MAGE4A, MAGE4B; NCBI Gene ID: 4103); MAGE family member A11 (MAGEA11; CT1.11, MAGE-11, MAGE11, MAGEA-11; NCBI Gene ID: 4110); MAGE family member C1 (MAGEC1; CT7, CT7.1; NCBI Gene ID: 9947); MAGE family member C2 (MAGEC2; CT10, HCA587, MAGEE1; NCBI Gene ID: 51438); MAGE family member D1 (MAGED1; DLXIN-1, NRAGE; NCBI Gene ID: 9500); MAGE family member D2 (MAGED2; 11B6, BARTS5, BCG-1, BCG1, HCA10, MAGE-D2; NCBI Gene ID: 10916), kinesin family member 20B (KIF20B; CT90, KRMP1, MPHOSPH1, MPP-1, MPP1; NCBI Gene ID: 9585), NUF2 component of NDC80 kinetochore complex (NUF2; CDCA1, CT106, NUF2R; NCBI Gene ID: 83540), nuclear RNA export factor 2 (NXF2; CT39, TAPL-2, TCP11X2; NCBI Gene ID: 56001), PAS domain containing repressor 1 (PASD1; CT63, CT64, OXTES1; NCBI Gene ID: 139135), PDZ binding kinase (PBK; CT84, HEL164, Nori-3, SPK, TOPK; NCBI Gene ID: 55872), piwi like RNA-mediated gene silencing 2 (PIWIL2; CT80, HILI, PIWIL1L, mili; NCBI Gene ID: 55124), preferentially expressed antigen in melanoma (PRAME; CT130, MAPE, OIP-4, OIP4; NCBI Gene ID: 23532), sperm associated antigen 9 (SPAG9; CT89, HLC-6, HLC4, HLC6, JIP-4, JIP4, JLP, PHET, PIG6; NCBI Gene ID: 9043), sperm protein associated with the nucleus, X-linked, family member A1 (SPANXA1; CT11.1, CT11.3, NAP-X, SPAN-X, SPAN-Xa, SPAN-Xb, SPANX, SPANX-A; NCBI Gene ID: 30014), SPANX family member A2 (SPANXA2; CT11.1, CT11.3, SPANX, SPANX-A, SPANX-C, SPANXA, SPANXC; NCBI Gene ID: 728712), SPANX family member C (SPANXC; CT11.3, CTp11, SPANX-C, SPANX-E, SPANXE; NCBI Gene ID: 64663), SPANX family member D (SPANXD; CT11.3, CT11.4, SPANX-C, SPANX-D, SPANX-E, SPANXC, SPANXE, dJ171K16.1; NCBI Gene ID: 64648), SSX family member 1 (SSX1; CT5.1, SSRC; NCBI Gene ID: 6756), SSX family member 2 (SSX2; CT5.2, CT5.2A, HD21, HOM-MEL-40, SSX; NCBI Gene ID: 6757), synaptonemal complex protein 3 (SYCP3; COR1, RPRGL4, SCP3, SPGF4; NCBI Gene ID: 50511), testis expressed 14, intercellular bridge forming factor (TEX14; CT113, SPGF23; NCBI Gene ID: 56155), transcription factor Dp family member 3 (TFDP3; CT30, DP4, HCA661; NCBI Gene ID: 51270), serine protease 50 (PRSS50; CT20, TSP50; NCBI Gene ID: 29122), TTK protein kinase (TTK; CT96, ESK, MPH1, MPS1, MPS1L1, PYT; NCBI Gene ID: 7272) and zinc finger protein 165 (ZNF165; CT53, LD65, ZSCAN7; NCBI Gene ID: 7718). T cell receptors (TCRs) and TCR-like antibodies that bind to an epitope of a cancer testis antigen presented in a major histocompatibility complex (MHC) molecule are known in the art and can be used in the herein described heterodimers. Cancer testis antigens associated with neoplasia are summarized, e.g., in Gibbs, et al., Trends Cancer 2018 October; 4(10):701-712 and the CT database website at cta.lncc.br/index.php. Illustrative TCRs and TCR-like antibodies that bind to an epitope of NY-ESO-1 presented in an MHC are described, e.g., in Stewart-Jones, et al., Proc Natl Acad Sci USA. 2009 Apr. 7; 106(14):5784-8; WO2005113595, WO2006031221, WO2010106431, WO2016177339, WO2016210365, WO2017044661, WO2017076308, WO2017109496, WO2018132739, WO2019084538, WO2019162043, WO2020086158 and WO2020086647. Illustrative TCRs and TCR-like antibodies that bind to an epitope of PRAME presented in an MHC are described, e.g., in WO2011062634, WO2016142783, WO2016191246, WO2018172533, WO2018234319 and WO2019109821. Illustrative TCRs and TCR-like antibodies that bind to an epitope of a MAGE variant presented in an MHC are described, e.g., in WO2007032255, WO2012054825, WO2013039889, WO2013041865, WO2014118236, WO2016055785, WO2017174822, WO2017174823, WO2017174824, WO2017175006, WO2018097951, WO2018170338, WO2018225732 and WO2019204683. Illustrative TCRs and TCR-like antibodies that bind to an epitope of alpha fetoprotein (AFP) presented in an MHC are described, e.g., in WO2015011450. Illustrative TCRs and TCR-like antibodies that bind to an epitope of SSX2 presented in an MHC are described, e.g., in WO2020063488. Illustrative TCRs and TCR-like antibodies that bind to an epitope of KK-LC-1 (CT83) presented in an MHC are described, e.g., in WO2017189254.

Examples of cell therapies include: Algenpantucel-L, Sipuleucel-T, (BPX-501) rivogenlecleucel U.S. Pat. No. 9,089,520, WO2016100236, AU-105, ACTR-087, activated allogeneic natural killer cells CNDO-109-AANK, MG-4101, AU-101, BPX-601, FATE-NK100, LFU-835 hematopoietic stem cells, Imilecleucel-T, baltaleucel-T, PNK-007, UCARTCS1, ET-1504, ET-1501, ET-1502, ET-190, CD19-ARTEMIS, ProHema, FT-1050-treated bone marrow stem cell therapy, CD4CARNK-92 cells, CryoStim, AlloStim, lentiviral transduced huCART-meso cells, CART-22 cells, EGFRt/19-28z/4-1BBL CAR T cells, autologous 4H11-28z/fIL-12/EFGRt T cell, CCR5-SBC-728-HSPC, CAR4-1BBZ, CH-296, dnTGFbRII-NY-ESOc259T, Ad-RTS-IL-12, IMA-101, IMA-201, CARMA-0508, TT-18, CMD-501, CMD-503, CMD-504, CMD-502, CMD-601, CMD-602, and CSG-005.

In some embodiments the one or more additional co-administered therapeutic agents can be categorized by their mechanism of action, e.g., into the following groups:
agents targeting adenosine deaminase, such as pentostatin or cladribine;
agents targeting ATM, such as AZD1390;
agents targeting MET, such as savolitinib, capmatinib, tepotinib, ABT-700, AG213, JNJ-38877618 (OMO-1), merestinib, HQP-8361, BMS-817378, or TAS-115;
agents targeting mitogen-activated protein kinase, such as antroquinonol, binimetinib, cobimetinib, selumetinib, trametinib, uprosertib, mirdametinib (PD-0325901), pimasertib, refametinib, or compounds disclosed in WO2011008709, WO2013112741, WO2006124944, WO2006124692, WO2014064215, WO2018005435, Zhou, et al., Cancer Lett. 2017 Nov. 1, 408:130-137, Teli, et al., J Enzyme Inhib Med Chem. (2012) 27(4):558-70; Gangwall, et al., Curr Top Med Chem. (2013) 13(9):1015-35; Wu, et al., Bioorg Med Chem Lett. (2009) 19(13):3485-8; Kaila, et al., Bioorg Med Chem. (2007) 15(19):6425-42, or Hu, et al., Bioorg Med Chem Lett. (2011) 21(16):4758-61;
agents targeting thymidine kinase, such as aglatimagene besadenovec (ProstAtak, PancAtak, GliAtak, GMCI, or AdV-tk);
agents targeting an interleukin pathway, such as pegilodecakin (AM-0010) (pegylated IL10), CA-4948 (IRAK4 inhibitor);
agents targeting cytochrome P450 family members, such as letrozole, anastrozole, aminoglutethimide, megestrol acetate (MEGACE®), exemestane, formestane, fadrozole, vorozole (RIVISOR®), letrozole (FEMARA®), or anastrozole (ARIMIDEX®);
agents targeting CD73, such as a CD73 inhibitor (e.g., quemliclustat (AB680)) or an anti-CD73 antibody (e.g., oleclumab);
agents targeting DKK3, such as MTG-201;
agents targeting EEF1A2, such as plitidepsin;
agents targeting EIF4A1, such as rohinitib;
agents targeting endoglin, such as TRC105 (carotuximab);
agents targeting exportin-1, such as eltanexor;
agents targeting fatty acid amide hydrolase, such as compounds disclosed in WO2017160861;
agents targeting heat shock protein 90 beta family member 1, such as anlotinib;
agents targeting lactotransferrin, such as ruxotemitide (LTX-315);
agents targeting lysyl oxidase, such as compounds disclosed in U.S. Pat. Nos. 4,965,288, 4,997,854, 4,943,593, 5,021,456, 5,059,714, 5,120,764, 5,182,297, 5,252,608, or US20040248871;
agents targeting MAGE family members, such as KITE-718, MAGE-A10C796T, or MAGE-A10 TCR;
agents targeting MDM2, such as ALRN-6924, CMG-097, milademetan monotosylate monohydrate (DS-3032b), or AMG-232;
agents targeting MDM4, such as ALRN-6924;
agents targeting melan-A, such as MART-1 F5 TCR engineered PBMCs;
agents targeting mesothelin, such as CSG-MESO or TC-210;
agents targeting METAP2, such as M8891 or APL-1202;
agents targeting NLRP3, such as BMS-986299;
agents targeting C═Oglutarate dehydrogenase, such as devimistat (CPI-613);
agents targeting placenta growth factor, such as aflibercept;
agents targeting SLC10A3, such as compounds disclosed in WO2015148954, WO2012082647, or WO2017160861;
agents targeting transforming growth factor alpha (TGFα), such as compounds disclosed in WO2019103203;
agents targeting tumor protein p53, such as kevetrin (stimulator);
agents targeting vascular endothelial growth factor A, such as aflibercept;
agents targeting vascular endothelial growth factor receptor, such as fruquintinib or MP0250;
agents targeting VISTA, such as CA-170, or HMBD-002;
agents targeting WEE1, such as adavosertib (AZD-1775);
small molecule inhibitors targeting ABL1, such as imatinib, rebastinib, asciminib, or ponatinib (ICLUSIG®);
small molecule antagonists targeting adenosine receptor, such as CPI-444, AZD-4635, preladenant, etrumadenant (AB928), or PBF-509;
small molecule inhibitors targeting arachidonate 5-lipoxygenase, such as meclofenamate sodium or zileuton;
small molecule inhibitors targeting ATR serine/threonine kinase, such as BAY-937, ceralasertib (AZD6738), AZD6783, VX-803, or VX-970 (berzosertib);
small molecule inhibitors targeting AXL receptor tyrosine kinase, such as bemcentinib (BGB-324), SLC-0211, or gilteritinib (Axl/Flt3);
small molecule inhibitors targeting Bruton's tyrosine kinase (BTK), such as (S)-6-amino-9-(1-(but-2-ynoyl)pyrrolidin-3-yl)-7-(4-phenoxyphenyl)-7H-purin-8(9H)-one, acalabrutinib (ACP-196), zanubrutinib (BGB-3111), CB988, poseltinib (HM71224), ibrutinib (Imbruvica), M-2951 (evobrutinib), tirabrutinib (ONO-4059), rilzabrutinib (PRN-1008), spebrutinib (CC-292), vecabrutinib, ARQ-531 (MK-1026), SHR-1459, DTRMWXHS-12, or TAS-5315;
small molecule inhibitors targeting neurotrophic receptor tyrosine kinase such as larotrectinib, entrectinib, or selitrectinib (LOXO-195);
small molecule inhibitors targeting ROS proto-oncogene 1, receptor tyrosine kinase, such as entrectinib, repotrectinib (TPX-0005), or lorlatinib;
small molecule inhibitors targeting SRC proto-oncogene, non-receptor tyrosine kinase, such as VAL-201, tirbanibulin (KX2-391), or ilginatinib maleate (NS-018);
small molecule inhibitors targeting B-cell lymphoma 2, such as navitoclax (ABT-263), venetoclax (ABT-199, RG-7601), or AT-101 (gossypol);
small molecule inhibitors targeting bromodomain and external domain (BET) bromodomain containing protein, such as ABBV-744, INCB-054329, INCB057643, AZD-5153, ABT-767, BMS-986158, CC-90010, NHWD-870, ODM-207, ZBC246, ZEN3694, CC-95775 (FT-1101), mivebresib, BI-894999, PLX-2853, PLX-51107, CPI-0610, or GS-5829;
small molecule inhibitors targeting carbohydrate sulfotransferase 15, such as STNM-01;
small molecule inhibitors targeting carbonic anhydrase, such as polmacoxib, acetazolamide, or methazolamide;
small molecule inhibitors targeting catenin beta 1, such as CWP-291, or PRI-724;
small molecule antagonists targeting a C-C motif chemokine receptor, such as CCX-872, BMS-813160 (CCR2/CCR5) or MK-7690 (vicriviroc);
small molecule antagonists targeting a C-X-C motif chemokine receptor (e.g., CXCR4), blixafortide;
small molecule inhibitors targeting cereblon, such as avadomide (CC-122), CC-92480, CC-90009, or iberdomide;
small molecule inhibitors targeting checkpoint kinase 1, such as SRA737;
small molecule inhibitors targeting a complement component, such as Imprime PGG (Biothera Pharmaceuticals);
small molecule inhibitor targeting a C-X-C motif chemokine ligand (e.g., CXCL12), such as olaptesed pegol (NOX-A12);
small molecule inhibitors targeting cytochrome P450 family, such as ODM-209, LAE-201, seviteronel (VT-464), CFG920, abiraterone, or abiraterone acetate;
small molecule inhibitors targeting DEAD-box helicase 5, such as supinoxin (RX-5902);
small molecule inhibitors targeting DGKα, e.g., such as described in WO2021130638;
small molecule inhibitors targeting diablo IAP-binding mitochondrial protein, such as BI-891065;
small molecule inhibitors targeting dihydrofolate reductase, such as pralatrexate or pemetrexed disodium;
small molecule inhibitors targeting DNA dependent protein kinase, such as MSC2490484A (nedisertib), VX-984, AsiDNA (DT-01), LXS-196, or sotrastaurin;
small molecule inhibitors targeting MARCKS, such as BIO-11006;
small molecule inhibitors targeting RIPK1, such as GSK-3145094;
small molecule inhibitors targeting Rho associated coiled-coil containing protein kinase, such as AT13148 or KD025;
small molecule inhibitors targeting DNA topoisomerase, such as irinotecan, firtecan pegol, or amrubicin;
small molecule inhibitors targeting dopamine receptor D2, such as ONC-201;
small molecule inhibitors targeting DOT1 like histone lysine methyltransferase, such as pinometostat (EPZ-5676);
small molecule inhibitors targeting EZH2, such as tazemetostat, CPI-1205, or PF-06821497;
small molecule inhibitors targeting fatty acid synthase, such as TVB-2640 (Sagimet Biosciences);
small molecule inhibitors targeting fibroblast growth factor receptor 2 (FGFR2), such as bemarituzumab (FPA144);
small molecule inhibitors targeting focal adhesion kinase (FAK, PTK2), such as VS-4718, defactinib, or GSK2256098;
small molecule inhibitors targeting folate receptor 1, such as pralatrexate;
small molecule inhibitors targeting FOXM1, such as thiostrepton;
small molecule inhibitors targeting galectin 3, such as belapectin (GR-MD-02);
small molecule antagonists targeting glucocorticoid receptor, such as relacorilant (CORT-125134);
small molecule inhibitors targeting glutaminase include without limitation CB-839 (telaglenastat), or bis-2-(5-phenylacetamido-1,3,4-thiadiazol-2-yl)ethyl sulfide (BPTES);
small molecule inhibitors targeting GNRHR, such as elagolix, relugolix, or degarelix;
small molecule inhibitors targeting EPAS1, such as belzutifan (PT-2977 (Merck & Co.));
small molecule inhibitors targeting isocitrate dehydrogenase (NADP(+)), such as limitation ivosidenib (AG-120), vorasidenib (AG-881) (IDH1 and IDH2), IDH-305, or enasidenib (AG-221);
small molecule inhibitors targeting lysine demethylase 1A, such as CC-90011;
small molecule inhibitors targeting MAPK interacting serine/threonine kinase, such as tomivosertib (eFT-508);
small molecule inhibitors targeting notch receptor, such as AL-101 (BMS-906024);
small molecule inhibitors targeting polo like kinase 1 (PLK1), such as volasertib or onvansertib;
small molecule inhibitors targeting poly(ADP-ribose) polymerase (PARP), such as olaparib (MK7339), rucaparib, veliparib, talazoparib, ABT-767, pamiparib (BGB-290), fluazolepali (SHR-3162), niraparib (JNJ-64091742), stenoparib (2X-121 (e-7499)), simmiparib, IMP-4297, SC-10914, IDX-1197, HWH-340, CEP 9722, CEP-8983, E7016, 3-aminobenzamide, or CK-102;
small molecule inhibitors targeting polycomb protein EED, such as MAK683;
small molecule inhibitors targeting porcupine O-acyltransferase, such as WNT-974;
small molecule inhibitors targeting prostaglandin-endoperoxide synthase, such as HP-5000, lornoxicam, ketorolac tromethamine, bromfenac sodium, otenaproxesul (ATB-346), mofezolac, GLY-230, TRK-700, diclofenac, meloxicam, parecoxib, etoricoxib, celecoxib, AXS-06, diclofenac potassium, reformulated celecoxib (DRGT-46), AAT-076, meisuoshuli, lumiracoxib, meloxicam, valdecoxib, zaltoprofen, nimesulide, anitrazafen, apricoxib, cimicoxib, deracoxib, flumizole, firocoxib, mavacoxib, pamicogrel, parecoxib, robenacoxib, rofecoxib, rutecarpine, tilmacoxib, zaltoprofen, or imrecoxib;
small molecule inhibitors targeting protein arginine N methyltransferase, such as MS203, PF-06939999, GSK3368715, or GSK3326595;
small molecule inhibitors targeting PTPN11, such as TNO155 (SHP-099), RMC-4550, JAB-3068, RMC-4630 (SAR442720), or compounds disclosed in WO2018172984 or WO2017211303;
small molecule antagonist targeting retinoic acid receptor, such as tamibarotene (SY-1425);
small molecule inhibitors targeting ribosomal protein S6 kinase B1, such as MSC2363318A;
small molecule inhibitors targeting 5100 calcium binding protein A9, such as tasquinimod;
small molecule inhibitors targeting selectin E, such as uproleselan sodium (GMI-1271);
small molecule inhibitors targeting SF3B1, such as H3B-8800;
small molecule inhibitors targeting Sirtuin-3, such as YC8-02;
small molecule inhibitors targeting SMO, such as sonidegib (Odomzo®, formerly LDE-225), vismodegib (GDC-0449), glasdegib (PF-04449913), itraconazole, or patidegib, taladegib;
small molecule antagonists targeting somatostatin receptor, such as OPS-201;
small molecule inhibitors targeting sphingosine kinase 2, such as opaganib (Yeliva®, ABC294640);
small molecule inhibitors targeting STAT3, such as napabucasin (BBI-608);
small molecule inhibitors targeting tankyrase, such as G007-LK or stenoparib (2X-121 (e-7499));
small molecule inhibitors targeting TFGBR1, such as galunisertib, PF-06952229;
small molecule inhibitors targeting thymidylate synthase, such as idetrexed (ONX-0801);
small molecule inhibitors targeting tumor protein p53, such as CMG-097;
small molecule inhibitors targeting valosin-containing protein, such as CB-5083;
small molecule inhibitors targeting WT1, such as ombipepimut-S (DSP-7888);
small molecule agonists targeting adenosine receptor, such as namodenoson (CF102);
small molecule agonist(s) targeting asparaginase, such as crisantaspase (Erwinase®), GRASPA (ERY-001, ERY-ASP), calaspargase pegol, or pegaspargase;
small molecule agonists targeting CCAAT enhancer binding protein alpha, such as MTL-501;
small molecule agonists targeting cytochrome P450 family, such as mitotane;
small molecule agonists targeting DExD/H-box helicase 58, such as RGT-100;
small molecule agonists targeting GNRHR, such as leuprorelin acetate, leuprorelin acetate sustained release depot (ATRIGEL), triptorelin pamoate, or goserelin acetate;
small molecule agonists targeting GRB2, such as prexigebersen (BP1001);
small molecule agonists targeting NFE2L2, such as omavelC═Olone (RTA-408);
small molecule agonists targeting NOD2, such as mifamurtide (liposomal);
small molecule agonists targeting RAR-related orphan receptor gamma, such as cintirorgon (LYC-55716);
small molecule agonists targeting retinoic acid receptor (RAR), such as tretinoin;
small molecule agonists targeting STING1, such as ADU-S100 (MIW-815), SB-11285, MK-1454, SR-8291, AdVCA0848, GSK-532, SYN-STING, MSA-1, SR-8291, cyclic-GAMP (cGAMP), or cyclic-di-AMP;
small molecule agonists targeting thyroid hormone receptor beta, such as levothyroxine sodium;
small molecule agonists targeting tumor necrosis factor, such as tasonermin;
antisense agents targeting baculoviral IAP repeat containing 5, such as EZN-3042;
antisense agents targeting GRB2, such as prexigebersen;
antisense agents targeting heat shock protein 27, such as apatorsen;
antisense agents targeting STAT3, such as danvatirsen (IONIS-STAT3-2.5Rx);
gene therapies targeting a C-C motif chemokine receptor, such as SB-728-T;
gene therapies targeting an interleukin, such as EGENE-001, tavokinogene telseplasmid, nogapendekin alfa (ALT-803), NKTR-255, NIZ-985 (hetIL-15), SAR441000, or MDNA-55;
antibodies targeting claudin 18, such as claudiximab;
antibodies targeting clusterin, such as AB-16B5;
antibodies targeting a complement component, such as ravulizumab (ALXN-1210);
antibodies targeting a C-X-C motif chemokine ligand, such as BMS-986253 (HuMax-Inflam);
antibodies targeting delta like canonical Notch ligand 4 (DLL4), such as demcizumab, navicixizumab (DLL4/VEGF);
antibodies targeting EPH receptor A3, such as fibatuzumab (KB-004);
antibodies targeting epithelial cell adhesion molecule, such as oportuzumab monatox (VB4-845);
antibodies targeting fibroblast growth factor, such as GAL-F2, B-701 (vofatamab);
antibodies targeting hepatocyte growth factor, such as MP-0250;
antibodies targeting an interleukin, such as canakinumab (ACZ885), gevokizumab (VPM087), CJM-112, guselkumab, talacotuzumab (JNJ-56022473), siltuximab, or tocilizumab;
antibodies targeting LRRC15, such as ABBV-085 or cusatuzumab (ARGX-110);
antibodies targeting mesothelin, such as BMS-986148, SEL-403, or anti-MSLN-MMAE;
antibodies targeting myostatin, such as landogrozumab;
antibodies targeting notch receptor, such as tarextumab;
antibodies targeting TGFB1 (TGFβ1), such as SAR439459, ABBV-151, NIS793, SRK-181, XOMA089, or compounds disclosed in WO2019103203;
vaccines targeting fins related receptor tyrosine kinase, such as HLA-A2402/HLA-A0201 restricted epitope peptide vaccine;
vaccines targeting heat shock protein 27, such as PSV-AML (PhosphoSynVax);
vaccines targeting PD-L1, such as IO-120+IO-103 (PD-L1/PD-L2 vaccines) or IO-103;
vaccines targeting tumor protein p53, such as MVA-p53;
vaccines targeting WT1, such as WT-1 analog peptide vaccine (WT1-CTL);
cell therapies targeting baculoviral IAP repeat containing 5, such as tumor lysate/MUC1/survivin PepTivator-loaded dendritic cell vaccine;
cell therapies targeting carbonic anhydrase, such as DC-Ad-GMCAIX;
cell therapies targeting C-C motif chemokine receptor, such as CCR5-SBC-728-HSPC;
cell therapies targeting folate hydrolase 1, such as CIK-CAR.PSMA or CART-PSMA-TGFβRDN;
cell therapies targeting GSTP1, such as CPG3-CAR (GLYCAR);
cell therapies targeting HLA-A, such as FH-MCVA2TCR or NeoTCR-P1;
cell therapies targeting an interleukin, such as CST-101;
cell therapies targeting KRAS, such as anti-KRAS G12D mTCR PBL;
cell therapies targeting MET, such as anti-cMet RNA CAR T;
cell therapies targeting MUC16, such as JCAR-020;
cell therapies targeting PD-1, such as PD-1 knockout T cell therapy (esophageal cancer/NSCLC);
cell therapies targeting PRAME, such as BPX-701;
cell therapies targeting transforming protein E7, such as KITE-439;
cell therapies targeting WT1, such as WT1-CTL, ASP-7517, or JTCR-016.

### Exemplified Combination Therapies

### Lymphoma or Leukemia Combination Therapy

Some chemotherapy agents are suitable for treating lymphoma or leukemia. These agents include aldesleukin, alvocidib, amifostine trihydrate, aminocamptothecin, antineoplaston A10, antineoplaston AS2-1, anti-thymocyte globulin, arsenic trioxide, Bcl-2 family protein inhibitor ABT-263, beta alethine, BMS-345541, bortezomib (VELCADE®), bortezomib (VELCADE®, PS-341), bryostatin 1, bulsulfan, campath-1H, carboplatin, carfilzomib (Kyprolis®), carmustine, caspofungin acetate, CC-5103, chlorambucil, CHOP (cyclophosphamide, dC═Orubicin, vincristine, and prednisone), cisplatin, cladribine, clofarabine, curcumin, CVP (cyclophosphamide, vincristine, and prednisone), cyclophosphamide, cyclosporine, cytarabine, denileukin diftitox, dexamethasone, docetaxel, dolastatin 10, dC═Orubicin, dC═Orubicin hydrochloride, DT-PACE (dexamethasone, thalidomide, cisplatin, dC═Orubicin, cyclophosphamide, and etoposide), enzastaurin, epoetin alfa, etoposide, everolimus (RAD001), FCM (fludarabine, cyclophosphamide, and mitoxantrone), FCR (fludarabine, cyclophosphamide, and rituximab), fenretinide, filgrastim, flavopiridol, fludarabine, FR (fludarabine and rituximab), geldanamycin (17 AAG), hyperCVAD (hyperfractionated cyclophosphamide, vincristine, dC═Orubicin, dexamethasone, methotrexate, and cytarabine), ICE (iphosphamide, carboplatin, and etoposide), ifosfamide, irinotecan hydrochloride, interferon alpha-2b, ixabepilone, lenalidomide (REVLIMID®, CC-5013), lymphokine-activated killer cells, MCP (mitoxantrone, chlorambucil, and prednisolone), melphalan, mesna, methotrexate, mitoxantrone hydrochloride, motexafin gadolinium, mycophenolate mofetil, nelarabine, obatoclax (GX15-070), oblimersen, octreotide acetate, omega-3 fatty acids, Omr-IgG-am (WNIG, Omrix), oxaliplatin, paclitaxel, palbociclib (PD0332991), pegfilgrastim, PEGylated liposomal dC═Orubicin hydrochloride, perifosin, prednisolone, prednisone, recombinant flt3 ligand, recombinant human thrombopoietin, recombinant interferon alfa, recombinant interleukin-11, recombinant interleukin-12, rituximab, R-CHOP (rituximab and CHOP), R-CVP (rituximab and CVP), R-FCM (rituximab and FCM), R-ICE (rituximab and ICE), and R MCP (rituximab and MCP), R-roscovitine (seliciclib, CYC202), sargramostim, sildenafil citrate, simvastatin, sirolimus, styryl sulphones, tacrolimus, tanespimycin, temsirolimus (CCl-779), thalidomide, therapeutic allogeneic lymphocytes, thiotepa, tipifarnib, vincristine, vincristine sulfate, vinorelbine ditartrate, SAHA (suberanilohydroxamic acid, or suberoyl, anilide, and hydroxamic acid), vemurafenib (Zelboraf®), venetoclax (ABT-199).

One modified approach is radioimmunotherapy, wherein a monoclonal antibody is combined with a radioisotope particle, such as indium-111, yttrium-90, and iodine-131. Examples of combination therapies include, but are not limited to, iodine-131 tositumomab (BEXXAR®), yttrium-90 ibritumomab tiuxetan (ZEVALIN®), and BEXXAR® with CHOP.

The abovementioned therapies can be supplemented or combined with stem cell transplantation or treatment. Therapeutic procedures include peripheral blood stem cell transplantation, autologous hematopoietic stem cell transplantation, autologous bone marrow transplantation, antibody therapy, biological therapy, enzyme inhibitor therapy, total body irradiation, infusion of stem cells, bone marrow ablation with stem cell support, in vitro-treated peripheral blood stem cell transplantation, umbilical cord blood transplantation, immunoenzyme technique, low-LET cobalt-60 gamma ray therapy, bleomycin, conventional surgery, radiation therapy, and nonmyeloablative allogeneic hematopoietic stem cell transplantation.

### Non-Hodgkin's Lymphomas Combination Therapy

Treatment of non-Hodgkin's lymphomas (NHL), especially those of B cell origin, includes using monoclonal antibodies, standard chemotherapy approaches (e.g., CHOP (cyclophosphamide, dC═Orubicin, vincristine, and prednisone), CVP (cyclophosphamide, vincristine, and prednisone), FCM (fludarabine, cyclophosphamide, and mitoxantrone), MCP (Mitoxantrone, Chlorambucil, Prednisolone), all optionally including Rituximab® and the like), radioimmunotherapy, and combinations thereof, especially integration of an antibody therapy with chemotherapy.

Examples of unconjugated monoclonal antibodies for the treatment of NHL/B-cell cancers include rituximab, alemtuzumab, human or humanized anti-CD20 antibodies, lumiliximab, anti-TNF-related apoptosis-inducing ligand (anti-TRAIL), bevacizumab, galiximab, epratuzumab, SGN-40, and anti-CD74.

Examples of experimental antibody agents used in treatment of NHL/B-cell cancers include ofatumumab, ha20, PRO131921, alemtuzumab, galiximab, SGN-40, CHIR-12.12, epratuzumab, lumiliximab, apolizumab, milatuzumab, and bevacizumab.

Examples of standard regimens of chemotherapy for NHL/B-cell cancers include CHOP, FCM, CVP, MCP, R-CHOP (rituximab, cyclophosphamide, dC═Orubicin, vincristine, and prednisone), R-FCM, R-CVP, and R MCP.

Examples of radioimmunotherapy for NHL/B-cell cancers include yttrium-90 ibritumomab tiuxetan (ZEVALIN®) and iodine-131 tositumomab (BEXXAR®).

### Mantle Cell Lymphoma Combination Therapy

Therapeutic treatments for mantle cell lymphoma (MCL) include combination chemotherapies such as CHOP, hyperCVAD, and FCM. These regimens can also be supplemented with the monoclonal antibody rituximab to form combination therapies R-CHOP, hyperCVAD-R, and R-FCM. Any of the abovementioned therapies may be combined with stem cell transplantation or ICE in order to treat MCL.

An alternative approach to treating MCL is immunotherapy. One immunotherapy uses monoclonal antibodies like rituximab. Another uses cancer vaccines, such as GTOP-99, which are based on the genetic makeup of an individual patient's tumor.

A modified approach to treat MCL is radioimmunotherapy, wherein a monoclonal antibody is combined with a radioisotope particle, such as iodine-131 tositumomab (BEXXAR®) and yttrium-90 ibritumomab tiuxetan (ZEVALIN®). In another example, BEXXAR® is used in sequential treatment with CHOP.

Other approaches to treating MCL include autologous stem cell transplantation coupled with high-dose chemotherapy, administering proteasome inhibitors such as bortezomib (VELCADE® or PS-341), or administering antiangiogenesis agents such as thalidomide, especially in combination with rituximab.

Another treatment approach is administering drugs that lead to the degradation of Bcl-2 protein and increase cancer cell sensitivity to chemotherapy, such as oblimersen, in combination with other chemotherapeutic agents.

A further treatment approach includes administering mTOR inhibitors, which can lead to inhibition of cell growth and even cell death. Non-limiting examples are sirolimus, temsirolimus (TORISEL®, CCI-779), CC-115, CC-223, SF-1126, PQR-309 (bimiralisib), voxtalisib, GSK-2126458, and temsirolimus in combination with RITUXAN®, VELCADE®, or other chemotherapeutic agents.

Other recent therapies for MCL have been disclosed. Such examples include flavopiridol, palbociclib (PD0332991), R-roscovitine (selicicilib, CYC202), styryl sulphones, obatoclax (GX15-070), TRAIL, Anti-TRAIL death receptors DR4 and DR5 antibodies, temsirolimus (TORISEL®, CCl-779), everolimus (RAD001), BMS-345541, curcumin, SAHA, thalidomide, lenalidomide (REVLIMID®, CC-5013), and geldanamycin (17 AAG).

### Waldenstrom's Macroglobulinemia Combination Therapy

Therapeutic agents used to treat Waldenstrom's Macroglobulinemia (WM) include aldesleukin, alemtuzumab, alvocidib, amifostine trihydrate, aminocamptothecin, antineoplaston A10, antineoplaston AS2-1, anti-thymocyte globulin, arsenic trioxide, autologous human tumor-derived HSPPC-96, Bcl-2 family protein inhibitor ABT-263, beta alethine, bortezomib (VELCADE®), bryostatin 1, busulfan, campath-1H, carboplatin, carmustine, caspofungin acetate, CC-5103, cisplatin, clofarabine, cyclophosphamide, cyclosporine, cytarabine, denileukin diftitox, dexamethasone, docetaxel, dolastatin 10, dC═Orubicin hydrochloride, DT-PACE, enzastaurin, epoetin alfa, epratuzumab (hLL2-anti-CD22 humanized antibody), etoposide, everolimus, fenretinide, filgrastim, fludarabine, ibrutinib, ifosfamide, indium-111 monoclonal antibody MN-14, iodine-131 tositumomab, irinotecan hydrochloride, ixabepilone, lymphokine-activated killer cells, melphalan, mesna, methotrexate, mitoxantrone hydrochloride, monoclonal antibody CD19 (such as tisagenlecleucel-T, CART-19, CTL-019), monoclonal antibody CD20, motexafin gadolinium, mycophenolate mofetil, nelarabine, oblimersen, octreotide acetate, omega-3 fatty acids, oxaliplatin, paclitaxel, pegfilgrastim, PEGylated liposomal dC═Orubicin hydrochloride, pentostatin, perifosine, prednisone, recombinant flt3 ligand, recombinant human thrombopoietin, recombinant interferon alfa, recombinant interleukin-11, recombinant interleukin-12, rituximab, sargramostim, sildenafil citrate (VIAGRA®), simvastatin, sirolimus, tacrolimus, tanespimycin, thalidomide, therapeutic allogeneic lymphocytes, thiotepa, tipifarnib, tositumomab, ulocuplumab, veltuzumab, vincristine sulfate, vinorelbine ditartrate, vorinostat, WT1 126-134 peptide vaccine, WT-1 analog peptide vaccine, yttrium-90 ibritumomab tiuxetan, yttrium-90 humanized epratuzumab, and any combination thereof.

Examples of therapeutic procedures used to treat WM include peripheral blood stem cell transplantation, autologous hematopoietic stem cell transplantation, autologous bone marrow transplantation, antibody therapy, biological therapy, enzyme inhibitor therapy, total body irradiation, infusion of stem cells, bone marrow ablation with stem cell support, in vitro-treated peripheral blood stem cell transplantation, umbilical cord blood transplantation, immunoenzyme techniques, low-LET cobalt-60 gamma ray therapy, bleomycin, conventional surgery, radiation therapy, and nonmyeloablative allogeneic hematopoietic stem cell transplantation.

### Diffuse Large B-Cell Lymphoma (DLBCL) Combination Therapy

Therapeutic agents used to treat diffuse large B-cell lymphoma (DLBCL) include cyclophosphamide, dC═Orubicin, vincristine, prednisone, anti-CD20 monoclonal antibodies, etoposide, bleomycin, many of the agents listed for WM, and any combination thereof, such as ICE and RICE. In some embodiments therapeutic agents used to treat DLBCL include rituximab (Rituxan®), cyclophosphamide, dC═Orubicin hydrochloride (hydroxydaunorubicin), vincristine sulfate (Oncovin®), prednisone, bendamustine, ifosfamide, carboplatin, etoposide, ibrutinib, polatuzumab vedotin piiq, bendamustine, copanlisib, lenalidomide (Revlimid®), dexamethasone, cytarabine, cisplatin, Yescarta®, Kymriah®, Polivy® (polatuzumab vedotin), BR (bendamustine (Treanda®), gemcitabine, oxiplatin, oxaliplatin, tafasitamab, polatuzumab, cyclophosphamide, or combinations thereof. In some embodiments therapeutic agents used to treat DLBCL include R-CHOP (rituximab+cyclophosphamide+dC═Orubicin hydrochloride (hydroxydaunorubicin)+vincristine sulfate (Oncovin®), +prednisone), rituximab+bendamustine, R-ICE (Rituximab+Ifosfamide+Carboplatin+Etoposide), rituximab+lenalomide, R-DHAP (rituximab+dexamethasone+high-dose cytarabine (Ara C)+cisplatin), Polivy® (polatuzumab vedotin)+BR (bendamustine (Treanda®) and rituximab (Rituxan®), R-GemOx (Gemcitabine+oxaliplatin+rituximab), Tafa-Len (tafasitamab+lenalidomide), Tafasitamab+Revlimid®, polatuzumab+bendamustine, Gemcitabine+oxaliplatin, R-EPOCH (rituximab+etoposide phosphate+prednisone+vincristine sulfate (Oncovin®)+cyclophosphamide+dC═Orubicin hydrochloride (hydroxydaunorubicin)), or CHOP (cyclophosphamide+dC═Orubicin hydrochloride (hydroxydaunorubicin)+vincristine sulfate (Oncovin®)+prednisone). In some embodiments therapeutic agents used to treat DLBCL include tafasitamab, glofitamab, epcoritamab, Lonca-T (loncastuximab tesirine), Debio-1562, polatuzumab, Yescarta, JCAR017, ADCT-402, brentuximab vedotin, MT-3724, odronextamab, Auto-03, Allo-501A, or TAK-007.

### Chronic Lymphocytic Leukemia Combination Therapy

Therapeutic agents used to treat chronic lymphocytic leukemia (CLL) include chlorambucil, cyclophosphamide, fludarabine, pentostatin, cladribine, dC═Orubicin, vincristine, prednisone, prednisolone, alemtuzumab, many of the agents listed for WM, and combination chemotherapy and chemoimmunotherapy, including the following common combination regimens: CVP, R-CVP, ICE, R-ICE, FCR, and FR.

### High Risk Myelodysplastic Syndrome (HR MDS) Combination Therapy

Therapeutic agents used to treat HR MDS include azacitidine (Vidaza®), decitabine (Dacogen®), lenalidomide (Revlimid®), cytarabine, idarubicin, daunorubicin, and combinations thereof. In some embodiments combinations include cytarabine+daunorubicin and cytarabine+idarubicin. In some embodiments therapeutic agents used to treat HR MDS include pevonedistat, venetoclax, sabatolimab, guadecitabine, rigosertib, ivosidenib, enasidenib, selinexor, BGB324, DSP-7888, or SNS-301.

### Low Risk Myelodysplastic Syndrome (LR MDS) Combination Therapy

Therapeutic agents used to treat LR MDS include lenalidomide, azacytidine, and combinations thereof. In some embodiments therapeutic agents used to treat LR MDS include roxadustat, luspatercept, imetelstat, LB-100, or rigosertib.

### Acute Myeloid Leukemia (AML) Combination Therapy

Therapeutic agents used to treat AML include cytarabine, idarubicin, daunorubicin, midostaurin (Rydapt®), venetoclax, azacitidine, ivasidenib, gilteritinib, enasidenib, low-dose cytarabine (LoDAC), mitoxantrone, fludarabine, granulocyte-colony stimulating factor, idarubicin, gilteritinib (Xospata®), enasidenib (Idhifa®), ivosidenib (Tibsovo®), decitabine (Dacogen®), mitoxantrone, etoposide, Gemtuzumab ozogamicin (Mylotarg®), glasdegib (Daurismo®), and combinations thereof. In some embodiments therapeutic agents used to treat AML include FLAG-Ida (fludarabine, cytarabine (Ara-C), granulocyte-colony stimulating factor (G-CSF) and idarubicin), cytarabine+idarubicin, cytarabine+daunorubicin+midostaurin, venetoclax+azacitidine, cytarabine+daunorubicin, or MEC (mitoxantrone, etoposide, and cytarabine). In some embodiments, therapeutic agents used to treat AML include pevonedistat, venetoclax, sabatolimab, eprenetapopt, or lemzoparlimab.

### Multiple Myeloma (MM) Combination Therapy

Therapeutic agents used to treat MM include lenalidomide, bortezomib, dexamethasone, daratumumab (Darzalex®), pomalidomide, Cyclophosphamide, Carfilzomib (Kyprolis®), Elotuzumab (Empliciti), and combinations thereof. In some embodiments therapeutic agents used to treat MM include RVS (lenalidomide+bortezomib+dexamethasone), RevDex (lenalidomide plus dexamethasone), CYBORD (Cyclophosphamide+Bortezomib+Dexamethasone), Vel/Dex (bortezomib plus dexamethasone), or PomDex (Pomalidomide+low-dose dexamethasone). In some embodiments therapeutic agents used to treat MM include JCARH125, TAK-573, belantamab-m, ide-cel (CAR-T).

### Breast Cancer Combination Therapy

Therapeutic agents used to treat breast cancer include albumin-bound paclitaxel, anastrozole, atezolizumab, capecitabine, carboplatin, cisplatin, cyclophosphamide, docetaxel, dC═Orubicin, epirubicin, everolimus, exemestane, fluorouracil, fulvestrant, gemcitabine, Ixabepilone, lapatinib, letrozole, methotrexate, mitoxantrone, paclitaxel, pegylated liposomal dC═Orubicin, pertuzumab, tamoxifen, toremifene, trastuzumab, vinorelbine, and any combinations thereof. In some embodiments therapeutic agents used to treat breast cancer (e.g., HR+/−/HER2+/−) include trastuzumab (Herceptin®), pertuzumab (Perjeta®), docetaxel, carboplatin, palbociclib (Ibrance®), letrozole, trastuzumab emtansine (Kadcyla®), fulvestrant (Faslodex®), olaparib (Lynparza®), eribulin, tucatinib, capecitabine, lapatinib, everolimus (Afinitor®), exemestane, eribulin mesylate (Halaven®), and combinations thereof. In some embodiments therapeutic agents used to treat breast cancer include trastuzumab+pertuzumab+docetaxel, trastuzumab+pertuzumab+docetaxel+carboplatin, palbociclib+letrozole, tucatinib+capecitabine, lapatinib+capecitabine, palbociclib+fulvestrant, or everolimus+exemestane. In some embodiments therapeutic agents used to treat breast cancer include trastuzumab deruxtecan (Enhertu®), datopotamab deruxtecan (DS-1062), enfortumab vedotin (Padcev®), balixafortide, elacestrant, or a combination thereof. In some embodiments therapeutic agents used to treat breast cancer include balixafortide+eribulin.

### Triple Negative Breast Cancer (TNBC) Combination Therapy

Therapeutic agents used to treat TNBC include atezolizumab, cyclophosphamide, docetaxel, dC═Orubicin, epirubicin, fluorouracil, paclitaxel, and combinations thereof. In some embodiments therapeutic agents used to treat TNBC include olaparib (Lynparza®), atezolizumab (Tecentriq®), paclitaxel (Abraxane®), eribulin, bevacizumab (Avastin®), carboplatin, gemcitabine, eribulin mesylate (Halaven®), sacituzumab govitecan (Trodelvy®), pembrolizumab (Keytruda®), cisplatin, dC═Orubicin, epirubicin, or a combination thereof. In some embodiments therapeutic agents to treat TNBC include atezolizumab+paclitaxel, bevacizumab+paclitaxel, carboplatin+paclitaxel, carboplatin+gemcitabine, or paclitaxel+gemcitabine. In some embodiments therapeutic agents used to treat TNBC include eryaspase, capivasertib, alpelisib, rucaparib+nivolumab, atezolumab+paclitaxel+gemcitabine+capecitabine+carboplatin, ipatasertib+paclitaxel, ladiratuzumab vedotin+pembrolimab, durvalumab+DS-8201a, trilaciclib+gemcitabine+carboplatin. In some embodiments therapeutic agents used to treat TNBC include trastuzumab deruxtecan (Enhertu®), datopotamab deruxtecan (DS-1062), enfortumab vedotin (Padcev®), balixafortide, adagloxad simolenin, nelipepimut-s (NeuVax®), nivolumab (Opdivo®), rucaparib, toripalimab (Tuoyi®), camrelizumab, capivasertib, durvalumab (Imfinzi®), and combinations thereof. In some embodiments therapeutic agents use to treat TNBC include nivolumab+rucaparib, bevacizumab (Avastin®)+chemotherapy, toripalimab+paclitaxel, toripalimab+albumin-bound paclitaxel, camrelizumab+chemotherapy, pembrolizumab+chemotherapy, balixafortide+eribulin, durvalumab+trastuzumab deruxtecan, durvalumab+paclitaxel, or capivasertib+paclitaxel.

### Bladder Cancer Combination Therapy

Therapeutic agents used to treat bladder cancer include datopotamab deruxtecan (DS-1062), trastuzumab deruxtecan (Enhertu®), erdafitinib, eganelisib, lenvatinib, bempegaldesleukin (NKTR-214), or a combination thereof. In some embodiments therapeutic agents used to treat bladder cancer include eganelisib+nivolumab, pembrolizumab (Keytruda®)+enfortumab vedotin (Padcev®), nivolumab+ipilimumab, duravalumab+tremelimumab, lenvatinib+pembrolizumab, enfortumab vedotin (Padcev®)+pembrolizumab, and bempegaldesleukin+nivolumab.

### Colorectal Cancer (CRC) Combination Therapy

Therapeutic agents used to treat CRC include bevacizumab, capecitabine, cetuximab, fluorouracil, irinotecan, leucovorin, oxaliplatin, panitumumab, ziv-aflibercept, and any combinations thereof. In some embodiments therapeutic agents used to treat CRC include bevacizumab (Avastin®), leucovorin, 5-FU, oxaliplatin (FOLFOX), pembrolizumab (Keytruda®), FOLFIRI, regorafenib (Stivarga®), aflibercept (Zaltrap®), cetuximab (Erbitux®), Lonsurf (Orcantas®), XELOX, FOLFOXIRI, or a combination thereof. In some embodiments therapeutic agents used to treat CRC include bevacizumab+leucovorin+5-FU+oxaliplatin (FOLFOX), bevacizumab+FOLFIRI, bevacizumab+FOLFOX, aflibercept+FOLFIRI, cetuximab+FOLFIRI, bevacizumab+XELOX, and bevacizumab+FOLFOXIRI. In some embodiments therapeutic agents used to treat CRC include binimetinib+encorafenib+cetuximab, trametinib+dabrafenib+panitumumab, trastuzumab+pertuzumab, napabucasin+FOLFIRI+bevacizumab, nivolumab+ipilimumab.

### Esophageal and Esophagogastric Junction Cancer Combination Therapy

Therapeutic agents used to treat esophageal and esophagogastric junction cancer include capecitabine, carboplatin, cisplatin, docetaxel, epirubicin, fluoropyrimidine, fluorouracil, irinotecan, leucovorin, oxaliplatin, paclitaxel, ramucirumab, trastuzumab, and any combinations thereof. In some embodiments therapeutic agents used to treat gastroesophageal junction cancer (GEJ) include herceptin, cisplatin, 5-FU, ramicurimab, or paclitaxel. In some embodiments therapeutic agents used to treat GEJ cancer include ALX-148, AO-176, or IBI-188.

### Gastric Cancer Combination Therapy

Therapeutic agents used to treat gastric cancer include capecitabine, carboplatin, cisplatin, docetaxel, epirubicin, fluoropyrimidine, fluorouracil, Irinotecan, leucovorin, mitomycin, oxaliplatin, paclitaxel, ramucirumab, trastuzumab, and any combinations thereof.

### Head and Neck Cancer Combination Therapy

Therapeutic agents used to treat head & neck cancer include afatinib, bleomycin, capecitabine, carboplatin, cetuximab, cisplatin, docetaxel, fluorouracil, gemcitabine, hydroxyurea, methotrexate, nivolumab, paclitaxel, pembrolizumab, vinorelbine, and any combinations thereof.

Therapeutic agents used to treat head and neck squamous cell carcinoma (HNSCC) include pembrolizumab, carboplatin, 5-FU, docetaxel, cetuximab (Erbitux®), cisplatin, nivolumab (Opdivo®), and combinations thereof. In some embodiments therapeutic agents used to treat HNSCC include pembrolizumab+carboplatin+5-FU, cetuximab+cisplatin+5-FU, cetuximab+carboplatin+5-FU, cisplatin+5-FU, and carboplatin+5-FU. In some embodiments therapeutic agents used to treat HNSCC include durvalumab, durvalumab+tremelimumab, nivolumab+ipilimumab, rovaluecel, pembrolizumab, pembrolizumab+epacadostat, GSK3359609+pembrolizumab, lenvatinib+pembrolizumab, retifanlimab, retifanlimab+enobituzumab, ADU-S100+pembrolizumab, epacadostat+nivolumab+ipilimumab/lirilumab.

### Non-Small Cell Lung Cancer Combination Therapy

Therapeutic agents used to treat non-small cell lung cancer (NSCLC) include afatinib, albumin-bound paclitaxel, alectinib, atezolizumab, bevacizumab, bevacizumab, cabozantinib, carboplatin, cisplatin, crizotinib, dabrafenib, docetaxel, erlotinib, etoposide, gemcitabine, nivolumab, paclitaxel, pembrolizumab, pemetrexed, ramucirumab, trametinib, trastuzumab, vandetanib, vemurafenib, vinblastine, vinorelbine, and any combinations thereof. In some embodiments therapeutic agents used to treat NSCLC include alectinib (Alecensa®), dabrafenib (Tafinlar®), trametinib (Mekinist®), osimertinib (Tagrisso®), entrectinib (Tarceva®), crizotinib (Xalkori®), pembrolizumab (Keytruda®), carboplatin, pemetrexed (Alimta®), nab-paclitaxel (Abraxane®), ramucirumab (Cyramza®), docetaxel, bevacizumab (Avastin®), brigatinib, gemcitabine, cisplatin, afatinib (Gilotrif®), nivolumab (Opdivo®), gefitinib (Iressa®), and combinations thereof. In some embodiments therapeutic agents used to treat NSCLC include dabrafenib+trametinib, pembrolizumab+carboplatin+pemetrexed, pembrolizumab+carboplatin+nab-paclitaxel, ramucirumab+docetaxel, bevacizumab+carboplatin+pemetrexed, pembrolizumab+pemetrexed+carboplatin, cisplatin+pemetrexed, bevacizumab+carboplatin+nab-paclitaxel, cisplatin+gemcitabine, nivolumab+docetaxel, carboplatin+pemetrexed, carboplatin+nab-paclitaxel, or pemetrexed+cisplatin+carboplatin. In some embodiments therapeutic agents used to NSCLC include datopotamab deruxtecan (DS-1062), trastuzumab deruxtecan (Enhertu®), enfortumab vedotin (Padcev®), durvalumab, canakinumab, cemiplimab, nogapendekin alfa, avelumab, tiragolumab, domvanalimab, vibostolimab, ociperlimab, or a combination thereof. In some embodiments therapeutic agents used to treat NSCLC include datopotamab deruxtecan+pembrolizumab, datopotamab deruxtecan+durvalumab, durvalumab+tremelimumab, pembrolizumab+lenvatinib+pemetrexed, pembrolizumab+olaparib, nogapendekin alfa (N-803)+pembrolizumab, tiragolumab+atezolizumab, vibostolimab+pembrolizumab, or ociperlimab+tislelizumab.

### Small Cell Lung Cancer Combination Therapy

Therapeutic agents used to treat small cell lung cancer (SCLC) include atezolizumab, bendamustime, carboplatin, cisplatin, cyclophosphamide, docetaxel, dC═Orubicin, etoposide, gemcitabine, ipillimumab, irinotecan, nivolumab, paclitaxel, temozolomide, topotecan, vincristine, vinorelbine, and any combinations thereof. In some embodiments therapeutic agents used to treat SCLC include atezolizumab, carboplatin, cisplatin, etoposide, paclitaxel, topotecan, nivolumab, durvalumab, trilaciclib, or combinations thereof. In some embodiments therapeutic agents used to treat SCLC include atezolizumab+carboplatin+etoposide, atezolizumab+carboplatin, atezolizumab+etoposide, or carboplatin+paclitaxel.

### Ovarian Cancer Combination Therapy

Therapeutic agents used to treat ovarian cancer include 5-flourouracil, albumin bound paclitaxel, altretamine, anastrozole, bevacizumab, capecitabine, carboplatin, cisplatin, cyclophosphamide, docetaxel, dC═Orubicin, etoposide, exemestane, gemcitabine, ifosfamide, irinotecan, letrozole, leuprolide acetate, liposomal dC═Orubicin, megestrol acetate, melphalan, olaparib, oxaliplatin, paclitaxel, pazopanib, pemetrexed, tamoxifen, topotecan, vinorelbine, and any combinations thereof.

### Pancreatic Cancer Combination Therapies

Therapeutic agents used to treat pancreatic cancer include 5-FU, leucovorin, oxaliplatin, irinotecan, gemcitabine, nab-paclitaxel (Abraxane®), FOLFIRINOX, and combinations thereof. In some embodiments therapeutic agents used to treat pancreatic cancer include 5-FU+leucovorin+oxaliplatin+irinotecan, 5-FU+nanoliposomal irinotecan, leucovorin+nanoliposomal irinotecan, and gemcitabine+nab-paclitaxel.

### Prostate Cancer Combination Therapies

Therapeutic agents used to treat prostate cancer include enzalutamide (Xtandi®), leuprolide, trifluridine, tipiracil (Lonsurf), cabazitaxel, prednisone, abiraterone (Zytiga®), docetaxel, mitoxantrone, bicalutamide, LHRH, flutamide, ADT, sabizabulin (Veru-111), and combinations thereof. In some embodiments therapeutic agents used to treat prostate cancer include enzalutamide+leuprolide, trifluridine+tipiracil (Lonsurf), cabazitaxel+prednisone, abiraterone+prednisone, docetaxel+prednisone, mitoxantrone+prednisone, bicalutamide+LHRH, flutamide+LHRH, leuprolide+flutamide, and abiraterone+prednisone+ADT.

### Additional Exemplified Combination Therapies

In some embodiments the compound provided herein is administered with one or more therapeutic agents selected from a PI3K inhibitor, a Trop-2 binding agent, CD47 antagonist, a SIRPα antagonist, a FLT3R agonist, a PD-1 antagonist, a PD-L1 antagonist, an MCL1 inhibitor, a CCR8 binding agent, an HPK1 antagonist, a DGKα inhibitor, a CISH inhibitor, a PARP-7 inhibitor, a Cbl-b inhibitor, a KRAS inhibitor (e.g., a KRAS G12C or G12D inhibitor), a KRAS degrader, a beta-catenin degrader, a helios degrader, a CD73 inhibitor, an adenosine receptor antagonist, a TIGIT antagonist, a TREM1 binding agent, a TREM2 binding agent, a CD137 agonist, a GITR binding agent, an OX40 binding agent, and a CAR-T cell therapy.

In some embodiments the compound provided herein is administered with one or more therapeutic agents selected from a PI3K6 inhibitor (e.g., idealisib), an anti-Trop-2 antibody drug conjugate (e.g., sacituzumab govitecan, datopotamab deruxtecan (DS-1062)), an anti-CD47 antibody or a CD47-blocking agent (e.g., magrolimab, DSP-107, AO-176, ALX-148, letaplimab (IBI-188), lemzoparlimab, TTI-621, TTI-622), an anti-SIRPα antibody (e.g., GS-0189), a FLT3L-Fc fusion protein (e.g., GS-3583), an anti-PD-1 antibody (pembrolizumab, nivolumab, zimberelimab), a small molecule PD-L1 inhibitor (e.g., GS-4224), an anti-PD-L1 antibody (e.g., atezolizumab, avelumab), a small molecule MCL1 inhibitor (e.g., GS-9716), a small molecule HPK1 inhibitor (e.g., GS-6451), a HPK1 degrader (PROTAC; e.g., ARV-766), a small molecule DGKα inhibitor, a small molecule CD73 inhibitor (e.g., quemliclustat (AB680)), an anti-CD73 antibody (e.g., oleclumab), a dual A₂ₐ/A_{2b} adenosine receptor antagonist (e.g., etrumadenant (AB928)), an anti-TIGIT antibody (e.g., tiragolumab, vibostolimab, domvanalimab, AB308), an anti-TREM1 antibody (e.g., PY159), an anti-TREM2 antibody (e.g., PY314), a CD137 agonist (e.g., AGEN-2373), a GITR/OX40 binding agent (e.g., AGEN-1223) and a CAR-T cell therapy (e.g., axicabtagene ciloleucel, brexucabtagene autoleucel, tisagenlecleucel).

In some embodiments the compound provided herein is administered with one or more therapeutic agents selected from idealisib, sacituzumab govitecan, magrolimab, GS-0189, GS-3583, zimberelimab, GS-4224, GS-9716, GS-6451, quemliclustat (AB680), etrumadenant (AB928), domvanalimab, AB308, PY159, PY314, AGEN-1223, AGEN-2373, axicabtagene ciloleucel and brexucabtagene autoleucel.

### III. Pharmaceutical Compositions

While it is possible for the active ingredients to be administered alone it may be preferable to present them as pharmaceutical formulations (compositions). The formulations, both for veterinary and for human use, of the invention comprise at least one active ingredient, as above defined, together with one or more acceptable carriers therefor and optionally other therapeutic ingredients. The carrier(s) must be “acceptable” in the sense of being compatible with the other ingredients of the formulation and physiologically innocuous to the recipient thereof.

The formulations include those suitable for the foregoing administration routes. The formulations may conveniently be presented in unit dosage form and may be prepared by any of the methods well known in the art of pharmacy. Techniques and formulations generally are found in Remington's Pharmaceutical Sciences (Mack Publishing Co., Easton, PA). Such methods include the step of bringing into association the active ingredient with inactive ingredients (e.g., a carrier, pharmaceutical excipient, etc.) which constitutes one or more accessory ingredients. In general the formulations are prepared by uniformly and intimately bringing into association the active ingredient with liquid carriers or finely divided solid carriers or both, and then, if necessary, shaping the product.

In certain embodiments, formulations suitable for oral administration are presented as discrete units such as capsules, cachets or tablets each containing a predetermined amount of the active ingredient.

In certain embodiments, the pharmaceutical formulations include one or more compounds of the invention together with one or more pharmaceutically acceptable carriers or excipients and optionally other therapeutic agents. Pharmaceutical formulations containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, lactose monohydrate, croscarmellose sodium, povidone, calcium or sodium phosphate; granulating and disintegrating agents, such as maize starch, or alginic acid; binding agents, such as cellulose, microcrystalline cellulose, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

The amount of active ingredient that is combined with the inactive ingredients to produce a dosage form will vary depending upon the host treated and the particular mode of administration. For example, in some embodiments, a dosage form for oral administration to humans contains approximately 1 to 1000 mg of active material formulated with an appropriate and convenient amount of carrier material (e.g., inactive ingredient or excipient material). In certain embodiments, the carrier material varies from about 5 to about 95% of the total compositions (weight:weight). In some embodiments, the pharmaceutical compositions described herein contain about 1 to 800 mg, 1 to 600 mg, 1 to 400 mg, 1 to 200 mg, 1 to 100 mg or 1 to 50 mg of the compound of Formula I, or a pharmaceutically acceptable salt thereof. In some embodiments, the pharmaceutical compositions described herein contain not more than about 400 mg of the compound of Formula I. In some embodiments, the pharmaceutical compositions described herein contain about 100 mg of the compound of Formula I, or a pharmaceutically acceptable salt thereof.

It should be understood that in addition to the ingredients particularly mentioned above the formulations disclosed herein may include other agents conventional in the art having regard to the type of formulation in question, for example those suitable for oral administration may include flavoring agents.

Veterinary compositions comprising at least one active ingredient as above defined together with a veterinary carrier are further provided. Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

Effective dose of active ingredient depends at least on the nature of the condition being treated, toxicity, whether the compound is being used prophylactically (lower doses), the method of delivery, and the pharmaceutical formulation, and will be determined by the clinician using conventional dose escalation studies.

### IV. Routes of Administration

One or more compounds of Formula I (herein referred to as the active ingredients), or a pharmaceutically acceptable salt thereof, are administered by any route appropriate to the condition to be treated. Suitable routes include oral, rectal, nasal, topical (including buccal and sublingual), vaginal and parenteral (including subcutaneous, intramuscular, intravenous, intradermal, intrathecal and epidural), and the like. It will be appreciated that the preferred route may vary with for example the condition of the recipient. An advantage of the compounds of this invention is that they are orally bioavailable and can be dosed orally. Accordingly, in one embodiment, the pharmaceutical compositions described herein are oral dosage forms. In certain embodiments, the pharmaceutical compositions described herein are oral solid dosage forms.

### Formulation Example 1

Hard gelatin capsules containing the following ingredients are prepared:

| | Quantity |
|---|---|
| Ingredient | (mg/capsule) |
| Active Ingredient | 30.0 |
| Starch | 305.0 |
| Magnesium stearate | 5.0 |

The above ingredients are mixed and filled into hard gelatin capsules.

### Formulation Example 2

A tablet Formula is prepared using the ingredients below:

| | Quantity |
|---|---|
| Ingredient | (mg/tablet) |
| Active Ingredient | 25.0 |
| Cellulose, microcrystalline | 200.0 |
| Colloidal silicon dioxide | 10.0 |
| Stearic acid | 5.0 |

The components are blended and compressed to form tablets.

### Formulation Example 3

A dry powder inhaler formulation is prepared containing the following components:

| Ingredient | Weight % |
|---|---|
| Active Ingredient | 5 |
| Lactose | 95 |

The active ingredient is mixed with the lactose and the mixture is added to a dry powder inhaling appliance.

### Formulation Example 4

Tablets, each containing 30 mg of active ingredient, are prepared as follows:

| | Quantity | |
|---|---|---|
| Ingredient | (mg/tablet) | |
| Active Ingredient | 30.0 | mg |
| Starch | 45.0 | mg |
| Microcrystalline cellulose | 35.0 | mg |
| Polyvinylpyrrolidone | 4.0 | mg |
| (as 10% solution in sterile water) | | |
| Sodium carboxymethyl starch | 4.5 | mg |
| Magnesium stearate | 0.5 | mg |
| Talc | 1.0 | mg |
| Total | 120 | mg |

The active ingredient, starch and cellulose are passed through a No. 20 mesh U.S. sieve and mixed thoroughly. The solution of polyvinylpyrrolidone is mixed with the resultant powders, which are then passed through a 16 mesh U.S. sieve. The granules so produced are dried at 50° C. to 60° C. and passed through a 16 mesh U.S. sieve. The sodium carboxymethyl starch, magnesium stearate and talc, previously passed through a No. 30 mesh U.S. sieve, are then added to the granules which, after mixing, are compressed on a tablet machine to yield tablets each weighing 120 mg.

### Formulation Example 5

Suppositories, each containing 25 mg of active ingredient are made as follows:

| Ingredient | Amount | |
|---|---|---|
| Active Ingredient | 25 | mg |
| Saturated fatty acid glycerides to | 2,000 | mg |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

### Formulation Example 6

Suspensions, each containing 50 mg of active ingredient per 5.0 mL dose are made as follows:

| Ingredient | Amount | |
|---|---|---|
| Active Ingredient | 50.0 | mg |
| Xanthan gum | 4.0 | mg |
| Sodium carboxymethyl cellulose (11%) | 50.0 | mg |
| Microcrystalline cellulose (89%) | | |
| Sucrose | 1.75 | g |
| Sodium benzoate | 10.0 | mg |
| Flavor and Color | q.v. | |
| Purified water to | 5.0 | mL |

The active ingredient, sucrose and xanthan gum are blended, passed through a No. 10 mesh U.S. sieve and then mixed with a previously made solution of the microcrystalline cellulose and sodium carboxymethyl cellulose in water. The sodium benzoate, flavor and color are diluted with some of the water and added with stirring. Sufficient water is then added to produce the required volume.

### Formulation Example 7

A subcutaneous formulation may be prepared as follows:

| Ingredient | Quantity | |
|---|---|---|
| Active Ingredient | 5.0 | mg |
| Corn Oil | 1.0 | mL |

### Formulation Example 8

An injectable preparation is prepared having the following composition:

| Ingredients | Amount | |
|---|---|---|
| Active ingredient | 2.0 | mg/mL |
| Mannitol, USP | 50 | mg/mL |

| | |
|---|---|
| Gluconic acid, USP | q.s. (pH 5-6) |
| water (distilled, sterile) | q.s. to 1.0 mL |
| Nitrogen Gas, NF | q.s. |

### Formulation Example 9

A topical preparation is prepared having the following composition:

| Ingredients | grams |
|---|---|
| Active ingredient | 0.2-10 |
| Span 60 | 2.0 |
| Tween 60 | 2.0 |
| Mineral oil | 5.0 |
| Petrolatum | 0.10 |
| Methyl paraben | 0.15 |
| Propyl paraben | 0.05 |
| BHA (butylated hydroxy anisole) | 0.01 |
| Water | q.s. to100 |

All of the above ingredients, except water, are combined and heated to 60° C. with stirring. A sufficient quantity of water at 60° C. is then added with vigorous stirring to emulsify the ingredients and water then added q.s. 100 g.

### Formulation Example 10

Sustained Release Composition

| Ingredient | Weight Range % |
|---|---|
| Active ingredient | 50-95 |
| Microcrystalline cellulose (filler) | 1-35 |
| Methacrylic acid copolymer | 1-35 |
| Sodium hydroxide | 0.1-1.0 |
| Hydroxypropyl methylcellulose | 0.5-5.0 |
| Magnesium stearate | 0.5-5.0 |

Sustained release formulations of this disclosure may be prepared as follows: compound and pH-dependent binder and any optional excipients are intimately mixed (dry-blended). The dry-blended mixture is then granulated in the presence of an aqueous solution of a strong base which is sprayed into the blended powder. The granulate is dried, screened, mixed with optional lubricants (such as talc or magnesium stearate) and compressed into tablets. Preferred aqueous solutions of strong bases are solutions of alkali metal hydroxides, such as sodium or potassium hydroxide, preferably sodium hydroxide, in water (optionally containing up to 25% of water-miscible solvents such as lower alcohols).

The resulting tablets may be coated with an optional film-forming agent, for identification, taste-masking purposes and to improve ease of swallowing. The film forming agent will typically be present in an amount ranging from between 2% and 4% of the tablet weight. Suitable film-forming agents are well known to the art and include hydroxypropyl methylcellulose, cationic methacrylate copolymers (dimethylaminoethyl methacrylate/methyl-butyl methacrylate copolymers—Eudragit® E—Röhm. Pharma) and the like. These film-forming agents may optionally contain colorants, plasticizers and other supplemental ingredients.

The compressed tablets preferably have a hardness sufficient to withstand 8 Kp compression. The tablet size will depend primarily upon the amount of compound in the tablet. The tablets will include from 300 to 1100 mg of compound free base. Preferably, the tablets will include amounts of compound free base ranging from 400-600 mg, 650-850 mg and 900-1100 mg.

a. In order to influence the dissolution rate, the time during which the compound containing powder is wet mixed is controlled. Preferably the total powder mix time, i.e. the time during which the powder is exposed to sodium hydroxide solution, will range from 1 to 10 minutes and preferably from 2 to 5 minutes. Following granulation, the particles are removed from the granulator and placed in a fluid bed dryer for drying at about 60° C.

### Formulation Example 11

A tablet Formula Is prepared using the ingredients below:

| | Quantity |
|---|---|
| Ingredient | (mg/tablet) |
| Active Ingredient | 300.0 |
| Cellulose, microcrystalline | 100.0 |
| Colloidal silicon dioxide | 10.0 |
| Stearic acid | 5.0 |

The components are blended and compressed to form tablets.

### V. List of Abbreviations and Acronyms

| Abbreviation | Meaning |
|---|---|
| ° C. | Degree Celsius |
| Ac | Acetyl |
| AD-H | Adipic acid dihydrazide |
| ADP | Adenosine diphosphate |
| aq. | Aqueous |
| ATP | Adenosine triphosphate |
| D | Doublet |
| DCM | Dichloromethane |
| dd | Doublet of doublets |
| ddd | Doublet of doublet of doublets |
| DIEA | N,N-Diisopropylethyamine |
| DIPEA | N,N-Diisopropylethylamine (Hünig's Base) |
| DMA | Dimethylacetamide |
| DME | 1,2-Dimethoxyethane |
| DMF | Dimethylformamide |
| DMP | Dess-Martin periodinane |
| DMSO | Dimethylsulfoxide |
| Dt | Doublet-triplet |
| EC₅₀ | The half maximal effective concentration |
| EDC | 1-(3-dimethylaminopropyl)-3-ethylcarbodiimide |
| EDTA | Ethylenediaminetetraacetic acid |
| Eq | Equivalents |
| ES/MS | Electrospray mass spectrometry |
| Et | Ethyl |
| EtOAc | Ethyl acetate |
| EtOH | Ethanol (Ethyl alcohol) |
| FBS | Fetal bovine serum |
| G | Grams |
| h | hours |
| HATU | 1-[Bis(dimethylamino)methylene]-1H-1,2,3- |
| | triazolo[4,5-b]pyridinium 3-oxid |
| | hexafluorophosphate |
| HEPES | 2-[4-(2-hydroxyethyl)piperazin-1-yl] |
| | ethanesulfonic acid |
| HCl | Hydrochloric acid |
| HOAc | Acetic acid |
| HPLC | High performance liquid chromatography |
| Hrs | Hours |
| Hz | Hertz |
| IC₅₀ | Half-maximal inhibitory concentration |
| i-pr | Isopropyl |
| Isolute ® | polystyrene-divinylbenzene copolymer |
| J | Coupling constant (MHz) |
| Li HMDS | Lithium bis(trimethylsilyl)amide |
| LiOH | Lithium hydroxide |
| LiI | Lithium iodide |
| M | Molar |
| M | multiplet |
| M+ | Mass peak |
| M + H+ | Mass peak plus hydrogen |
| Me | Methyl |
| MeCN | Acetonitrile |
| MeOH | Methanol (Methyl alcohol) |
| Me₆Sn₂ | Hexamethyldistannane (hexamethylditin) |
| Mg | Milligram |
| MgSO₄ | Magnesium sulfate |
| MHz | Megahertz |
| Min | Minute |
| ml/mL | Milliliter |
| mM | Millimolar |
| Mmol | Millimole |
| NaHCO₃ | Sodium bicarbonate |
| NBS | N-Bromosuccinimide |
| n- | Normal |
| nBu/Bu | n-Butyl (normal Butyl) |
| NaH | Sodium hydride |
| NaHCO₃ | Sodium bicarbonate |
| nL | Nanoliter |
| Nm | Nanometer |
| NMP | 1-methylpyrrolidin-2-one |
| NMR | Nuclear magnetic resonance |
| Ph | Phenyl |
| psi | Pounds per square inch |
| Q | Quartet |
| q.s. | Quantity sufficient to achieve a stated function |
| rac-BINAP Pd G3 | Methanesulfonato[2,2′-bis(diphenylphosphino)- |
| | 1,1′-binaphthyl](2′-amino-1,1′-biphenyl-2- |
| | yl)palladium(II) |
| RP | Reverse phase |
| Rt | Room temperature |
| RuPhos ® | Methanesulfonato-2′-methylamino-1,1′-biphenyl- |
| | 2-yl- Palladacycles 4^{th} generation |
| S | Singlet |
| T | Triplet |
| t-BuBrettPhos Pd G3 | 2-(Di-tert-butylphosphino)-2′,4′,6′- triisopropyl- |
| | 3,6-dimethoxy-1,1′-biphenyl)-2-(2′-amino-1,1′- |
| | biphenyl)]palladium(II) methanesulfonate |
| TEA | Trimethylamine |
| THF | Tetrahydrofuran |
| TFA | Trifluoroacetic acid |
| XPhos Pd G4 ® | dicyclohexyl-[2-[2,4,6-tri(propan-2- |
| | yl)phenyl]phenyl]phosphanium; methanesulfonic |
| | acid; N-methyl-2-phenylaniline; palladium |

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

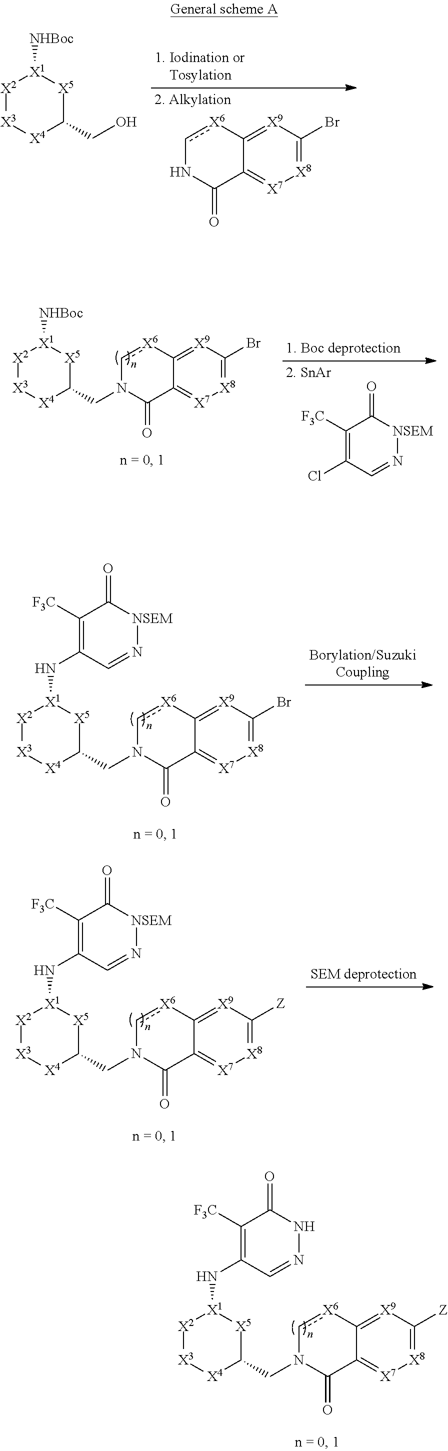

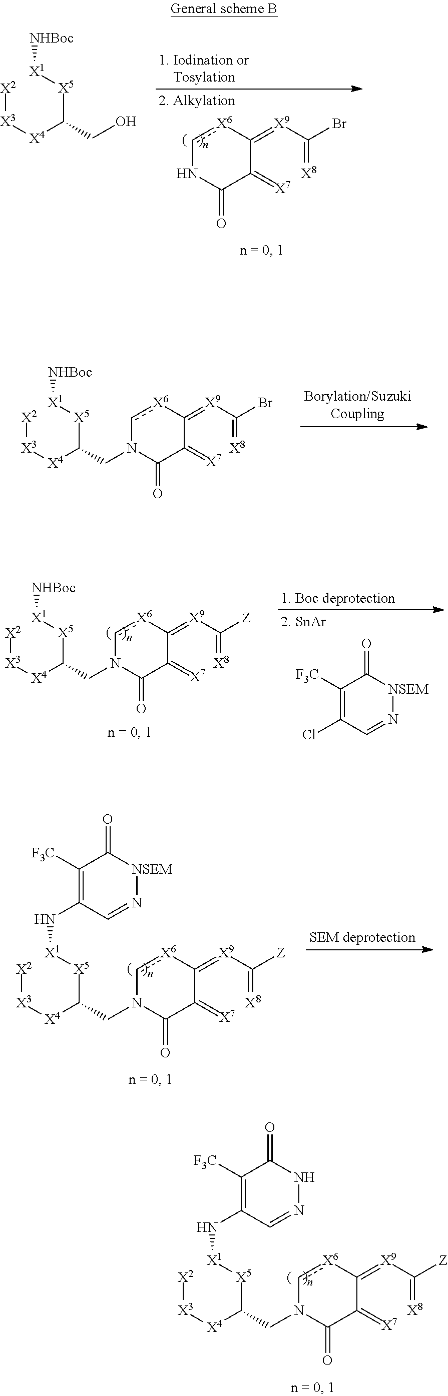

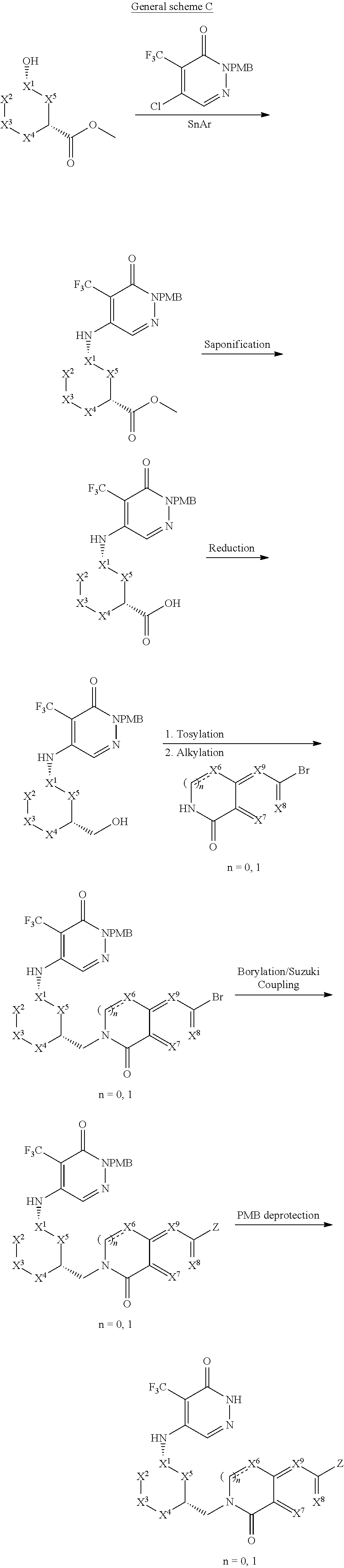

### Example 1: Preparation of 7-fluoro-2-[[(1R,3S)-3-[[6-oxo-5-(trifluoromethyl)-1H-pyridazin-4-yl]amino]cyclohexyl]methyl]-6-(5-pyrazol-1-ylpyrimidin-2-yl)isoquinolin-1-one

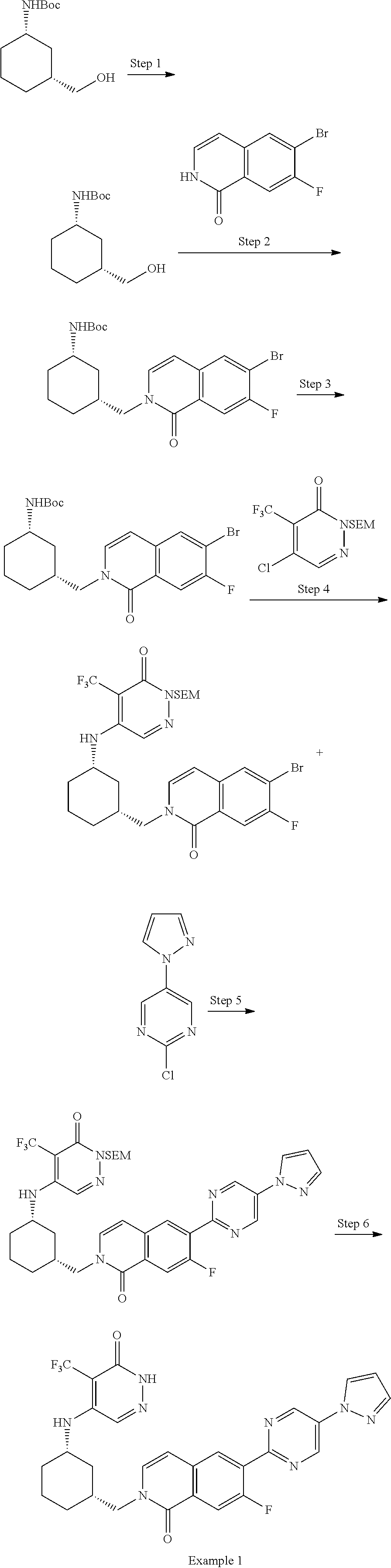

Step 1. To a stirred solution of tert-butyl N-[(1S,3R)-3-(hydroxymethyl)cyclohexyl]carbamate (1500 mg, 6.54 mmol) and triethylamine (1.82 mL, 13.1 mmol), in dichloromethane (10.0 mL) was added p-Toluenesulfonyl chloride (1434 mg, 7.52 mmol) dropwise, followed by DMAP (20.0 mg). The mixture was stirred at room temperature and stirred for 16 h. Upon completion, the mixture was diluted with water and extracted with dicholoromethane. The combined organic layers were washed with brine, dried over Na₂SO₄ and concentrated in vacuo to give the crude product. The crude residue was purified using column chromatography eluting with EtOAc in hexanes 0-100% to afford [(1R,3S)-3-(tert-butoxycarbonylamino)cyclohexyl]methyl 4-methylbenzenesulfonate ES/MS: m/z 384.1 [M+H]⁺.

Step 2. To a mixture of 6-bromo-2H-isoquinolin-1-one (694 mg, 2.87 mmol) and [(1R,3S)-3-(tert-butoxycarbonylamino)cyclohexyl]methyl 4-methylbenzenesulfonate (1000 mg, 2.61 mmol) in DMF (12.0 mL) was added Cs₂CO₃ (2550 mg, 7.82 mmol) and the reaction was stirred at room temperature for 18 h. Upon completion, the mixture was diluted with EtOAc, washed with water, washed with brine, dried over Na₂SO₄ and concentrated in vacuo to give the crude product. The crude residue was purified using column chromatography eluting with EA in hexanes 0-100% to afford tert-butyl N-[(1S,3R)-3-[(6-bromo-7-fluoro-1-oxo-2-isoquinolyl)methyl]cyclohexyl]carbamate. ES/MS: m/z 455.1 [M+H]⁺.

Step 3. In a vial were placed tert-butyl N-[(1S,3R)-3-[(6-bromo-7-fluoro-1-oxo-2-isoquinolyl)methyl]cyclohexyl]carbamate (230 mg, 0.57 mmol), trifluoroacetic acid (0.51 mL) and DCM (4.0 mL). The mixture was stirred at room temperature for 1 hr. Upon completion, the mixture was concentrated in vacuo, and the resultant crude material was partitioned between EtOAc and sat. aq. Sodium bicarbonate solution. The organic layer was washed with brine, dried over Na₂SO₄ and concentrated in vacuo to give 2-[[(1R,3S)-3-aminocyclohexyl]methyl]-6-bromo-7-fluoro-isoquinolin-1-one ES/MS m/z: 355.5[M+H]⁺.

Step 4. A mixture of 2-[[(1R,3S)-3-aminocyclohexyl]methyl]-6-bromo-7-fluoro-isoquinolin-1-one (170 mg, 0.48 mmol), 5-chloro-4-(trifluoromethyl)-2-(2-trimethylsilylethoxymethyl)pyridazin-3-one (237 mg, 0.722 mmol), and N,N-diisopropylethylamine (0.5 mL, 2.9 mmol) in DMF (5.0 mL) was heated at 80° C. for 1 hr. Upon completion, the reaction was diluted with EtOAc, washed with water, washed with brine, dried over Na₂SO₄ and concentrated in vacuo to give the crude product. The crude residue was purified using column chromatography eluting with EtOAc in hexanes 0-100% to provide 6-bromo-7-fluoro-2-[[(1R,3S)-3-[[6-oxo-5-(trifluoromethyl)-1-(2-trimethylsilylethoxymethyl)pyridazin-4-yl]amino]cyclohexyl]methyl]isoquinolin-1-one. ES/MS: m/z 647.1 [M+H]⁺.

Step 5. A vial was charged with 6-bromo-7-fluoro-2-[[(1R,3S)-3-[[6-oxo-5-(trifluoromethyl)-1-(2-trimethylsilylethoxymethyl)pyridazin-4-yl]amino]cyclohexyl]methyl]isoquinolin-1-one (140 mg, 0.22 mmol), CataCXium Pd G4 (8 mg, 0.01 mmol), potassium acetate (64 mg, 0.65 mmol), bis(pinacolato)diboron (72 mg, 0.28 mmol), and dioxane (1 mL). The mixture was bubbled with dry nitrogen, then heated to 80° C. for 2 hours. After cooling, 2-chloro-5-pyrazol-1-yl-pyrimidine (51 mg, 0.28 mmol) and aqueous sodium carbonate (2.0 M, 0.22 mL) were added and the mixture bubbled with dry nitrogen briefly. The reaction was then stirred at 80° C. for 3 hours until complete by LCMS (1-18 hours). The crude reaction was purified by chromatography on silica gel to provide 7-fluoro-2-[[(1R,3S)-3-[[6-oxo-5-(trifluoromethyl)-1-(2-trimethylsilylethoxymethyl)pyridazin-4-yl]amino]cyclohexyl]methyl]-6-(5-pyrazol-1-ylpyrimidin-2-yl)isoquinolin-1-one ES/MS: m/z 711.2 [M+H]⁺.

Step 6. To a solution of 7-fluoro-2-[[(1R,3S)-3-[[6-oxo-5-(trifluoromethyl)-1-(2-trimethylsilylethoxymethyl)pyridazin-4-yl]amino]cyclohexyl]methyl]-6-(5-pyrazol-1-ylpyrimidin-2-yl)isoquinolin-1-one (138 mg, 0.19 mmol) in dichloromethane (3.0 mL) was added trifluoroacetic acid (0.75 mL, 9.7 mmol) at room temperature and the mixture was stirred for 1 hr. The excess trifluoroacetic acid and solvent were removed under reduced pressure and the residue was dissolved in MeOH (2.0 mL). To this was added ethylenediamine (0.04 mL, 0.58 mmol) and the resulting mixture was stirred at room temperature for 2 hr, then at 50° C. for an additional 4 hr. [note—using a larger excess of ethylenediamine allows for shorter reaction times without heating.] Upon completion, the mixture was concentrated in vacuo and purified via reverse phase prep-HPLC (5-100% MeCN in water, 0.1% TFA) to afford 7-fluoro-2-[[(1(R),3(S))-3-[[6-oxo-5-(trifluoromethyl)-1(H)-pyridazin-4-yl]amino]cyclohexyl]methyl]-6-(5-pyrazol-1-ylpyrimidin-2-yl)isoquinolin-1-one. ¹H NMR (400 MHz, DMSO-d6) δ 12.42 (s, 1H), 9.50 (s, 2H), 8.77 (d, J=2.6 Hz, 1H), 8.41 (d, J=7.3 Hz, 1H), 8.01 (d, J=11.4 Hz, 1H), 7.95 (d, J=1.7 Hz, 1H), 7.91 (s, 1H), 7.48 (d, J=7.4 Hz, 1H), 6.83 (d, J=7.4 Hz, 1H), 6.72 (dd, J=2.6, 1.8 Hz, 1H), 6.37-6.29 (m, 1H), 3.98-3.81 (m, 3H), 2.06-1.96 (m, 1H), 1.81 (d, J=10.5 Hz, 2H), 1.73 (d, J=11.7 Hz, 1H), 1.58 (d, J=12.3 Hz, 1H), 1.46-1.21 (m, 3H), 0.96 (q, J=12.1 Hz, 1H). ES/MS m/z=581.1 [M+H]⁺.

### Intermediate 1: 6-bromo-4,7-difluoroisoquinolin-1(2H)-one

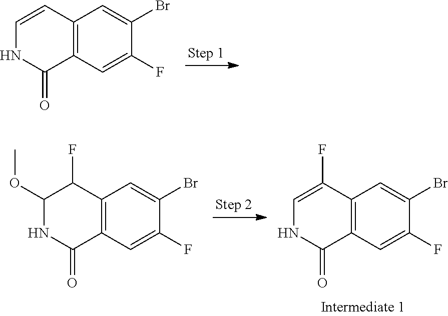 [SMILES: O=c1[nH]ccc2cc(Br)c(F)cc12 ; COC1NC(=O)c2cc(F)c(Br)cc2C1F ; O=c1[nH]cc(F)c2cc(Br)c(F)cc12]

Step 1: Selectfluor (7.68 g, 21.7 mmol) was added to a solution of 6-bromo-7-fluoroisoquinolin-1(2H)-one (5 g, 20.7 mmol) in acetonitrile (100 mL) and methanol (100 mL) at room temperature. The resulting solution was heated to 60° C. for 40 min, then cooled to room temperature and concentrated. The resulting material was carried forward without further purification.

Step 2: The Crude material from Step 1 was taken up in DCM (75 mL) and a solution of hydrochloric acid in dioxane (4 M, 25 mL) was added. The resulting solution was stirred for 1 h at room temperature, then diluted with EtOAc:EtOH (3:1) and water, washed with water and brine, dried with anhydrous magnesium sulfate, filtered, and concentrated. Purification via flash chromatography (elution with 0-100% v/v ethyl acetate in hexanes) afforded 4,7-difluoroisoquinolin-1(2H)-one ES/MS: m/z 259.8 [M+H]⁺.

### Intermediate 2: Preparation of 2-(2-chloropyrimidin-5-yl)-1,1-difluoro-propan-2-ol

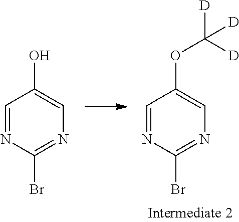 [SMILES: Oc1cnc(Br)nc1 ; [2H]C([2H])([2H])Oc1cnc(Br)nc1]

2-Bromopyrimidin-5-ol (5.0 g, 29 mmol) and K₂CO₃ (7.9 g, 57 mmol) were taken up in DMF (40 mL) and stirred at ambient temperature. Trideuterio(iodo)methane (3.6 mL, 57 mmol) was then added. After 1 hour, the reaction was diluted w/water and extracted 3× with EtOAc. The combined organics were washed w/10% aq. LiCl, brine, and then were dried over MgSO₄. The solution was filtered and evaporated to provide crude material which was purified via flash chromatography (100% hex→100% EtOAc) to afford 2-bromo-5-(trideuteriomethoxy)pyrimidine. ES/MS m/z=192.0/194.0 [M]⁺.

### Intermediate 3: Preparation of 2-(2-chloropyrimidin-5-yl)-1,1-difluoro-propan-2-ol

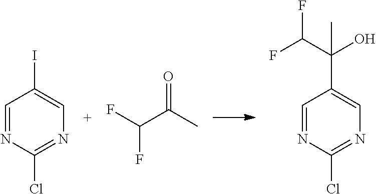

2-Chloro-5-iodo-pyrimidine (1.0 g, 4.2 mmol) was flushed with dry N₂ and dissolved in THF (20 mL). The resultant solution was then cooled to −78° C. in a dry ice/acetone bath. n-BuLi (2.5 M, 0.51 mL) was then added slowly and the reaction maintained in the cooling bath. After 40 minutes, 1,1-difluoroacetone was added. The reaction was stirred a further 2 hr at −78° C. at which point it was quenched with 10% aq. KHSO₄ and warmed to ambient temperature. The reaction was then extracted 3× with EtOAc and the combined organics were dried over MgSO₄, filtered, and evaporated. The crude material was purified using column chromatography (100% Hex→70% EtOAc/Hex) to give 2-(2-chloropyrimidin-5-yl)-1,1-difluoro-propan-2-ol. ES/MS m/z=209.4 [M+H]⁺.

### Intermediate 4: Preparation of 5-(2-((tert-butyldimethylsilyl)oxy)ethoxy)-2-chloropyrimidine

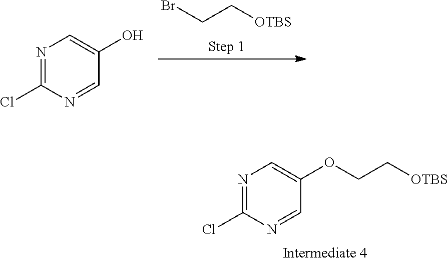 [SMILES: CC(C)(C)[Si](C)(C)OCCBr ; Oc1cnc(Cl)nc1 ; CC(C)(C)[Si](C)(C)OCCOc1cnc(Cl)nc1]

Step 1. A stirred solution of 2-chloropyrimidin-5-ol (500 mg, 3.83 mmol), (2-bromoethoxy)(tert-butyl)dimethylsilane (0.986 mL. 4.6 mmol), and potassium carbonate (1627 mg, 4.98 mmol) in DMF (5 mL) was heated to 50° C. Solution allowed to stir 16 h and diluted with water. Mixture extracted with EtOAc, washed with water, washed with brine, dried over Na₂SO₄ and concentrated. The crude residue was purified using column chromatography eluting with EtOAc in hexanes 0-100% to afford 5-(2-((tert-butyldimethylsilyl)oxy)ethoxy)-2-chloropyrimidine. ES/MS: m/z 291.5 [M+H]⁺.

### Intermediate 5: 6-bromo-4,7,8-trifluoroisociuinolin-1(2H)-one

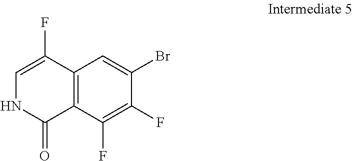 [SMILES: O=c1[nH]cc(F)c2cc(Br)c(F)c(F)c12]

The title compound was synthesized as described for Intermediate 1, with the following changes:

Step 1: 6-bromo-7,8-difluoroisoquinolin-1(2H)-one was used instead of 6-bromo-7-fluoroisoquinolin-1(2H)-one. ES/MS: m/z 277.8 [M+H]⁺.

The following compounds were synthesized using the procedure described in Example 1 from the appropriate starting materials.

| Example | | |
|---|---|---|
| No. | Structure | ¹H NMR, MS [M + H]⁺ |
| 2 | 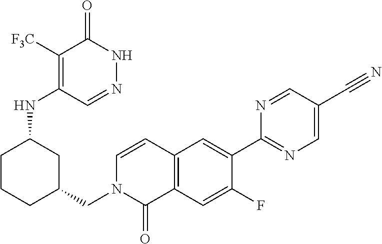 [SMILES: N#Cc1cnc(-c2cc3ccn(C[C@@H]4CCC[C@H](Nc5cn[nH]c(=O)c5C(F)(F)F)C4)c(=O)c3cc2F)nc1] | ¹H NMR (400 MHz, DMSO-d6) δ 12.41 (s, 1H), 9.48 (s, 2H), 8.45 (d, J = 7.2 Hz, 1H), 8.01 (d, J = 11.5 Hz, 1H), 7.89 (s, 1H), 7.49 (d, J = 7.4 Hz, 1H), 6.82 (d, J = 7.4 Hz, 1H), 6.36-6.27 (m, 1H), 3.93- 3.83 (m, 2H), 3.78-3.65 (m, 1H), 2.08-1.94 (m, 1H), 1.79 (d, J = 9.7 Hz, 2H), 1.75-1.67 (m, 1H), 1.56 (d, J = 12.4 Hz, 1H), 1.44-1.19 (m, 3H), 1.01- 0.87 (m, 1H). ES/MS: m/z 540.0 [M + H]⁺. |
| 3 | 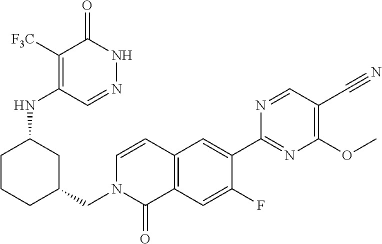 [SMILES: COc1nc(-c2cc3ccn(C[C@@H]4CCC[C@H](Nc5cn[nH]c(=O)c5C(F)(F)F)C4)c(=O)c3cc2F)ncc1C#N] | ¹H NMR (400 MHz, DMSO-d6) δ 12.41 (s, 1H), 9.24 (s, 1H), 8.50 (d, J = 7.2 Hz, 1H), 8.01 (d, J = 11.5 Hz, 1H), 7.89 (s, 1H), 7.49 (d, J = 7.4 Hz, 1H), 6.82 (d, J = 7.4 Hz, 1H), 6.35-6.27 (m, 1H), 4.18 (s, 3H), 3.95-3.73 (m, 3H), 2.06-1.91 (m, 1H), 1.84-1.74 (m, 2H), 1.71 (d, J = 11.5 Hz, 1H), 1.56 (d, J = 12.5 Hz, 1H), 1.44-1.19 (m, 3H), 1.01- 0.87 (m, 1H). ES/MS: m/z 570.1 [M + H]⁺. |
| 4 | 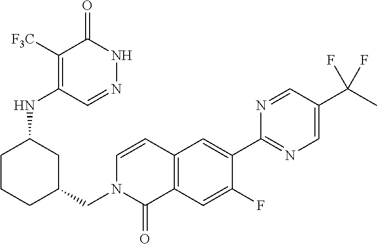 [SMILES: CC(F)(F)c1cnc(-c2cc3ccn(C[C@@H]4CCC[C@H](Nc5cn[nH]c(=O)c5C(F)(F)F)C4)c(=O)c3cc2F)nc1] | ¹H NMR (400 MHz, DMSO-d6) δ 12.42 (s, 1H), 9.25 (s, 1H), 8.39 (d, J = 7.2 Hz, 1H), 8.02 (d, J = 11.4 Hz, 1H), 7.91 (s, 1H), 7.49 (d, J = 7.4 Hz, 1H), 6.82 (d, J = 7.4 Hz, 1H), 6.39-6.27 (m, 1H), 3.92- 3.80 (m, 2H), 3.80-3.59 (m, 1H), 2.14 (t, J = 19.4 Hz, 2H), 2.07-1.96 (m, 0H), 1.85-1.76 (m, 2H), 1.76-1.67 (m, 1H), 1.61-1.53 (m, 1H), 1.45- 1.21 (m, 3H), 1.02-0.89 (m, 1H). ES/MS: m/z 579.1 [M + H]⁺. |
| 5 | 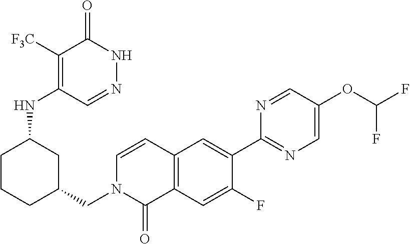 [SMILES: O=c1[nH]ncc(N[C@H]2CCC[C@@H](Cn3ccc4cc(-c5ncc(OC(F)F)cn5)c(F)cc4c3=O)C2)c1C(F)(F)F] | ¹H NMR (400 MHz, DMSO-d6) δ 12.42 (s, 1H), 8.99 (s, 2H), 8.33 (d, J = 7.2 Hz, 1H), 8.00 (d, J = 11.3 Hz, 1H), 7.91 (s, 1H), 7.70-7.27 (m, 2H), 6.80 (d, J = 7.3 Hz, 1H), 6.39-6.26 (m, 1H), 3.88 (d, J = 7.0 Hz, 2H), 3.80-3.65 (m, 1H), 2.08-1.95 (m, 1H), 1.85-1.68 (m, 3H), 1.61-1.53 (m, 1H), 1.45- 1.20 (m, 3H), 1.04-0.88 (m, 1H). ES/MS: m/z 581.1 [M + H]⁺. |
| 6 | 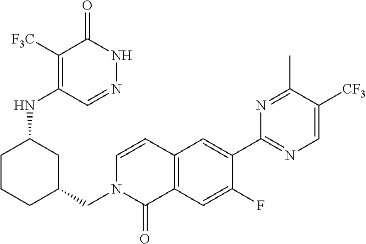 [SMILES: Cc1nc(-c2cc3ccn(C[C@@H]4CCC[C@H](Nc5cn[nH]c(=O)c5C(F)(F)F)C4)c(=O)c3cc2F)ncc1C(F)(F)F] | ¹H NMR (400 MHz, DMSO-d6) δ 12.42 (s, 1H), 9.26 (d, J = 0.9 Hz, 1H), 8.40 (d, J = 7.2 Hz, 1H), 8.02 (d, J = 11.3 Hz, 1H), 7.91 (s, 1H), 7.50 (d, J = 7.4 Hz, 1H), 6.83 (d, J = 7.4 Hz, 1H), 6.32 (dd, J = 8.3, 3.9 Hz, 1H), 3.89 (d, J = 7.0 Hz, 2H), 3.73 (br s, 2H), 2.75 (q, J = 1.5 Hz, 3H), 2.03 (br s, 1H), 1.84- 1,68 (m, 3H), 1.57 (d, J = 12.7 Hz, 1H), 1.45-1.21 (m, 3H), 1.02-0.89 (m, 1H). ES/MS: m/z 597.1 [M + H]⁺. |
| 7 | 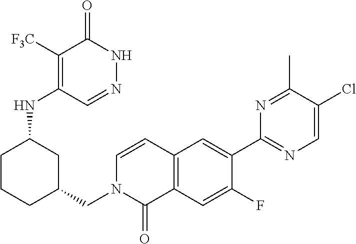 [SMILES: Cc1nc(-c2cc3ccn(C[C@@H]4CCC[C@H](Nc5cn[nH]c(=O)c5C(F)(F)F)C4)c(=O)c3cc2F)ncc1Cl] | ¹H NMR (400 MHz, DMSO-d6) δ 12.42 (s, 1H), 8.99 (s, 1H), 8.32 (d, J = 7.2 Hz, 1H), 7.99 (d, J = 11.3 Hz, 1H), 7.91 (s, 1H), 7.48 (d, J = 7.4 Hz, 1H), 6.81 (d, J = 7.4 Hz, 1H), 6.32 (dd, J = 8.5, 3.9 Hz, 1H), 3.88 (d, J = 7.0 Hz, 2H), 3.73 (d, J = 10.7 Hz, 1H), 2.66 (s, 3H), 2.02 (br s, 1H), 1.86-1.68 (m, 3H), 1.57 (d, J = 12.7 Hz, 1H), 1.45-1.20 (m, 3H), 1.01-0.89 (m, 1H). ES/MS: m/z 563.1 [M + H]⁺. |
| 8 | 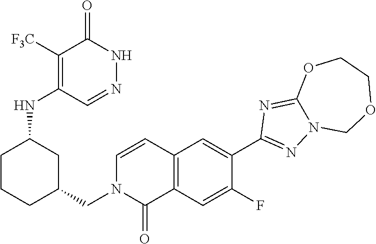 [SMILES: O=c1[nH]ncc(N[C@H]2CCC[C@@H](Cn3ccc4cc(-c5nc6n(n5)COCCO6)c(F)cc4c3=O)C2)c1C(F)(F)F] | ¹H NMR (400 MHz, DMSO-d6) δ 12.42 (s, 1H), 8.26 (d, J = 7.0 Hz, 1H), 8.02-7.83 (m, 2H), 7.45 (d, J = 7.4 Hz, 1H), 6.77 (d, J = 7.4 Hz, 1H), 6.32 (m, 1H), 5.66 (s, 2H), 4.56-4.38 (m, 2H), 4.28- 4.09 (m, 2H), 3.87 (m, 2H), 3.73 (m, 1H), 2.14- 1.87 (m, 1H), 1.87-1.69 (m, 3H), 1.56 (m, 1H), 1.31 (m, 3H), 1.07-0.76 (m, 1H). ES/MS: m/z 576.0 [M + H]⁺. |
| 9 | 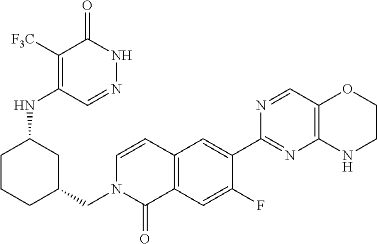 [SMILES: O=c1[nH]ncc(N[C@H]2CCC[C@@H](Cn3ccc4cc(-c5ncc6c(n5)NCCO6)c(F)cc4c3=O)C2)c1C(F)(F)F] | ¹H NMR (400 MHz, DMSO-d6) δ 12.43 (s, 1H), 8.81 (s, 1H), 8.11 (d, J = 7.1 Hz, 1H), 8.07 (s, 1H), 7.97 (d, J = 11.1 Hz, 1H), 7.90 (s, 1H), 7.47 (d, J = 7.4 Hz, 1H), 6.74 (d, J = 7.4 Hz, 1H), 6.32 (s, 1H), 4.25 (t, J = 4.4 Hz, 2H), 3.88 (d, J = 6.9 Hz, 2H), 3.73 (d, J = 6.1 Hz, 1H), 3.58 (s, 2H), 2.08-1.95 (m, 1H), 1.78 (d, J = 11.4 Hz, 2H), 1.71 (s, 1H), 1.58 (s, 1H), 1.32 (dt, J = 35.4, 11.2 Hz, 3H), 0.95 (q, J = 12.6 Hz, 1H). ES/MS: m/z 572.1 [M + H]⁺. |
| 10 | 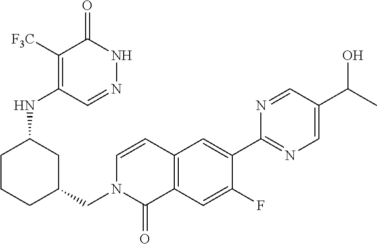 [SMILES: CC(O)c1cnc(-c2cc3ccn(C[C@@H]4CCC[C@H](Nc5cn[nH]c(=O)c5C(F)(F)F)C4)c(=O)c3cc2F)nc1] | ¹H NMR (400 MHz, DMSO-d6) δ 12.42 (s, 1H), 8.96 (s, 2H), 8.32 (d, J = 7.2 Hz, 1H), 7.98 (d, J = 11.3 Hz, 1H), 7.91 (s, 1H), 7.47 (d, J = 7.4 Hz, 1H), 6.79 (d, J = 7.4 Hz, 1H), 6.35-6.28 (m, 1H), 4.92 (q, J = 6.5 Hz, 1H), 3.88 (d, J = 6.9 Hz, 2H), 3.78- 3.67 (m, 1H), 2.08-1.96 (m, 1H), 1.80 (d, J = 7.7 Hz, 2H), 1.72 (d, J = 12.2 Hz, 1H), 1.57 (d, J = 12.3 Hz, 1H), 1.46 (d, J = 6.5 Hz, 3H), 1.31 (dq, J = 35.5, 11.9, 10.7 Hz, 3H), 1.02-0.88 (m, 1H). ES/MS: m/z 559.2 [M + H]⁺. |
| 11 | 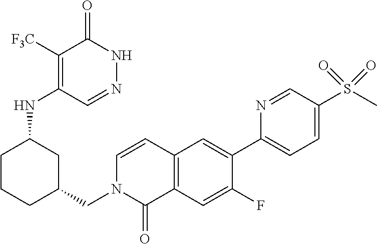 [SMILES: CS(=O)(=O)c1ccc(-c2cc3ccn(C[C@@H]4CCC[C@H](Nc5cn[nH]c(=O)c5C(F)(F)F)C4)c(=O)c3cc2F)nc1] | ¹H NMR (400 MHz, DMSO-d6) δ 12.42 (s, 1H), 9.26-9.21 (m, 1H), 8.50 (dd, J = 8.4, 2.4 Hz, 1H), 8.31 (d, J = 7.4 Hz, 1H), 8.16 (dd, J = 8.4, 1.9 Hz, 1H), 8.04 (d, J = 11.6 Hz, 1H), 7.91 (s, 1H), 7.49 (d, J = 7.4 Hz, 1H), 6.81 (d, J = 7.4 Hz, 1H), 6.36-6.29 (m, 1H), 3.89 (d, J = 7.0 Hz, 2H), 3.76-3.67 (m, 1H), 3.41 (s, 3H), 2.05 (d, J = 20.7 Hz, 1H), 1.85- 1.65 (m, 3H), 1.57 (d, J = 12.7 Hz, 1H), 1.46-1.20 (m, 3H), 0.95 (q, J = 12.6, 11.7 Hz, 1H). ES/MS: m/z 592.1 [M + H]⁺. |
| 12 | 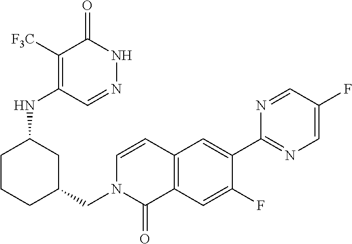 [SMILES: O=c1[nH]ncc(N[C@H]2CCC[C@@H](Cn3ccc4cc(-c5ncc(F)cn5)c(F)cc4c3=O)C2)c1C(F)(F)F] | ¹H NMR (400 MHz, DMSO-d6) δ 12.43 (s, 1H), 9.23-8.93 (m, 2H), 8.32 (d, J = 7.2 Hz, 1H), 8.00 (d, J = 11.3 Hz, 1H), 7.91 (s, 1H), 7.48 (d, J = 7.4 Hz, 1H), 6.80 (d, J = 7.3 Hz, 1H), 6.37-6.24 (m, 1H), 3.89 (d, J = 7.0 Hz, 2H), 3.80-3.64 (m, 1H), 2.10-1.95 (m, 1H), 1.86-1.66 (m, 3H), 1.57 (d, J = 12.6 Hz, 1H), 1.46-1.14 (m, 3H), 1.02-0.80 (m, 1H). ES/MS: m/z 533.1 [M + H]⁺. |
| 13 | 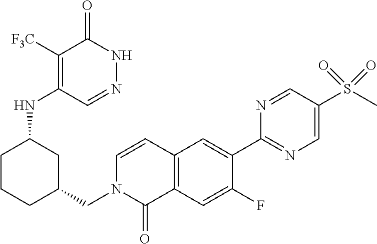 [SMILES: CS(=O)(=O)c1cnc(-c2cc3ccn(C[C@@H]4CCC[C@H](Nc5cn[nH]c(=O)c5C(F)(F)F)C4)c(=O)c3cc2F)nc1] | ¹H NMR (400 MHz, DMSO-d6) δ 12.42 (s, 1H), 9.46 (s, 2H), 8.46 (d, J = 7.1 Hz, 1H), 8.04 (d, J = 11.4 Hz, 1H), 7.91 (s, 1H), 7.51 (d, J = 7.4 Hz, 1H), 6.85 (d, J = 7.4 Hz, 1H), 6.33 (s, 1H), 3.89 (d, J = 7.0 Hz, 3H), 3.79-3.68 (m, 1H), 2.03 (s, 1H), 1.76 (dd, J = 30.0, 10.5 Hz, 4H), 1.57 (d, J = 12.7 Hz, 1H), 1.32 (dq, J = 34.5, 11.8 Hz, 3H), 1.05-0.84 (m, 1H). ES/MS: m/z 593.1 [M + H]⁺. |
| 14 | 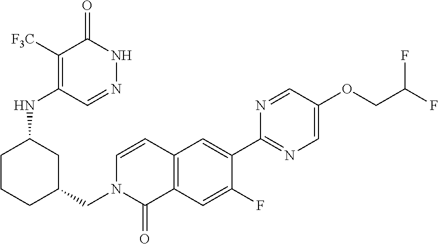 [SMILES: O=c1[nH]ncc(N[C@H]2CCC[C@@H](Cn3ccc4cc(-c5ncc(OCC(F)F)cn5)c(F)cc4c3=O)C2)c1C(F)(F)F] | ¹H NMR (400 MHz, DMSO-d6) δ 12.42 (s, 1H), 8.83 (s, 2H), 8.30 (d, J = 7.3 Hz, 1H), 8.08-7.82 (m, 2H), 7.46 (d, J = 7.4 Hz, 1H), 6.79 (d, J = 7.4 Hz, 1H), 6.35 (t, J = 3.4 Hz, 1H), 6.32 (dd, J = 8.4, 4.0 Hz, 1H), 4.64 (td, J = 14.7, 3.4 Hz, 2H), 3.88 (d, J = 7.0 Hz, 2H), 3.73 (s, 1H), 2.02 (s, 1H), 1.90- 1.65 (m, 3H), 1.57 (d, J = 12.5 Hz, 1H), 1.33 (dp, J = 34.1, 12.4, 11.6 Hz, 3H), 0.95 (q, J = 12.7, 12.2 Hz, 1H). ES/MS: m/z 595.1 [M + H]⁺. |
| 15 | 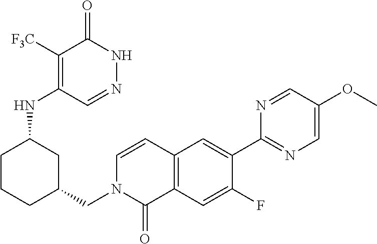 [SMILES: COc1cnc(-c2cc3ccn(C[C@@H]4CCC[C@H](Nc5cn[nH]c(=O)c5C(F)(F)F)C4)c(=O)c3cc2F)nc1] | ¹H NMR (400 MHz, DMSO-d6) δ 12.42 (s, 1H), 8.72 (dd, J = 2.3, 1.4 Hz, 1H), 8.52 (d, J = 1.4 Hz, 1H), 8.23 (d, J = 7.4 Hz, 1H), 8.00 (d, J = 11.7 Hz, 1H), 7.90 (s, 1H), 7.46 (d, J = 7.4 Hz, 1H), 6.77 (d, J = 7.3 Hz, 1H), 6.38-6.26 (m, 1H), 4.00 (s, 3H), 3.90-3.86 (m, 2H), 2.08-1.93 (m, 1H), 1.86- 1.66 (m, 3H), 1.64-1.51 (m, 1H), 1.43-1.21 (m, 3H), 1.01-0.88 (m, 1H). Note-one methine peak overlaps with water signal. ES/MS: m/z 545.1 [M + H]⁺. |
| 16 | 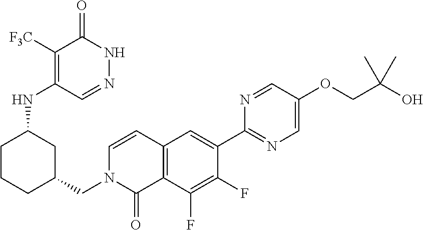 [SMILES: CC(C)(O)COc1cnc(-c2cc3ccn(C[C@@H]4CCC[C@H](Nc5cn[nH]c(=O)c5C(F)(F)F)C4)c(=O)c3c(F)c2F)nc1] | ¹H NMR (400 MHz, DMSO-d6) δ 12.42 (s, 1H), 8.77 (s, 2H), 8.12-8.07 (m, 1H), 7.92 (s, 1H), 7.48 (d, J = 7.4 Hz, 1H), 6.75 (dd, J = 7.5, 1.8 Hz, 1H), 6.33 (dd, J = 8.5, 3.9 Hz, 1H), 4.02 (s, 2H), 3.88- 3.68 (m, 3H), 2.01 (br s, 0H), 1.87-1.67 (m, 3H), 1.58 (d, J = 12.6 Hz, 1H), 1.45-1.26 (m, 2H), 1.24 (s, 6H), 1.01-0.87 (q, J = 12.8, 12.1 Hz, 1H). ES/MS: m/z 621.1 [M + H]⁺. |
| 17 | 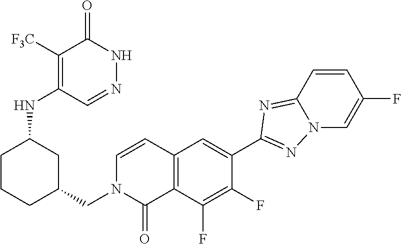 [SMILES: O=c1[nH]ncc(N[C@H]2CCC[C@@H](Cn3ccc4cc(-c5nc6ccc(F)cn6n5)c(F)c(F)c4c3=O)C2)c1C(F)(F)F] | ¹H NMR (400 MHz, DMSO-d6) δ 12.42 (s, 1H), 9.48 (m, 1H), 8.31 (m, 1H), 8.05 (m, 1H), 7.97- 7.81 (m, 2H), 7.51 (d, J = 7.4 Hz, 1H), 6.81 (m, 1H), 6.33 (m, 1H), 3.94-3.59 (m, 3H), 2.02 (m, 1H), 1.77 (m, 3H), 1.58 (m, 1H), 1.31 (m, 3H), 0.95 (m, 1H). ES/MS: m/z 590.0 [M + H]⁺. |
| 18 | 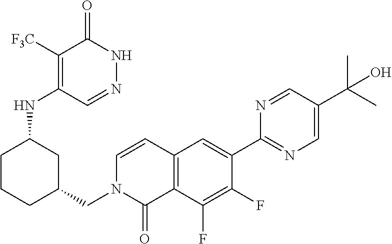 [SMILES: CC(C)(O)c1cnc(-c2cc3ccn(C[C@@H]4CCC[C@H](Nc5cn[nH]c(=O)c5C(F)(F)F)C4)c(=O)c3c(F)c2F)nc1] | ¹H NMR (400 MHz, DMSO-d6) δ 12.42 (s, 1H), 9.08 (s, 2H), 8.19-8.07 (m, 1H), 7.92 (s, 1H), 7.49 (d, J = 7.4 Hz, 1H), 6.76 (dd, J = 7.5, 1.8 Hz, 1H), 6.33 (dd, J = 8.4, 4.0 Hz, 1H), 3.91-3.63 (m, 3H), 2.02 (s, 1H), 1.85-1.65 (m, 3H), 1.54 (s, 7H), 1.31 (dq, J = 36.7, 11.7 Hz, 3H), 0.94 (q, J = 12.0 Hz, 1H). ES/MS: m/z 591.1 [M + H]⁺. |
| 19 | 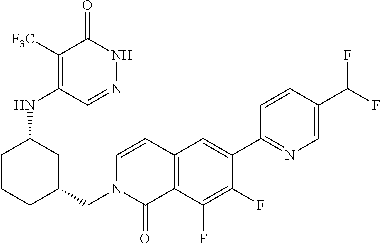 [SMILES: O=c1[nH]ncc(N[C@H]2CCC[C@@H](Cn3ccc4cc(-c5ccc(C(F)F)cn5)c(F)c(F)c4c3=O)C2)c1C(F)(F)F] | ¹H NMR (400 MHz, DMSO-d6) δ 12.42 (s, 1H), 8.99 (dt, J = 2.5, 1.1 Hz, 1H), 8.27-8.18 (m, 1H), 8.05 (dt, J = 6.2, 2.9 Hz, 2H), 7.91 (s, 1H), 7.50 (d, J = 7.4 Hz, 1H), 7.26 (t, J = 55.2 Hz, 1H), 6.75 (dd, J = 7.5, 1.8 Hz, 1H), 6.33 (dd, J = 8.3, 4.0 Hz, 1H), 3.91-3.78 (m, 2H), 3.74 (s, 1H), 2.02 (s, 1H), 1.87- 1.67 (m, 3H), 1.58 (d, J = 12.7 Hz, 1H), 1.33 (dp, J = 35.9, 12.0 Hz, 3H), 0.95 (q, J = 12.5, 11.8 Hz, 1H). ES/MS: m/z 582.1 [M + H]⁺. |
| 20 | 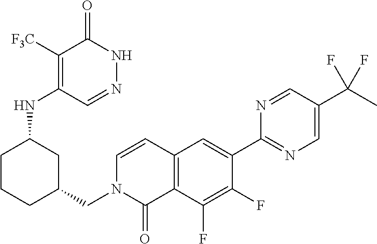 [SMILES: CC(F)(F)c1cnc(-c2cc3ccn(C[C@@H]4CCC[C@H](Nc5cn[nH]c(=O)c5C(F)(F)F)C4)c(=O)c3c(F)c2F)nc1] | ¹H NMR (400 MHz, DMSO-d6) δ 12.42 (s, 1H), 9.27 (s, 2H), 8.20 (dd, J = 6.7, 1.7 Hz, 1H), 7.92 (s, 1H), 7.51 (d, J = 7.4 Hz, 1H), 6.79 (dd, J = 7.5, 1.8 Hz, 1H), 6.37-6.28 (m, 1H), 3.91-3.78 (m, 2H), 2.14 (t, J = 19.4 Hz, 3H), 2.07-1.96 (m, 1H), 1.86- 1.68 (m, 3H), 1.61-1.53 (m, 1H), 1.45-1.19 (m, 3H), 1.02-0.86 (m, 1H). ES/MS: m/z 597.1 [M + H]⁺. |
| 21 | 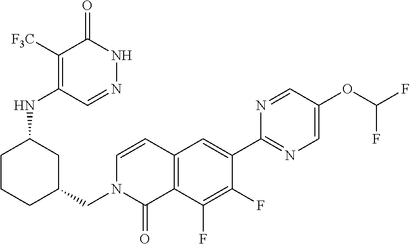 [SMILES: O=c1[nH]ncc(N[C@H]2CCC[C@@H](Cn3ccc4cc(-c5ncc(OC(F)F)cn5)c(F)c(F)c4c3=O)C2)c1C(F)(F)F] | ¹H NMR (400 MHz, DMSO-d6) δ 12.42 (s, 1H), 9.26 (d, J = 0.9 Hz, 1H), 8.40 (d, J = 7.2 Hz, 1H), 8.02 (d, J = 11.3 Hz, 1H), 7.91 (s, 1H), 7.50 (d, J = 7.4 Hz, 1H), 6.83 (d, J = 7.4 Hz, 1H), 6.32 (dd, J = 8.3, 3.9 Hz, 1H), 3.89 (d, J = 7.0 Hz, 2H), 3.73 (br s, 2H), 2.75 (q, J = 1.5 Hz, 3H), 2.03 (br s, 1H), 1.84- 1,68 (m, 3H), 1.57 (d, J = 12.7 Hz, 1H), 1.45-1.21 (m, 3H), 1.02-0.89 (m, 1H). ES/MS: m/z 599.1 [M + H]⁺. |
| 22 | 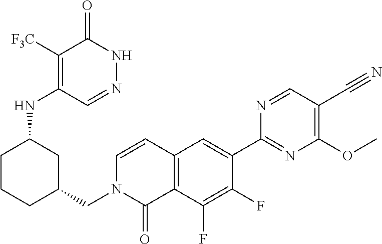 [SMILES: COc1nc(-c2cc3ccn(C[C@@H]4CCC[C@H](Nc5cn[nH]c(=O)c5C(F)(F)F)C4)c(=O)c3c(F)c2F)ncc1C#N] | ¹H NMR (400 MHz, DMSO-d6) δ 12.42 (s, 1H), 8.99 (s, 1H), 8.32 (d, J = 7.2 Hz, 1H), 7.99 (d, J = 11.3 Hz, 1H), 7.91 (s, 1H), 7.48 (d, J = 7.4 Hz, 1H), 6.81 (d, J = 7.4 Hz, 1H), 6.32 (dd, J = 8.5, 3.9 Hz, 1H), 3.88 (d, J = 7.0 Hz, 2H), 3.73 (d, J = 10.7 Hz, 1H), 2.66 (s, 3H), 2.02 (br s, 1H), 1.86-1.68 (m, 3H), 1.57 (d, J = 12.7 Hz, 1H), 1.45-1.20 (m, 3H), 1.01-0.89 (m, 1H). ES/MS: m/z 588.1 [M + H]⁺. |
| 23 | 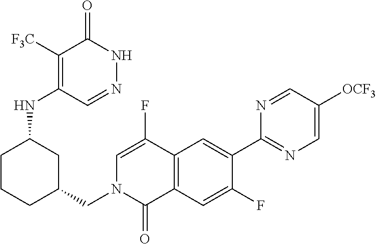 [SMILES: O=c1[nH]ncc(N[C@H]2CCC[C@@H](Cn3cc(F)c4cc(-c5ncc(OC(F)(F)F)cn5)c(F)cc4c3=O)C2)c1C(F)(F)F] | ¹H NMR (400 MHz, DMSO-d6) δ 12.42 (s, 1H), 8.26 (d, J = 7.0 Hz, 1H), 8.02-7.83 (m, 2H), 7.45 (d, J = 7.4 Hz, 1H), 6.77 (d, J = 7.4 Hz, 1H), 6.32 (m, 1H), 5.66 (s, 2H), 4.56-4.38 (m, 2H), 4.28- 4.09 (m, 2H), 3.87 (m, 2H), 3.73 (m, 1H), 2.14- 1.87 (m, 1H), 1.87-1.69 (m, 3H), 1.56 (m, 1H), 1.31 (m, 3H), 1.07-0.76 (m, 1H). ES/MS: m/z 617.1 [M + H]⁺. |
| 24 | 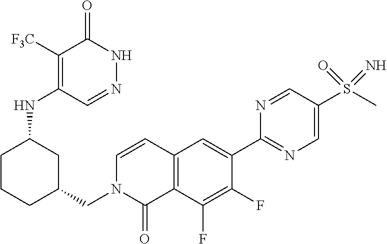 [SMILES: CS(=N)(=O)c1cnc(-c2cc3ccn(C[C@@H]4CCC[C@H](Nc5cn[nH]c(=O)c5C(F)(F)F)C4)c(=O)c3c(F)c2F)nc1] | ¹H NMR (400 MHz, DMSO-d6) δ 12.43 (s, 1H), 8.81 (s, 1H), 8.11 (d, J = 7.1 Hz, 1H), 8.07 (s, 1H), 7.97 (d, J = 11.1 Hz, 1H), 7.90 (s, 1H), 7.47 (d, J = 7.4 Hz, 1H), 6.74 (d, J = 7.4 Hz, 1H), 6.32 (s, 1H), 4.25 (t, J = 4.4 Hz, 2H), 3.88 (d, J = 6.9 Hz, 2H), 3.73 (d, J = 6.1 Hz, 1H), 3.58 (s, 2H), 2.08-1.95 (m, 1H), 1.78 (d, J = 11.4 Hz, 2H), 1.71 (s, 1H), 1.58 (s, 1H), 1.32 (dt, J = 35.4, 11.2 Hz, 3H), 0.95 (q, J = 12.6 Hz, 1H). ES/MS: m/z 610.1 [M + H]⁺. |
| 25 | 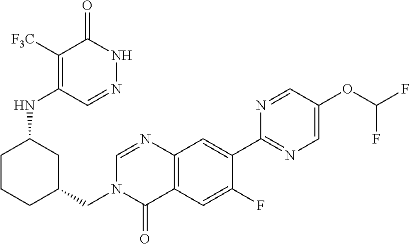 [SMILES: O=c1[nH]ncc(N[C@H]2CCC[C@@H](Cn3cnc4cc(-c5ncc(OC(F)F)cn5)c(F)cc4c3=O)C2)c1C(F)(F)F] | ¹H NMR (400 MHz, DMSO-d6) δ 12.42 (s, 1H), 8.96 (s, 2H), 8.32 (d, J = 7.2 Hz, 1H), 7.98 (d, J = 11.3 Hz, 1H), 7.91 (s, 1H), 7.47 (d, J = 7.4 Hz, 1H), 6.79 (d, J = 7.4 Hz, 1H), 6.35-6.28 (m, 1H), 4.92 (q, J = 6.5 Hz, 1H), 3.88 (d, J = 6.9 Hz, 2H), 3.78- 3.67 (m, 1H), 2.08-1.96 (m, 1H), 1.80 (d, J = 7.7 Hz, 2H), 1.72 (d, J = 12.2 Hz, 1H), 1.57 (d, J = 12.3 Hz, 1H), 1.46 (d, J = 6.5 Hz, 3H), 1.31 (dq, J = 35.5, 11.9, 10.7 Hz, 3H), 1.02-0.88 (m, 1H). ES/MS: m/z 582.1 [M + H]⁺. |
| 26 | 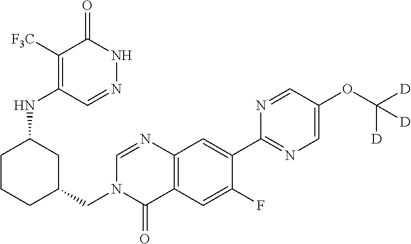 [SMILES: [2H]C([2H])([2H])Oc1cnc(-c2cc3ncn(C[C@@H]4CCC[C@H](Nc5cn[nH]c(=O)c5C(F)(F)F)C4)c(=O)c3cc2F)nc1] | ¹H NMR (400 MHz, DMSO-d6) δ 12.42 (s, 1H), 9.26-9.21 (m, 1H), 8.50 (dd, J = 8.4, 2.4 Hz, 1H), 8.31 (d, J = 7.4 Hz, 1H), 8.16 (dd, J = 8.4, 1.9 Hz, 1H), 8.04 (d, J = 11.6 Hz, 1H), 7.91 (s, 1H), 7.49 (d, J = 7.4 Hz, 1H), 6.81 (d, J = 7.4 Hz, 1H), 6.36-6.29 (m, 1H), 3.89 (d, J = 7.0 Hz, 2H), 3.76-3.67 (m, 1H), 3.41 (s, 3H), 2.05 (d, J = 20.7 Hz, 1H), 1.85- 1.65 (m, 3H), 1.57 (d, J = 12.7 Hz, 1H), 1.46-1.20 (m, 3H), 0.95 (q, J = 12.6, 11.7 Hz, 1H). ES/MS: m/z 549.1 [M + H]⁺. |
| 27 | 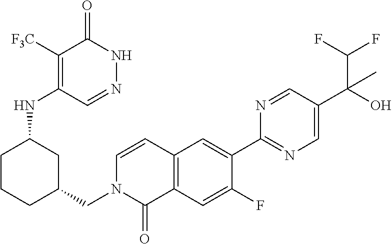 [SMILES: CC(O)(c1cnc(-c2cc3ccn(C[C@@H]4CCC[C@H](Nc5cn[nH]c(=O)c5C(F)(F)F)C4)c(=O)c3cc2F)nc1)C(F)F] | ¹H NMR (400 MHz, DMSO-d6) δ 12.43 (s, 1H), 9.23-8.93 (m, 2H), 8.32 (d, J = 7.2 Hz, 1H), 8.00 (d, J = 11.3 Hz, 1H), 7.91 (s, 1H), 7.48 (d, J = 7.4 Hz, 1H), 6.80 (d, J = 7.3 Hz, 1H), 6.37-6.24 (m, 1H), 3.89 (d, J = 7.0 Hz, 2H), 3.80-3.64 (m, 1H), 2.10-1.95 (m, 1H), 1.86-1.66 (m, 3H), 1.57 (d, J = 12.6 Hz, 1H), 1.46-1.14 (m, 3H), 1.02-0.80 (m, 1H). ES/MS: m/z 609.1 [M + H]⁺. |
| 28 | 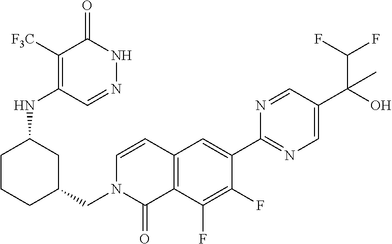 [SMILES: CC(O)(c1cnc(-c2cc3ccn(C[C@@H]4CCC[C@H](Nc5cn[nH]c(=O)c5C(F)(F)F)C4)c(=O)c3c(F)c2F)nc1)C(F)F] | ¹H NMR (400 MHz, DMSO-d6) δ 12.42 (s, 1H), 9.46 (s, 2H), 8.46 (d, J = 7.1 Hz, 1H), 8.04 (d, J = 11.4 Hz, 1H), 7.91 (s, 1H), 7.51 (d, J = 7.4 Hz, 1H), 6.85 (d, J = 7.4 Hz, 1H), 6.33 (s, 1H), 3.89 (d, J = 7.0 Hz, 3H), 3.79-3.68 (m, 1H), 2.03 (s, 1H), 1.76 (dd, J = 30.0, 10.5 Hz, 4H), 1.57 (d, J = 12.7 Hz, 1H), 1.32 (dq, J = 34.5, 11.8 Hz, 3H), 1.05-0.84 (m, 1H). ES/MS: m/z 627.2 [M + H]⁺. |
| 29 | 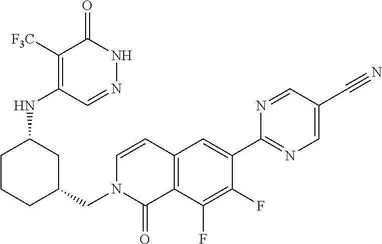 [SMILES: N#Cc1cnc(-c2cc3ccn(C[C@@H]4CCC[C@H](Nc5cn[nH]c(=O)c5C(F)(F)F)C4)c(=O)c3c(F)c2F)nc1] | ¹H NMR (400 MHz, DMSO-d6) δ 12.42 (s, 1H), 8.83 (s, 2H), 8.30 (d, J = 7.3 Hz, 1H), 8.08-7.82 (m, 2H), 7.46 (d, J = 7.4 Hz, 1H), 6.79 (d, J = 7.4 Hz, 1H), 6.35 (t, J = 3.4 Hz, 1H), 6.32 (dd, J = 8.4, 4.0 Hz, 1H), 4.64 (td, J = 14.7, 3.4 Hz, 2H), 3.88 (d, J = 7.0 Hz, 2H), 3.73 (s, 1H), 2.02 (s, 1H), 1.90- 1.65 (m, 3H), 1.57 (d, J = 12.5 Hz, 1H), 1.33 (dp, J = 34.1, 12.4, 11.6 Hz, 3H), 0.95 (q, J = 12.7, 12.2 Hz, 1H). ES/MS: m/z 558.1 [M + H]⁺. |
| 30 | 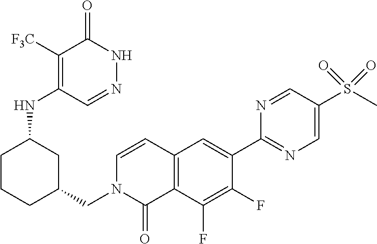 [SMILES: CS(=O)(=O)c1cnc(-c2cc3ccn(C[C@@H]4CCC[C@H](Nc5cn[nH]c(=O)c5C(F)(F)F)C4)c(=O)c3c(F)c2F)nc1] | ¹H NMR (400 MHz, DMSO-d6) δ 12.42 (s, 1H), 8.72 (dd, J = 2.3, 1.4 Hz, 1H), 8.52 (d, J = 1.4 Hz, 1H), 8.23 (d, J = 7.4 Hz, 1H), 8.00 (d, J = 11.7 Hz, 1H), 7.90 (s, 1H), 7.46 (d, J = 7.4 Hz, 1H), 6.77 (d, J = 7.3 Hz, 1H), 6.38-6.26 (m, 1H), 4.00 (s, 3H), 3.90-3.86 (m, 2H), 2.08-1.93 (m, 1H), 1.86- 1.66 (m, 3H), 1.64-1.51 (m, 1H), 1.43-1.21 (m, 3H), 1.01-0.88 (m, 1H). Note-one methine peak overlaps with water signal. ES/MS: m/z 611.1 [M + H]⁺. |
| 31 | 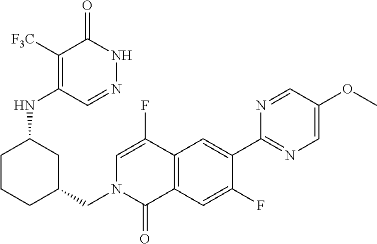 [SMILES: COc1cnc(-c2cc3c(F)cn(C[C@@H]4CCC[C@H](Nc5cn[nH]c(=O)c5C(F)(F)F)C4)c(=O)c3cc2F)nc1] | ¹H NMR (400 MHz, DMSO-d6) δ 12.42 (s, 1H), 8.77 (s, 2H), 8.12-8.07 (m, 1H), 7.92 (s, 1H), 7.48 (d, J = 7.4 Hz, 1H), 6.75 (dd, J = 7.5, 1.8 Hz, 1H), 6.33 (dd, J = 8.5, 3.9 Hz, 1H), 4.02 (s, 2H), 3.88- 3.68 (m, 3H), 2.01 (br s, 0H), 1.87-1.67 (m, 3H), 1.58 (d, J = 12.6 Hz, 1H), 1.45-1.26 (m, 2H), 1.24 (s, 6H), 1.01-0.87 (q, J = 12.8, 12.1 Hz, 1H). ES/MS: m/z 563.0 [M + H]⁺. |
| 32 | 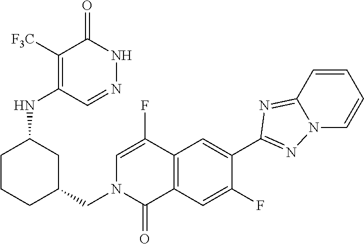 [SMILES: O=c1[nH]ncc(N[C@H]2CCC[C@@H](Cn3cc(F)c4cc(-c5nc6ccccn6n5)c(F)cc4c3=O)C2)c1C(F)(F)F] | ¹H NMR (400 MHz, DMSO-d6) δ 12.42 (s, 1H), 9.48 (m, 1H), 8.31 (m, 1H), 8.05 (m, 1H), 7.97- 7.81 (m, 2H), 7.51 (d, J = 7.4 Hz, 1H), 6.81 (m, 1H), 6.33 (m, 1H), 3.94-3.59 (m, 3H), 2.02 (m, 1H), 1.77 (m, 3H), 1.58 (m, 1H), 1.31 (m, 3H), 0.95 (m, 1H). ES/MS: m/z 572.0 [M + H]⁺. |
| 33 | 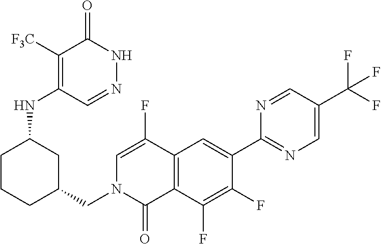 [SMILES: O=c1[nH]ncc(N[C@H]2CCC[C@@H](Cn3cc(F)c4cc(-c5ncc(C(F)(F)F)cn5)c(F)c(F)c4c3=O)C2)c1C(F)(F)F] | ¹H NMR (400 MHz, DMSO-d6) δ 12.42 (s, 1H), 9.08 (s, 2H), 8.19-8.07 (m, 1H), 7.92 (s, 1H), 7.49 (d, J = 7.4 Hz, 1H), 6.76 (dd, J = 7.5, 1.8 Hz, 1H), 6.33 (dd, J = 8.4, 4.0 Hz, 1H), 3.91-3.63 (m, 3H), 2.02 (s, 1H), 1.85-1.65 (m, 3H), 1.54 (s, 7H), 1.31 (dq, J = 36.7, 11.7 Hz, 3H), 0.94 (q, J = 12.0 Hz, 1H). ES/MS: m/z 619.1 [M + H]⁺. |
| 34 | 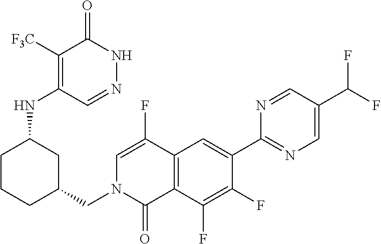 [SMILES: O=c1[nH]ncc(N[C@H]2CCC[C@@H](Cn3cc(F)c4cc(-c5ncc(C(F)F)cn5)c(F)c(F)c4c3=O)C2)c1C(F)(F)F] | ¹H NMR (400 MHz, DMSO-d6) δ 12.42 (s, 1H), 8.99 (dt, J = 2.5, 1.1 Hz, 1H), 8.27-8.18 (m, 1H), 8.05 (dt, J = 6.2, 2.9 Hz, 2H), 7.91 (s, 1H), 7.50 (d, J = 7.4 Hz, 1H), 7.26 (t, J = 55.2 Hz, 1H), 6.75 (dd, J = 7.5, 1.8 Hz, 1H), 6.33 (dd, J = 8.3, 4.0 Hz, 1H), 3.91-3.78 (m, 2H), 3.74 (s, 1H), 2.02 (s, 1H), 1.87- 1.67 (m, 3H), 1.58 (d, J = 12.7 Hz, 1H), 1.33 (dp, J = 35.9, 12.0 Hz, 3H), 0.95 (q, J = 12.5, 11.8 Hz, 1H). ES/MS: m/z 601.1 [M + H]⁺. |
| 35 | 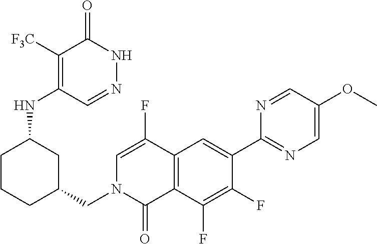 [SMILES: COc1cnc(-c2cc3c(F)cn(C[C@@H]4CCC[C@H](Nc5cn[nH]c(=O)c5C(F)(F)F)C4)c(=O)c3c(F)c2F)nc1] | ¹H NMR (400 MHz, DMSO-d6) δ 12.42 (s, 1H), 9.27 (s, 2H), 8.20 (dd, J = 6.7, 1.7 Hz, 1H), 7.92 (s, 1H), 7.51 (d, J = 7.4 Hz, 1H), 6.79 (dd, J = 7.5, 1.8 Hz, 1H), 6.37-6.28 (m, 1H), 3.91-3.78 (m, 2H), 2.14 (t, J = 19.4 Hz, 3H), 2.07-1.96 (m, 1H), 1.86- 1.68 (m, 3H), 1.61-1.53 (m, 1H), 1.45-1.19 (m, 3H), 1.02-0.86 (m, 1H). ES/MS: m/z 581.0 [M + H]⁺. |
| 36 | 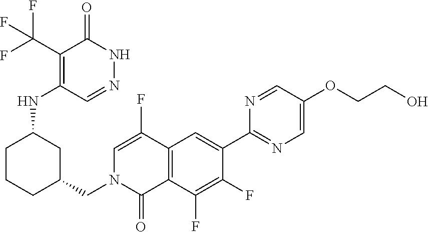 [SMILES: O=c1[nH]ncc(N[C@H]2CCC[C@@H](Cn3cc(F)c4cc(-c5ncc(OCCO)cn5)c(F)c(F)c4c3=O)C2)c1C(F)(F)F] | ¹H NMR (400 MHz, DMSO-d6) δ 12.42 (s, 1H), 9.26 (d, J = 0.9 Hz, 1H), 8.40 (d, J = 7.2 Hz, 1H), 8.02 (d, J = 11.3 Hz, 1H), 7.91 (s, 1H), 7.50 (d, J = 7.4 Hz, 1H), 6.83 (d, J = 7.4 Hz, 1H), 6.32 (dd, J = 8.3, 3.9 Hz, 1H), 3.89 (d, J = 7.0 Hz, 2H), 3.73 (br s, 2H), 2.75 (q, J = 1.5 Hz, 3H), 2.03 (br s, 1H), 1.84- 1,68 (m, 3H), 1.57 (d, J = 12.7 Hz, 1H), 1.45-1.21 (m, 3H), 1.02-0.89 (m, 1H). ES/MS: m/z 611.1 [M + H]⁺. |
| 37 | 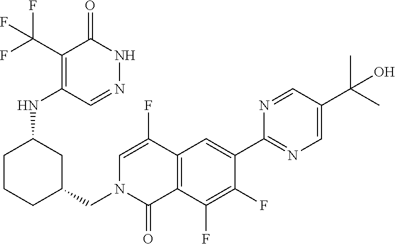 [SMILES: CC(C)(O)c1cnc(-c2cc3c(F)cn(C[C@@H]4CCC[C@H](Nc5cn[nH]c(=O)c5C(F)(F)F)C4)c(=O)c3c(F)c2F)nc1] | ¹H NMR (400 MHz, DMSO-d6) δ 12.42 (s, 1H), 8.99 (s, 1H), 8.32 (d, J = 7.2 Hz, 1H), 7.99 (d, J = 11.3 Hz, 1H), 7.91 (s, 1H), 7.48 (d, J = 7.4 Hz, 1H), 6.81 (d, J = 7.4 Hz, 1H), 6.32 (dd, J = 8.5, 3.9 Hz, 1H), 3.88 (d, J = 7.0 Hz, 2H), 3.73 (d, J = 10.7 Hz, 1H), 2.66 (s, 3H), 2.02 (br s, 1H), 1.86-1.68 (m, 3H), 1.57 (d, J = 12.7 Hz, 1H), 1.45-1.20 (m, 3H), 1.01-0.89 (m, 1H). ES/MS: m/z 609.2 [M + H]⁺. |
| 38 | 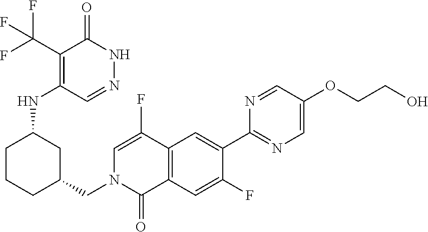 [SMILES: O=c1[nH]ncc(N[C@H]2CCC[C@@H](Cn3cc(F)c4cc(-c5ncc(OCCO)cn5)c(F)cc4c3=O)C2)c1C(F)(F)F] | ¹H NMR (400 MHz, DMSO-d6) δ 12.42 (s, 1H), 8.26 (d, J = 7.0 Hz, 1H), 8.02-7.83 (m, 2H), 7.45 (d, J = 7.4 Hz, 1H), 6.77 (d, J = 7.4 Hz, 1H), 6.32 (m, 1H), 5.66 (s, 2H), 4.56-4.38 (m, 2H), 4.28- 4.09 (m, 2H), 3.87 (m, 2H), 3.73 (m, 1H), 2.14- 1.87 (m, 1H), 1.87-1.69 (m, 3H), 1.56 (m, 1H), 1.31 (m, 3H), 1.07-0.76 (m, 1H). ES/MS: m/z 593.1 [M + H]⁺. |
| 39 | 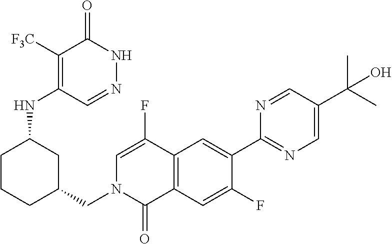 [SMILES: CC(C)(O)c1cnc(-c2cc3c(F)cn(C[C@@H]4CCC[C@H](Nc5cn[nH]c(=O)c5C(F)(F)F)C4)c(=O)c3cc2F)nc1] | ¹H NMR (400 MHz, DMSO-d6) δ 12.43 (s, 1H), 8.81 (s, 1H), 8.11 (d, J = 7.1 Hz, 1H), 8.07 (s, 1H), 7.97 (d, J = 11.1 Hz, 1H), 7.90 (s, 1H), 7.47 (d, J = 7.4 Hz, 1H), 6.74 (d, J = 7.4 Hz, 1H), 6.32 (s, 1H), 4.25 (t, J = 4.4 Hz, 2H), 3.88 (d, J = 6.9 Hz, 2H), 3.73 (d, J = 6.1 Hz, 1H), 3.58 (s, 2H), 2.08-1.95 (m, 1H), 1.78 (d, J = 11.4 Hz, 2H), 1.71 (s, 1H), 1.58 (s, 1H), 1.32 (dt, J = 35.4, 11.2 Hz, 3H), 0.95 (q, J = 12.6 Hz, 1H). ES/MS: m/z 591.2 [M + H]⁺. |
| 40 | 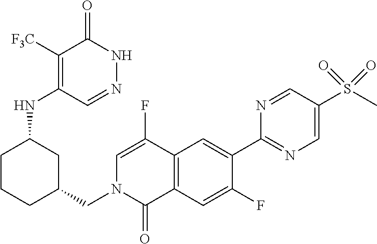 [SMILES: CS(=O)(=O)c1cnc(-c2cc3c(F)cn(C[C@@H]4CCC[C@H](Nc5cn[nH]c(=O)c5C(F)(F)F)C4)c(=O)c3cc2F)nc1] | ¹H NMR (400 MHz, DMSO-d6) δ 12.42 (s, 1H), 8.96 (s, 2H), 8.32 (d, J = 7.2 Hz, 1H), 7.98 (d, J = 11.3 Hz, 1H), 7.91 (s, 1H), 7.47 (d, J = 7.4 Hz, 1H), 6.79 (d, J = 7.4 Hz, 1H), 6.35-6.28 (m, 1H), 4.92 (q, J = 6.5 Hz, 1H), 3.88 (d, J = 6.9 Hz, 2H), 3.78- 3.67 (m, 1H), 2.08-1.96 (m, 1H), 1.80 (d, J = 7.7 Hz, 2H), 1.72 (d, J = 12.2 Hz, 1H), 1.57 (d, J = 12.3 Hz, 1H), 1.46 (d, J = 6.5 Hz, 3H), 1.31 (dq, J = 35.5, 11.9, 10.7 Hz, 3H), 1.02-0.88 (m, 1H). ES/MS: m/z 611.1 [M + H]⁺. |
| 41 | 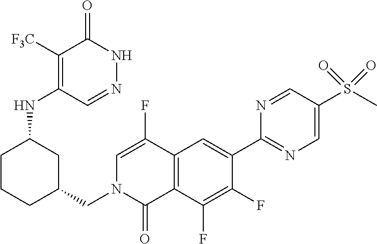 [SMILES: CS(=O)(=O)c1cnc(-c2cc3c(F)cn(C[C@@H]4CCC[C@H](Nc5cn[nH]c(=O)c5C(F)(F)F)C4)c(=O)c3c(F)c2F)nc1] | ¹H NMR (400 MHz, DMSO-d6) δ 12.42 (s, 1H), 9.26-9.21 (m, 1H), 8.50 (dd, J = 8.4, 2.4 Hz, 1H), 8.31 (d, J = 7.4 Hz, 1H), 8.16 (dd, J = 8.4, 1.9 Hz, 1H), 8.04 (d, J = 11.6 Hz, 1H), 7.91 (s, 1H), 7.49 (d, J = 7.4 Hz, 1H), 6.81 (d, J = 7.4 Hz, 1H), 6.36-6.29 (m, 1H), 3.89 (d, J = 7.0 Hz, 2H), 3.76-3.67 (m, 1H), 3.41 (s, 3H), 2.05 (d, J = 20.7 Hz, 1H), 1.85- 1.65 (m, 3H), 1.57 (d, J = 12.7 Hz, 1H), 1.46-1.20 (m, 3H), 0.95 (q, J = 12.6, 11.7 Hz, 1H). ES/MS: m/z 629.1 [M + H]⁺. |
| 42 | 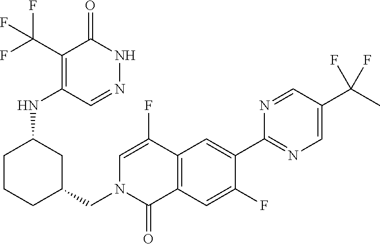 [SMILES: CC(F)(F)c1cnc(-c2cc3c(F)cn(C[C@@H]4CCC[C@H](Nc5cn[nH]c(=O)c5C(F)(F)F)C4)c(=O)c3cc2F)nc1] | ¹H NMR (400 MHz, DMSO-d6) δ 12.43 (s, 1H), 9.23-8.93 (m, 2H), 8.32 (d, J = 7.2 Hz, 1H), 8.00 (d, J = 11.3 Hz, 1H), 7.91 (s, 1H), 7.48 (d, J = 7.4 Hz, 1H), 6.80 (d, J = 7.3 Hz, 1H), 6.37-6.24 (m, 1H), 3.89 (d, J = 7.0 Hz, 2H), 3.80-3.64 (m, 1H), 2.10-1.95 (m, 1H), 1.86-1.66 (m, 3H), 1.57 (d, J = 12.6 Hz, 1H), 1.46-1.14 (m, 3H), 1.02-0.80 (m, 1H). ES/MS: m/z 597.2 [M + H]⁺. |
| 43 | 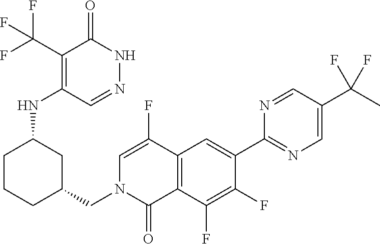 [SMILES: CC(F)(F)c1cnc(-c2cc3c(F)cn(C[C@@H]4CCC[C@H](Nc5cn[nH]c(=O)c5C(F)(F)F)C4)c(=O)c3c(F)c2F)nc1] | ¹H NMR (400 MHz, DMSO-d6) δ 12.42 (s, 1H), 9.46 (s, 2H), 8.46 (d, J = 7.1 Hz, 1H), 8.04 (d, J = 11.4 Hz, 1H), 7.91 (s, 1H), 7.51 (d, J = 7.4 Hz, 1H), 6.85 (d, J = 7.4 Hz, 1H), 6.33 (s, 1H), 3.89 (d, J = 7.0 Hz, 3H), 3.79-3.68 (m, 1H), 2.03 (s, 1H), 1.76 (dd, J = 30.0, 10.5 Hz, 4H), 1.57 (d, J = 12.7 Hz, 1H), 1.32 (dq, J = 34.5, 11.8 Hz, 3H), 1.05-0.84 (m, 1H). ES/MS: m/z 615.1 [M + H]⁺. |
| 44 | 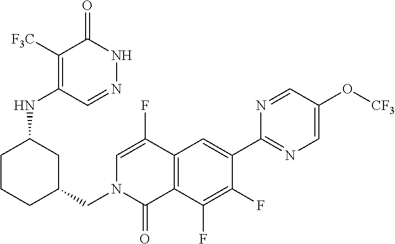 [SMILES: O=c1[nH]ncc(N[C@H]2CCC[C@@H](Cn3cc(F)c4cc(-c5ncc(OC(F)(F)F)cn5)c(F)c(F)c4c3=O)C2)c1C(F)(F)F] | ¹H NMR (400 MHz, DMSO-d6) δ 12.42 (s, 1H), 8.83 (s, 2H), 8.30 (d, J = 7.3 Hz, 1H), 8.08-7.82 (m, 2H), 7.46 (d, J = 7.4 Hz, 1H), 6.79 (d, J = 7.4 Hz, 1H), 6.35 (t, J = 3.4 Hz, 1H), 6.32 (dd, J = 8.4, 4.0 Hz, 1H), 4.64 (td, J = 14.7, 3.4 Hz, 2H), 3.88 (d, J = 7.0 Hz, 2H), 3.73 (s, 1H), 2.02 (s, 1H), 1.90- 1.65 (m, 3H), 1.57 (d, J = 12.5 Hz, 1H), 1.33 (dp, J = 34.1, 12.4, 11.6 Hz, 3H), 0.95 (q, J = 12.7, 12.2 Hz, 1H). ES/MS: m/z 635.1 [M + H]⁺. |
| 45 | 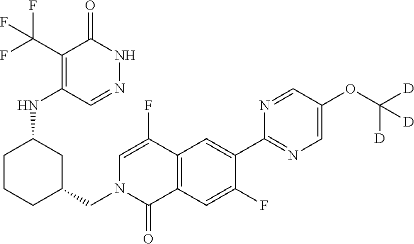 [SMILES: [2H]C([2H])([2H])Oc1cnc(-c2cc3c(F)cn(C[C@@H]4CCC[C@H](Nc5cn[nH]c(=O)c5C(F)(F)F)C4)c(=O)c3cc2F)nc1] | ¹H NMR (400 MHz, DMSO-d6) δ 12.42 (s, 1H), 8.72 (dd, J = 2.3, 1.4 Hz, 1H), 8.52 (d, J = 1.4 Hz, 1H), 8.23 (d, J = 7.4 Hz, 1H), 8.00 (d, J = 11.7 Hz, 1H), 7.90 (s, 1H), 7.46 (d, J = 7.4 Hz, 1H), 6.77 (d, J = 7.3 Hz, 1H), 6.38-6.26 (m, 1H), 4.00 (s, 3H), 3.90-3.86 (m, 2H), 2.08-1.93 (m, 1H), 1.86- 1.66 (m, 3H), 1.64-1.51 (m, 1H), 1.43-1.21 (m, 3H), 1.01-0.88 (m, 1H). Note-one methine peak overlaps with water signal. ES/MS: m/z 566.1 [M + H]⁺. |
| 46 | 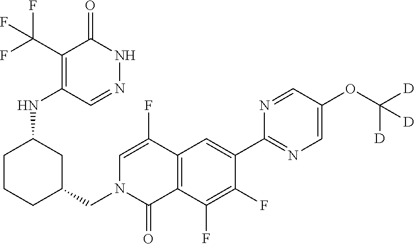 [SMILES: [2H]C([2H])([2H])Oc1cnc(-c2cc3c(F)cn(C[C@@H]4CCC[C@H](Nc5cn[nH]c(=O)c5C(F)(F)F)C4)c(=O)c3c(F)c2F)nc1] | ¹H NMR (400 MHz, DMSO-d6) δ 12.42 (s, 1H), 8.77 (s, 2H), 8.12-8.07 (m, 1H), 7.92 (s, 1H), 7.48 (d, J = 7.4 Hz, 1H), 6.75 (dd, J = 7.5, 1.8 Hz, 1H), 6.33 (dd, J = 8.5, 3.9 Hz, 1H), 4.02 (s, 2H), 3.88- 3.68 (m, 3H), 2.01 (br s, 0H), 1.87-1.67 (m, 3H), 1.58 (d, J = 12.6 Hz, 1H), 1.45-1.26 (m, 2H), 1.24 (s, 6H), 1.01-0.87 (q, J = 12.8, 12.1 Hz, 1H). ES/MS: m/z 584.1 [M + H]⁺. |
| 47 | 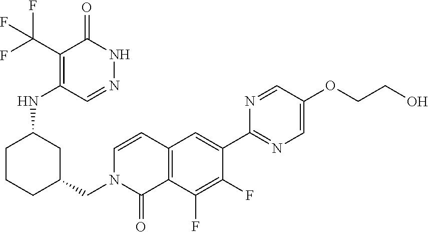 [SMILES: O=c1[nH]ncc(N[C@H]2CCC[C@@H](Cn3ccc4cc(-c5ncc(OCCO)cn5)c(F)c(F)c4c3=O)C2)c1C(F)(F)F] | ¹H NMR (400 MHz, DMSO-d6) δ 12.42 (s, 1H), 8.77 (s, 2H), 8.15-8.06 (m, 1H), 7.91 (s, 1H), 7.48 (d, J = 7.4 Hz, 1H), 6.75 (dd, J = 7.5, 1.9 Hz, 1H), 6.33 (dd, J = 8.4, 4.0 Hz, 1H), 4.32-4.22 (m, 2H), 3.88-3.66 (m, 5H), 2.01 (s, 1H), 1.77 (d, J = 25.4 Hz, 3H), 1.57 (d, J = 12.7 Hz, 1H), 1.32 (dp, J = 36.3, 12.3 Hz, 3H), 0.94 (q, J = 13.0, 12.2 Hz, 1H). ES/MS: m/z 593.1 [M + H]⁺. |

### Example 48: Preparation of 4,7,8-trifluoro-6-(5-(2-hydroxypropan-2-yl)pyrimidin-2-yl)-2-(((1R,3S)-3-((6-oxo-5-(trifluoromethyl)-1,6-dihydropyridazin-4-yl)oxy)cyclohexyl)methyl)isoquinolin-1(2H) one

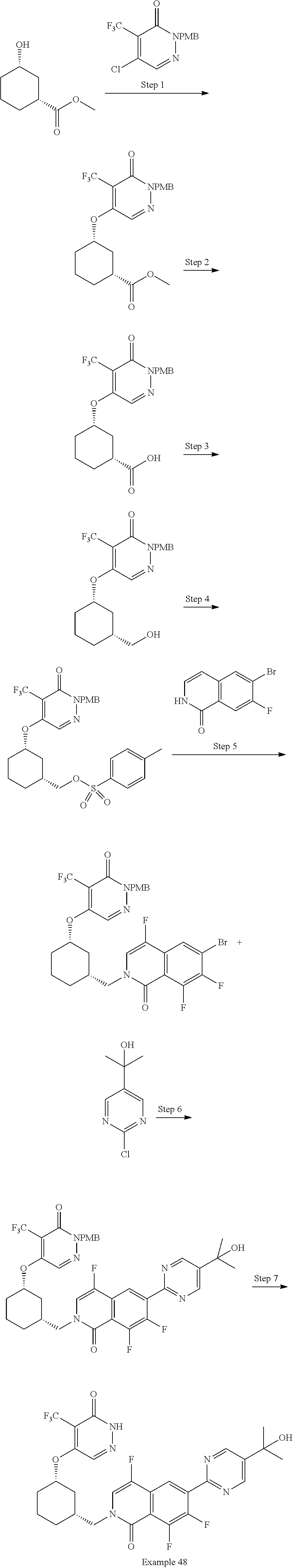

Step 1. Methyl (1R,3S)-3-hydroxycyclohexanecarboxylate (1.0 g, 6.3 mmol), 5-chloro-2-[(4-methoxyphenyl)methyl]-4-(trifluoromethyl)pyridazin-3-one (3.0 g, 9.5 mmol) and cesium carbonate (4.1 g, 13 mmol) were combined in MeCN (25 mL) and stirred at ambient temperature. After 4 days the reaction was filtered. The filtrate was concentrated and purified using column chromatography eluting with EtOAc in hexane to give methyl (1R,3S)-3-[1-[(4-methoxyphenyl)methyl]-6-oxo-5-(trifluoromethyl)pyridazin-4-yl]oxycyclohexanecarboxylate. ES/MS m/z=441.1 [M+H]⁺.

Step 2. Methyl (1R,3S)-3-[1-[(4-methoxyphenyl)methyl]-6-oxo-5-(trifluoromethyl)pyridazin-4-yl]oxycyclohexanecarboxylate (8.0 g, 18.2 mmol) was dissolved in a mixture of THF (75 mL) and water (25 mL) and was treated with LiOH (1M/aq, 29 mL, 29 mmol). The reaction was stirred at ambient temperature for 90 minutes, then was acidified with aq. HCl to pH˜3. The reaction was extracted 3× with EtOAc and the combined extracts were washed with brine, dried over Na₂SO₄ and concentrated in vacuo to give (1R,3S)-3-[1-[(4-methoxyphenyl)methyl]-6-oxo-5-(trifluoromethyl)pyridazin-4-yl]oxycyclohexanecarboxylic acid, which was used in the following step without purification. ES/MS m/z=426.9 [M+H]⁺.

Step 3. (1R,3S)-3-[1-[(4-methoxyphenyl)methyl]-6-oxo-5-(trifluoromethyl)pyridazin-4-yl]oxycyclohexanecarboxylic acid (7.76 g, 18.2 mmol) was combined with TEA (2.8 mL, 20 mmol) in THF (75 mL) and was cooled in an ice bath under a stream of nitrogen gas. Ethyl chloroformate (1.75 mL, 18.2 mmol) was added and the resulting mixture was stirred at 0° C. After 35 minutes, the reaction was filtered and the filtrate was transferred to an addition funnel. The anhydride was added dropwise to a stirred suspension of NaBH₄ (2.07 g, 54.6 mmol) in a mixture of THF (25 mL) and water (5 mL) in an ice bath. The reaction was allowed to stir at 0° C. for several hours, then warmed to ambient temperature. Aq. HCl was added to adjust the pH to 2-3, and the resulting mixture was extracted 3× with EtOAc. The combined extracts were washed with brine, concentrated, and purified using column chromatography eluting with 3:1 EtOAc/EtOH in heptane to give 5-(((1S,3R)-3-(hydroxymethyl)cyclohexyl)oxy)-2-(4-methoxybenzyl)-4-(trifluoromethyl)pyridazin-3(2H)-one. ES/MS m/z=413.1 [M+H]⁺.

Step 4. 5-(((1S,3R)-3-(hydroxymethyl)cyclohexyl)oxy)-2-(4-methoxybenzyl)-4-(trifluoromethyl)pyridazin-3(2H)-one was subjected to the conditions used for Example 1, Step 1 to give ((1R,3S)-3-((1-(4-methoxybenzyl)-6-oxo-5-(trifluoromethyl)-1,6-dihydropyridazin-4-yl)oxy)cyclohexyl)methyl 4-methylbenzenesulfonate. ES/MS m/z=567.0 [M+H]⁺.

Step 5. ((1R,3S)-3-((1-(4-methoxybenzyl)-6-oxo-5-(trifluoromethyl)-1,6-dihydropyridazin-4-yl)oxy)cyclohexyl)methyl 4-methylbenzenesulfonate was subjected to the conditions used for Example 1, Step 2 to give 6-bromo-4,7,8-trifluoro-2-(((1R,3S)-3-((1-(4-methoxybenzyl)-6-oxo-5-(trifluoromethyl)-1,6-dihydropyridazin-4-yl)oxy)cyclohexyl)methyl)isoquinolin-1(2H)-one. ES/MS m/z=672.1 [M+H]⁺.

Step 6. 6-bromo-4,7,8-trifluoro-2-(((1R,3S)-3-((1-(4-methoxybenzyl)-6-oxo-5-(trifluoromethyl)-1,6-dihydropyridazin-4-yl)oxy)cyclohexyl)methyl)isoquinolin-1(2H)-one was subjected to the conditions used for Example 1, Step 5 to give 4,7,8-trifluoro-6-(5-(2-hydroxypropan-2-yl)pyrimidin-2-yl)-2-(((1R,3S)-3-((1-(4-methoxybenzyl)-6-oxo-5-(trifluoromethyl)-1,6-dihydropyridazin-4-yl)oxy)cyclohexyl)methyl)isoquinolin-1(2H)-one. ES/MS m/z=730.3 [M+H]⁺.

Step 7. 4,7,8-trifluoro-6-(5-(2-hydroxypropan-2-yl)pyrimidin-2-yl)-2-(((1R,3S)-3-((1-(4-methoxybenzyl)-6-oxo-5-(trifluoromethyl)-1,6-dihydropyridazin-4-yl)oxy)cyclohexyl)methyl)isoquinolin-1(2H)-one was dissolved in TFA and cooled to 0° C. Triflic acid was added, dropwise, until the reaction color turned deep red. Once complete, the reaction was added dropwise to a stirring solution of aq. NaHCO₃, then extracted 3× with DCM. The combined extracts were concentrated in vacuo and purified via reverse phase prep-HPLC (5-100% MeCN in water, 0.1% TFA) to afford 4,7,8-trifluoro-6-(5-(2-hydroxypropan-2-yl)pyrimidin-2-yl)-2-(((1R,3S)-3-((6-oxo-5-(trifluoromethyl)-1,6-dihydropyridazin-4-yl)oxy)cyclohexyl)methyl)isoquinolin-1(2H)-one. ¹H NMR (400 MHz, DMSO-d6) δ 13.25 (s, 1H), 9.11 (s, 2H), 8.32 (s, 1H), 8.20-8.13 (m, 1H), 7.88 (d, J=6.8 Hz, 1H), 4.88-4.70 (i, 1H), 3.92-3.75 (m, 2H), 2.10-2.02 (i, 3H), 1.83-1.75 (m, 1H), 1.65-1.52 (m, 1H), 1.55 (s, 6H), 1.41-1.15 (i, 3H), 1.09-10.97 (m, 1H). ES/MS m/z=610.1 [M+H]⁺.

The following compounds were synthesized using the procedure described in Example 48 from the appropriate starting materials.

| Example | | |
|---|---|---|
| No. | Structure | ¹H NMR, MS [M + H]⁺ |
| 49 | 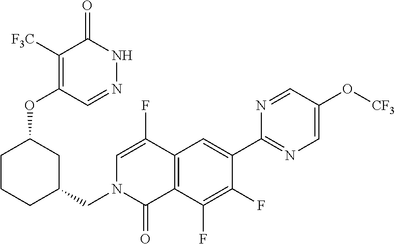 [SMILES: O=c1[nH]ncc(O[C@H]2CCC[C@@H](Cn3cc(F)c4cc(-c5ncc(OC(F)(F)F)cn5)c(F)c(F)c4c3=O)C2)c1C(F)(F)F] | ¹H NMR (400 MHz, DMSO-d6) δ 13.26 (s, 1H), 9.27 (s, 2H), 8.32 (s, 1H), 8.19 (d, J = 6.4 Hz, 1H), 7.90 (d, J = 6.8 Hz, 1H), 4.87-4.74 (m, 1H), 3.94-3.70 (m, 2H), 2.15-1.97 (m, 3H), 1.85-1.73 (m, 1H), 1.66-1.54 (m, 1H), 1.42- 1.17 (m, 3H), 1.10-0.89 (m, 1H). ES/MS: m/z 636.1 [M + H]⁺. |
| 50 | 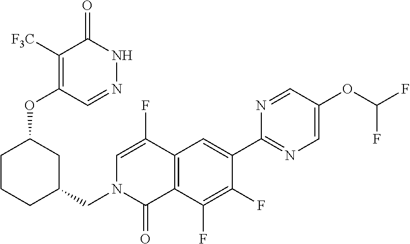 [SMILES: O=c1[nH]ncc(O[C@H]2CCC[C@@H](Cn3cc(F)c4cc(-c5ncc(OC(F)F)cn5)c(F)c(F)c4c3=O)C2)c1C(F)(F)F] | ¹H NMR (400 MHz, DMSO-d6) δ 13.25 (s, 1H), 9.04 (s, 2H), 8.32 (s, 1H), 8.16 (d, J = 6.2 Hz, 1H), 7.89 (d, J = 6.8 Hz, 1H), 7.51 (t, J = 72.5 Hz, 1H), 4.89-4.70 (m, 1H), 3.95-3.68 (m, 2H), 2.16-1.94 (m, 4H), 1.88-1.71 (m, 1H), 1.71- 1.54 (m, 1H), 1.51-1.16 (m, 3H), 1.16-0.94 (m, 1H). ES/MS: m/z 618.0 [M + H]⁺. |
| 51 | 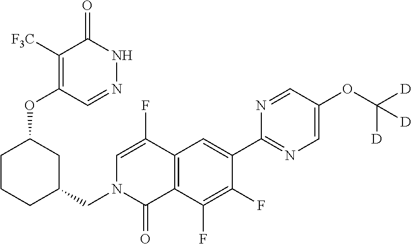 [SMILES: [2H]C([2H])([2H])Oc1cnc(-c2cc3c(F)cn(C[C@@H]4CCC[C@H](Oc5cn[nH]c(=O)c5C(F)(F)F)C4)c(=O)c3c(F)c2F)nc1] | ¹H NMR (400 MHz, DMSO-d6) δ 13.25 (s, 2H), 8.79 (s, 2H), 8.32 (s, 1H), 8.24-8.03 (m, 1H), 7.95-7.75 (m, 2H), 4.91-4.69 (m, 2H), 3.93- 3.72 (m, 2H), 2.15-1.97 (m, 5H), 1.86-1.68 (m, 2H), 1.68-1.54 (m, 2H), 1.46-1.13 (m, 5H), 1.13-0.91 (m, 1H). ES/MS: m/z 585.1 [M + H]⁺. |
| 52 | 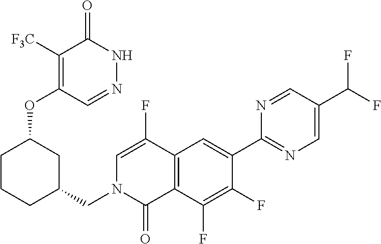 [SMILES: O=c1[nH]ncc(O[C@H]2CCC[C@@H](Cn3cc(F)c4cc(-c5ncc(C(F)F)cn5)c(F)c(F)c4c3=O)C2)c1C(F)(F)F] | ¹H NMR (400 MHz, DMSO-d6) δ 13.25 (s, 1H), 9.29 (s, 1H), 8.31 (s, 1H), 8.22 (d, J = 6.2 Hz, 1H), 7.90 (d, J = 6.8 Hz, 1H), 7.32 (t, J = 54.7 Hz, 1H), 4.86-4.74 (m, 1H), 3.94-3.73 (m, 2H), 2.14-1.98 (m, 4H), 1.87-1.73 (m, 1H), 1.65- 1.55 (m, 1H), 1.44-1.18 (m, 3H), 1.11-0.96 (m, 1H). ES/MS: m/z 602.1 [M + H]⁺. |
| 53 | 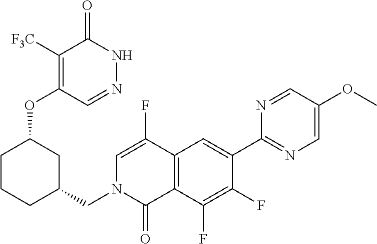 [SMILES: COc1cnc(-c2cc3c(F)cn(C[C@@H]4CCC[C@H](Oc5cn[nH]c(=O)c5C(F)(F)F)C4)c(=O)c3c(F)c2F)nc1] | ¹H NMR (400 MHz, DMSO-d6) δ 13.25 (s, 1H), 8.79 (s, 2H), 8.31 (s, 1H), 8.12 (d, J = 6.3 Hz, 1H), 7.86 (d, J = 6.8 Hz, 1H), 4.90-4.69 (m, 1H), 4.01 (s, 3H), 3.91-3.74 (m, 2H), 2.13- 1.98 (m, 3H), 1.84-1.73 (m, 1H), 1.65-1.54 (m, 1H), 1.43-1.15 (m, 3H), 1.08-0.95 (m, 1H). ES/MS: m/z 582.1 [M + H]⁺. |
| 54 | 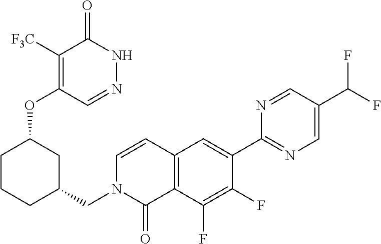 [SMILES: O=c1[nH]ncc(O[C@H]2CCC[C@@H](Cn3ccc4cc(-c5ncc(C(F)F)cn5)c(F)c(F)c4c3=O)C2)c1C(F)(F)F] | ¹H NMR (400 MHz, DMSO-d6) δ 13.24 (s, 1H), 9.27 (s, 1H), 8.30 (s, 1H), 8.21 (d, J = 6.4 Hz, 1H), 7.52 (d, J = 7.4 Hz, 1H), 7.31 (t, J = 54.7 Hz, 1H), 6.83-6.76 (m, 1H), 4.90-4.73 (m, 1H), 3.98-3.88 (m, 1H), 3.88-3.74 (m, 1H), 2.18- 1.93 (m, 4H), 1.87-1.68 (m, 1H), 1.68-1.48 (m, 1H), 1.44-1.12 (m, 3H), 1.12-0.85 (m, 1H). ES/MS: m/z 584.1 [M + H]⁺. |
| 55 | 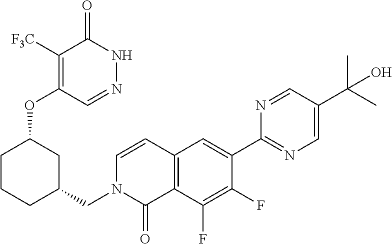 [SMILES: CC(C)(O)c1cnc(-c2cc3ccn(C[C@@H]4CCC[C@H](Oc5cn[nH]c(=O)c5C(F)(F)F)C4)c(=O)c3c(F)c2F)nc1] | ¹H NMR (400 MHz, DMSO-d6) δ 13.24 (s, 1H), 9.08 (s, 2H), 8.30 (s, 1H), 8.19-8.04 (m, 1H), 7.50 (d, J = 7.4 Hz, 1H), 6.83-6.65 (m, 1H), 3.98-3.76 (m, 2H), 2.14-1.96 (m, 4H), 1.84- 1.71 (m, 1H), 1.61-1.48 (m, 6H), 1.44-1.17 (m, 3H), 1.12-0.96 (m, 1H). ES/MS: m/z 592.1 [M + H]⁺. |
| 56 | 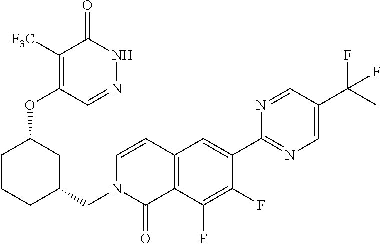 [SMILES: CC(F)(F)c1cnc(-c2cc3ccn(C[C@@H]4CCC[C@H](Oc5cn[nH]c(=O)c5C(F)(F)F)C4)c(=O)c3c(F)c2F)nc1] | ¹H NMR (400 MHz, DMSO-d6) δ 13.24 (s, 1H), 9.27 (s, 2H), 8.30 (s, 1H), 8.26-8.09 (m, 1H), 7.52 (d, J = 7.4 Hz, 1H), 6.88-6.70 (m, 1H), 4.94-4.64 (m, 1H), 3.99-3.78 (m, 2H), 2.14 (t, J = 19.3 Hz, 3H), 2.07-1.94 (m, 4H), 1.84-1.69 (m, 1H), 1.66-1.51 (m, 1H), 1.44-1.15 (m, 3H), 1.11-0.93 (m, 1H). ES/MS: m/z 598.1 [M + H]⁺. |
| 57 | 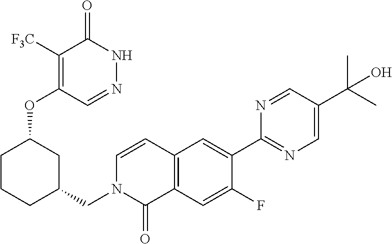 [SMILES: CC(C)(O)c1cnc(-c2cc3ccn(C[C@@H]4CCC[C@H](Oc5cn[nH]c(=O)c5C(F)(F)F)C4)c(=O)c3cc2F)nc1] | ¹H NMR (400 MHz, DMSO-d6) δ 13.24 (s, 1H), 9.06 (s, 2H), 8.32 (d, J = 7.3 Hz, 1H), 8.30 (s, 1H), 7.99 (d, J = 11.3 Hz, 1H), 7.48 (d, J = 7.4 Hz, 1H), 6.79 (d, J = 7.4 Hz, 1H), 4.00-3.81 (m, 2H), 2.17-1.96 (m, 3H), 1.83-1.74 (m, 1H), 1.62-1.47 (m, 7H), 1.44-1.19 (m, 3H), 1.11- 0.94 (m, 1H). ES/MS: m/z 574.2 [M + H]⁺. |

### Example 58: Preparation of 4-chloro-7-fluoro-6-(5-methoxypyrimidin-2-yl)-2-[[(1R,3S)-3-[[6-oxo-5-(trifluoromethyl)-1H-pyridazin-4-yl]amino]cyclohexyl]methyl]isoquinolin-1l-one

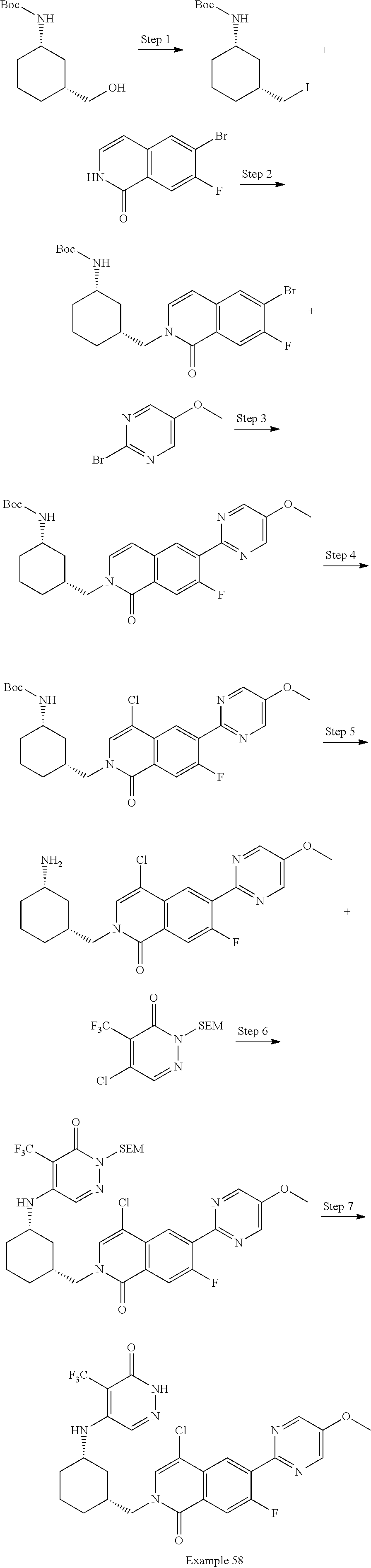

Step 1. Iodine (2.02 g, 7.96 mmol) was added to a solution of triphenylphosphine (2.09 g, 7.96 mmol) and imidazole (0.542 g, 7.96 mmol) in DCM (27 ml). The mixture is stirred at RT until a solution forms (30 minutes) then tert-butyl ((1S,3R)-3-(hydroxymethyl)cyclohexyl)carbamate (1.522 g, 6.64 mmol) is added. After stirring the mixture at room temperature for 18 h, the mixture is filtered, and the filtrate is evaporated. The residue is dissolved in EtOAc and washed with sat. aq. sodium bisulfite and then brine. The organic phase is dried over MgSO₄ and the solvent is removed under reduced pressure. The residue is purified by flash chromatography using a gradient of EtOAc/hexane (0 to 100%) as eluent to give tert-butyl N-[(1S,3R)-3-(iodomethyl)cyclohexyl]carbamate. ES/MS: m/z 340.0 [M+H]⁺.

Step 2. To a mixture of 6-bromo-7-fluoro-2H-isoquinolin-1-one (785 mg, 3.24 mmol) and tert-butyl N-[(1S,3R)-3-(iodomethyl)cyclohexyl]carbamate (1000 mg, 2.95 mmol) in DMF (19 mL) was added Cs₂CO₃ (1921 mg, 5.90 mmol) and the reaction was stirred at room temperature for 18 h. Upon completion, the mixture was diluted with EtOAc, washed with water, washed with brine, dried over MgSO₄ and concentrated in vacuo to give the crude product. The crude residue was purified using column chromatography eluting with EtOAc in hexanes 0-100% to afford tert-butyl N-[(1S,3R)-3-[(6-bromo-7-fluoro-1-oxo-2-isoquinolyl)methyl]cyclohexyl]carbamate. ES/MS: m/z 454.9 [M+H]⁺.

Step 3. In a flask was placed tert-butyl N-[(1S,3R)-3-[(6-bromo-7-fluoro-1-oxo-2-isoquinolyl)methyl]cyclohexyl]carbamate (400 mg, 0.882 mmol), 1,1′-Bis(diphenylphosphino)ferrocene-palladium(II)dichloride dichloromethane complex (72.1 mg, 0.088 mmol), potassium acetate (260 mg, 2.65 mmol), and bis(pinacolato)diboron (336 mg, 1.32 mmol) in dioxane (8 mL). The mixture was sparged with nitrogen gas and then stirred at 80° C. for 18 hr. The reaction was cooled to ambient temperature and cataCXium Pd G4 (65.5 mg, 0.088 mmol), 2 M aqueous sodium carbonate (0.97 mL, 2.19 mmol), and 2-bromo-5-methoxy-pyrimidine (250 mg, 1.32 mmol) were added. The mixture was then stirred at 90° C. for 3 h. The reaction was then cooled, diluted with EtOAc and filtered through a plug of Celite®. The volatiles were evaporated in vacuo and the residue purified via flash chromatography (100% Hexanes to 100% EtOAc) to give tert-butyl N-[(1S,3R)-3-[[7-fluoro-6-(5-methoxypyrimidin-2-yl)-1-oxo-2-isoquinolyl]methyl]cyclohexyl]carbamate. ES/MS m/z: 483.1 [M+H]⁺.

Step 4. To a solution tert-butyl N-[(1S,3R)-3-[[7-fluoro-6-(5-methoxypyrimidin-2-yl)-1-oxo-2-isoquinolyl]methyl]cyclohexyl]carbamate (426 mg, 0.883 mmol) in DMF (14 mL) was added N-chlorosuccinimide (118 mg, 0.883 mmol). The reaction mixture was stirred at 60° C. for 6 hr. Upon cooling to room temperature, the reaction was poured into water and the aqueous layer was extracted with EtOAc. The organic layer was washed with brine, dried (MgSO₄), filtered, and concentrated under reduced pressure to afford tert-butyl N-[(1S,3R)-3-[[4-chloro-7-fluoro-6-(5-methoxypyrimidin-2-yl)-1-oxo-2-isoquinolyl]methyl]cyclohexyl]carbamate which was used without further purification. ES/MS: m/z 517.0 [M+H]⁺.

Step 5. To a solution tert-butyl N-[(1S,3R)-3-[[4-chloro-7-fluoro-6-(5-methoxypyrimidin-2-yl)-1-oxo-2-isoquinolyl]methyl]cyclohexyl]carbamate (456 mg, 0.882 mmol) in dichloromethane (10 mL) was added trifluoroacetic acid (1 mL) at room temperature and the mixture was stirred for 1 hr. Upon completion, the solvent was removed under reduced pressure to afford 2-[[(1R,3S)-3-aminocyclohexyl]methyl]-4-chloro-7-fluoro-6-(5-methoxypyrimidin-2-yl)isoquinolin-1-one which was used without further purification. ES/MS: m/z 419.1 [M+H]⁺.

Step 6. A mixture of 2-[[(1R,3S)-3-aminocyclohexyl]methyl]-4-chloro-7-fluoro-6-(5-methoxypyrimidin-2-yl)isoquinolin-1-one (368 mg, 0.883 mmol), 5-chloro-4-(trifluoromethyl)-2-(2-trimethylsilylethoxymethyl)pyridazin-3-one (387 mg, 1.06 mmol), and N,N-diisopropylethylamine (0.67 mL, 3.83 mmol) in DMF (11 mL) was stirred at room temperature for 2 h. Upon completion, the reaction was diluted with EtOAc, washed with water, washed with brine, dried over MgSO₄ and concentrated to give the crude product. The crude residue was purified using column chromatography eluting with EtOAc in hexanes 0-100% to provide 4-chloro-7-fluoro-6-(5-methoxypyrimidin-2-yl)-2-[[(1R,3S)-3-[[6-oxo-5-(trifluoromethyl)-1-(2-trimethylsilylethoxymethyl)pyridazin-4-yl]amino]cyclohexyl]methyl]isoquinolin-1-one. ES/MS: m/z 711.2 [M+H]⁺.

Step 7. In a vial was placed 4-chloro-7-fluoro-6-(5-methoxypyrimidin-2-yl)-2-[[(1R,3S)-3-[[6-oxo-5-(trifluoromethyl)-1-(2-trimethylsilylethoxymethyl)pyridazin-4-yl]amino]cyclohexyl]methyl]isoquinolin-1-one (626 mg, 0.883 mmol), and trifluoroacetic acid (1.0 mL) in DCM (5.0 mL). The mixture was stirred for 1 hr, and then was concentrated under vacuum. The resulting crude product was dissolved in methanol (4.0 mL) and 2.0 M ammonia in methanol (1.0 mL, 2.29 mmol) was added and the mixture was stirred for 30 minutes and then concentrated under vacuum. The resulting crude product was purified by reverse phase prep-HPLC (5-100% MeCN in water, 0.1% TFA) to afford 4-chloro-7-fluoro-6-(5-methoxypyrimidin-2-yl)-2-[[(1R,3S)-3-[[6-oxo-5-(trifluoromethyl)-1H-pyridazin-4-yl]amino]cyclohexyl]methyl]isoquinolin-1-one. ES/MS: m/z 580.3 [M+H]⁺.

The following compounds were synthesized using the procedure described in Example 58 from the appropriate starting materials.

| Example | | |
|---|---|---|
| No. | Structure | ¹H NMR, MS [M + H]⁺ |
| 59 | 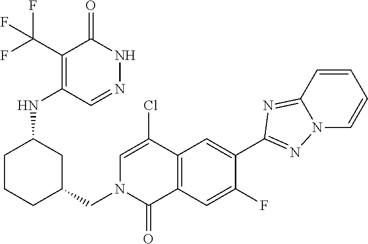 [SMILES: O=c1[nH]ncc(N[C@H]2CCC[C@@H](Cn3cc(Cl)c4cc(-c5nc6ccccn6n5)c(F)cc4c3=O)C2)c1C(F)(F)F] | ¹H NMR (400 MHz, DMSO-d6) δ 12.42 (s, 1H), 9.15-9.03 (m, 1H), 8.70 (d, J = 6.6 Hz, 1H), 8.13 (d, J = 11.0 Hz, 1H), 8.02-7.96 (m, 1H), 7.93 (d, J = 3.5 Hz, 2H), 7.81-7.70 (m, 1H), 7.35-7.25 (m, 1H), 6.43-6.24 (m, 1H), 3.99- 3.81 (m, 2H), 3.81-3.63 (m, 1H), 2.13-1.93 (m, 1H), 1.89-1.77 (m, 2H), 1.77-1.67 (m, 1H), 1.68-1.50 (m, 1H), 1.46-1.20 (m, 3H), 1.06-0.83 (m, 1H). ES/MS: m/z 588.0 [M + H]⁺. |
| 60 | 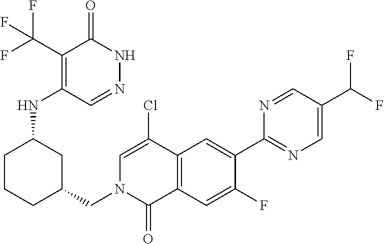 [SMILES: O=c1[nH]ncc(N[C@H]2CCC[C@@H](Cn3cc(Cl)c4cc(-c5ncc(C(F)F)cn5)c(F)cc4c3=O)C2)c1C(F)(F)F] | ¹H NMR (400 MHz, DMSO-d6) δ 12.42 (s, 1H), 9.32-9.23 (m, 2H), 8.54 (d, J = 6.9 Hz, 1H), 8.12 (d, J = 11.0 Hz, 1H), 7.93 (d, J = 7.7 Hz, 2H), 7.32 (t, J = 54.7 Hz, 1H), 6.40-6.26 (m, 1H), 3.92-3.87 (m, 3H), 2.13-1.95 (m, 1H), 1.87-1.68 (m, 3H), 1.63-1.52 (m, 1H), 1.41- 1.22 (m, 3H), 1.02-0.88 (m, 1H). ES/MS: m/z 599.1 [M + H]⁺. |
| 61 | 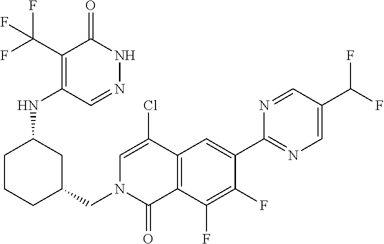 [SMILES: O=c1[nH]ncc(N[C@H]2CCC[C@@H](Cn3cc(Cl)c4cc(-c5ncc(C(F)F)cn5)c(F)c(F)c4c3=O)C2)c1C(F)(F)F] | ¹H NMR (400 MHz, DMSO-d6) δ 12.43 (s, 1H), 9.34-9.18 (m, 2H), 8.47-8.28 (m, 1H), 8.02- 7.89 (m, 2H), 7.32 (t, J = 54.7 Hz, 1H), 6.39- 6.27 (m, 1H), 3.92-3.79 (m, 2H), 3.74 (s, 1H), 2.11-1.95 (m, 1H), 1.88-1.67 (m, 3H), 1.66- 1.52 (m, 1H), 1.45-1.20 (m, 3H), 1.02-0.84 (m, 1H). ES/MS: m/z 617.1 [M + H]⁺. |

### Example 62: Preparation of 7-fluoro-2-[[(1S,3S,5R)-3-hydroxy-5-[[6-oxo-5-(trifluoromethyl)-1H-pyridazin-4-yl]amino]cyclohexyl]methyl]-6-[5-(trifluoromethyl)pyrimidin-2-yl]isoquinolin-1-one

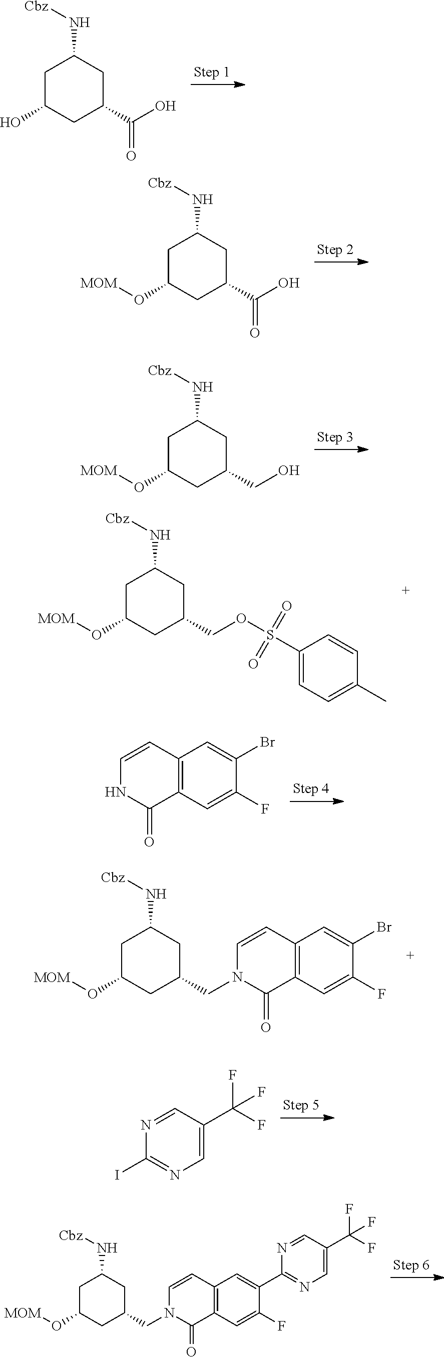

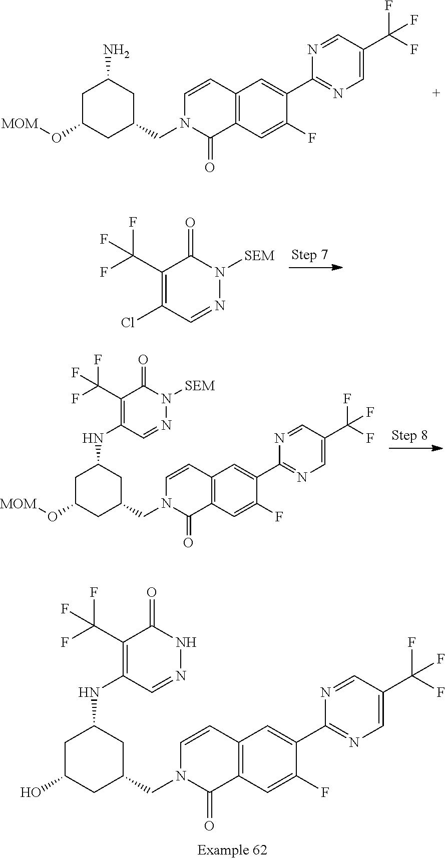

Step 1. Bromomethyl methyl ether (0.56 ml, 6.82 mmol) was added to a solution of (1S,3R,5S)-3-(benzyloxycarbonylamino)-5-hydroxy-cyclohexanecarboxylic acid (1000 mg, 3.41 mmol) and DIPEA (1.48 ml, 8.52 mmol) in THF (34.0 ml) and was stirred at room temperature for 2 hr. Once complete, NaOH (1200 mg, 30 mmol) and water (5.0 ml) was added at stirred at room temperature for 16 hr. Upon completion, the mixture was diluted with water and extracted with EtOAc. The aqueous layer was acidified to pH˜4 and extracted with EtOAc, washed with water. Organic layer was washed with brine, dried over MgSO₄ and concentrated to give the crude product. The residue was purified using column chromatography eluting with EtOAc in hexanes 0-100% to afford (1S,3R,5S)-3-(benzyloxycarbonylamino)-5-(methoxymethoxy)cyclohexanecarboxylic acid. ES/MS: m/z 338.0 [M+H]⁺.

Step 2. In a flask was placed (1S,3R,5S)-3-(benzyloxycarbonylamino)-5-(methoxymethoxy)cyclohexanecarboxylic acid (1390 mg, 4.12 mmol), and triethylamine (1.72 mL, 12.4 mmol) in THF (20 mL). The mixture was cooled to 0° C. and placed under nitrogen atmosphere. To this solution was added ethyl chloroformate (0.59 mL, 6.18 mmol) and the reaction was stirred for 30 minutes at 0° C. The mixture was then filtered to remove the precipitated triethylamine hydrochloride. The filtrate containing the mixed anhydride was slowly added to a stirred suspension of sodium borohydride (468 mg, 12.4 mmol) in 20% aqueous THF (10.0 mL) maintained at 10° C. After the mixture was stirred for 30 minutes and then quenched with sat. aq. NH₄Cl. The mixture was then extracted with EtOAc, washed with brine, dried (MgSO₄), and concentrated to afford benzyl N-[(1R,3S,5S)-3-(hydroxymethyl)-5-(methoxymethoxy)cyclohexyl]carbamate which was used without further purification. ES/MS: m/z 324.0 [M+H]⁺.

Step 3. To a stirred solution of benzyl N-[(1R,3S,5S)-3-(hydroxymethyl)-5-(methoxymethoxy)cyclohexyl]carbamate (1330 mg, 4.11 mmol) and triethylamine (1.15 mL, 8.23 mmol) in dichloromethane (22.0 mL) at 0° C. was added p-toluenesulfonyl chloride (823 mg, 0.32 mmol) and the mixture was warmed to room temperature and stirred for 18 h. Upon completion, the mixture was diluted with water and extracted with dichloromethane. The combined organic layers were washed with brine, dried over MgSO₄ and concentrated to give the crude product. The residue was purified using column chromatography eluting with EtOAc in hexanes 0-100% to afford [(1S,3R,5S)-3-(benzyloxycarbonylamino)-5-(methoxymethoxy)cyclohexyl]methyl 4-methylbenzenesulfonate ES/MS: m/z 478.0 [M+H]⁺.

Step 4. To a mixture of 6-bromo-7-fluoro-2H-isoquinolin-1-one (486 mg, 1.99 mmol) and [(1S,3R,5S)-3-(benzyloxycarbonylamino)-5-(methoxymethoxy)cyclohexyl]methyl 4-methylbenzenesulfonate (866 mg, 1.81 mmol) in DMF (11.0 mL) was added Cs₂CO₃ (1180 mg, 3.63 mmol) and the reaction was stirred at 60° C. for 3 hr. Upon completion, the mixture was cooled to room temperature and diluted with EtOAc, washed with water, and brine. The organic layer was dried over MgSO₄ and concentrated in vacuo to give the crude product. The residue was purified using column chromatography eluting with EtOAc in hexanes 0-100% to afford benzyl N-[(1R,3S,5S)-3-[(6-bromo-7-fluoro-1-oxo-2-isoquinolyl)methyl]-5-(methoxymethoxy)cyclohexyl]carbamate. ES/MS: m/z 548.9 [M+H]⁺.

Step 5. In a flask was placed benzyl N-[(1R,3S,5S)-3-[(6-bromo-7-fluoro-1-oxo-2-isoquinolyl)methyl]-5-(methoxymethoxy)cyclohexyl]carbamate (756 mg, 1.38 mmol), 1,1′-Bis(diphenylphosphino)ferrocene-palladium(II)dichloride dichloromethane complex (113 mg, 0.138 mmol), potassium acetate (407 mg, 4.14 mmol), and bis(pinacolato)diboron (438 mg, 1.73 mmol) in dioxane (14 mL). The mixture was sparged with nitrogen gas and then stirred at 80° C. for 18 hr. The reaction was cooled to ambient temperature and cataCXium Pd G4 (51.2 mg, 0.069 mmol), 2 M aqueous sodium carbonate (2 mL, 4.14 mmol), and 2-iodo-5-(trifluoromethyl)pyrimidine (454 mg, 1.66 mmol), were added and the mixture was purged with nitrogen gas. The mixture was then stirred at 90° C. for 3 hr. The reaction was then cooled, diluted with EtOAc and filtered through a plug of Celite®. The volatiles were evaporated and the residue purified via flash chromatography with EtOAc in hexanes 0-100% to give benzyl N-[(1R,3S,5S)-3-[[7-fluoro-1-oxo-6-[5-(trifluoromethyl)pyrimidin-2-yl]-2-isoquinolyl]methyl]-5-(methoxymethoxy)cyclohexyl]carbamate. ES/MS m/z: 615.1 [M+H]⁺.

Step 6. To a solution of benzyl N-[(1R,3S,5S)-3-[[7-fluoro-1-oxo-6-[5-(trifluoromethyl)pyrimidin-2-yl]-2-isoquinolyl]methyl]-5-(methoxymethoxy)cyclohexyl]carbamate (150 mg, 0.244 mmol) in MeOH (3.0 ml) was added 10% palladium on carbon (9.0 mg), palladium (II) acetate (2.7 mg, 0.012 mmol), and the flask was equipped with a H² gas balloon. The reaction was then stirred at rt for 16 hr, filtered through a plug of Celite® and then concentrated under reduced pressure to afford 2-[[(1S,3R,5S)-3-amino-5-(methoxymethoxy)cyclohexyl]methyl]-7-fluoro-6-[5-(trifluoromethyl)pyrimidin-2-yl]isoquinolin-1-one which was used without further purification. ES/MS m/z: 482.5 [M+H]⁺.

Step 7. A mixture of 2-[[(1S,3R,5S)-3-amino-5-(methoxymethoxy)cyclohexyl]methyl]-7-fluoro-6-[5-(trifluoromethyl)pyrimidin-2-yl]isoquinolin-1-one (117 mg, 0.244 mmol), 5-chloro-4-(trifluoromethyl)-2-(2-trimethylsilylethoxymethyl)pyridazin-3-one (107 mg, 0.292 mmol), and N,N-diisopropylethylamine (0.2 mL, 1.06 mmol) in DMF (3 mL) was stirred at room temperature for 2 hr. Upon completion, the reaction was diluted with EtOAc, washed with water, washed with brine, dried over MgSO₄ and concentrated in vacuo to give the crude product. The crude residue was purified using column chromatography eluting with EtOAc in hexanes 0-100% to provide 7-fluoro-2-[[(1S,3S,5R)-3-(methoxymethoxy)-5-[[6-oxo-5-(trifluoromethyl)-1-(2-trimethylsilylethoxymethyl)pyridazin-4-yl]amino]cyclohexyl]methyl]-6-[5-(trifluoromethyl)pyrimidin-2-yl]isoquinolin-1-one. ES/MS: m/z 773.2 [M+H]⁺.

Step 8. In a vial was placed 7-fluoro-2-[[(1S,3S,5R)-3-(methoxymethoxy)-5-[[6-oxo-5-(trifluoromethyl)-1-(2-trimethylsilylethoxymethyl)pyridazin-4-yl]amino]cyclohexyl]methyl]-6-[5-(trifluoromethyl)pyrimidin-2-yl]isoquinolin-1-one (188 mg, 0.243 mmol), and trifluoroacetic acid (1.0 ml) in DCM (5.0 mL). The mixture was stirred for 1 hr, and then was concentrated under vacuum. The resulting crude product was dissolved in methanol (4 mL) and 2.0 M ammonia in methanol (1.0 mL) was added and the mixture was stirred for 30 minutes and then concentrated under vacuum. The resulting crude product was purified by reverse phase prep-HPLC (5-100% MeCN in water, 0.1% TFA) to afford 7-fluoro-2-[[(1S,3S,5R)-3-hydroxy-5-[[6-oxo-5-(trifluoromethyl)-1H-pyridazin-4-yl]amino]cyclohexyl]methyl]-6-[5-(trifluoromethyl)pyrimidin-2-yl]isoquinolin-1-one. ¹H NMR (400 MHz, DMSO-d6) δ 12.43 (s, 1H), 9.48 (d, J=1.0 Hz, 2H), 8.45 (d, J=7.1 Hz, 1H), 8.04 (d, J=11.3 Hz, 1H), 7.91 (s, 1H), 7.51 (d, J=7.4 Hz, 1H), 6.85 (d, J=7.4 Hz, 1H), 6.47-6.35 (m, 1H), 4.03-3.85 (m, 3H), 3.83-3.72 (m, 2H), 2.11-1.91 (m, 2H), 1.78-1.63 (m, 2H), 1.45-1.30 (m, 1H), 1.28-1.16 (m, 1H), 1.00-0.83 (m, 1H). ES/MS: m/z 599.1 [M+H]⁺.

### Example 63: Preparation of 7-fluoro-2-[[(1S,3S)-1-hydroxy-3-[[6-oxo-5-(trifluoromethyl)-1H-pyridazin-4-yl]amino]cyclohexyl]methyl]-6-[5-(trifluoromethyl)pyrimidin-2-yl]isoquinolin-1-one

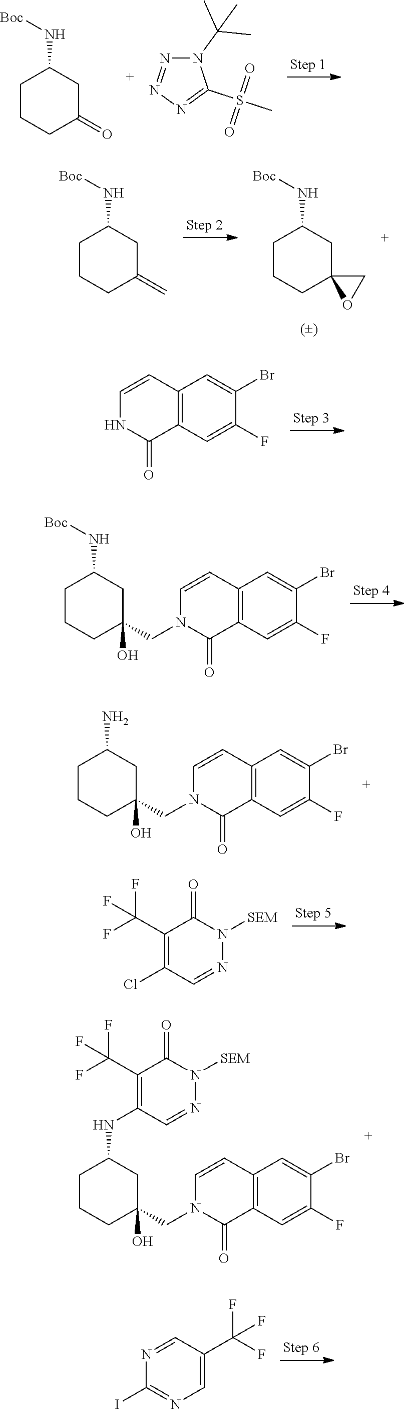

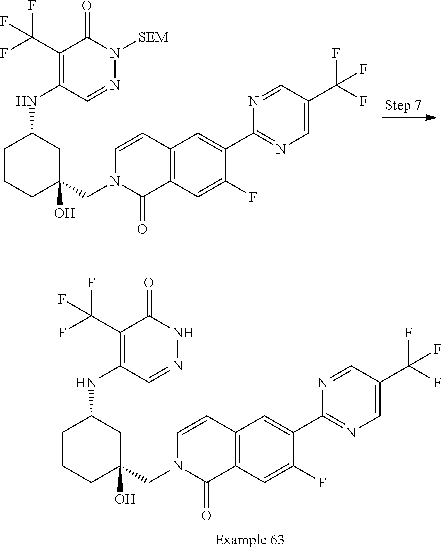 [SMILES: C[Si](C)(C)CCOCn1ncc(N[C@H]2CCC[C@@](O)(Cn3ccc4cc(-c5ncc(C(F)(F)F)cn5)c(F)cc4c3=O)C2)c(C(F)(F)F)c1=O ; O=c1[nH]ncc(N[C@H]2CCC[C@@](O)(Cn3ccc4cc(-c5ncc(C(F)(F)F)cn5)c(F)cc4c3=O)C2)c1C(F)(F)F]

Step 1. To a solution of tert-butyl N-[(1S)-3-oxocyclohexyl]carbamate (3000 mg, 14.1 mmol) in DMF (64 mL) was added 1-tert-butyl-5-methylsulfonyl-tetrazole (3730 mg, 18.3 mmol) and potassium tert-butoxide (42.2 ml, 1.0 M in THF). The reaction mixture was stirred at room temperature for 1 hr and then saturated aq. NH₄Cl was added, and the aqueous layer was extracted with EtOAc. The organic layer was washed with brine, dried (MgSO₄), filtered, and concentrated under reduced pressure to afford tert-butyl N-[(1S)-3-methylenecyclohexyl]carbamate which was used without further purification. ES/MS: m/z 212.0 [M+H]⁺.

Step 2. In a flask tert-butyl N-[(1S)-3-methylenecyclohexyl]carbamate (2970 mg, 14.1 mmol) was dissolved in DCM (69 mL) and cooled to 0° C. and meta-chloroperbenzoic acid (9450 mg, 42.2 mmol) was added. The resulting suspension was slowly warmed to rt and stirred for 3 hr. Reaction was quenched by the addition of sat. aq. NaHCO₃. Aqueous layer was extracted with DCM and the organic layer was washed with brine, dried (MgSO₄), filtered, and concentrated under reduced pressure. The residue was purified by flash column chromatography eluting with EtOAc in hexanes 0-100% to afford tert-butyl ((3S,5S)-1-oxaspiro[2.5]octan-5-yl)carbamate. ES/MS: m/z 228.3 [M+H]⁺.

Step 3. To a mixture of 6-bromo-7-fluoro-2H-isoquinolin-1-one (738 mg, 3.05 mmol) and tert-butyl N tert-butyl ((3S,5S)-1-oxaspiro[2.5]octan-5-yl)carbamate (630 mg, 2.77 mmol) in DMF (17 mL) was added Cs₂CO₃ (1810 mg, 5.54 mmol) and the reaction was stirred at 60° C. for 18 hr. Upon completion, the mixture was diluted with EtOAc, washed with water, washed with brine, dried over MgSO₄ and concentrated in vacuo to give the crude product. The crude residue was purified using column chromatography eluting with EtOAc in hexanes 0-100% to afford tert-butyl ((1S,3S)-3-((6-bromo-7-fluoro-1-oxoisoquinolin-2(1H)-yl)methyl)-3-hydroxycyclohexyl)carbamate ES/MS: m/z 470.9 [M+H]⁺.

Step 4. To a solution of tert-butyl ((1S,3S)-3-((6-bromo-7-fluoro-1-oxoisoquinolin-2(1H)-yl)methyl)-3-hydroxycyclohexyl)carbamat (497 mg, 1.06 mmol) in DCM (5.0 mL) was added trifluoroacetic acid (1.0 mL) at room temperature and the mixture was stirred for 1 hr. Upon completion, the solvent was removed under reduced pressure to afford 2-(((1S,3S)-3-amino-1-hydroxycyclohexyl)methyl)-6-bromo-7-fluoroisoquinolin-1(2H)-one which was used without further purification. ES/MS: m/z 371.5 [M+H]⁺.

Step 5. A mixture of 2-(((1S,3S)-3-amino-1-hydroxycyclohexyl)methyl)-6-bromo-7-fluoroisoquinolin-1(2H)-one (391 mg, 1.06 mmol), 5-chloro-4-(trifluoromethyl)-2-(2-trimethylsilylethoxymethyl)pyridazin-3-one (464 mg, 1.27 mmol), and N,N-diisopropylethylamine (0.8 mL, 4.60 mmol) in DMF (6 mL) was stirred at room temperature for 2 hr. Upon completion, the reaction was diluted with EtOAc, washed with water, washed with brine, dried over MgSO₄ and concentrated to give the crude product. The residue was purified using column chromatography eluting with EtOAc in hexanes 0-100% to provide 6-bromo-7-fluoro-2-(((1S,3S)-1-hydroxy-3-((6-oxo-5-(trifluoromethyl)-1-((2-(trimethylsilyl)ethoxy)methyl)-1,6-dihydropyridazin-4-yl)amino)cyclohexyl)methyl)isoquinolin-1(2H)-one. ES/MS: m/z 663.0 [M+H]⁺.

Step 6. In a flask was placed 6-bromo-7-fluoro-2-(((1S,3S)-1-hydroxy-3-((6-oxo-5-(trifluoromethyl)-1-((2-(trimethylsilyl)ethoxy)methyl)-1,6-dihydropyridazin-4-yl)amino)cyclohexyl)methyl)isoquinolin-1(2H)-one (305 mg, 0.460 mmol), 1,1′-Bis(diphenylphosphino)ferrocene-palladium(II)dichloride dichloromethane complex (37.6 mg, 0.046 mmol), potassium acetate (136 mg, 1.38 mmol), and bis(pinacolato)diboron (175 mg, 0.690 mmol) in dioxane (5.0 mL). The mixture was sparged with nitrogen gas and then stirred at 80° C. for 18 hr. The reaction was cooled to ambient temperature and cataCXium Pd G4 (34.2 mg, 0.046 mmol), 2 M aqueous sodium carbonate (0.50 mL, 1.01 mmol), and 2-iodo-5-(trifluoromethyl)pyrimidine (189 mg, 0.691 mmol), were added. The mixture was then stirred at 90° C. for 3 hr. The reaction was then cooled, diluted with EtOAc and filtered through a plug of Celite®. The volatiles were evaporated and the residue purified via flash chromatography (EtOAc in hexanes 0-100%) to give 7-fluoro-2-(((1S,3S)-1-hydroxy-3-((6-oxo-5-(trifluoromethyl)-1-((2-(trimethylsilyl)ethoxy)methyl)-1,6-dihydropyridazin-4-yl)amino)cyclohexyl)methyl)-6-(5-(trifluoromethyl)pyrimidin-2-yl)isoquinolin-1(2H)-one. ES/MS m/z: 729.1 [M+H]⁺.

Step 7. In a vial was placed 7-fluoro-2-(((1S,3S)-1-hydroxy-3-((6-oxo-5-(trifluoromethyl)-1-((2-(trimethylsilyl)ethoxy)methyl)-1,6-dihydropyridazin-4-yl)amino)cyclohexyl)methyl)-6-(5-(trifluoromethyl)pyrimidin-2-yl)isoquinolin-1(2H)-one (313 mg, 0.430 mmol), and trifluoroacetic acid (1 ml) in DCM (5 mL). The mixture was stirred for 1 hr, and then was concentrated under vacuum. The resulting crude product was dissolved in methanol (4 mL) and 2.0 M ammonia in methanol (1.0 mL, 2.29 mmol) was added and the mixture was stirred for 30 minutes and then concentrated under vacuum. The resulting crude product was purified by reverse phase prep-HPLC (5-100% MeCN in water, 0.1% TFA) to afford 7-fluoro-2-[[(1S,3S)-1-hydroxy-3-[[6-oxo-5-(trifluoromethyl)-1H-pyridazin-4-yl]amino]cyclohexyl]methyl]-6-[5-(trifluoromethyl)pyrimidin-2-yl]isoquinolin-1-one. ¹H NMR (400 MHz, DMSO-d6) δ 12.44 (s, 1H), 9.75-9.30 (m, 2H), 8.44 (d, J=7.1 Hz, 1H), 8.05 (d, J=11.4 Hz, 1H), 7.84 (s, 1H), 7.48 (d, J=7.4 Hz, 1H), 6.82 (d, J=7.5 Hz, 1H), 6.43-6.24 (m, 1H), 4.15-3.94 (m, 2H), 3.95-3.82 (m, 1H), 1.77 (s, 1H), 1.71-1.39 (m, 7H), 1.35-1.22 (m, 1H). ES/MS m/z: 599.1 [M+H]⁺.

### Example 64: 7-fluoro-2-[[(2R,4S)-4-[[6-oxo-5-(trifluoromethyl)-1H-pyridazin-4-yl]amino]tetrahydropyran-2-yl]methyl]-6-([1,2,4]triazolo[1,5-a]pyridin-2-yl)isoquinolin-1-one

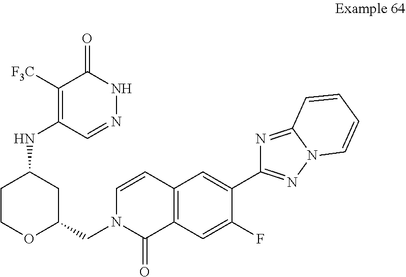 [SMILES: O=c1[nH]ncc(N[C@H]2CCO[C@@H](Cn3ccc4cc(-c5nc6ccccn6n5)c(F)cc4c3=O)C2)c1C(F)(F)F]

The title compound was synthesized as described in Example 1 with the following modification in Step 1. ¹H NMR (400 MHz, DMSO-d₆) δ 12.48 (s, 1H), 9.08 (d, J=6.9 Hz, 1H), 8.57 (d, J=6.9 Hz, 1H), 8.08-8.00 (m, 2H), 7.98-7.91 (m, 1H), 7.76 (ddd, J=8.7, 7.0, 1.3 Hz, 1H), 7.43 (d, J=7.4 Hz, 1H), 7.29 (td, J=6.9, 1.3 Hz, 1H), 6.81 (d, J=7.5 Hz, 1H), 6.48 (d, J=6.9 Hz, 1H), 4.14 (dd, J=13.6, 3.6 Hz, 1H), 4.05-3.95 (m, 2H), 3.94-3.86 (m, 1H), 3.83-3.75 (m, 1H), 3.40-3.36 (m, 1H), 1.89 (d, J=12.4 Hz, 1H), 1.74-1.68 (m, 1H), 1.69-1.62 (m, 1H), 1.46 (q, J=11.6 Hz, 1H). ES/MS: m/z 556.2 [M+H]⁺.

Step 1. To a solution of (2R,4S)-4-(tert-butoxycarbonylamino)tetrahydropyran-2-carboxylic acid (1000 mg, 4.1 mmol) in THF (40.0 mL) at 0° C. was added triethylamine (0.57 mL, 4.1 mmol). To this mixture was added ethyl chloroformate (0.40 mL, 4.1 mmol) and the reaction was stirred for 30 minutes at 0° C. The mixture was then filtered to remove the precipitated triethylamine hydrochloride. The filtrate containing the mixed anhydride was slowly added to a stirred suspension of sodium borohydride (463 mg, 12.2 mmol) in 20% aqueous THF (20.0 mL) maintained at 10° C. After the mixture was stirred for 30 minutes, it was acidified with 1N HCl to pH˜4. The mixture was then extracted with EtOAc, washed with 2M NaOH_{(aq)} solution, washed with brine, dried (Na₂SO₄), and concentrated in vacuo. The crude product was dissolved in dichloromethane (30.0 mL) and to this mixture was added triethylamine (0.85 mL, 3.19 mmol), followed by p-Toluenesulfonyl chloride (0.61 g, 6.10 mmol) and the reaction was stirred at room temperature for 16 h. Upon completion, the mixture was diluted with water and extracted with dicholoromethane (×3). The combined organic layers were washed with brine, dried over Na₂SO₄ and concentrated in vacuo to give the crude product. The crude residue was purified using column chromatography eluting with EtOAc in hexanes 0-100% to afford [(2R,4S)-4-(tert-butoxycarbonylamino)tetrahydropyran-2-yl]methyl 4-methylbenzenesulfonate. ES/MS: m/z 386.1 [M+H]⁺.

### Example 65: 7,8-difluoro-2-[[(2R,4S)-4-[[6-oxo-5-(trifluoromethyl)-1H-pyridazin-4-yl]amino]tetrahydropyran-2-yl]methyl]-6-([1,2,4]triazolo[1,5-a]pyridin-2-yl)isoquinolin-1-one1-one

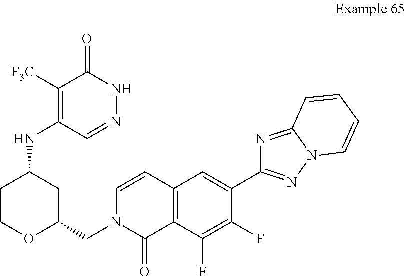 [SMILES: O=c1[nH]ncc(N[C@H]2CCO[C@@H](Cn3ccc4cc(-c5nc6ccccn6n5)c(F)c(F)c4c3=O)C2)c1C(F)(F)F]

The title compound was synthesized as described in Example 64 using 6-bromo-7,8-difluoro-2H-isoquinolin-1-one in place of 6-bromo-7-fluoro-2H-isoquinolin-1-one in step 2 of the synthesis. ¹H NMR (400 MHz, DMSO-d₆) δ 12.49 (s, 1H), 9.10 (d, J=6.8 Hz, 1H), 8.34 (d, J=6.1 Hz, 1H), 8.03 (s, 1H), 7.96 (d, J=8.9 Hz, 1H), 7.81-7.72 (m, 1H), 7.45 (d, J=7.4 Hz, 1H), 7.30 (d, J=7.0 Hz, 1H), 6.78 (d, J=7.4 Hz, 1H), 6.48 (dd, J=8.3, 3.4 Hz, 1H), 4.12 (d, J=3.3 Hz, 1H), 4.07-4.02 (m, 1H), 3.98-3.86 (m, 2H), 3.83-3.74 (m, 1H), 1.89 (d, J=15.8 Hz, 1H), 1.80-1.60 (m, 2H), 1.46 (d, J=11.9 Hz, 1H). ES/MS: m/z 574.2 [M+H]⁺.

### Intermediate 6: Preparation of cis tert-butyl N-[(1R,5S)-3,3-difluoro-5-(hydroxymethyl)cyclohexyl]carbamate

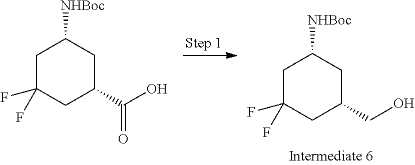 [SMILES: CC(C)(C)OC(=O)N[C@@H]1C[C@H](C(=O)O)CC(F)(F)C1 ; CC(C)(C)OC(=O)N[C@@H]1C[C@H](CO)CC(F)(F)C1]

To a stirred solution of cis-5-(tert-butoxycarbonylamino)-3,3-difluoro-cyclohexanecarboxylic acid (1000 mg, 3.58 mmol) in THF (28 mL) at 0° C. under N₂ atmosphere was added ethyl chloroformate (0.343 mL, 3.58 mmol). The Solution was allowed to stir for 30 minutes at 0° C. and then filtered to remove the precipitated triethylamine hydrochloride. The filtrate containing the mixed anhydride was slowly added to a stirred suspension of NaBH₄ (406 mg, 10.7 mmol) in 20% aqueous THF (10 mL) maintained at 10° C. and allowed to stir for 30 minutes, then acidified with 1N HCl to pH˜4. The mixture was extracted with EtOAc, washed with 2M NaOH solution, washed with brine, dried over Na₂SO₄ and concentrated. The crude residue was purified using column chromatography eluting with EtOAc in hexanes 0-100% to afford cis tert-butyl N-[3,3-difluoro-5-(hydroxymethyl)cyclohexyl]carbamate. ES/MS: m/z 265.9 [M+H]⁺.

Examples 66 and Example 67: 2-(((1S,5R)-3,3-difluoro-5-((6-oxo-5-(trifluoromethyl)-1,6-dihydropyridazin-4-yl)amino)cyclohexyl)methyl)-7-fluoro-6-(5-(trifluoromethyl)pyrimidin-2-yl)isoquinolin-1(2H)-one and 2-(((1R,5S)-3,3-difluoro-5-((6-oxo-5-(trifluoromethyl)-1,6-dihydropyridazin-4-yl)amino)cyclohexyl)methyl)-7-fluoro-6-(5-(trifluoromethyl)pyrimidin-2-yl)isoquinolin-1(2H)-one

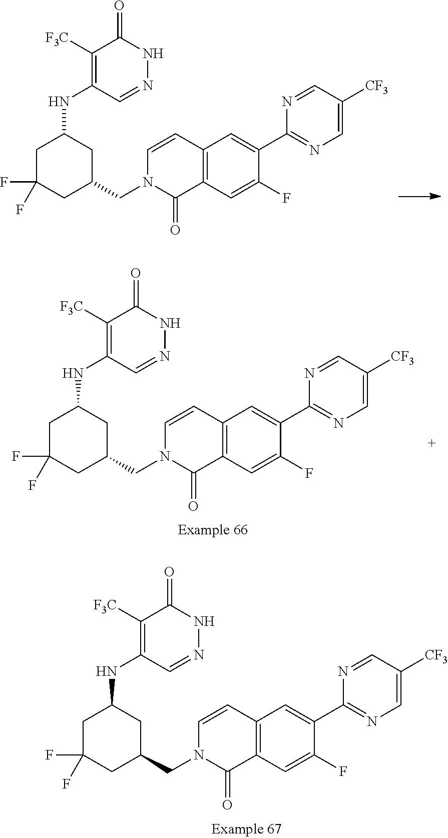 [SMILES: O=c1[nH]ncc(N[C@@H]2C[C@H](Cn3ccc4cc(-c5ncc(C(F)(F)F)cn5)c(F)cc4c3=O)CC(F)(F)C2)c1C(F)(F)F ; O=c1[nH]ncc(N[C@@H]2C[C@H](Cn3ccc4cc(-c5ncc(C(F)(F)F)cn5)c(F)cc4c3=O)CC(F)(F)C2)c1C(F)(F)F ; O=c1[nH]ncc(N[C@H]2C[C@@H](Cn3ccc4cc(-c5ncc(C(F)(F)F)cn5)c(F)cc4c3=O)CC(F)(F)C2)c1C(F)(F)F]

The title compounds were prepared as for Example 1 using Intermediate 6, with an additional purification: 2-(((rac-1S,5R)-3,3-difluoro-5-((6-oxo-5-(trifluoromethyl)-1,6-dihydropyridazin-4-yl)amino)cyclohexyl)methyl)-7-fluoro-6-(5-(trifluoromethyl)pyrimidin-2-yl)isoquinolin-1(2H)-one was separated via chiral SFC (IB 5u 21×250 mm column; 30% EtOH as co-solvent; 100 bar; 40° C.). The first eluting peak was assigned as the (1S,5R)-configuration (Example 66), and the second eluting peak was assigned as the (1R,5S)-configuration (Example 67).

### Example 66: 2-(((1S,5R)-3,3-difluoro-5-((6-oxo-5-(trifluoromethyl)-1,6-dihydropyridazin-4-yl)amino)cyclohexyl)methyl)-7-fluoro-6-(5-(trifluoromethyl)pyrimidin-2-yl)isoquinolin-1(2H)-one

¹H NMR (400 MHz, DMSO-d6) δ 12.50 (s, 1H), 9.49 (d, J=0.9 Hz, 2H), 8.45 (d, J=7.1 Hz, 1H), 8.05 (d, J=11.3 Hz, 1H), 7.93 (s, 1H), 7.53 (d, J=7.4 Hz, 1H), 6.87 (d, J=7.4 Hz, 1H), 6.52 (dd, J=8.6, 3.9 Hz, 1H), 4.00 (d, J=6.7 Hz, 2H), 2.31 (d, J=18.0 Hz, 1H), 2.14 (t, J=11.5 Hz, 1H), 2.01 (d, J=11.6 Hz, 1H), 1.90 (d, J=12.2 Hz, 1H), 1.60 (dt, J=35.6, 12.8 Hz, 1H), 1.39 (q, J=12.1 Hz, 1H), 1.23 (s, 2H). ES/MS m/z: 619.1 [M+H]⁺.

### Example 67: 2-(((1R,5S)-3,3-difluoro-5-((6-oxo-5-(trifluoromethyl)-1,6-dihydropyridazin-4-yl)amino)cyclohexyl)methyl)-7-fluoro-6-(5-(trifluoromethyl)pyrimidin-2-yl)isoquinolin-1(2H)-one

¹H NMR (400 MHz, DMSO-d6) δ 12.51 (s, 1H), 9.49 (d, J=1.0 Hz, 2H), 8.45 (d, J=7.1 Hz, 1H), 8.05 (d, J=11.3 Hz, 1H), 7.93 (s, 1H), 7.53 (d, J=7.4 Hz, 1H), 6.87 (d, J=7.4 Hz, 1H), 6.64-6.40 (m, 1H), 4.00 (d, J=6.7 Hz, 2H), 2.31 (d, J=18.5 Hz, 1H), 2.14 (t, J=11.3 Hz, 1H), 2.02 (s, 1H), 1.90 (d, J=12.2 Hz, 1H), 1.60 (dt, J=34.8, 13.1 Hz, 1H), 1.39 (q, J=12.0 Hz, 1H), 1.23 (s, 2H). ES/MS m/z: 619.0 [M+H]⁺.

### Example 68: 7-fluoro-2-(((rac-1R,3R,4S)-4-hydroxy-3-((6-oxo-5-(trifluoromethyl)-1,6-dihydropyridazin-4-yl)amino)cyclohexyl)methyl)-6-(5-(trifluoromethyl)pyrimidin-2-yl)isoquinolin-1(2H)-one

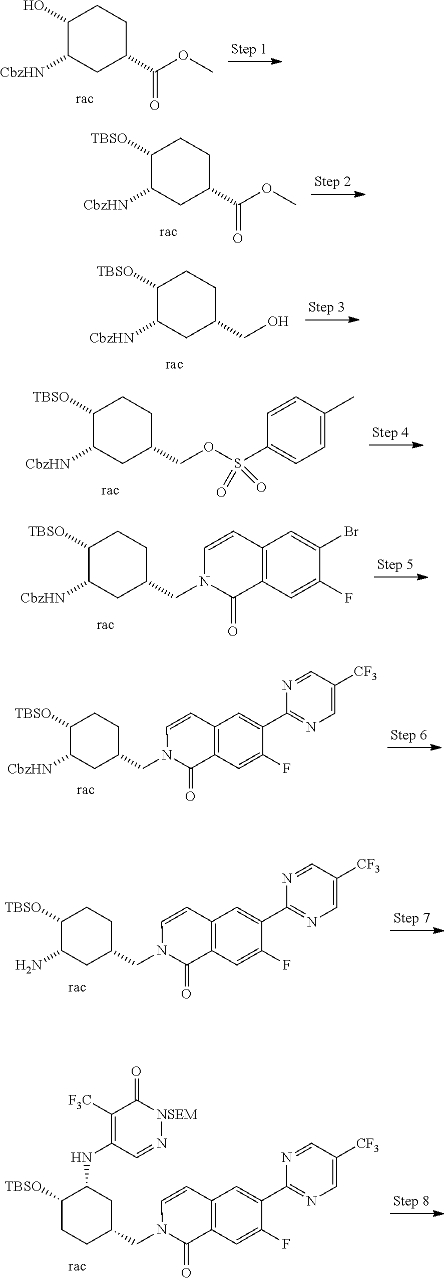

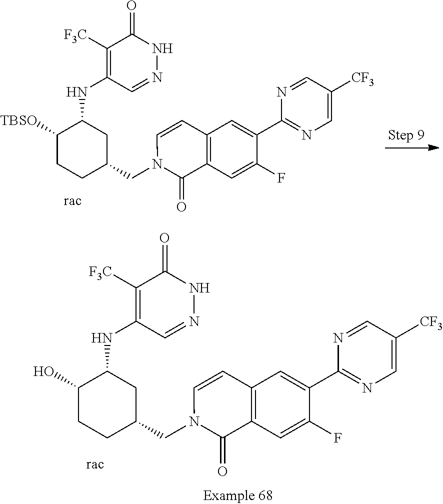

Step 1. In a vial were placed methyl (rac-1S,3S,4R)-3-(((benzyloxy) carbonyl)amino)-4-hydroxycyclohexane-1-carboxylate (100 mg, 0.33 mmol), tert-butyldimethylsilyl chloride (98 mg, 0.65 mmol), imidazole (111 mg, 1.6 mmol), and 4-(dimethylamino) pyridine (4.0 mg, 0.033 mmol) in dichloromethane (2.0 mL). After the mixture was stirred at room temperature for 72 hr, it was purified by flash chromatography to give methyl (rac-1S,3S,4R)-3-(((benzyloxy) carbonyl)amino)-4-((tert-butyldimethylsilyl)oxy) cyclohexane-1-carboxylate. ES/MS m/z=422.0 [M+H]⁺.

Step 2. In a 100 mL round-bottom, single necked flask was placed methyl (rac-1S,3S,4R)-3-(((benzyloxy)carbonyl) amino)-4-((tert-butyldimethylsilyl)oxy)cyclohexane-1I-carboxylate (310 mg, 0.74 mmol) in THF (8 mL), and the solution was cooled to 0° C. To this was added lithium borohydride (1.5 mL, 2.0 M in THF, 2.9 mmol). The resulting mixture was slowly warmed to room temperature and stirred for 72 hr. The reaction was then cooled to 0° C., quenched with sat. NaHCO₃ and extracted with EtOAc. The combined organic layers were washed with water and brine, dried (Na₂SO₄), and purified by flash chromatography to give benzyl ((rac-1S,2R,5S)-2-((tert-butyldimethylsilyl)oxy)-5-(hydroxymethyl)cyclohexyl)carbamate. ES/MS m/z=393.6 [M+H]⁺.

Step 3. In a vial were placed benzyl ((rac-1S,2R,5S)-2-((tert-butyldimethylsilyl)oxy)-5-(hydroxymethyl)cyclohexyl)carbamate (395 mg, 1.0 mmol), p-toluenesulfonyl chloride, (230 mg, 1.2 mmol), triethylamine (0.42 mL, 3.0 mmol), and 4-dimethylaminopyridine (3.1 mg, 0.025 mmol) in dichloromethane (5.0 mL). After the mixture was stirred at room temperature for 16 hr, it was purified by flash chromatograph to give ((rac-1S,3S,4R)-3-(((benzyloxy)carbonyl)amino)-4-((tert-butyldimethylsilyl)oxy)cyclohexyl)methyl 4-methylbenzenesulfonate. ES/MS m/z=548.4 [M+H]⁺.

Step 4. In a vial were placed ((rac-1S,3S,4R)-3-(((benzyloxy)carbonyl)amino)-4-((tert-butyldimethylsilyl)oxy)cyclohexyl)methyl 4-methylbenzenesulfonate (130 mg, 0.237 mmol), 6-bromo-7-fluoroisoquinolin-1(2H)-one (86.1 mg, 0.356 mmol), and cesium carbonate (232 mg, 0.712 mmol) in DMF (2.0 mL). After the mixture was stirred at room temperature for 48 hr, it was purified by flash chromatography to give benzyl ((rac-1S,2R,5S)-5-((6-bromo-7-fluoro-1-oxoisoquinolin-2(1H)-yl)methyl)-2-((tert-butyldimethylsilyl)oxy)cyclohexyl)carbamate. ES/MS m/z=617.0 [M+H]⁺.

Step 5. In a vial were placed benzyl ((rac-1S,2R,5S)-5-((6-bromo-7-fluoro-1-oxoisoquinolin-2(1H)-yl)methyl)-2-((tert-butyldimethylsilyl)oxy)cyclohexyl)carbamate (57.0 mg, 0.0877 mmol), bis(pinacolato)diboron (66.8 mg, 0.263 mmol), [1,1′-bis(diphenylphosphino)ferrocene]dichloropalladium(II) (6.42 mg, 0.00877 mmol), and potassium acetate (34.4 mg, 0.351 mmol) in dioxane (2.0 mL). The mixture was purged with N₂ gas and stirred at 100° C. for 2 hr. The mixture was then cooled to room temperature followed by the addition of 2-chloro-5-(trifluoromethyl)pyrimidine (51.9 mg, 0.284 mmol), CataCXium Pd G4 (3.51 mg, 0.00473 mmol) and 2M Na₂CO₃ (0.14 mL, 0.28 mmol). The resulting mixture was purged with N₂ gas and stirred at 80° C. for 2 hr. Upon completion, the mixture was cooled to room temperature and purified by flash chromatography to give benzyl ((rac-1S,2R,5S)-2-((tert-butyldimethylsilyl)oxy)-5-((7-fluoro-1-oxo-6-(5-(trifluoromethyl)pyrimidin-2-yl)isoquinolin-2(1H)-yl)methyl)cyclohexyl)carbamate. ES/MS m/z=685.2 [M+H]⁺.

Step 6. In a vial was placed benzyl ((rac-1S,2R,5S)-2-((tert-butyldimethylsilyl)oxy)-5-((7-fluoro-1-oxo-6-(5-(trifluoromethyl)pyrimidin-2-yl)isoquinolin-2(1H)-yl)methyl)cyclohexyl)carbamate (50 mg, 0.073 mmol) and palladium on carbon (16 mg, 10% wt) in MeOH (1.0 mL). After the mixture was stirred under a H₂ gas balloon at 1 atm for 16 hr, it filtered through a pad of Celite® and concentrated to give 2-(((rac-1S,3S,4R)-3-amino-4-((tert-butyldimethylsilyl)oxy)cyclohexyl)methyl)-7-fluoro-6-(5-(trifluoromethyl)pyrimidin-2-yl)isoquinolin-1(2H)-one. ES/MS m/z=550.3 [M+H]⁺.

Step 7. In a vial were placed 2-(((rac-1S,3S,4R)-3-amino-4-((tert-butyldimethylsilyl)oxy)cyclohexyl)methyl)-7-fluoro-6-(5-(trifluoromethyl)pyrimidin-2-yl)isoquinolin-1(2H)-one (40 mg, 0.073 mmol), 5-chloro-4-(trifluoromethyl)-2-(2-trimethylsilylethoxymethyl)pyridazin-3-one (36 mg, 0.11 mmol), and N,N-diisopropylethylamine (0.13 mL, 0.73 mmol) in DMF (1.0 mL). After the mixture was stirred at 80° C. for 1 hr, it was cooled to room temperature and purified by flash chromatography to give 2-(((rac-1R,3R,4S)-4-((tert-butyldimethylsilyl)oxy)-3-((6-oxo-5-(trifluoromethyl)-1-((2-(trimethylsilyl)ethoxy)methyl)-1,6-dihydropyridazin-4-yl)amino)cyclohexyl)methyl)-7-fluoro-6-(5-(trifluoromethyl)pyrimidin-2-yl)isoquinolin-1(2H)-one. ES/MS m/z=843.2 [M+H]⁺.

Step 8. In a vial was placed 2-(((rac-1R,3R,4S)-4-((tert-butyldimethylsilyl)oxy)-3-((6-oxo-5-(trifluoromethyl)-1-((2-(trimethylsilyl)ethoxy)methyl)-1,6-dihydropyridazin-4-yl)amino)cyclohexyl)methyl)-7-fluoro-6-(5-(trifluoromethyl)pyrimidin-2-yl)isoquinolin-1(2H)-one (40 mg, 0.047 mmol) in dichloromethane (1.0 mL). To this was added trifluoroacetic acid (0.072 mL, 0.95 mmol) and the mixture was stirred at room temperature for 3 hr. The excess trifluoroacetic acid and solvent were removed under reduced pressure and the residue was dissolved in MeOH (1.0 mL). To this was added ethylenediamine (0.038 mL, 0.57 mmol). After the resulting mixture was stirred at room temperature for 1 hr, it was purified by flash chromatography to give 2-(((rac-1R,3R,4S)-4-((tert-butyldimethylsilyl)oxy)-3-((6-oxo-5-(trifluoromethyl)-1,6-dihydropyridazin-4-yl)amino)cyclohexyl)methyl)-7-fluoro-6-(5-(trifluoromethyl)pyrimidin-2-yl)isoquinolin-1(2H)-one. ES/MS m/z=713.1 [M+H]⁺.

Step 9. In a vial was placed 2-(((rac-1R,3R,4S)-4-((tert-butyldimethylsilyl)oxy)-3-((6-oxo-5-(trifluoromethyl)-1,6-dihydropyridazin-4-yl)amino)cyclohexyl)methyl)-7-fluoro-6-(5-(trifluoromethyl)pyrimidin-2-yl)isoquinolin-1(2H)-one in 4M HCl in dioxane (1 mL). The mixture was stirred at room temperature for 72 hr. Then it was concentrated and purified by reverse phase chromatography to give the title compound. ¹H NMR (400 MHz, DMSO-d6) δ 12.48 (s, 1H), 9.48 (s, 2H), 8.44 (d, J=7.2 Hz, 1H), 8.03 (d, J=11.4 Hz, 1H), 7.92 (s, 1H), 7.51 (d, J=7.4 Hz, 1H), 6.82 (d, J=7.4 Hz, 1H), 6.27 (m, 1H), 5.31 (m, 1H), 4.09-3.71 (m, 4H), 2.06 (m, 1H), 1.87-1.74 (m, 1H), 1.62 (m, 1H), 1.37 (n, 4H). ES/MS m/z=599.0 [M+H]⁺.

### Intermediate 8: Preparation of 6-bromo-7-fluoro-2-[[(1R,3S)-3-[[6-oxo-5-(trifluoromethyl)-1-(2-trimethylsilylethoxymethyl)pyridazin-4-yl]amino]cyclohexyl]methyl]-3,4-dihydroisoquinolin-1-one

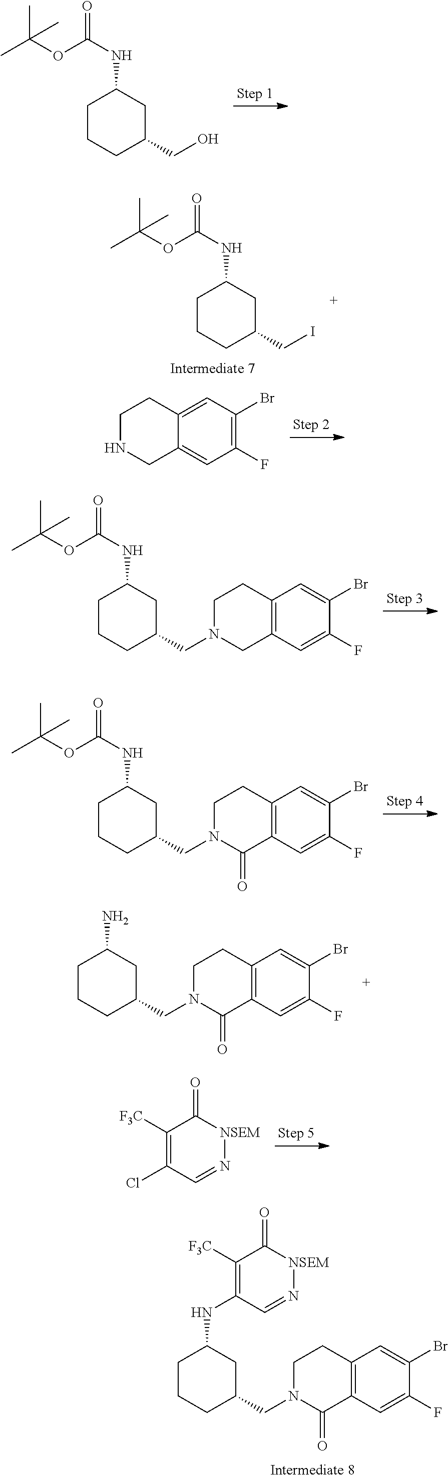

Step 1. Triphenylphosphine (15.1 g, 57.6 mmol) and imidazole (3.92 g, 57.6 mmol) were dissolved in DCM (200 mL) and stirred at ambient temperature. Iodine (14.6 g, 57.6 mmol) was then added and the mixture stirred for 30 minutes before addition of tert-butyl N-[(1S,3R)-3-(hydroxymethyl)cyclohexyl]carbamate (11.0 g, 48.0 mmol). After an additional 18 hours, the mixture was filtered, and the filtrate evaporated. The crude residue was taken up in EtOAc and filtered a second time. This filtrate was washed with a saturated solution of sodium bisulfite followed by brine. The organics were then dried over Na₂SO₄, filtered, and evaporated. The crude material was purified via flash chromatography (100% hex→100% EtOAc) to afford Intermediate 7, tert-butyl N-[(1S,3R)-3-(iodomethyl)cyclohexyl]carbamate. ES/MS m/z: 339.9 [M]⁺.

Step 2. tert-butyl N-[(1S,3R)-3-(iodomethyl)cyclohexyl]carbamate (200 mg, 0.59 mmol), 6-bromo-7-fluoro-1,2,3,4-tetrahydroisoquinoline (160 mg, 0.71 mmol), and Cs₂CO₃ (480 mg, 1.5 mmol) were taken up in DMF (1.4 mL) and stirred at ambient temperature for 16 hours at which point the reaction was heated to 65° C. After an additional 4.5 hours, the reaction was deemed complete by LC/MS analysis and was cooled to ambient temperature. The reaction was then diluted with water and extracted 3× with EtOAc The combined organics were washed with brine, dried over Na₂SO₄, filtered, and evaporated. The crude material was purified via flash chromatography (100% DCM→10% [7N NH3 in MeOH]/DCM) to afford tert-butyl N-[(1S,3R)-3-[(6-bromo-7-fluoro-3,4-dihydro-1H-isoquinolin-2-yl)methyl]cyclohexyl]carbamate. ES/MS m/z: 441.6/443.4 [M]⁺.

Step 3. tert-butyl N-[(1S,3R)-3-[(6-bromo-7-fluoro-3,4-dihydro-1H-isoquinolin-2-yl)methyl]cyclohexyl]carbamate (150 mg, 0.31 mmol) was dissolved in THF (8.7 mL) and water (3.6 mL) and stirred at ambient temperature. NaHCO₃ (260 mg, 3.1 mmol) and 12 (580 mg, 2.3 mmol) were then added sequentially. After 1.5 hours, the reaction was quenched with saturated Na₂S₂O₃ and extracted 3× with DCM. The combined organics were dried over MgSO₄, filtered, and evaporated. The crude material was purified via flash chromatography (100% hex→100% EtOAc) to afford tert-butyl N-[(1S,3R)-3-[(6-bromo-7-fluoro-1-oxo-3,4-dihydroisoquinolin-2-yl)methyl]cyclohexyl]carbamate. ES/MS m/z: 455.0/457.0 [M]⁺.

Step 4. tert-butyl N-[(1S,3R)-3-[(6-bromo-7-fluoro-1-oxo-3,4-dihydroisoquinolin-2-yl)methyl]cyclohexyl]carbamate (170 mg, 0.34 mmol) was dissolved in DCM (3.0 mL) and the solution stirred at ambient temp. TFA (0.26 mL, 3.4 mmol) was then added. After 1 hour, the reaction was evaporated to dryness and the crude 2-[[(1R,3S)-3-aminocyclohexyl]methyl]-6-bromo-7-fluoro-3,4-dihydroisoquinolin-1-one was carried forward without purification.

Step 5. 2-[[(1R,3S)-3-aminocyclohexyl]methyl]-6-bromo-7-fluoro-3,4-dihydroisoquinolin-1-one (0.34 mmol) and 5-chloro-4-(trifluoromethyl)-2-(2-trimethylsilylethoxymethyl)pyridazin-3-one (0.41 mmol) were dissolved in DMF (2.0 mL). DIPEA (0.59 mL, 3.4 mmol) was then added and the reaction was stirred at 60° C. for 2.5 hours at which point it was cooled to ambient temperature, diluted w/10% aq. KHSO₄, and extracted 3× with EtOAc. The combined organics were washed with brine, dried over MgSO₄, filtered, and evaporated. The crude material was purified via flash chromatography (100% hex→100% EtOAc) to afford 6-bromo-7-fluoro-2-[[(1R,3S)-3-[[6-oxo-5-(trifluoromethyl)-1-(2-trimethylsilylethoxymethyl)pyridazin-4-yl]amino]cyclohexyl]methyl]-3,4-dihydroisoquinolin-1-one. ES/MS m/z: 647.1/649.1 [M]⁺.

### Example 69: Preparation of 7-fluoro-2-[[(1R,3S)-3-[[6-oxo-5-(trifluoromethyl)-1H-pyridazin-4-yl]amino]cyclohexyl]methyl]-6-[5-(trifluoromethyl)pyrimidin-2-yl]-3,4-dihydroisoquinolin-1-one

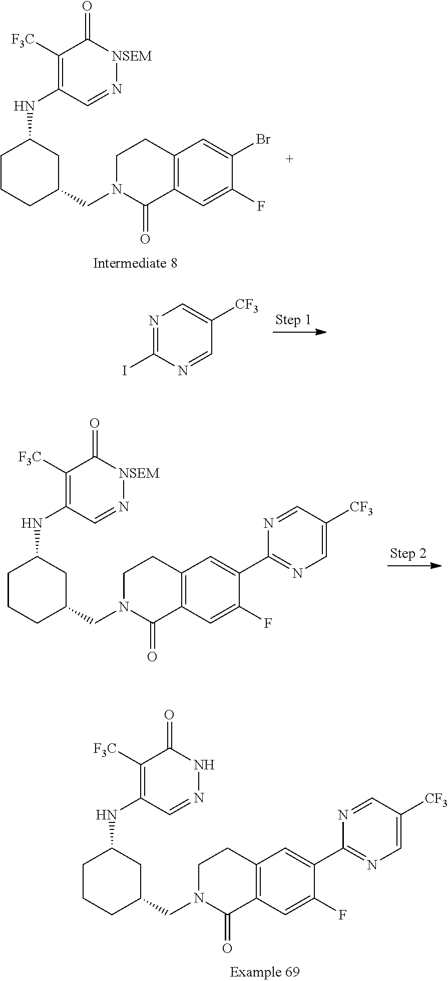 [SMILES: C[Si](C)(C)CCOCn1ncc(N[C@H]2CCC[C@@H](CN3CCc4cc(Br)c(F)cc4C3=O)C2)c(C(F)(F)F)c1=O ; FC(F)(F)c1cnc(I)nc1 ; C[Si](C)(C)CCOCn1ncc(N[C@H]2CCC[C@@H](CN3CCc4cc(-c5ncc(C(F)(F)F)cn5)c(F)cc4C3=O)C2)c(C(F)(F)F)c1=O ; O=C1c2cc(F)c(-c3ncc(C(F)(F)F)cn3)cc2CCN1C[C@@H]1CCC[C@H](Nc2cn[nH]c(=O)c2C(F)(F)F)C1]

Step 1. Intermediate 8, 6-bromo-7-fluoro-2-[[(1R,3S)-3-[[6-oxo-5-(trifluoromethyl)-1-(2-trimethylsilylethoxymethyl)pyridazin-4-yl]amino]cyclohexyl]methyl]-3,4-dihydroisoquinolin-1-one (65 mg, 0.10 mmol), Bis(pinacolato)diboron (36 mg, 0.14 mmol), (dppf)PdCl₂ (3.5 mg, 0.005 mmol), and potassium acetate (28 mg, 0.29 mmol) were flushed with N₂ and taken up in dioxane (1.1 mL). The reaction was then stirred at 100° C. until complete consumption of the bromide was observed (20 hours). At this time, the reaction was cooled to ambient temperature and 2-iodo-5-(trifluoromethyl)pyrimidine (39 mg, 0.14 mmol), CataCXium Pd G4 (3.5 mg, 0.005 mmol) and 2M aq. Na₂CO₃ (0.14 mL, 0.29 mmol) were added. The mixture was bubbled with N₂ for ˜5 minutes and then heated to 80° C. for 40 minutes. After cooling to ambient temp, the reaction was diluted with EtOAc and filtered through a plug of celite which was then eluted with additional EtOAc. The filtrate was evaporated and the crude residue was purified via flash chromatography (100% heptanes→80% [3:1 EtOAc:EtOH]/hept) to afford 7-fluoro-2-[[(1R,3S)-3-[[6-oxo-5-(trifluoromethyl)-1-(2-trimethylsilylethoxymethyl)pyridazin-4-yl]amino]cyclohexyl]methyl]-6-[5-(trifluoromethyl)pyrimidin-2-yl]-3,4-dihydroisoquinolin-1-one. ES/MS m/z: 715.2 [M+H]⁺.

Step 2. 7-fluoro-2-[[(1R,3S)-3-[[6-oxo-5-(trifluoromethyl)-1-(2-trimethylsilylethoxymethyl)pyridazin-4-yl]amino]cyclohexyl]methyl]-6-[5-(trifluoromethyl)pyrimidin-2-yl]-3,4-dihydroisoquinolin-1-one (48 mg, 0.064 mmol) was dissolved in DCM (2.0 mL) and the solution stirred at ambient temperature. TFA (0.05 mL, 0.64 mmol) was then added. After 1 hour, the reaction was evaporated to dryness. The residue was dissolved in MeOH (0.5 mL) and was treated with 2N NH₃ in MeOH (0.64 mL, 1.3 mmol). The reaction was shaken briefly and then left to stand at ambient temperature for ten minutes before evaporating to dryness. The residue was purified by reverse phase prep-HPLC (5-100% MeCN in water, 0.1% TFA) to afford 7-fluoro-2-[[(1R,3S)-3-[[6-oxo-5-(trifluoromethyl)-1H-pyridazin-4-yl]amino]cyclohexyl]methyl]-6-[5-(trifluoromethyl)pyrimidin-2-yl]-3,4-dihydroisoquinolin-1-one. ¹H NMR (400 MHz, DMSO-d6) δ 12.42 (s, 1H), 9.44 (d, J=0.9 Hz, 2H), 8.05 (d, J=7.1 Hz, 1H), 7.94 (s, 1H), 7.71 (d, J=11.3 Hz, 1H), 6.36-6.28 (7, 1H), 3.80-3.67 (1H), 3.67-3.54 (i, 2H), 3.48-3.32 (2, 2H), 3.07 (t, J=6.7 Hz, 2H), 1.99-1.60 (i, 5H), 1.46-1.29 (m, 2H), 1.21 (q, J=11.8 Hz, 1H), 0.98-0.82 (m, 1H). ES/MS: m/z 585.1 [M+H]⁺.

The Following compounds were synthesized using the procedure as described in Example 70 from the corresponding starting materials.

| Ex. | | |
|---|---|---|
| No. | Structure | ¹H NMR/MS [M + H]⁺ |
| 70 | 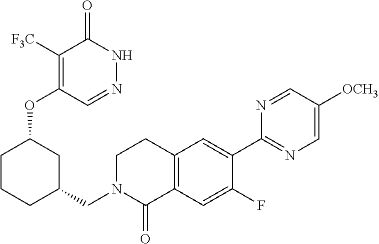 [SMILES: COc1cnc(-c2cc3c(cc2F)C(=O)N(C[C@@H]2CCC[C@H](Oc4cn[nH]c(=O)c4C(F)(F)F)C2)CC3)nc1] | 1H NMR (400 MHz, DMSO-d6) δ 12.42 (s, 1H), 8.71 (s, 2H), 7.94 (s, 1H), 7.89 (d, J = 7.3 Hz, 1H), 7.64 (d, J = 11.3 Hz, 1H), 6.36-6.27 (m, 1H), 3.98 (s, 3H), 3.81- 3.66 (m, 1H), 3.66-3.52 (m, 2H), 3.46-3.31 (m, 2H), 3.11-2.97 (m, 2H), 1.97-1.58 (m, 5H), 1.46-1.28 (m, 2H), 1.21 (q, J = 11.8 Hz, 1H), 0.97-0.82 (m, 1H). ES/MS: m/z 547.1 [M + H]⁺. |
| 71 | 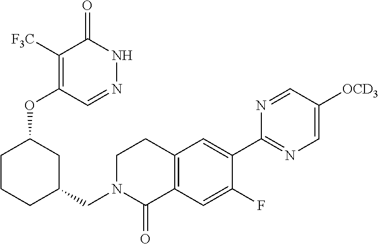 | ¹H NMR (400 MHz, DMSO-d6) δ 12.42 (s, 1H), 8.71 (s, 2H), 7.94 (s, 1H), 7.89 (d, J = 7.3 Hz, 1H), 7.64 (d, J = 11.3 Hz, 1H), 6.36-6.27 (m, 1H), 3.82-3.67 (m, 1H), 3.66-3.52 (m, J = 6.9 Hz, 2H), 3.46-3.31 (m, 2H), 3.03 (t, J = 6.8 Hz, 2H), 1.97-1.70 (m, 4H), 1.69-1.58 (m, 1H), 1.45-1.29 (m, 2H), 1.29-1.13 (m, 1H), 0.97- 0.81 (m, 1H). ES/MS: m/z 550.2 [M + H]⁺. |
| 72 | 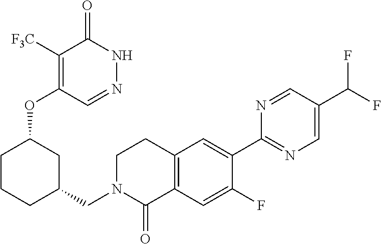 [SMILES: O=C1c2cc(F)c(-c3ncc(C(F)F)cn3)cc2CCN1C[C@@H]1CCC[C@H](Oc2cn[nH]c(=O)c2C(F)(F)F)C1] | ¹H NMR (400 MHz, DMSO-d6) δ 12.43 (s, 1H), 9.21 (d, J = 1.2 Hz, 2H), 8.01 (d, J = 7.1 Hz, 1H), 7.95 (s, 1H), 7.70 (d, J = 11.2 Hz, 1H), 7.29 (t, J = 54.8 Hz, 1H), 6.37-6.26 (m, 1H), 3.79-3.68 (m, 1H), 3.68-3.54 (m, 2H), 3.48-3.33 (m, 2H), 3.11-2.99 (m, 2H), 1.98- 1.71 (m, 4H), 1.70-1.60 (m, 1H), 1.47-1.29 (m, 2H), 1.28-1.13 (m, 1H), 0.98-0.82 (m, 1H). ES/MS: m/z 567.1 [M + H]⁺. |
| 73 | 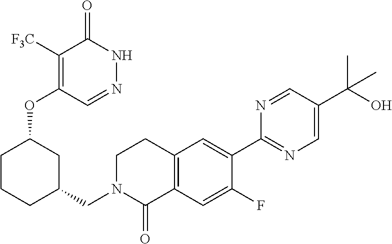 [SMILES: CC(C)(O)c1cnc(-c2cc3c(cc2F)C(=O)N(C[C@@H]2CCC[C@H](Oc4cn[nH]c(=O)c4C(F)(F)F)C2)CC3)nc1] | ¹H NMR (400 MHz, DMSO-d6) δ 12.42 (s, 1H), 9.03 (s, 2H), 8.00-7.88 (m, 2H), 7.66 (d, J = 11.2 Hz, 1H), 6.37-6.26 (m, 1H), 3.82-3.66 (m, 1H), 3.66-3.54 (m, 2H), 3.47-3.31 (m, 2H), 3.04 (t, J = 6.8 Hz, 2H), 1.97- 1.60 (m, 5H), 1.53 (s, 6H), 1.47-1.28 (m, 2H), 1.28- 1.14 (m, 1H), 0.97-0.83 (m, 1H). ES/MS: m/z 575.2 [M + H]⁺. |

### Example 74: Preparation of 6-fluoro-2-(((1R,3S)-3-((6-oxo-5-(trifluoromethyl)-1,6-dihydropyridazin-4-yl)amino)cyclohexyl)methyl)-5-(5-(trifluoromethyl)pyrimidin-2-yl)isoindolin-1-one

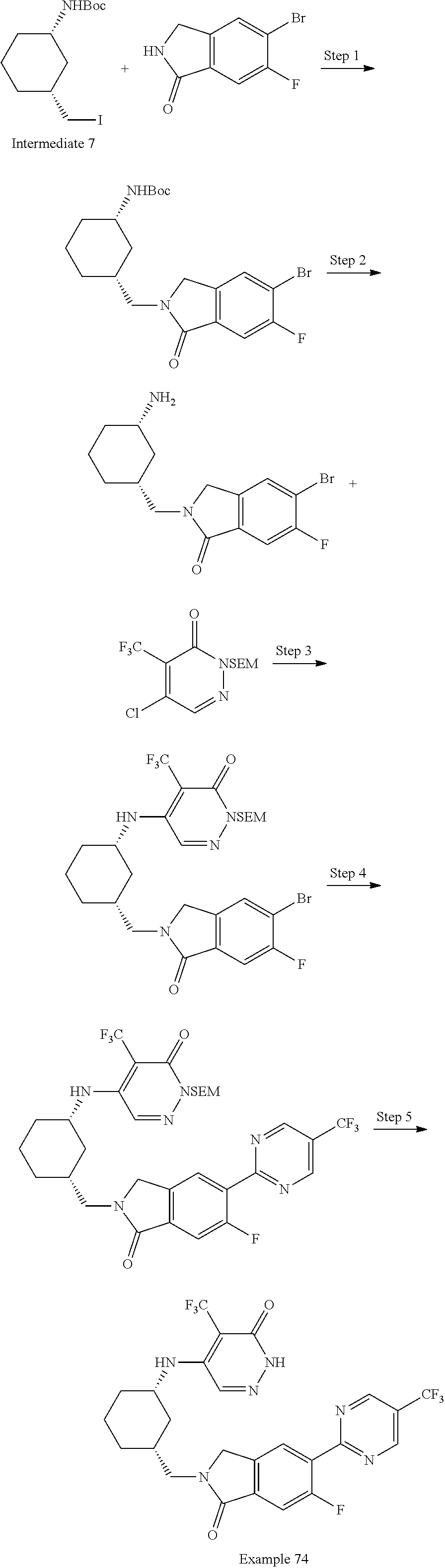

Step 1. To a mixture of 5-bromo-6-fluoro-isoindolin-1-one (325.0 mg, 1.41 mmol) and Intermediate 7, tert-butyl N-[(1S,3R)-3-(iodomethyl)cyclohexyl]carbamate (719 mg, 2.12 mmol) in DMF (14 mL) was added Cs₂CO₃ (3069 mg, 5.65 mmol) and the reaction was stirred at room temperature for 18 h. Upon completion, the mixture was diluted with EtOAc, washed with water, washed with brine, dried over MgSO₄ and concentrated in vacuo to give the crude product. The crude residue was purified using column chromatography eluting with EtOAc in hexanes 0-100% to afford tert-butyl N-[(1S,3R)-3-[(5-bromo-6-fluoro-1-oxo-isoindolin-2-yl)methyl]cyclohexyl]carbamate. ES/MS: m/z 442.88 [M+H]⁺.

Step 2. To a solution of tert-butyl N-[(1S,3R)-3-[(5-bromo-6-fluoro-1-oxo-isoindolin-2-yl)methyl]cyclohexyl]carbamate (62.1 mg, 0.141 mmol) in dichloromethane (2 mL) was added trifluoroacetic acid (0.19 mL, 2.50 mmol) at room temperature and the mixture was stirred for 1 hr. Upon completion, the solvent was removed under reduced pressure to afford 2-[[(1R,3S)-3-aminocyclohexyl]methyl]-5-bromo-6-fluoro-isoindolin-1-one which was used without further purification. ES/MS: m/z 343.446 [M+H]⁺.

Step 3. A mixture of 2-[[(1R,3S)-3-aminocyclohexyl]methyl]-5-bromo-6-fluoro-isoindolin-1-on (48.0 mg, 0.141 mmol), 5-chloro-4-(trifluoromethyl)-2-(2-trimethylsilylethoxymethyl)pyridazin-3-one (61.7 mg, 0.169 mmol), and N,N-diisopropylethylamine (0.10 mL, 0.611 mmol) in DMF (2.0 mL) was stirred at RT for 2 h. Upon completion, the reaction was diluted with EtOAc, washed with water, washed with brine, dried over MgSO₄ and concentrated in vacuo to give the crude product. The crude residue was purified using column chromatography eluting with EtOAc in hexanes 0-100% to provide 5-bromo-6-fluoro-2-[[(1R,3S)-3-[[6-oxo-5-(trifluoromethyl)-1-(2-trimethylsilylethoxymethyl)pyridazin-4-yl]amino]cyclohexyl]methyl]isoindolin-1-one. ES/MS: m/z 634.96 [M+H]⁺.

Step 4. In a vial were placed methyl 5-bromo-6-fluoro-2-[[(1R,3S)-3-[[6-oxo-5-(trifluoromethyl)-1-(2-trimethylsilylethoxymethyl)pyridazin-4-yl]amino]cyclohexyl]methyl]isoindolin-1-one (56.1 mg, 0.089 mmol), 1,1′-Bis(diphenylphosphino)ferrocene-palladium(II)dichloride dichloromethane complex (7.2 mg, 0.008 mmol), potassium acetate (26.1 mg, 0.266 mmol), and bis(pinacolato)diboron (28.2 mg, 0.266 mmol) in dioxane (2 mL). The mixture was sparged with Nitrogen and then stirred at 80° C. for 18 hr. The reaction was cooled to ambient temperature and cataCXium Pd G4 (3.3 mg, 0.004 mmol), 2 M aqueous sodium carbonate (0.13 mL, 0.226 mmol), and 2-iodo-5-(trifluoromethyl)pyrimidine (29.1 mg, 0.106 mmol), were added. Mixture was then stirred at 90° C. for 3 h. The reaction was then cooled, diluted with EtOAc and filtered through a plug of Celite®. The volatiles were evaporated in vacuo and the residue purified via flash chromatography (100% hexanes to 100% EtOAc) to give 6-fluoro-2-[[(1R,3S)-3-[[6-oxo-5-(trifluoromethyl)-1-(2-trimethylsilylethoxymethyl)pyridazin-4-yl]amino]cyclohexyl]methyl]-5-[5-(trifluoromethyl)pyrimidin-2-yl]isoindolin-1-one. ES/MS m/z: 701.14 [M+H]⁺.

Step 5. In a vial was placed 6-fluoro-2-[[(1R,3S)-3-[[6-oxo-5-(trifluoromethyl)-1-(2-trimethylsilylethoxymethyl)pyridazin-4-yl]amino]cyclohexyl]methyl]-5-[5-(trifluoromethyl)pyrimidin-2-yl]isoindolin-1-one (48.0 mg, 0.069 mmol), and trifluoroacetic acid (0.19 mL, 2.44 mmol) in DCM (5 mL). The mixture was stirred for 1 hr, and then was concentrated under vacuum. The resulting crude product was dissolved in methanol (5.0 mL) and 2.0 M ammonia in methanol (0.38 mL, 0.766 mmol) was added and the mixture was stirred for 30 minutes and then concentrated under vacuum. The resulting crude product was purified by reverse phase prep-HPLC (5-100% MeCN in water, 0.1% TFA) to afford Example 74, 6-fluoro-2-[[(1R,3S)-3-[[6-oxo-5-(trifluoromethyl)-1H-pyridazin-4-yl]amino]cyclohexyl]methyl]-5-[5-(trifluoromethyl)pyrimidin-2-yl]isoindolin-1-one. ¹H NMR (400 MHz, DMSO-d6) δ 12.42 (s, 1H), 9.51-9.40 (m, 2H), 8.30 (d, J=6.4 Hz, 1H), 7.93 (s, 1H), 7.64 (d, J=9.7 Hz, 1H), 6.36-6.24 (m, 1H), 4.67-4.50 (m, 2H), 3.66-3.53 (m, 1H), 3.51-3.38 (m, 2H), 2.05-1.86 (m, 1H), 1.87-1.77 (m, 2H), 1.77-1.68 (m, 1H), 1.68-1.57 (m, 1H), 1.46-1.29 (m, 2H), 1.28-1.13 (m, 1H), 0.98-0.82 (m, 1H). ES/MS m/z: 571.100 [M+H]⁺.

Compound Table

| Example No. | Name |
|---|---|
| 1 | 6-(5-(1H-pyrazol-1-yl)pyrimidin-2-yl)-7-fluoro-2-(((1R,3S)-3-((6-oxo-5- |
| | (trifluoromethyl)-1,6-dihydropyridazin-4-yl)amino)cyclohexyl)methyl)isoquinolin- |
| | 1(2H)-one |
| 2 | 2-(7-fluoro-1-oxo-2-(((1R,3S)-3-((6-oxo-5-(trifluoromethyl)-1,6-dihydropyridazin- |
| | 4-yl)amino)cyclohexyl)methyl)-1,2-dihydroisoquinolin-6-yl)pyrimidine-5- |
| | carbonitrile |
| 3 | 2-(7-fluoro-1-oxo-2-(((1R,3S)-3-((6-oxo-5-(trifluoromethyl)-1,6-dihydropyridazin- |
| | 4-yl)amino)cyclohexyl)methyl)-1,2-dihydroisoquinolin-6-yl)-4- |
| | methoxypyrimidine-5-carbonitrile |
| 4 | 6-(5-(1,1-difluoroethyl)pyrimidin-2-yl)-7-fluoro-2-(((1R,3S)-3-((6-oxo-5- |
| | (trifluoromethyl)-1,6-dihydropyridazin-4-yl)amino)cyclohexyl)methyl)isoquinolin- |
| | 1(2H)-one |
| 5 | 6-(5-(difluoromethoxy)pyrimidin-2-yl)-7-fluoro-2-(((1R,3S)-3-((6-oxo-5- |
| | (trifluoromethyl)-1,6-dihydropyridazin-4-yl)amino)cyclohexyl)methyl)isoquinolin- |
| | 1(2H)-one |
| 6 | 7-fluoro-6-(4-methyl-5-(trifluoromethyl)pyrimidin-2-yl)-2-(((1R,3S)-3-((6-oxo-5- |
| | (trifluoromethyl)-1,6-dihydropyridazin-4-yl)amino)cyclohexyl)methyl)isoquinolin- |
| | 1(2H)-one |
| 7 | 6-(5-chloro-4-methylpyrimidin-2-yl)-7-fluoro-2-(((1R,3S)-3-((6-oxo-5- |
| | (trifluoromethyl)-1,6-dihydropyridazin-4-yl)amino)cyclohexyl)methyl)isoquinolin- |
| | 1(2H)-one |
| 8 | 6-(7,8-dihydro-5H-[1,2,4]triazolo[5,1-b][1,5,3]dioxazepin-2-yl)-7-fluoro-2- |
| | (((1R,3S)-3-((6-oxo-5-(trifluoromethyl)-1,6-dihydropyridazin-4- |
| | yl)amino)cyclohexyl)methyl)isoquinolin-1(2H)-one |
| 9 | 6-(7,8-dihydro-6H-pyrimido[5,4-b][1,4]oxazin-2-yl)-7-fluoro-2-(((1R,3S)-3-((6- |
| | oxo-5-(trifluoromethyl)-1,6-dihydropyridazin-4- |
| | yl)amino)cyclohexyl)methyl)isoquinolin-1(2H)-one |
| 10 | 7-fluoro-6-(5-(1-hydroxyethyl)pyrimidin-2-yl)-2-(((1R,3S)-3-((6-oxo-5- |
| | (trifluoromethyl)-1,6-dihydropyridazin-4-yl)amino)cyclohexyl)methyl)isoquinolin- |
| | 1(2H)-one |
| 11 | 7-fluoro-6-(5-(methylsulfonyl)pyridin-2-yl)-2-(((1R,3S)-3-((6-oxo-5- |
| | (trifluoromethyl)-1,6-dihydropyridazin-4-yl)amino)cyclohexyl)methyl)isoquinolin- |
| | 1(2H)-one |
| 12 | 7-fluoro-6-(5-fluoropyrimidin-2-yl)-2-(((1R,3S)-3-((6-oxo-5-(trifluoromethyl)-1,6- |
| | dihydropyridazin-4-yl)amino)cyclohexyl)methyl)isoquinolin-1(2H)-one |
| 13 | 7-fluoro-6-(5-(methylsulfonyl)pyrimidin-2-yl)-2-(((1R,3S)-3-((6-oxo-5- |
| | (trifluoromethyl)-1,6-dihydropyridazin-4-yl)amino)cyclohexyl)methyl)isoquinolin- |
| | 1(2H)-one |
| 14 | 6-(5-(2,2-difluoroethoxy)pyrimidin-2-yl)-7-fluoro-2-(((1R,3S)-3-((6-oxo-5- |
| | (trifluoromethyl)-1,6-dihydropyridazin-4-yl)amino)cyclohexyl)methyl)isoquinolin- |
| | 1(2H)-one |
| 15 | 7-fluoro-6-(5-methoxypyrazin-2-yl)-2-(((1R,3S)-3-((6-oxo-5-(trifluoromethyl)-1,6- |
| | dihydropyridazin-4-yl)amino)cyclohexyl)methyl)isoquinolin-1(2H)-one |
| 16 | 7,8-difluoro-6-(5-(2-hydroxy-2-methylpropoxy)pyrimidin-2-yl)-2-(((1R,3S)-3-((6- |
| | oxo-5-(trifluoromethyl)-1,6-dihydropyridazin-4- |
| | yl)amino)cyclohexyl)methyl)isoquinolin-1(2H)-one |
| 17 | 7,8-difluoro-6-(6-fluoro-[1,2,4]triazolo[1,5-a]pyridin-2-yl)-2-(((1R,3S)-3-((6-oxo- |
| | 5-(trifluoromethyl)-1,6-dihydropyridazin-4- |
| | yl)amino)cyclohexyl)methyl)isoquinolin-1(2H)-one |
| 18 | 7,8-difluoro-6-(5-(2-hydroxypropan-2-yl)pyrimidin-2-yl)-2-(((1R,3S)-3-((6-oxo-5- |
| | (trifluoromethyl)-1,6-dihydropyridazin-4-yl)amino)cyclohexyl)methyl)isoquinolin- |
| | 1(2H)-one |
| 19 | 6-(5-(difluoromethyl)pyridin-2-yl)-7,8-difluoro-2-(((1R,3S)-3-((6-oxo-5- |
| | (trifluoromethyl)-1,6-dihydropyridazin-4-yl)amino)cyclohexyl)methyl)isoquinolin- |
| | 1(2H)-one |
| 20 | 6-(5-(1,1-difluoroethyl)pyrimidin-2-yl)-7,8-difluoro-2-(((1R,3S)-3-((6-oxo-5- |
| | (trifluoromethyl)-1,6-dihydropyridazin-4-yl)amino)cyclohexyl)methyl)isoquinolin- |
| | 1(2H)-one |
| 21 | 6-(5-(difluoromethoxy)pyrimidin-2-yl)-7,8-difluoro-2-(((1R,3S)-3-((6-oxo-5- |
| | (trifluoromethyl)-1,6-dihydropyridazin-4-yl)amino)cyclohexyl)methyl)isoquinolin- |
| | 1(2H)-one |
| 22 | 2-(7,8-difluoro-1-oxo-2-(((1R,3S)-3-((6-oxo-5-(trifluoromethyl)-1,6- |
| | dihydropyridazin-4-yl)amino)cyclohexyl)methyl)-1,2-dihydroisoquinolin-6-yl)-4- |
| | methoxypyrimidine-5-carbonitrile |
| 23 | 4,7-difluoro-2-(((1R,3S)-3-((6-oxo-5-(trifluoromethyl)-1,6-dihydropyridazin-4- |
| | yl)amino)cyclohexyl)methyl)-6-(5-(trifluoromethoxy)pyrimidin-2-yl)isoquinolin- |
| | 1(2H)-one |
| 24 | 7,8-difluoro-6-(5-(methylsulfonimidoyl)pyrimidin-2-yl)-2-(((1R,3S)-3-((6-oxo-5- |
| | (trifluoromethyl)-1,6-dihydropyridazin-4-yl)amino)cyclohexyl)methyl)isoquinolin- |
| | 1(2H)-one |
| 25 | 7-(5-(difluoromethoxy)pyrimidin-2-yl)-6-fluoro-3-(((1R,3S)-3-((6-oxo-5- |
| | (trifluoromethyl)-1,6-dihydropyridazin-4-yl)amino)cyclohexyl)methyl)quinazolin- |
| | 4(3H)-one |
| 26 | 6-fluoro-7-(5-(methoxy-d3)pyrimidin-2-yl)-3-(((1R,3S)-3-((6-oxo-5- |
| | (trifluoromethyl)-1,6-dihydropyridazin-4-yl)amino)cyclohexyl)methyl)quinazolin- |
| | 4(3H)-one |
| 27 | 6-(5-(1,1-difluoro-2-hydroxypropan-2-yl)pyrimidin-2-yl)-7-fluoro-2-(((1R,3S)-3- |
| | ((6-oxo-5-(trifluoromethyl)-1,6-dihydropyridazin-4- |
| | yl)amino)cyclohexyl)methyl)isoquinolin-1(2H)-one |
| 28 | 6-(5-(1,1-difluoro-2-hydroxypropan-2-yl)pyrimidin-2-yl)-7,8-difluoro-2-(((1R,3S)- |
| | 3-((6-oxo-5-(trifluoromethyl)-1,6-dihydropyridazin-4- |
| | yl)amino)cyclohexyl)methyl)isoquinolin-1(2H)-one |
| 29 | 2-(7,8-difluoro-1-oxo-2-(((1R,3S)-3-((6-oxo-5-(trifluoromethyl)-1,6- |
| | dihydropyridazin-4-yl)amino)cyclohexyl)methyl)-1,2-dihydroisoquinolin-6- |
| | yl)pyrimidine-5-carbonitrile |
| 30 | 7,8-difluoro-6-(5-(methylsulfonyl)pyrimidin-2-yl)-2-(((1R,3S)-3-((6-oxo-5- |
| | (trifluoromethyl)-1,6-dihydropyridazin-4-yl)amino)cyclohexyl)methyl)isoquinolin- |
| | 1(2H)-one |
| 31 | 4,7-difluoro-6-(5-methoxypyrimidin-2-yl)-2-(((1R,3S)-3-((6-oxo-5- |
| | (trifluoromethyl)-1,6-dihydropyridazin-4-yl)amino)cyclohexyl)methyl)isoquinolin- |
| | 1(2H)-one |
| 32 | 6-([1,2,4]triazolo[1,5-a]pyridin-2-yl)-4,7-difluoro-2-(((1R,3S)-3-((6-oxo-5- |
| | (trifluoromethyl)-1,6-dihydropyridazin-4-yl)amino)cyclohexyl)methyl)isoquinolin- |
| | 1(2H)-one |
| 33 | 4,7,8-trifluoro-2-(((1R,3S)-3-((6-oxo-5-(trifluoromethyl)-1,6-dihydropyridazin-4- |
| | yl)amino)cyclohexyl)methyl)-6-(5-(trifluoromethyl)pyrimidin-2-yl)isoquinolin- |
| | 1(2H)-one |
| 34 | 6-(5-(difluoromethyl)pyrimidin-2-yl)-4,7,8-trifluoro-2-(((1R,3S)-3-((6-oxo-5- |
| | (trifluoromethyl)-1,6-dihydropyridazin-4-yl)amino)cyclohexyl)methyl)isoquinolin- |
| | 1(2H)-one |
| 35 | 4,7,8-trifluoro-6-(5-methoxypyrimidin-2-yl)-2-(((1R,3S)-3-((6-oxo-5- |
| | (trifluoromethyl)-1,6-dihydropyridazin-4-yl)amino)cyclohexyl)methyl)isoquinolin- |
| | 1(2H)-one |
| 36 | 4,7,8-trifluoro-6-(5-(2-hydroxyethoxy)pyrimidin-2-yl)-2-(((1R,3S)-3-((6-oxo-5- |
| | (trifluoromethyl)-1,6-dihydropyridazin-4-yl)amino)cyclohexyl)methyl)isoquinolin- |
| | 1(2H)-one |
| 37 | 4,7,8-trifluoro-6-(5-(2-hydroxypropan-2-yl)pyrimidin-2-yl)-2-(((1R,3S)-3-((6-oxo- |
| | 5-(trifluoromethyl)-1,6-dihydropyridazin-4- |
| | yl)amino)cyclohexyl)methyl)isoquinolin-1(2H)-one |
| 38 | 4,7-difluoro-6-[5-(2-hydroxyethoxy)pyrimidin-2-yl]-2-[[(1R,3S)-3-[6-oxo-5- |
| | (trifluoromethyl)-1H-pyridazin-4-yl]amino]cyclohexyl]methyl]isoquinolin-1-one |
| 39 | 4,7-difluoro-6-[5-(1-hydroxy-1-methyl-ethyl)pyrimidin-2-yl]-2-[(1R,3S)-3-[6- |
| | oxo-5-(trifluoromethyl)-1H-pyridazin-4-yl]amino]cyclohexyl]methyl]isoquinolin- |
| | 1-one |
| 40 | 4,7-difluoro-6-(5-methylsulfonylpyrimidin-2-yl)-2-[[(1R,3S)-3-[[6-oxo-5- |
| | (trifluoromethyl)-1H-pyridazin-4-yl]amino]cyclohexyl]methyl]isoquinolin-1-one |
| 41 | 4,7,8-trifluoro-6-(5-methylsulfonylpyrimidin-2-yl)-2-[(1R,3S)-3-[6-oxo-5- |
| | (trifluoromethyl)-1H-pyridazin-4-yl]amino]cyclohexyl]methyl]isoquinolin-1-one |
| 42 | 6-[5-(1,1-difluoroethyl)pyrimidin-2-yl]-4,7-difluoro-2-[(1R,3S)-3-[6-oxo-5- |
| | (trifluoromethyl)-1H-pyridazin-4-yl]amino]cyclohexyl]methyl]isoquinolin-1-one |
| 43 | 6-[5-(1,1-difluoroethyl)pyrimidin-2-yl]-4,7,8-trifluoro-2-[[(1R,3S)-3-[6-oxo-5- |
| | (trifluoromethyl)-1H-pyridazin-4-yl]amino]cyclohexyl]methyl]isoquinolin-1-one |
| 44 | 4,7,8-trifluoro-2-[[(1R,3S)-3-[[6-oxo-5-(trifluoromethyl)-1H-pyridazin-4- |
| | yl]amino]cyclohexyl]methyl]-6-[5-(trifluoromethoxy)pyrimidin-2-yl]isoquinolin-1- |
| | one |
| 45 | 4,7-difluoro-2-[[(1R,3S)-3-[[6-oxo-5-(trifluoromethyl)-1H-pyridazin-4- |
| | yl]amino]cyclohexyl]methyl]-6-[5-(trideuteriomethoxy)pyrimidin-2-yl]isoquinolin- |
| | 1-one |
| 46 | 4,7,8-trifluoro-2-[[(1R,3S)-3-[[6-oxo-5-(trifluoromethyl)-1H-pyridazin-4- |
| | yl]amino]cyclohexyl]methyl]-6-[5-(trideuteriomethoxy)pyrimidin-2-yl]isoquinolin- |
| | 1-one |
| 47 | 7,8-difluoro-6-(5-(2-hydroxyethoxy)pyrimidin-2-yl)-2-(((1R,3S)-3-((6-oxo-5- |
| | (trifluoromethyl)-1,6-dihydropyridazin-4-yl)amino)cyclohexyl)methyl)isoquinolin- |
| | 1(2H)-one |
| 48 | 4,7,8-trifluoro-6-(5-(2-hydroxypropan-2-yl)pyrimidin-2-yl)-2-(((1R,3S)-3-((6-oxo- |
| | 5-(trifluoromethyl)-1,6-dihydropyridazin-4-yl)oxy)cyclohexyl)methyl)isoquinolin- |
| | 1(2H)-one |
| 49 | 4,7,8-trifluoro-2-(((1R,3S)-3-((6-oxo-5-(trifluoromethyl)-1,6-dihydropyridazin-4- |
| | yl)oxy)cyclohexyl)methyl)-6-(5-(trifluoromethoxy)pyrimidin-2-yl)isoquinolin- |
| | 1(2H)-one |
| 50 | 6-(5-(difluoromethoxy)pyrimidin-2-yl)-4,7,8-trifluoro-2-(((1R,3S)-3-((6-oxo-5- |
| | (trifluoromethyl)-1,6-dihydropyridazin-4-yl)oxy)cyclohexyl)methyl)isoquinolin- |
| | 1(2H)-one |
| 51 | 4,7,8-trifluoro-6-(5-(methoxy-d3)pyrimidin-2-yl)-2-(((1R,3S)-3-((6-oxo-5- |
| | (trifluoromethyl)-1,6-dihydropyridazin-4-yl)oxy)cyclohexyl)methyl)isoquinolin- |
| | 1(2H)-one |
| 52 | 6-(5-(difluoromethyl)pyrimidin-2-yl)-4,7,8-trifluoro-2-(((1R,3S)-3-((6-oxo-5- |
| | (trifluoromethyl)-1,6-dihydropyridazin-4-yl)oxy)cyclohexyl)methyl)isoquinolin- |
| | 1(2H)-one |
| 53 | 4,7,8-trifluoro-6-(5-methoxypyrimidin-2-yl)-2-(((1R,3S)-3-((6-oxo-5- |
| | (trifluoromethyl)-1,6-dihydropyridazin-4-yl)oxy)cyclohexyl)methyl)isoquinolin- |
| | 1(2H)-one |
| 54 | 6-(5-(difluoromethyl)pyrimidin-2-yl)-7,8-difluoro-2-(((1R,3S)-3-((6-oxo-5- |
| | (trifluoromethyl)-1,6-dihydropyridazin-4-yl)oxy)cyclohexyl)methyl)isoquinolin- |
| | 1(2H)-one |
| 55 | 7,8-difluoro-6-(5-(2-hydroxypropan-2-yl)pyrimidin-2-yl)-2-(((1R,3S)-3-((6-oxo-5- |
| | (trifluoromethyl)-1,6-dihydropyridazin-4-yl)oxy)cyclohexyl)methyl)isoquinolin- |
| | 1(2H)-one |
| 56 | 6-(5-(1,1-difluoroethyl)pyrimidin-2-yl)-7,8-difluoro-2-(((1R,3S)-3-((6-oxo-5- |
| | (trifluoromethyl)-1,6-dihydropyridazin-4-yl)oxy)cyclohexyl)methyl)isoquinolin- |
| | 1(2H)-one |
| 57 | 7-fluoro-6-(5-(2-hydroxypropan-2-yl)pyrimidin-2-yl)-2-(((1R,3S)-3-((6-oxo-5- |
| | (trifluoromethyl)-1,6-dihydropyridazin-4-yl)oxy)cyclohexyl)methyl)isoquinolin- |
| | 1(2H)-one |
| 58 | 4-chloro-7-fluoro-6-(5-methoxypyrimidin-2-yl)-2-[(1R,3S)-3-[[6-oxo-5- |
| | (trifluoromethyl)-1H-pyridazin-4-yl]amino]cyclohexyl]methyl]isoquinolin-1-one |
| 59 | 6-([1,2,4]triazolo[1,5-a]pyridin-2-yl)-4-chloro-7-fluoro-2-(((1R,3S)-3-((6-oxo-5- |
| | (trifluoromethyl)-1,6-dihydropyridazin-4-yl)amino)cyclohexyl)methyl)isoquinolin- |
| | 1(2H)-one |
| 60 | 4-chloro-6-(5-(difluoromethyl)pyrimidin-2-yl)-7-fluoro-2-(((1R,3S)-3-((6-oxo-5- |
| | (trifluoromethyl)-1,6-dihydropyridazin-4-yl)amino)cyclohexyl)methyl)isoquinolin- |
| | 1(2H)-one |
| 61 | 4-chloro-6-[5-(difluoromethyl)pyrimidin-2-yl]-7,8-difluoro-2-[(1~{R},3~{S})-3- |
| | [[6-oxo-5-(trifluoromethyl)-1~{H}-pyridazin-4- |
| | yl]amino]cyclohexyl]methyl]isoquinolin-1-one |
| 62 | 7-fluoro-2-[[(1S,3S,5R)-3-hydroxy-5-[6-oxo-5-(trifluoromethyl)-1H-pyridazin-4- |
| | yl]amino]cyclohexyl]methyl]-6-[5-(trifluoromethyl)pyrimidin-2-yl]isoquinolin-1- |
| | one |
| 63 | 7-fluoro-2-[[(1S,3S)-1-hydroxy-3-[6-oxo-5-(trifluoromethyl)-1H-pyridazin-4- |
| | yl]amino]cyclohexyl]methyl]-6-[5-(trifluoromethyl)pyrimidin-2-yl]isoquinolin-1- |
| | one |
| 64 | 7-fluoro-2-[[(2R,4S)-4-[6-oxo-5-(trifluoromethyl)-1H-pyridazin-4- |
| | yl]amino]tetrahydropyran-2-yl]methyl]-6-([1,2,4]triazolo[1,5-a]pyridin-2- |
| | yl)isoquinolin-1-one |
| 65 | 7,8-difluoro-2-[[(2R,4S)-4-[[6-oxo-5-(trifluoromethyl)-1H-pyridazin-4- |
| | yl]amino]tetrahydropyran-2-yl]methyl]-6-([1,2,4]triazolo[1,5-a]pyridin-2- |
| | yl)isoquinolin-1-one1-one |
| 66 | 2-(((1S,5R)-3,3-difluoro-5-((6-oxo-5-(trifluoromethyl)-1,6-dihydropyridazin-4- |
| | yl)amino)cyclohexyl)methyl)-7-fluoro-6-(5-(trifluoromethyl)pyrimidin-2- |
| | yl)isoquinolin-1(2H)-one |
| 67 | 2-(((1R,5S)-3,3-difluoro-5-((6-oxo-5-(trifluoromethyl)-1,6-dihydropyridazin-4- |
| | yl)amino)cyclohexyl)methyl)-7-fluoro-6-(5-(trifluoromethyl)pyrimidin-2- |
| | yl)isoquinolin-1(2H)-one |
| 68 | 7-fluoro-2-(((rac-1R,3R,4S)-4-hydroxy-3-((6-oxo-5-(trifluoromethyl)-1,6- |
| | dihydropyridazin-4-yl)amino)cyclohexyl)methyl)-6-(5-(trifluoromethyl)pyrimidin- |
| | 2-yl)isoquinolin-1(2H)-one |
| 69 | 7-fluoro-2-(((1R,3S)-3-((6-oxo-5-(trifluoromethyl)-1,6-dihydropyridazin-4- |
| | yl)amino)cyclohexyl)methyl)-6-(5-(trifluoromethyl)pyrimidin-2-yl)-3,4- |
| | dihydroisoquinolin-1(2H)-one |
| 70 | 7-fluoro-6-(5-methoxypyrimidin-2-yl)-2-(((1R,3S)-3-((6-oxo-5-(trifluoromethyl)- |
| | 1,6-dihydropyridazin-4-yl)amino)cyclohexyl)methyl)-3,4-dihydroisoquinolin- |
| | 1(2H)-one |
| 71 | 7-fluoro-2-[[(1R,3S)-3-[[6-oxo-5-(trifluoromethyl)-1H-pyridazin-4- |
| | yl]amino]cyclohexyl]methyl]-6-[5-(trideuteriomethoxy)pyrimidin-2-yl]-3,4- |
| | dihydroisoquinolin-1-one |
| 72 | 6-(5-(difluoromethyl)pyrimidin-2-yl)-7-fluoro-2-(((1R,3S)-3-((6-oxo-5- |
| | (trifluoromethyl)-1,6-dihydropyridazin-4-yl)amino)cyclohexyl)methyl)-3,4- |
| | dihydroisoquinolin-1(2H)-one |
| 73 | 7-fluoro-6-(5-(2-hydroxypropan-2-yl)pyrimidin-2-yl)-2-(((1R,3S)-3-((6-oxo-5- |
| | (trifluoromethyl)-1,6-dihydropyridazin-4-yl)amino)cyclohexyl)methyl)-3,4- |
| | dihydroisoquinolin-1(2H)-one |
| 74 | 6-fluoro-2-(((1R,3S)-3-((6-oxo-5-(trifluoromethyl)-1,6-dihydropyridazin-4- |
| | yl)amino)cyclohexyl)methyl)-5-(5-(trifluoromethyl)pyrimidin-2-yl)isoindolin-1- |
| | one |

### Phospho-STAT1 (Tyr701 LANCE Ultra TR-FRET Detection Assay

LANCE Ultra phospho-STAT1 (Tyr701) kits are designed for the detection of phosphorylated STAT1 in cell lysates using a simple, homogeneous LANCE Ultra sandwich assay (Cat. #TRF4028M). This assay is intended for assessing compound induction of endogenous levels of cellular STAT1 (phosphorylated at Tyr701) in NCI-H1373 cells. The NCI-H1373 cells are cultured in RPMI 1640 media containing 10% heat inactivated FBS, GlutaMAX, 1% Penicillin-Streptomycin. An Echo acoustic liquid handler is used to transfer 60 nanoliters of compound dilutions using the Echo Qualified, 384-well polypropylene microplate clear flat bottom source plates into a Greiner (#781080) cell culture microplate. NCI-H1373 cells are seeded into these compound-spotted culture plates at 30,000 cells/well in a 60 uL volume in growth media. The plates are incubated in a 5% CO₂ humidified incubator at 37° C. for 48 hours. The media is removed and the cells are processed according to the manufacturer's suggested protocol. Briefly, 20 mL of supplemented lysis buffer is added to each well and allowed to shake for 1 hour at 400 rpm. Next, 5 ul of remixed antibody solutions (vol/vol) prepared in detection buffer are added to each well and allowed to incubate at room temperature overnight. After spinning the plate down at 300 rpm for 1 min, the plate is read on an EnVision plate reader set up for Eu3+ Cryptate and fluorescence emission is measured at two different wavelengths (665 nm and 620 nm). The HTRF ratio is then calculated (665 nM/620 nM) for each well to determine the amount of pSTAT1 in the cell lysate, and the data is then normalized to 10 uM (S)-5-((1-(3-oxo-3-(4-(5-(trifluoromethyl)pyrimidin-2-yl)piperazin-1-yl)propoxy)propan-2-yl)amino)-4-(trifluoromethyl)pyridazin-3(2H)-one positive and DMSO negative controls. The values were then plotted as a function of compound concentration and a 4-parameter fit was applied to derive the EC₅₀ values.

### Biological Data

| Example | NH1373 pSTAT1 EC₅₀ (nM) |
|---|---|
| 1 | 6.54 |
| 2 | 11.754 |
| 3 | 3.852 |
| 4 | 2.263 |
| 5 | 4.742 |
| 6 | 5.133 |
| 7 | 6.029 |
| 8 | 5.859 |
| 9 | 6.386 |
| 10 | 6.863 |
| 11 | 13.567 |
| 12 | 23.155 |
| 13 | 6.205 |
| 14 | 44.174 |
| 15 | 43.627 |
| 16 | 31.253 |
| 17 | 1.16 |
| 18 | 3.235 |
| 19 | 4.783 |
| 20 | 1.612 |
| 21 | 1.518 |
| 22 | 2.425 |
| 23 | 9.13 |
| 24 | 38.071 |
| 25 | 3.782 |
| 26 | 5.628 |
| 27 | 16.77 |
| 28 | 5.845 |
| 29 | 1.798 |
| 30 | 2.15 |
| 30 | 7.997 |
| 32 | 1.531 |
| 33 | 1.508 |
| 34 | 0.952 |
| 35 | 1.447 |
| 36 | 4.209 |
| 37 | 2.484 |
| 38 | 12.542 |
| 39 | 4.196 |
| 40 | 16.293 |
| 41 | 3.245 |
| 42 | 2.451 |
| 43 | 2.602 |
| 44 | 4.174 |
| 45 | 10.53 |
| 46 | 1.187 |
| 47 | 4.862 |
| 48 | 1.035 |
| 49 | 4.889 |
| 50 | 1.289 |
| 51 | 1.221 |
| 52 | 1.222 |
| 53 | 2.089 |
| 54 | 2.375 |
| 55 | 1.564 |
| 56 | 0.915 |
| 57 | 1.824 |
| 58 | 5.912 |
| 59 | 2.023 |
| 60 | 6.233 |
| 61 | 1.334 |
| 62 | 89.978 |
| 63 | 1.873 |
| 64 | 36.101 |
| 65 | 20.276 |
| 66 | 29.226 |
| 67 | 100 |
| 68 | 65.149 |
| 69 | 4.564 |
| 70 | 11.106 |
| 71 | 7.6 |
| 72 | 3.744 |
| 73 | 7.796 |
| 74 | 9.03 |

1. A compound of Formula I: 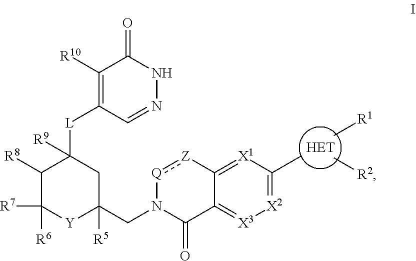
or a pharmaceutically acceptable salt or stereoisomer thereof, wherein:
HET is a 5-12 membered heteroaryl; C₆₋₁₀ aryl; C₃₋₁₂ cycloalkyl; or 4-12 membered heterocyclyl;
wherein any 5-12 membered heteroaryl, C₆₋₁₀ aryl, C₃₋₁₂ cycloalkyl, or 4-12 membered heterocyclyl, is monocyclic, bicyclic and 3-12 membered cycloalkyl or 4-12 membered heterocyclyl is monocyclic, bicyclic, fused bicyclic, spirocyclic or bridged.
Q is a bond to Z, CH₂, C═O, CR¹¹, or C(R¹¹)₂;
L is NH or O;
Y is NH, CH₂, CHR¹², CR¹²R¹² or O;
Z is N, CR¹¹ or C(R¹¹)₂,
X¹ is N or CR³;
X² is N or CR³;
X³ is N or CR⁴
R¹ and R² are each independently: H, OH, NH₂, CH₃, CHF₂, CF₃, —O—C₁₋₆ alkyl, —O—C₁₋₆ alkyl-F, —O—C₁₋₆ alkyl F₂, C₁₋₃ alkyl-O—C₁₋₃ alkyl, S(O)₂R¹¹, optionally substituted 5-12 membered heterocyclyl, or optionally substituted 6-10 membered heteroaryl;
R³ and R⁴ are each independently H, halo, —O(C₁₋₆ alkyl), OCH₃ or OCD₃;
R⁵ is H, halo, or OH;
R⁶ is H, halo or OH;
R⁷ is H. halo or OH;
R⁸ is H, halo or OH;
R⁹ is H or CH₃;
R¹⁰ is CH₃, CHF₂, or CF₃;
R¹¹ is H, halo, CHF₂ or CF₃, C₁₋₆ alkyl or C₃₋₁₀ cycloalkyl;
R¹² is H, halo, C₁₋₁₀ alkyl; C₃₋₁₀ cycloalkyl; 5-10 membered heteroaryl; C₆₋₁₀ aryl; 4-7 membered monocyclic heterocyclyl; 6-12 membered bicyclic or heterocyclyl;
or a pharmaceutically acceptable salt, tautomer or stereoisomer thereof.

2. The compound of claim 1, wherein R¹⁰ is CF₃.

3. The compound of claim 1 wherein HET is:

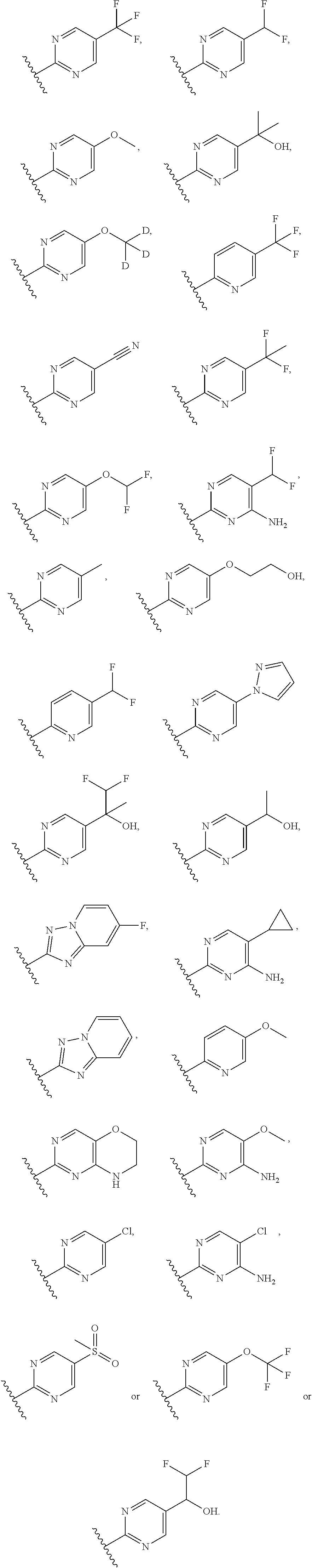

4. The compound of claim 1, wherein HET is 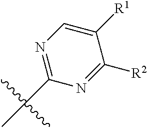
wherein R¹ is: H, OH, CHF₂, CF₃, C₁₋₆ alkyl-OH, C₁₋₆ alkyl-O—C₁₋₆ alkyl, S(O)₂R¹¹, optionally substituted 5-12 membered heterocyclyl, or 6-10 membered heteroaryl;
and R² is H or NH₂.

5. The compound of claim 4, wherein R¹ is: OH, O—C₁₋₆ alkyl, Cl, CHF₂ or CF₃.

6. The compound of claim 5, wherein R² is H or NH₂.

7. The compound of claim 4, wherein HET is:

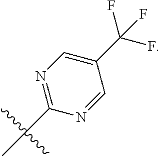 [SMILES: *c1ncc(C(F)(F)F)cn1]

8. The compound of claim 1, wherein Q is CH₂.

9. The compound of claim 8, wherein the compound is represented by Formula II: 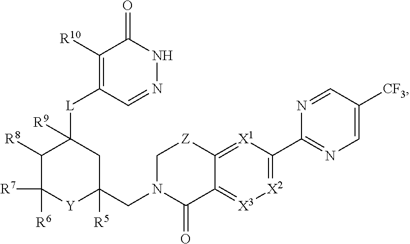
wherein
or a pharmaceutically acceptable salt or stereoisomer thereof, wherein:
HET is a 5-12 membered heteroaryl; C₆₋₁₀ aryl; C₃₋₁₂ cycloalkyl; or 4-12 membered heterocyclyl;
wherein any 5-12 membered heteroaryl, C₆₋₁₀ aryl, C₃₋₁₂ cycloalkyl, or 4-12 membered heterocyclyl, is monocyclic, bicyclic and 3-12 membered cycloalkyl or 4-12 membered heterocyclyl is monocyclic, bicyclic, fused bicyclic, spirocyclic or bridged.
Q is a bond to Z, CH₂, C═O, CR¹¹, or C(R¹¹)₂;
L is NH or O;
Y is NH, CH₂, CHR¹², CR¹²R¹² or O;
Z is N, CR¹¹ or C(R¹¹)₂,
X¹ is N or CR³;
X² is N or CR³;
X³ is N or CR⁴
R¹ and R² are each independently: H, OH, NH₂, CH₃, CHF₂, CF₃, —O—C₁₋₆ alkyl, —O—C₁₋₆ alkyl-F, —O—C₁₋₆ alkyl F₂, C₁₋₃ alkyl-O—C₁₋₃ alkyl, S(O)₂R¹¹, optionally substituted 5-12 membered heterocyclyl, or optionally substituted 6-10 membered heteroaryl;
R³ and R⁴ are each independently H, halo, —O(C₁₋₆ alkyl), OCH₃ or OCD₃;
R⁵ is H, halo, or OH;
R⁶ is H, halo or OH;
R⁷ is H. halo or OH;
R⁸ is H, halo or OH;
R⁹ is H or CH₃;
R¹⁰ is CH₃, CHF₂, or CF₃;
R¹¹ is H, halo, CHF₂ or CF₃, C₁₋₆ alkyl or C₃₋₁₀ cycloalkyl;
R¹² is H, halo, C₁₋₁₀ alkyl; C₃₋₁₀ cycloalkyl; 5-10 membered heteroaryl; C₆₋₁₀ aryl; 4-7 membered monocyclic heterocyclyl; 6-12 membered bicyclic or heterocyclyl;
or a pharmaceutically acceptable salt, tautomer or stereoisomer thereof.

10. The compound of claim 1, wherein Z is CR¹¹.

11. The compound of claim 10, wherein R¹¹ is halo.

12. The compound of claim 11, wherein R¹¹ is F.

13. The compound of claim 11, wherein R¹¹ is Cl.

14. The compound of claim 10, wherein R¹¹ is H.

15. The compound of claim 1, wherein Q is a bond to Z.

16. The compound of claim 1, wherein the compound is represented by Formula II: 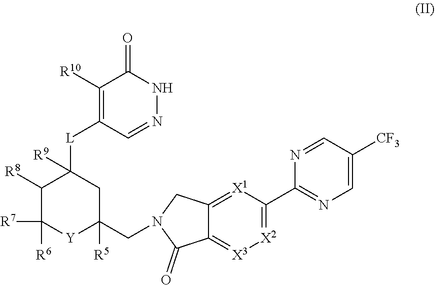
or a pharmaceutically acceptable salt, tautomer or stereoisomer thereof.

17. The compound of claim 1, wherein X² is CR².

18. The compound of claim 17, wherein X³ is CR².

19. The compound of claim 1, wherein X³ is CF.

20. The compound of claim 1, wherein X² is CH.

21. The compound of claim 1, wherein X² is CF.

22. The compound of claim 18, wherein X² is CH.

23. The compound of claim 1, wherein X³ is CH.

24. The compound of claim 16, wherein X² is CH.

25. A compound, or pharmaceutically acceptable salt, tautomer or stereoisomer thereof, selected from the group consisting of:

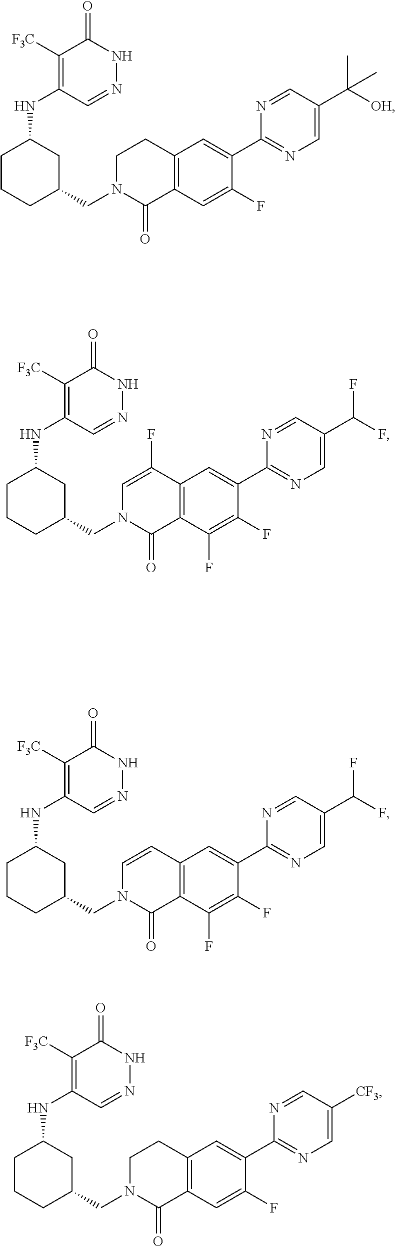 [SMILES: CC(C)(O)c1cnc(-c2cc3c(cc2F)C(=O)N(C[C@@H]2CCC[C@H](Nc4cn[nH]c(=O)c4C(F)(F)F)C2)CC3)nc1 ; O=c1[nH]ncc(N[C@H]2CCC[C@@H](Cn3cc(F)c4cc(-c5ncc(C(F)F)cn5)c(F)c(F)c4c3=O)C2)c1C(F)(F)F ; O=c1[nH]ncc(N[C@H]2CCC[C@@H](Cn3ccc4cc(-c5ncc(C(F)F)cn5)c(F)c(F)c4c3=O)C2)c1C(F)(F)F ; O=C1c2cc(F)c(-c3ncc(C(F)(F)F)cn3)cc2CCN1C[C@@H]1CCC[C@H](Nc2cn[nH]c(=O)c2C(F)(F)F)C1]

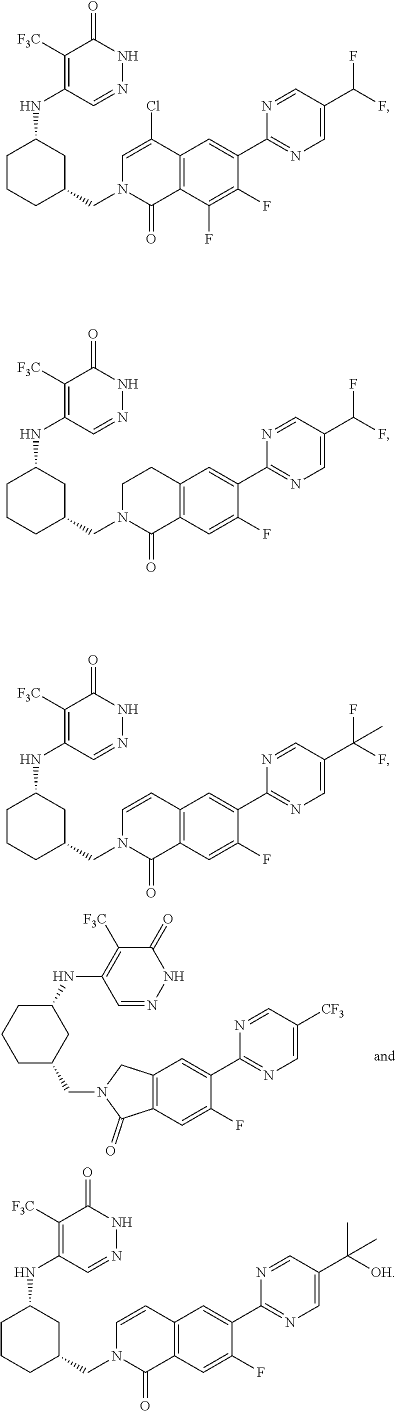

26. A pharmaceutical composition comprising a compound of claim 1, together with a pharmaceutically acceptable excipient.

27. A method of treating cancer, in a subject in need thereof, comprising administering to said patient an effective amount of a compound of claim 1.

28. A compound which is: 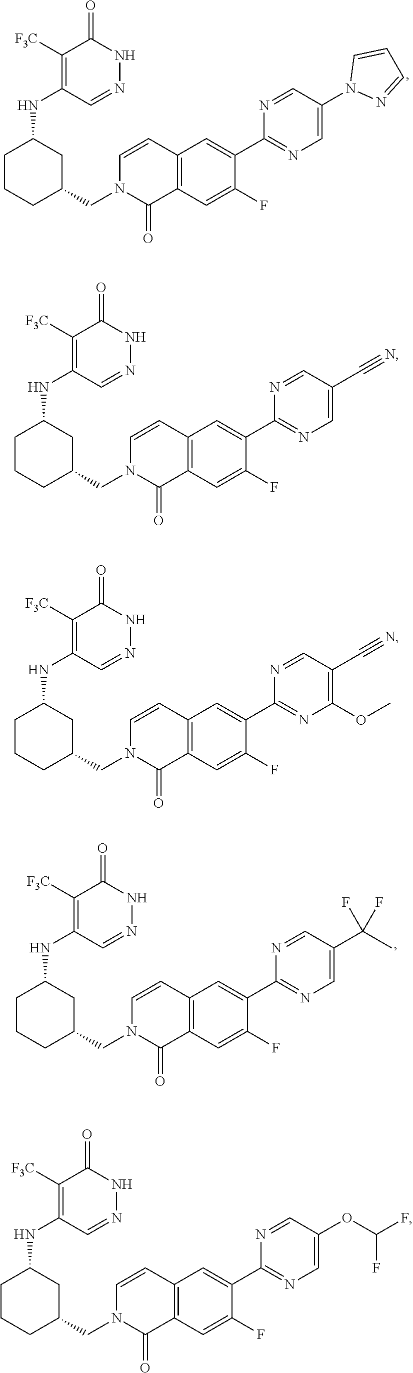 [SMILES: O=c1[nH]ncc(N[C@H]2CCC[C@@H](Cn3ccc4cc(-c5ncc(-n6cccn6)cn5)c(F)cc4c3=O)C2)c1C(F)(F)F ; N#Cc1cnc(-c2cc3ccn(C[C@@H]4CCC[C@H](Nc5cn[nH]c(=O)c5C(F)(F)F)C4)c(=O)c3cc2F)nc1 ; COc1nc(-c2cc3ccn(C[C@@H]4CCC[C@H](Nc5cn[nH]c(=O)c5C(F)(F)F)C4)c(=O)c3cc2F)ncc1C#N ; CC(F)(F)c1cnc(-c2cc3ccn(C[C@@H]4CCC[C@H](Nc5cn[nH]c(=O)c5C(F)(F)F)C4)c(=O)c3cc2F)nc1 ; O=c1[nH]ncc(N[C@H]2CCC[C@@H](Cn3ccc4cc(-c5ncc(OC(F)F)cn5)c(F)cc4c3=O)C2)c1C(F)(F)F] 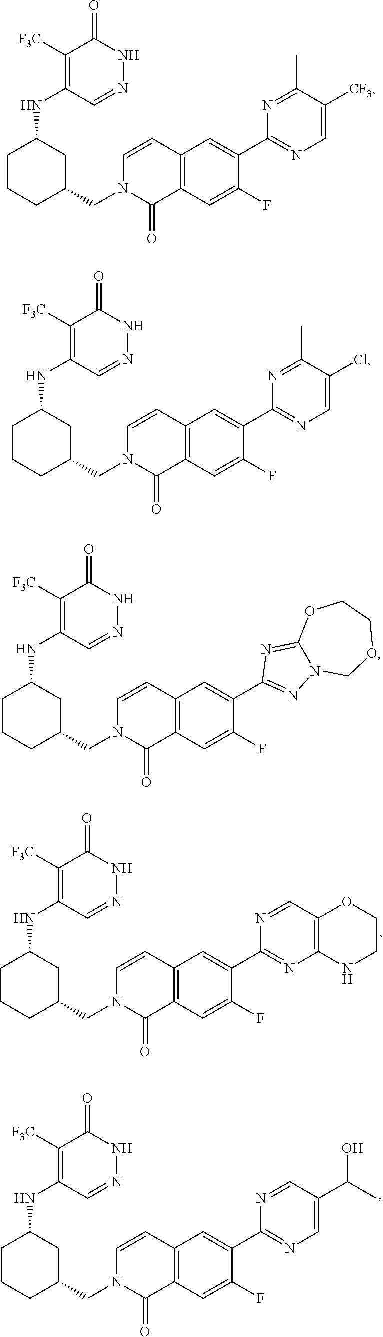 [SMILES: Cc1nc(-c2cc3ccn(C[C@@H]4CCC[C@H](Nc5cn[nH]c(=O)c5C(F)(F)F)C4)c(=O)c3cc2F)ncc1C(F)(F)F ; Cc1nc(-c2cc3ccn(C[C@@H]4CCC[C@H](Nc5cn[nH]c(=O)c5C(F)(F)F)C4)c(=O)c3cc2F)ncc1Cl ; O=c1[nH]ncc(N[C@H]2CCC[C@@H](Cn3ccc4cc(-c5nc6n(n5)COCCO6)c(F)cc4c3=O)C2)c1C(F)(F)F ; O=c1[nH]ncc(N[C@H]2CCC[C@@H](Cn3ccc4cc(-c5ncc6c(n5)NCCO6)c(F)cc4c3=O)C2)c1C(F)(F)F ; CC(O)c1cnc(-c2cc3ccn(C[C@@H]4CCC[C@H](Nc5cn[nH]c(=O)c5C(F)(F)F)C4)c(=O)c3cc2F)nc1] 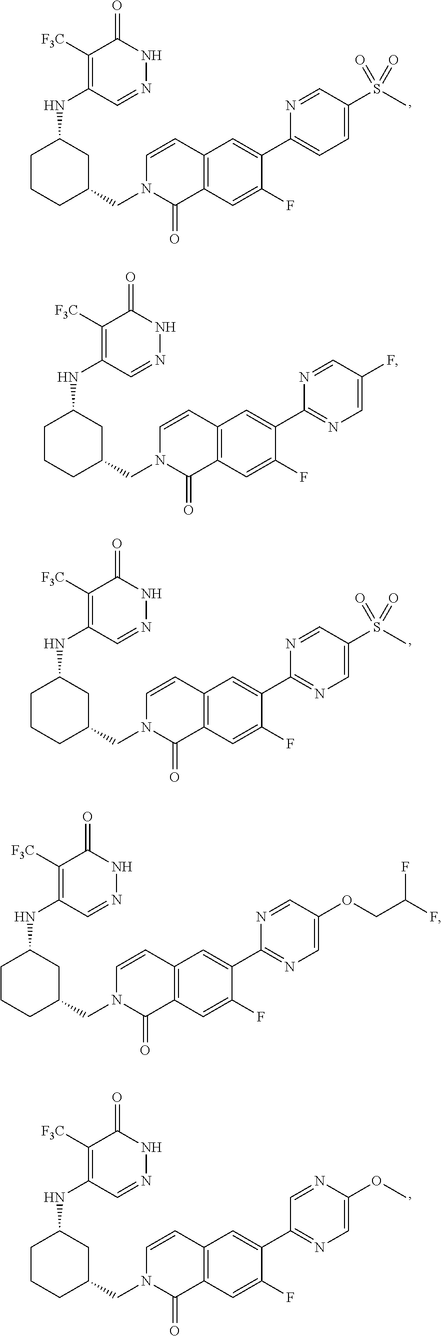 [SMILES: CS(=O)(=O)c1ccc(-c2cc3ccn(C[C@@H]4CCC[C@H](Nc5cn[nH]c(=O)c5C(F)(F)F)C4)c(=O)c3cc2F)nc1 ; O=c1[nH]ncc(N[C@H]2CCC[C@@H](Cn3ccc4cc(-c5ncc(F)cn5)c(F)cc4c3=O)C2)c1C(F)(F)F ; CS(=O)(=O)c1cnc(-c2cc3ccn(C[C@@H]4CCC[C@H](Nc5cn[nH]c(=O)c5C(F)(F)F)C4)c(=O)c3cc2F)nc1 ; O=c1[nH]ncc(N[C@H]2CCC[C@@H](Cn3ccc4cc(-c5ncc(OCC(F)F)cn5)c(F)cc4c3=O)C2)c1C(F)(F)F ; COc1cnc(-c2cc3ccn(C[C@@H]4CCC[C@H](Nc5cn[nH]c(=O)c5C(F)(F)F)C4)c(=O)c3cc2F)cn1] 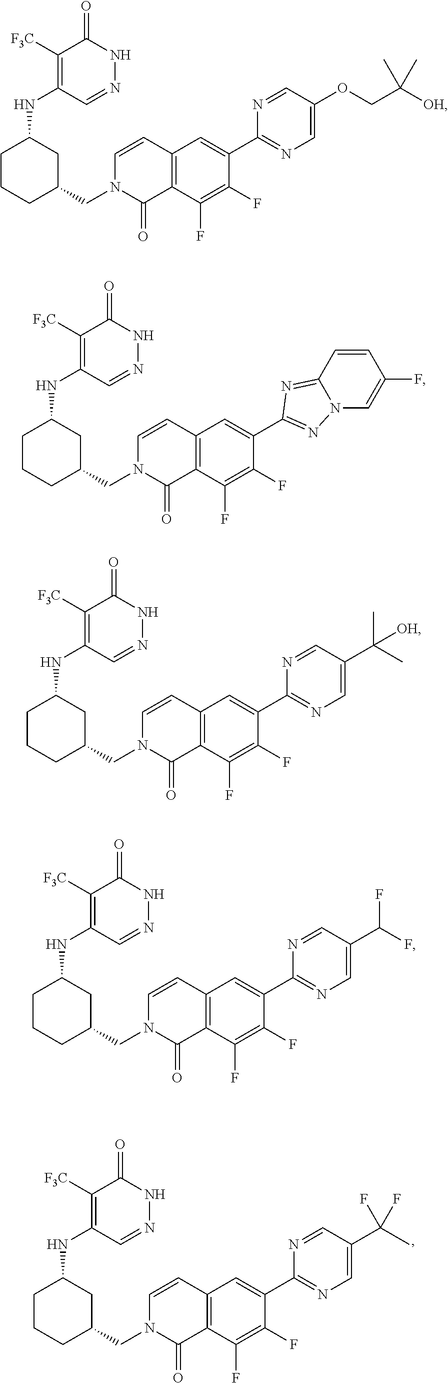 [SMILES: CC(C)(O)COc1cnc(-c2cc3ccn(C[C@@H]4CCC[C@H](Nc5cn[nH]c(=O)c5C(F)(F)F)C4)c(=O)c3c(F)c2F)nc1 ; O=c1[nH]ncc(N[C@H]2CCC[C@@H](Cn3ccc4cc(-c5nc6ccc(F)cn6n5)c(F)c(F)c4c3=O)C2)c1C(F)(F)F ; CC(C)(O)c1cnc(-c2cc3ccn(C[C@@H]4CCC[C@H](Nc5cn[nH]c(=O)c5C(F)(F)F)C4)c(=O)c3c(F)c2F)nc1 ; O=c1[nH]ncc(N[C@H]2CCC[C@@H](Cn3ccc4cc(-c5ncc(C(F)F)cn5)c(F)c(F)c4c3=O)C2)c1C(F)(F)F ; CC(F)(F)c1cnc(-c2cc3ccn(C[C@@H]4CCC[C@H](Nc5cn[nH]c(=O)c5C(F)(F)F)C4)c(=O)c3c(F)c2F)nc1] 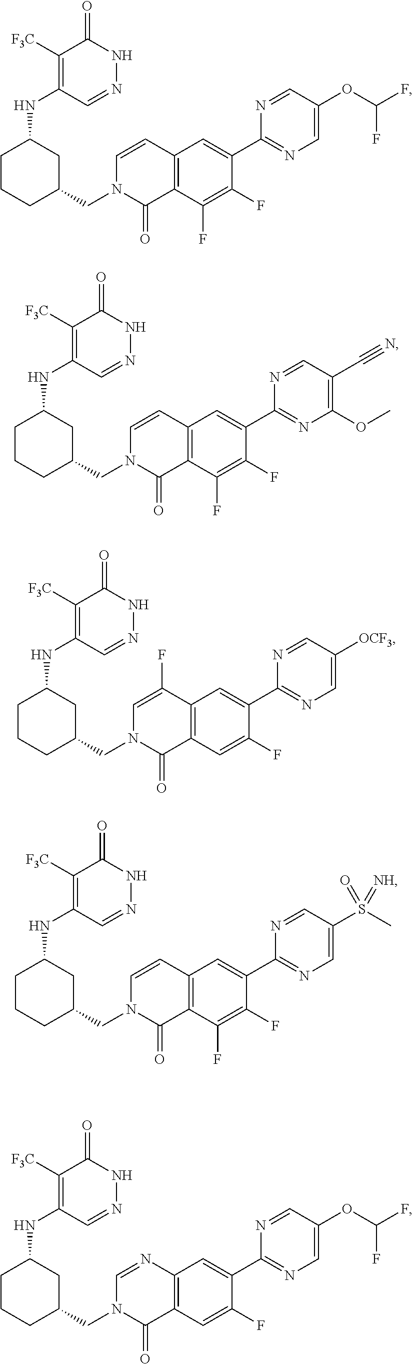 [SMILES: O=c1[nH]ncc(N[C@H]2CCC[C@@H](Cn3ccc4cc(-c5ncc(OC(F)F)cn5)c(F)c(F)c4c3=O)C2)c1C(F)(F)F ; COc1nc(-c2cc3ccn(C[C@@H]4CCC[C@H](Nc5cn[nH]c(=O)c5C(F)(F)F)C4)c(=O)c3c(F)c2F)ncc1C#N ; O=c1[nH]ncc(N[C@H]2CCC[C@@H](Cn3cc(F)c4cc(-c5ncc(OC(F)(F)F)cn5)c(F)cc4c3=O)C2)c1C(F)(F)F ; CS(=N)(=O)c1cnc(-c2cc3ccn(C[C@@H]4CCC[C@H](Nc5cn[nH]c(=O)c5C(F)(F)F)C4)c(=O)c3c(F)c2F)nc1 ; O=c1[nH]ncc(N[C@H]2CCC[C@@H](Cn3cnc4cc(-c5ncc(OC(F)F)cn5)c(F)cc4c3=O)C2)c1C(F)(F)F] 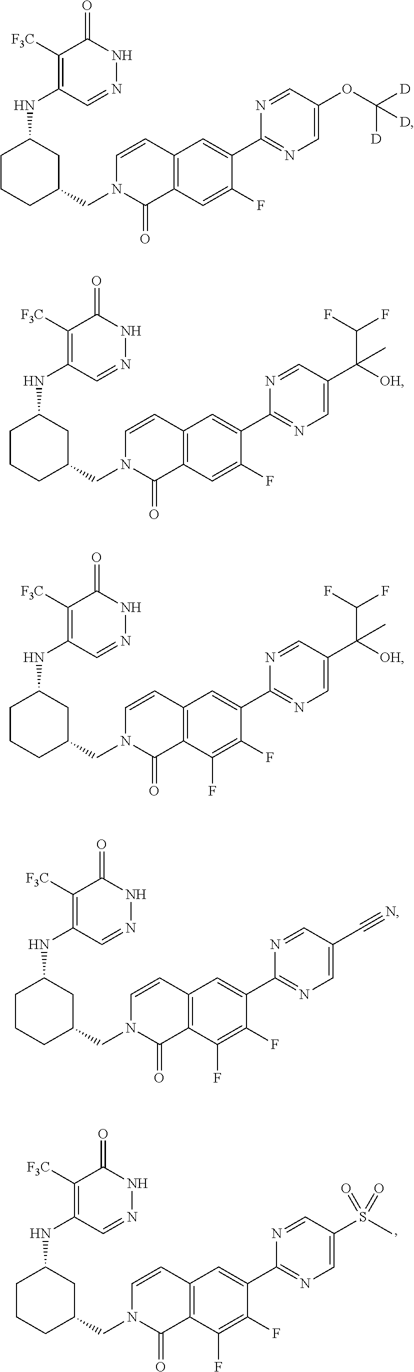 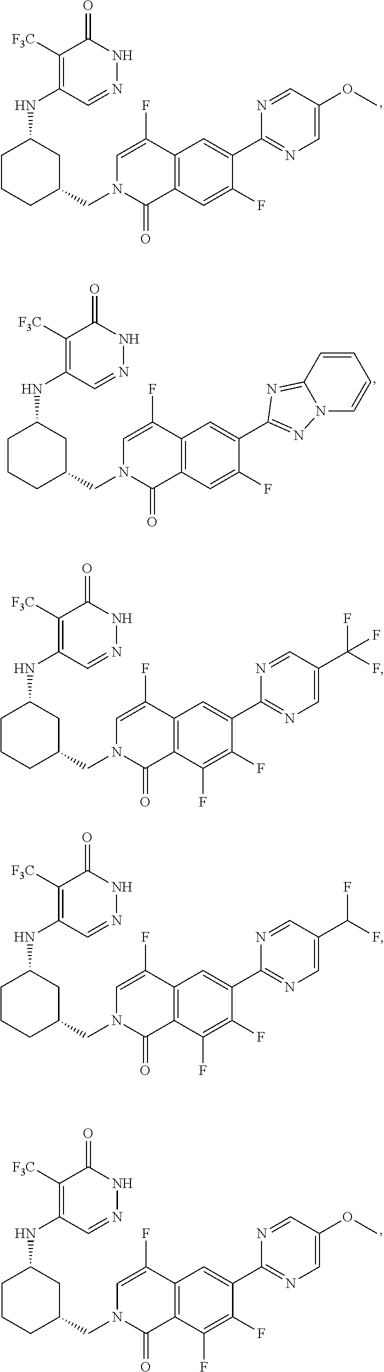 [SMILES: COc1cnc(-c2cc3c(F)cn(C[C@@H]4CCC[C@H](Nc5cn[nH]c(=O)c5C(F)(F)F)C4)c(=O)c3cc2F)nc1 ; O=c1[nH]ncc(N[C@H]2CCC[C@@H](Cn3cc(F)c4cc(-c5nc6ccccn6n5)c(F)cc4c3=O)C2)c1C(F)(F)F ; O=c1[nH]ncc(N[C@H]2CCC[C@@H](Cn3cc(F)c4cc(-c5ncc(C(F)(F)F)cn5)c(F)c(F)c4c3=O)C2)c1C(F)(F)F ; O=c1[nH]ncc(N[C@H]2CCC[C@@H](Cn3cc(F)c4cc(-c5ncc(C(F)F)cn5)c(F)c(F)c4c3=O)C2)c1C(F)(F)F ; COc1cnc(-c2cc3c(F)cn(C[C@@H]4CCC[C@H](Nc5cn[nH]c(=O)c5C(F)(F)F)C4)c(=O)c3c(F)c2F)nc1] 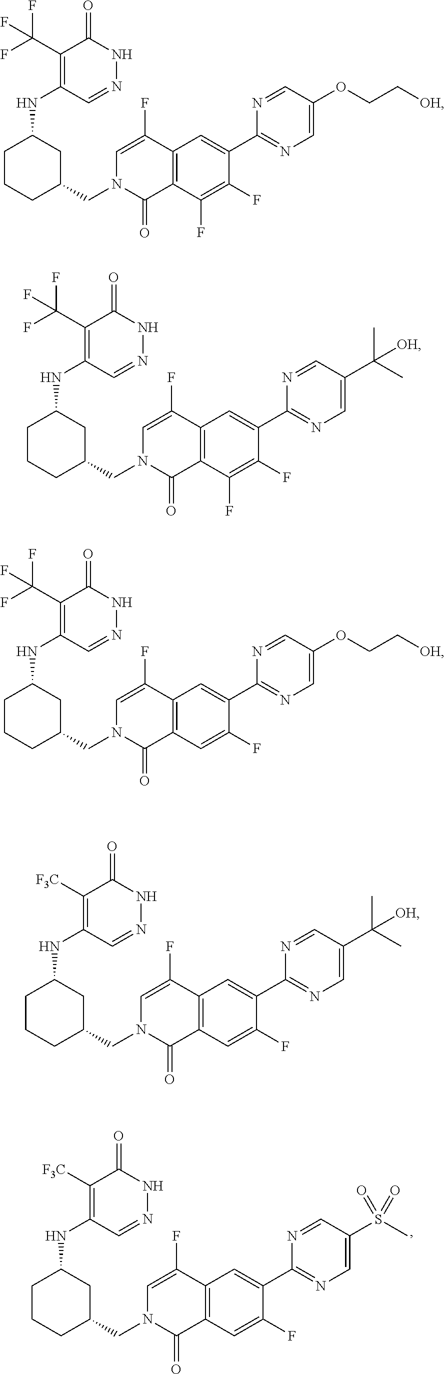 [SMILES: O=c1[nH]ncc(N[C@H]2CCC[C@@H](Cn3cc(F)c4cc(-c5ncc(OCCO)cn5)c(F)c(F)c4c3=O)C2)c1C(F)(F)F ; CC(C)(O)c1cnc(-c2cc3c(F)cn(C[C@@H]4CCC[C@H](Nc5cn[nH]c(=O)c5C(F)(F)F)C4)c(=O)c3c(F)c2F)nc1 ; O=c1[nH]ncc(N[C@H]2CCC[C@@H](Cn3cc(F)c4cc(-c5ncc(OCCO)cn5)c(F)cc4c3=O)C2)c1C(F)(F)F ; CC(C)(O)c1cnc(-c2cc3c(F)cn(C[C@@H]4CCC[C@H](Nc5cn[nH]c(=O)c5C(F)(F)F)C4)c(=O)c3cc2F)nc1 ; CS(=O)(=O)c1cnc(-c2cc3c(F)cn(C[C@@H]4CCC[C@H](Nc5cn[nH]c(=O)c5C(F)(F)F)C4)c(=O)c3cc2F)nc1] 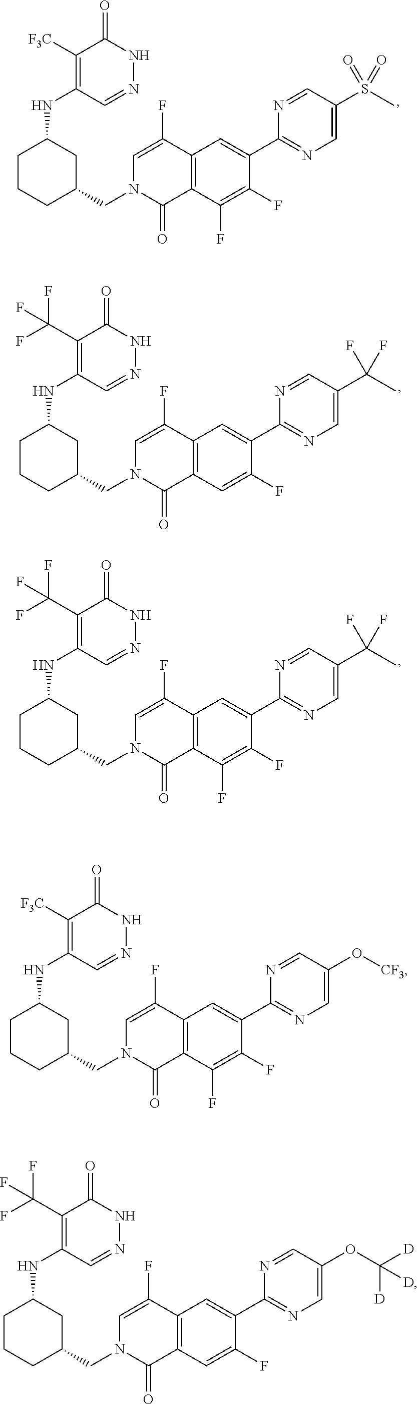 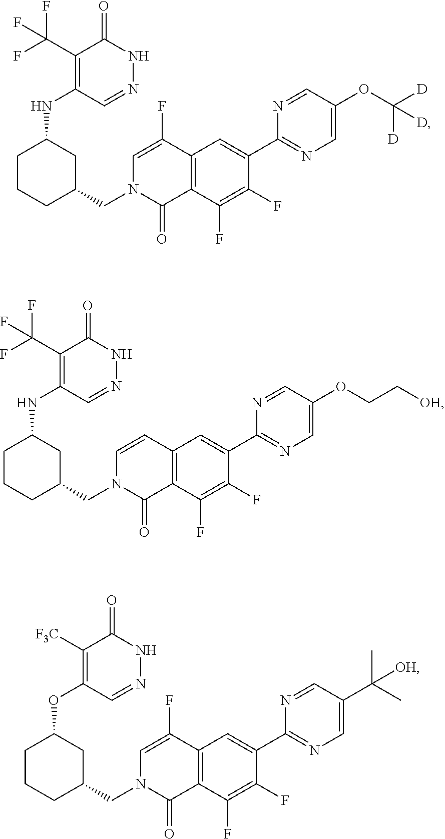
or pharmaceutically acceptable salt, tautomer or stereoisomer thereof.
